SEQUENCE LISTING (240 sequences)

SEQ ID NO: 1 - DNA, 738 bp, Escherichia coli
atgacgttaactgcttcatcttcttcccgcgctgttacgaattctcctgtggttgttgcccttgattatcataatcgtgatgacgcgctggcctttgtcgacaagatcgacccacgcgattgtcgtctgaaggtcggcaaagagatgtttacattgtttgggccacagtttgtgcgcgaacttcaacagcgtggttttgatatctttcttgacctgaaattccacgatatccccaacactgcagcgcacgctgtcgctgctgcagctgacttaggcgtgtggatggtgaatgttcatgcctctggtggggcgcgtatgatgaccgcagcgcgtgaggcactggttccgtttggcaaagatgcaccgcttttgattgctgtgacagtgttgaccagcatggaagccagcgacctggtcgatcttggcatgacactgtcacctgcagattatgcagaacgtctggcggcactgacgcaaaaatgtggccttgatggtgtggtgtgttctgctcaggaagctgtgcgctttaaacaggtattcggtcaggagttcaaactggttacgccgggcattcgtccgcaggggagtgaagctggtgaccagcgccgcattatgacgccagaacaggcgttgtcggctggtgttgattatatggtgattggtcgcccggtaacgcaatcggtagatccagcgcagacgctgaaagcgatcaacgcctctttacagcggagtgcatga

SEQ ID NO: 2 - DNA, 804 bp, Saccharomyces cerevisiae
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 3 - DNA, 201 bp, Escherichia coli
atggcaatccgaaaacgttttattgcgggcgcaaaatgcccggcctgtcaggcgcaggattcaatggcgatgtggcgcgaaaataatattgatattgttgaatgtgttaagtgcggacatcagatgcgagaagcagacaaagaagcccgcgatcacgttcgcaaagatgagcaagtgatcgggatttttcatccggactag

SEQ ID NO: 4 - DNA, 136 bp, Escherichia coli
gagggtagagcggggtttcccccgccctggtagtcttagtaagcggggaagcttatgactaagagcaccacgatgatgagtagcttcatcatgaccctttccttatttatggccccttcctcgggaggggctttcc

SEQ ID NO: 5 - RNA, 136 nt, Escherichia coli
gaggguagagcgggguuucccccgcccugguagucuuaguaagcggggaagcuuaugacuaagagcaccacgaugaugaguagcuucaucaugacccuuuccuuauuuauggccccuuccucgggaggggcuuucc

SEQ ID NO: 6 - protein, 18 aa, Escherichia coli
MMKLLIIVVLLVISFPAY

SEQ ID NO: 7 - protein, 506 aa, Flavobacterium johnsoniae
MNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 8 - protein, 552 aa, Herpetosiphon aurantiacus
MSTTLILTGEGLGIDDVVRVARHQDRVELTTDPAILAQIEASCAYINQAVKEHQPVYGVTTGFGGMANVIISPEEAAELQNNAIWYHKTGAGKLLPFTDVRAAMLLRANSHMRGASGIRLEIIQRMVTFLNANVTPHVREFGSIGASGDLVPLISITGALLGTDQAFMVDFNGETLDCISALERLGLPRLRLQPKEGLAMMNGTSVMTGIAANCVHDARILLALALEAHALMIQGLQGTNQSFHPFIHRHKPHTGQVWAADHMLELLQGSQLSRNELDGSHDYRDGDLIQDRYSLRCLPQFLGPIIDGMAFISHHLRVEINSANDNPLIDTASAASYHGGNFLGQYIGVGMDQLRYYMGLMAKHLDVQIALLVSPQFNNGLPASLVGNIQRKVNMGLKGLQLTANSIMPILTFLGNSLADRFPTHAEQFNQNINSQGFGSANLARQTIQTLQQYIAITLMFGVQAVDLRTHKLAGHYNAAELLSPLTAKIYHAVRSIVKHPPSPERPYIWNDDEQVLEAHISALAHDIANDGSLVSAVEQTLSGLRSIILFR

SEQ ID NO: 9 - protein, 552 aa, Herpetosiphon aurantiacus
MRHQVTLTGAGLTIEDVVRVARHHQPVGLTDNPEILQRIEDSCAYINDAVKASKPVYGVTTGFGGMADVVISSEEAADLQNNAIWYHKTGAGKLLPLADVRAAMLLRANSHMRGVSGIRLEIIQRMMTFLNANVTPHVREFGSIGASGDLVPLISITGALLGTDPAFRVDFDGENIDCLEALERLNLPRLELLPKEGLAMMNGTSVMTGIASNVLHDARILLGLALNIHGLMIQGLQGTNQSFHPFIHQHKAHTGQVWAADHMLQILEGSALSRDELDGRHEYREGDLIQDRYSLRCLPQFLGPIIDGMAYITHHLRVEINSANDNPLINTEAGASYHGGNFLGQYIGVGMDQLRYYMGLMAKHLDVQIALLVSPQFNNGLSASLVGNTDRKVNMGLKGLQISGNSIMPILGFLGNSLADRFPTHAEQFNQNINSQGFGSANLARQTIETLQQYIAIALIFGVQAVDLRTFKRTGHYNAVETLSPMTAKLYSAMREVVGKPISHERPYIWNDNEQALEQHISAIVSDITNDGIIPQAIQETLDSLRSIILFA

SEQ ID NO: 10 - protein, 523 aa, Rhodobacter sphaeroides
MLAMSPPKPAVELDRHIDLDEAHSVASGGARIVLAPPARDRCRASEARLGAVIREARHVYGLTTGFGPLANRLVSGENVRTLQANLVHHLASGVGPVLDWTTARAMVLARLVAIAQGASGASEGTIARLIDLLNSELAPAVPMRGTVGASGDLTPLAHMVLCLQGRGDFLDRDGTRLDGAEGLRRGRLQPLDLSHRDALALVNGTSAMTGIALVNAHACRHLGNWAVALTALLAECLGGRTEAWAAALSDLRPHPGQKDAAARLRARVDGSARVVRHVIAERRLGASDIGTEPEAGQDAYSLRCAPQVLGAGFDTLAWHDRVLTIELNAVTDNPVFPPDGSVPALHGGNFMGQHVALTSDALATAVTVLAGLAERQIARLTDERLNRGLPPFLHRGPAGLNSGFMGAQVTATALLAEMRATGPASIHSISTNAANQDVVSLGTIAARLCREKIDRWAEILAILALCLAQAAELRCGSGLDGVSPAGKKLVQALREQFPPLETDRPLGQEIAALATHLLQQSPV

SEQ ID NO: 11 - protein, 510 aa, Saccharothrix espanaensis
MTQVVERQADRLSSREYLARVVRSAGWDAGLTSCTDEEIVRMGASARTIEEYLKSDKPIYGLTQGFGPLVLFDADSELEQGGSLISHLGTGQGAPLAPEVSRLILWLRIQNMRKGYSAVSPVFWQKLADLWNKGFTPAIPRHGTVSASGDLQPLAHAALAFTGVGEAWTRDADGRWSTVPAVDALAALGAEPFDWPVREALAFVNGTGASLAVAVLNHRSALRLVRACAVLSARLATLLGANPEHYDVGHGVARGQVGQLTAAEWIRQGLPRGMVRDGSRPLQEPYSLRCAPQVLGAVLDQLDGAGDVLAREVDGCQDNPITYEGELLHGGNFHAMPVGFASDQIGLAMHMAAYLAERQLGLLVSPVTNGDLPPMLTPRAGRGAGLAGVQISATSFVSRIRQLVFPASLTTLPTNGWNQDHVPMALNGANSVFEALELGWLTVGSLAVGVAQLAAMTGHAAEGVWAELAGICPPLDADRPLGAEVRAARDLLSAHADQLLVDEADGKDFG

SEQ ID NO: 12 - protein, 519 aa, Rheinheimera sp. A13L
MRSEQLTLEDVEAIALGRQTLVVTEKQMHAVENAHKFLCRAISDRKRIYGVTTGYGPLATTEVDPRQSALLQQNLVHHLCSGVGDPLTHPQVRAMMVARLISLLSGHSGANPLLIKRMQEWLDADIVPFIPCRGTVGASGDLTPLAHLARALSGGGKVSIKGGLWIESRDAHQQLGWQPLVLKGKDAISLVNGTSATVGIAALNATAAERALKLSTLLVLLYAELLNGHREAFHPAIGQLRPHPGQQKLHSWLWSLSASSDALVPWCAESRNLNLMGEDIQQNQPLLQDAYTLRCAPQALGAALDVISQHATTVKIELSAVTDNPLLFAEDELILHGGNFFGQHLAFASDHLNNALIQMALYSERRIARITDPLRNKGLPAFMQPLDTGLHSGFMGAQVCATSLVAELRSQAMPASIQSIPTNADNQDIVPLGTIAARRASTSLTQLYQILAIEALVLVQGAELKNTHSFSHSSQVVCAWLRQYALPLKEDRALSEDITRVAEALIDPDKVKSLIELLA

SEQ ID NO: 13 - protein, 713 aa, Rhodotorula mucilaginosa
MAPSVDSIATSVANSLSNGLHAAAAANGGDVHKKTAGAGSLLPTTETTQLDIVERILADAGATDQIKLDGYTLTLGDVVGAARRGRSVKVADSPHIREKIDASVEFLRTQLDNSVYGVTTGFGGSADTRTEDAISLQKALLEHQLCGVLPTSMDGFALGRGLENSLPLEVVRGAMTIRVNSLTRGHSAVRIVVLEALTNFLNHGITPIVPLRGTISASGDLSPLSYIAASITGHPDSKVHVDGKIMSAQEAIALKGLQPVVLGPKEGLGLVNGTAVSASMATLALTDAHVLSLLAQALTALTVEAMVGHAGSFHPFLHDVTRPHPTQIEVARNIRTLLEGSKYAVHHETEVKVKDDEGILRQDRYPLRCSPQWLGPLVSDMIHAHAVLSLEAGQSTTDNPLIDLENKMTHHGGAFMASSVGNTMEKTRLAVALMGKVSFTQLTEMLNAGMNRALPSCLAAEDPSLSYHCKGLDIAAAAYTSELGHLANPVSTHVQPAEMGNQAINSLALISARRTAEANDVLSLLLATHLYCVLQAVDLRAMEFEHTKAFEPMVTELLKQHFGALATAEVEDKVRKSIYKRLQQNNSYDLEQRWHDTFSVATGAVVEALAGQEVSLASLNAWKVACAEKAIALTRSVRDSFWAAPSSSSPALKYLSPRTRVLYSFVREEVGVKARRGDVYLGKQEVTIGTNVSRIYEAIKSGCIAPVLVKMMA

SEQ ID NO: 14 - protein, 689 aa, Trichosporon cutaneum
MFIETNVAKPASTKAMNAGSAKAAPVEPFATYAHSQATKTVSIDGHTMKVGDVVAVARHGAKVELAASVAGPVRASVDFKESKKHTSIYGVTTGFGGSADTRTSDTEALQISLLEHQLCGFLPTDATYEGMLLAAMPIPIVRGAMAVRVNSCVRGHSGVRLEVLQSFADFINRGLVPCVPLRGTISASGDLSPLSYIAGAICGHPDVKVFDTAASPPTVLTSPEAIAKYGLKTVKLASKEGLGLVNGTAVSAAAGALALYDAECLAIMSQTNTVLTVEALDGHVGSFAPFIQEIRPHAGQIEAARNIRHMLGGSKLAVHEESELLADQDAGILRQDRYALRTSAQWIGPQLEALGLARQQIETELNSTTDNPLIDVEGGMFHHGGNFQAMAVTSAMDSARIVLQNLGKLSFAQVTELINCEMNHGLPSNLAGSEPSTNYHCKGLDIHCGAYCAELGFLANPMSNHVQSTEMHNQSVNSMAFASARRTMEANEVLSLLLGSQMYCATQALDLRVMEVKFKMAIVKLLNETLTKHFAAFLTPEQLAKLNTHAAITLYKRLNQTPSWDSAPRFEDAAKHLVGVIMDALMVNDDITDLTNLPKWKKEFAKEAGNLYRSILVATTADGRNDLEPAEYLGQTRAVYEAVRSELGVKVRRGDVAEGKSGKSIGSSVAKIVEAMRDGRLMGAVGKMF

SEQ ID NO: 15 - protein, 716 aa, Rhodosporidium toruloides
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 16 - protein, 737 aa, Phanerochaete chrysosporium
MPSRIDYYTSSGNGYAQSRKSSAIYPASASTGHAAPSTERKPELLDKFVEAYDELQSYREGKPVIVDGHNLSIPAVAATARFGAAVVLDENPETHERVLQSRRVIVDKVSTQRSVYGVSTGFGGSADTRTSDPLQLGHALLQHQHVGVLPTQTESPLPALPLGDPLATTSMPEAWVRGAILIRMNSLIRGHSGVRWELIEKMGELLRENITPLVPLRGSISASGDLSPLSYIAGTLIGSPAIRVFDGPASYGARRILPSNIALANHGVAPIPLSSKEHLGILNGTAFSASVGALALNEAVHLSLLAQVCTAMGTEAMIGAVGSFDAFIHDTARPHPGQVEVARNVRTLLEDSQMAVKAEDEVHIAEDEGELRQDRYPLRTAAQFLGPQIEDILSAHETVTLECNSTTDNPLIDGETGTVHHGGNFQAMAVTNAMEKTRLAIHHIGKLLFAQATELINPMMNRGLPPNLAATDPSHNYFAKGVDIHLAAYVGELGFLASPVSSHIQSAEMHNQAVNSLALVSARYTISALDVLSLLTAAYLYVLCQALDLRAMHNDLQSSLSAIVRELLPKHFPSAAKRADALLPILERTIFRALNSSSSADCKARMVSVAASTTTPLVDFLSADAALASELANITAFRTELATRAADALTTLRTQYLEGARGAAPASKYLGKTRPVYEFVRVTLNVPMHGRENLHNFEMGPGVEDGIIGNNISTIYEAIRDGKMQNVVMQLVKSIKA

SEQ ID NO: 17 - protein, 540 aa, Rhodobacter capsulatus
MLDATIGRKRMTLQSQTAKDCLALDGALTLVQCEAIATHRSRISVTPALRERCARAHARLEHAIAEQRHIYGITTGFGPLANRLIGADQGAELQQNLIYHLATGVGPKLSWAEARALMLARLNSILQGASGASPETIDRIVAVLNAGFAPEVPAQGTVGASGDLTPLAHMVLALQGRGRMIDPSGRVQEAGAVMDRLCGGPLTLAARDGLALVNGTSAMTAIAALTGVEAARAIDAALRHSAVLMEVLSGHAEAWHPAFAELRPHPGQLRATERLAQALDGAGRVCRTLTAARRLTAADLRPEDHPAQDAYSLRVVPQLVGAVWDTLDWHDRVVTCELNSVTDNPIFPEGCAVPALHGGNFMGVHVALASDALNAALVTLAGLVERQIARLTDEKLNKGLPAFLHGGQAGLQSGFMGAQVTATALLAEMRANATPVSVQSLSTNGANQDVVSMGTIAARRARAQLLPLSQIQAILALALAQAMDLLDDPEGQAGWSLTARDLRDRIRAVSPGLRADRPLAGDIEAVAQGLRHPSAADPPA

SEQ ID NO: 18 - protein, 291 aa, Rattus norvegicus
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 19 - protein, 295 aa, Homo sapiens [sequence omitted]

SEQ ID NO: 20 - protein, 295 aa, Equus caballus
MELIQDTSRPPLKYVKGVPLIKYFAEALGPLQSFQARPDDLLISTYPKSGTTWVSEILDMIYHGGDLEKCRRAPIFIRVPFLEFKAPEIPSGVEVLKDTPAPRLLKTHLPLSLLPQTLLDQKVKVVYLARNAKDVAVSYYHFYRMAKVHPDPGTWDSFLEKFMAGEVSYGSWYKHVQEWWELSHTHPVLYLFYEDMKENPKKEIQKILEFVGRSLPEETLDRIVQHTSFKEMKKNPMANYSTIPCDIMDHNISAFMRKGIAGDWKNTFTVAQNEHFDTDYAEKMAGCKLSFRSEV

SEQ ID NO: 21 - protein, 295 aa, Sus scrofa
MEPVQDTYRPPLEYVKGVPLIKYFAEALGPLESFQAWPDDVLISTYPKSGTTWVSEILDLIYQGGDLQKCQRAPIFVRVPFLEFKIPGCPTGFELLKDTPAPRLLKTHLPLALLPQTLLDQKVKVVYVARNAKDVAVSYYHFYRMAKVHPNPGTWDSFLEDFMAGEVSYGSWYQHVQEWWELRHTHPVLYLFYEDMKENPKREIQKILEFVGRSLPEETVEDIVQHTSFQEMKNNAMTNYRTLPSDLLDHSISAFMRKGITGDWKSTFTVAQNERFEADYAEKMAGCNLRFRSEL

SEQ ID NO: 22 - protein, 295 aa, Canis lupus
MEDIPDTSRPPLKYVKGIPLIKYFAEALESLQDFQAQPDDLLISTYPKSGTTWVSEILDMIYQDGDVEKCRRAPVFIRVPFLEFKAPGIPTGLEVLKDTPAPRLIKTHLPLALLPQTLLDQKVKVVYVARNAKDVAVSYYHFYRMAKVHPDPDTWDSFLEKFMAGEVSYGSWYQHVQEWWELSHTHPVLYLFYEDMKENPKREIQKILKFVGRSLPEETVDLIVQHTSFKEMKNNSMANYTTLSPDIMDHSISAFMRKGISGDWKTTFTVAQNERFDADYAKKMEGCGLSFRTQL

SEQ ID NO: 23 - protein, 294 aa, Gallus gallus
MGNDEVIRQDLGCLYDIPLYKCFVAGWPQVEAFQARPDDLLIATYPKSGTTWLSEILDAIYHDGDLEKCRRDAIYNRVPFLEMKAPGILSGVEQLEKIPSPRLVKTHLPVHLLPASFQEKDCKVIYMARNAKDVVISYYYFYQMAKIHPDPGTLSEFLQAFMDGKVAYGSWYKHVKGWWEKRHEKRLLYLFYEDMKKDPRREIQKILQFLGKEVAEETVARILHHTSFQEMKKNPATNYETMPTELMDHSLSPFMRKGISGDWANHFTVAQNERFDQHYQQQMAGSDLCFQMEA

SEQ ID NO: 24 - protein, 307 aa, Gallus gallus
MALDKMENLSLEENMLRSEMGEVQGIPVTKPTCDIWDQVWNFKARPDDLLVATYAKAGTTWTQEIVDMIQQNGDIEKCRRASTYKRHPFLEWYIPDSSPLGYSGLKLAEAMPSPRTMKTHLPVQLVPPSFWEQNCKIIYVARNAKDNLVSYYHFHRMNKVLPDPGTIEEFTEKFMNGEVLWGSWYDHVKGWWKAKDKHRILYLFYEDMKENPKREIQKIMKFLEKDLDEEVLNKIIYNTSFEIMKDNPMTNYTKDFVGVMDHSVSPFMRKGSVGDWKNYFTVALNKKFDQDYKKKMADTSLVFRMEL

SEQ ID NO: 25 - protein, 301 aa, Danio rerio
MDLPDISSIKLPSRPKIFEFEGISMISYFTDNWEKLKNFQARPDDILIATYPKAGTTWVSYILDLLYFGKVEPNGQSSLPIYMRVPFLESCFPGMPSGTELADNLPNSPRLIKTHLPVQLVPKSFWGQNSKVVYVARNAKDNVVSFFHFDRMNHGQPEPGDWDTFLQAFIKGERVFGSWFDHVCGWWEKKKTYPNLHYMFYEDIAKDINGEVESLCTFLKLSRSDEEKEKIINGVQFDAMKQNVMTNYSTIPTMDFTISPFMRKGKVGDWKNHFTVAQNEQFDEDYKEKMKNTTLNFRTKI

SEQ ID NO: 26 - protein, 300 aa, Danio rerio
MEIQGKSSTDLPDRPEIFEFEGISMVEHFTKNWENVKNFQARPDDILIATYPKAGTTWVSNILDLLYFGKEDPKRQTTKPIYKRVPFLESCFPEMQSGTELANNLPTSPRLIKTHLPVQLVPQSFWEKNSRVAYVARNAKDNAVSYFHFNRMNKAQPEPGDWNTFLEEFMKGKMVFGSWFDHVCGWWEKKKTYPNLHYMLYEDMAKDIKGEVESLCTFLKLSRSDEEKEKIINGIQFDAMKQNKMTNYSTVLVMDFTISPFMRKGKVGDWKNHFTVAQNEQFNEDYKQKMKNSTLKFPTE

SEQ ID NO: 27 - protein, 338 aa, Drosophila melanogaster
MPQSSFFAKSVPFEQIDKLAISGGYSSIFASSKPSVPVVGNWEQRFCRLADTFQPVLDRVYDFEVRDDDVWIVTLPKCGTTWMQELAWLVINECDFETAKSVDLTHRSPFLEFNGVVPNVPHDTIAAANALPSPRLIKSHLPAWMLPRQIWSKRPKIIYVYRNPKDAAISYFHHWRGMVGYQGTKSDFMHSFIDGYVNFTPCWPHILDFWQLRHEPNIFFTSYERMKGQLGQVISEVAQFLERSVSQEQMQQMQRHLSFESMRDNPACNHVKEFESMKAAAGREVEEFRFVRRGVVGSHKDELTADIIREFDLWSDSNLRDFKLNMDDFANYSKFAST

SEQ ID NO: 28 - protein, 313 aa, Drosophila melanogaster
MNRVQVTPRSYPTNLIDKDWGNRKLFYTKDSENFLRLVHDMKLRDDDVWIVTLPKCGTTWMQELLWLLLNNCDFEGALAKDQELRTPFLEFGYSVFHDPNRSFGPIEDLKSPRLIKSHLSLALLPSKLWEGKNKVIYVSRNPLDSYVSRYYHGVSFGFNYGKSLHQYFDEVLASDDFPTEFIEHAHEFYQLRNEPWVFYTSFEMMKKDLRGVINDVSRFLNKPINDQQMEKLLKHLSFAEMKKNPTTNHLWELAQVQHENAGKEMHPFVRRGDVNGYKDELKPEQIEKANVRIQEVLAKNGVTLDELLLLKDQ

SEQ ID NO: 29 - protein, 346 aa, Drosophila melanogaster
MENTPLKFPHEIRDVEESTNAELLDHFHGERTGFVQVGSEGYFFPHKYKDEAERYYNFEARPDDVWIATVPRSGTTWTQELIWLVANGLDFEHAQERPLTERFPFFEFPLFVHPKIKEELQEENRDSAEALEFIEKIARPGYEALSEIPRSQRRFIKTHFPFSLMPPSVLEKKCKVIYVVRDPKDVAVSYYHLNRLFRTQGYVGDFERYWHYFQNGLNPWLPYYSHVKEAREHAHLSNVLFLRYEDMLADLPGAINSIASFLECPPKPEDMDRLLDHLSIRSFRENKSVNMHEMASVGVLNKGEAGFVRSGAKTAYQPQQEFVENPKLLKSANEWVEQNIKSFKTI

SEQ ID NO: 30 - protein, 323 aa, Arabidopsis thaliana
MEMNLRIEDLNEETKTLISSLPSDKDFTGKTICKYQGCWYTHNVLQAVLNFQKSFKPQDTDIIVASFPKCGTTWLKALTFALLHRSKQPSHDDDHPLLSNNPHVLVPYFEIDLYLRSENPDLTKFSSSPRLFSTHVPSHTLQEGLKGSTCKIVYISRNVKDTLVSYWHFFTKKQTDEKIISSFEDTFEMFCRGVSIFGPFWDHVLSYWRGSLEDPNHVLFMKFEEMKAEPRDQIKKFAEFLGCPFTKEEEESGSVDEIIDLCSLRNLSSLEINKTGKLNSGRENKMFFRKGEVGDWKNYLTPEMENKIDMIIQEKLQNSGLKF

SEQ ID NO: 31 - DNA, 82 bp, Artificial Sequence
  sgRNA body sequence
gttttagagctagaaatagcaagttaaaataaggctagtccgttatcaacttgaaaaagtggcaccgagtcggtgctttttt

SEQ ID NO: 32 - DNA, 102 bp, Artificial Sequence
  Full sequence of sgRNA targeting purF (E. coli)
tcataaatcgactggttaacgttttagagctagaaatagcaagttaaaataaggctagtccgttatcaacttgaaaaagtggcaccgagtcggtgctttttt

SEQ ID NO: 33 - DNA, 102 bp, Artificial Sequence
  Full sequence of sgRNA targeting purD (E. coli)
cggcgactgggccgctttccgttttagagctagaaatagcaagttaaaataaggctagtccgttatcaacttgaaaaagtggcaccgagtcggtgctttttt

SEQ ID NO: 34 - DNA, 102 bp, Artificial Sequence
  Full sequence of sgRNA targeting purN (E. coli)
ctgtaaattacttccgttgcgttttagagctagaaatagcaagttaaaataaggctagtccgttatcaacttgaaaaagtggcaccgagtcggtgctttttt

SEQ ID NO: 35 - DNA, 102 bp, Artificial Sequence
  Full sequence of sgRNA targeting purL (E. coli)
attcggaatgccgacagtgcgttttagagctagaaatagcaagttaaaataaggctagtccgttatcaacttgaaaaagtggcaccgagtcggtgctttttt

SEQ ID NO: 36 - DNA, 102 bp, Artificial Sequence
  Full sequence of sgRNA targeting purM (E. coli)
ggcatctttgtagctaagaggttttagagctagaaatagcaagttaaaataaggctagtccgttatcaacttgaaaaagtggcaccgagtcggtgctttttt

SEQ ID NO: 37 - DNA, 102 bp, Artificial Sequence
  Full sequence of sgRNA targeting purK (E. coli)
ggcctaactgcccgttaccggttttagagctagaaatagcaagttaaaataaggctagtccgttatcaacttgaaaaagtggcaccgagtcggtgctttttt

SEQ ID NO: 38 - DNA, 102 bp, Artificial Sequence
  Full sequence of sgRNA targeting purE (E. coli)
gacacgcgccggattattgcgttttagagctagaaatagcaagttaaaataaggctagtccgttatcaacttgaaaaagtggcaccgagtcggtgctttttt

SEQ ID NO: 39 - DNA, 102 bp, Artificial Sequence
  Full sequence of sgRNA targeting purC (E. coli)
attcgagcaccaacaggtccgttttagagctagaaatagcaagttaaaataaggctagtccgttatcaacttgaaaaagtggcaccgagtcggtgctttttt

SEQ ID NO: 40 - DNA, 102 bp, Artificial Sequence
  Full sequence of sgRNA targeting purB (E. coli)
tccatcgacaggggaaacgggttttagagctagaaatagcaagttaaaataaggctagtccgttatcaacttgaaaaagtggcaccgagtcggtgctttttt

SEQ ID NO: 41 - DNA, 102 bp, Artificial Sequence
  Full sequence of sgRNA targeting purH (E. coli)
aaacactgagcagagcgcgggttttagagctagaaatagcaagttaaaataaggctagtccgttatcaacttgaaaaagtggcaccgagtcggtgctttttt

SEQ ID NO: 42 - DNA, 102 bp, Artificial Sequence
  Full sequence of sgRNA targeting purA (E. coli)
tttaccttcgtcaccccattgttttagagctagaaatagcaagttaaaataaggctagtccgttatcaacttgaaaaagtggcaccgagtcggtgctttttt

SEQ ID NO: 43 - DNA, 102 bp, Artificial Sequence
  Full sequence of sgRNA targeting guaA (E. coli)
gagaaccgaagtccagaatggttttagagctagaaatagcaagttaaaataaggctagtccgttatcaacttgaaaaagtggcaccgagtcggtgctttttt

SEQ ID NO: 44 - DNA, 102 bp, Artificial Sequence
  Full sequence of sgRNA targeting guaB (E. coli)
cggtagagtgagcaggaacggttttagagctagaaatagcaagttaaaataaggctagtccgttatcaacttgaaaaagtggcaccgagtcggtgctttttt

SEQ ID NO: 45 - DNA, 102 bp, Artificial Sequence
  Full sequence of sgRNA targeting adk (E. coli)
tgagtccctttccccgcgccgttttagagctagaaatagcaagttaaaataaggctagtccgttatcaacttgaaaaagtggcaccgagtcggtgctttttt

SEQ ID NO: 46 - DNA, 102 bp, Artificial Sequence
  Full sequence of sgRNA targeting gmk (E. coli)
tggatttacccgcgccactggttttagagctagaaatagcaagttaaaataaggctagtccgttatcaacttgaaaaagtggcaccgagtcggtgctttttt

SEQ ID NO: 47 - DNA, 102 bp, Artificial Sequence
  Full sequence of sgRNA targeting pykA (E. coli)
atctgttgctgggcctaacggttttagagctagaaatagcaagttaaaataaggctagtccgttatcaacttgaaaaagtggcaccgagtcggtgctttttt

SEQ ID NO: 48 - DNA, 102 bp, Artificial Sequence
  Full sequence of sgRNA targeting guaC (E. coli)
taagagtggagcgtttaggggttttagagctagaaatagcaagttaaaataaggctagtccgttatcaacttgaaaaagtggcaccgagtcggtgctttttt

SEQ ID NO: 49 - DNA, 102 bp, Artificial Sequence
  Full sequence of sgRNA targeting dgt (E. coli)
ttttaacgccctgcggtgaagttttagagctagaaatagcaagttaaaataaggctagtccgttatcaacttgaaaaagtggcaccgagtcggtgctttttt

SEQ ID NO: 50 - DNA, 102 bp, Artificial Sequence
  Full sequence of sgRNA targeting carA (E. coli)
tatggcccgaccgtgaaactgttttagagctagaaatagcaagttaaaataaggctagtccgttatcaacttgaaaaagtggcaccgagtcggtgctttttt

SEQ ID NO: 51 - DNA, 102 bp, Artificial Sequence
  Full sequence of sgRNA targeting carB (E. coli)
tcgggcccgcacccagaatcgttttagagctagaaatagcaagttaaaataaggctagtccgttatcaacttgaaaaagtggcaccgagtcggtgctttttt

SEQ ID NO: 52 - DNA, 102 bp, Artificial Sequence
  Full sequence of sgRNA targeting pyrB (E. coli)
atgatatgtttctgatataggttttagagctagaaatagcaagttaaaataaggctagtccgttatcaacttgaaaaagtggcaccgagtcggtgctttttt

SEQ ID NO: 53 - DNA, 102 bp, Artificial Sequence
  Full sequence of sgRNA targeting pyrC (E. coli)
cggatctttaatacctgggagttttagagctagaaatagcaagttaaaataaggctagtccgttatcaacttgaaaaagtggcaccgagtcggtgctttttt

SEQ ID NO: 54 - DNA, 102 bp, Artificial Sequence
  Full sequence of sgRNA targeting pyrD (E. coli)
aaaagggctttacgaacgaagttttagagctagaaatagcaagttaaaataaggctagtccgttatcaacttgaaaaagtggcaccgagtcggtgctttttt

SEQ ID NO: 55 - DNA, 102 bp, Artificial Sequence
  Full sequence of sgRNA targeting pyrE (E. coli)
taagcgcaaattcaataaacgttttagagctagaaatagcaagttaaaataaggctagtccgttatcaacttgaaaaagtggcaccgagtcggtgctttttt

SEQ ID NO: 56 - DNA, 102 bp, Artificial Sequence
  Full sequence of sgRNA targeting pyrF (E. coli)
caggagaattcgtaacagcggttttagagctagaaatagcaagttaaaataaggctagtccgttatcaacttgaaaaagtggcaccgagtcggtgctttttt

SEQ ID NO: 57 - DNA, 102 bp, Artificial Sequence
  Full sequence of sgRNA targeting pyrG (E. coli)
ggcaatgcctttacccagaggttttagagctagaaatagcaagttaaaataaggctagtccgttatcaacttgaaaaagtggcaccgagtcggtgctttttt

SEQ ID NO: 58 - DNA, 102 bp, Artificial Sequence
  Full sequence of sgRNA targeting pyrH (E. coli)
tttatagacgggttttgcatgttttagagctagaaatagcaagttaaaataaggctagtccgttatcaacttgaaaaagtggcaccgagtcggtgctttttt

SEQ ID NO: 59 - DNA, 102 bp, Artificial Sequence
  Full sequence of sgRNA targeting ndk (E. coli)
acgttttttgctaccgcgttgttttagagctagaaatagcaagttaaaataaggctagtccgttatcaacttgaaaaagtggcaccgagtcggtgctttttt

SEQ ID NO: 60 - DNA, 102 bp, Artificial Sequence
  Full sequence of sgRNA targeting cmk (E. coli)
ggccatcaatggtaataaccgttttagagctagaaatagcaagttaaaataaggctagtccgttatcaacttgaaaaagtggcaccgagtcggtgctttttt

SEQ ID NO: 61 - DNA, 102 bp, Artificial Sequence
  Full sequence of sgRNA targeting nrdA (E. coli)
gtgggagcgcagctcgacctgttttagagctagaaatagcaagttaaaataaggctagtccgttatcaacttgaaaaagtggcaccgagtcggtgctttttt

SEQ ID NO: 62 - DNA, 102 bp, Artificial Sequence
  Full sequence of sgRNA targeting nrdB (E. coli)
atttttcgtctgtgaaaagggttttagagctagaaatagcaagttaaaataaggctagtccgttatcaacttgaaaaagtggcaccgagtcggtgctttttt

SEQ ID NO: 63 - DNA, 102 bp, Artificial Sequence
  Full sequence of sgRNA targeting nrdD (E. coli)
ccgtctcgtttcatcacatggttttagagctagaaatagcaagttaaaataaggctagtccgttatcaacttgaaaaagtggcaccgagtcggtgctttttt

SEQ ID NO: 64 - DNA, 102 bp, Artificial Sequence
  Full sequence of sgRNA targeting tmk (E. coli)
ctcaaccaccacattacgcggttttagagctagaaatagcaagttaaaataaggctagtccgttatcaacttgaaaaagtggcaccgagtcggtgctttttt

SEQ ID NO: 65 - DNA, 102 bp, Artificial Sequence
  Full sequence of sgRNA targeting dut (E. coli)
gaaattccttcccaacgcgcgttttagagctagaaatagcaagttaaaataaggctagtccgttatcaacttgaaaaagtggcaccgagtcggtgctttttt

SEQ ID NO: 66 - DNA, 102 bp, Artificial Sequence
  Full sequence of sgRNA targeting thyA (E. coli)
aatggaaagcgttccggttcgttttagagctagaaatagcaagttaaaataaggctagtccgttatcaacttgaaaaagtggcaccgagtcggtgctttttt

SEQ ID NO: 67 - DNA, 102 bp, Artificial Sequence
  Full sequence of sgRNA targeting dcd (E. coli)
caagccaggcttcaatatctgttttagagctagaaatagcaagttaaaataaggctagtccgttatcaacttgaaaaagtggcaccgagtcggtgctttttt

SEQ ID NO: 68 - DNA, 102 bp, Artificial Sequence
  Full sequence of sgRNA targeting cdd (E. coli)
atccgcaagttgggcaaaaggttttagagctagaaatagcaagttaaaataaggctagtccgttatcaacttgaaaaagtggcaccgagtcggtgctttttt

SEQ ID NO: 69 - DNA, 102 bp, Artificial Sequence
  Full sequence of sgRNA targeting codA (E. coli)
cccctcttcgcctggtaaccgttttagagctagaaatagcaagttaaaataaggctagtccgttatcaacttgaaaaagtggcaccgagtcggtgctttttt

SEQ ID NO: 70 - DNA, 102 bp, Artificial Sequence
  Full sequence of sgRNA targeting udk (E. coli)
caattcacgataaagggtacgttttagagctagaaatagcaagttaaaataaggctagtccgttatcaacttgaaaaagtggcaccgagtcggtgctttttt

SEQ ID NO: 71 - DNA, 102 bp, Artificial Sequence
  Full sequence of sgRNA targeting tdk (E. coli)
cagtgcgcatgccgcgttccgttttagagctagaaatagcaagttaaaataaggctagtccgttatcaacttgaaaaagtggcaccgagtcggtgctttttt

SEQ ID NO: 72 - DNA, 102 bp, Artificial Sequence
  Full sequence of sgRNA targeting udp (E. coli)
gagatgaaaaacatcagactgttttagagctagaaatagcaagttaaaataaggctagtccgttatcaacttgaaaaagtggcaccgagtcggtgctttttt

SEQ ID NO: 73 - DNA, 102 bp, Artificial Sequence
  Full sequence of sgRNA targeting deoA (E. coli)
gaaaatggtcatcgcgaggggttttagagctagaaatagcaagttaaaataaggctagtccgttatcaacttgaaaaagtggcaccgagtcggtgctttttt

SEQ ID NO: 74 - DNA, 102 bp, Artificial Sequence
  Full sequence of sgRNA targeting atpB (E. coli)
tggtgtcctatgtaatcctggttttagagctagaaatagcaagttaaaataaggctagtccgttatcaacttgaaaaagtggcaccgagtcggtgctttttt

SEQ ID NO: 75 - DNA, 102 bp, Artificial Sequence
  Full sequence of sgRNA targeting atpF (E. coli)
acaggacaaacgcgatggccgttttagagctagaaatagcaagttaaaataaggctagtccgttatcaacttgaaaaagtggcaccgagtcggtgctttttt

SEQ ID NO: 76 - DNA, 102 bp, Artificial Sequence
  Full sequence of sgRNA targeting atpE (E. coli)
tgtacagcagatccatattcgttttagagctagaaatagcaagttaaaataaggctagtccgttatcaacttgaaaaagtggcaccgagtcggtgctttttt

SEQ ID NO: 77 - DNA, 102 bp, Artificial Sequence
  Full sequence of sgRNA targeting atpD (E. coli)
agggaattcgacgtcaactagttttagagctagaaatagcaagttaaaataaggctagtccgttatcaacttgaaaaagtggcaccgagtcggtgctttttt

SEQ ID NO: 78 - DNA, 102 bp, Artificial Sequence
  Full sequence of sgRNA targeting atpG (E. coli)
tagtgatcttttgcgtgttcgttttagagctagaaatagcaagttaaaataaggctagtccgttatcaacttgaaaaagtggcaccgagtcggtgctttttt

SEQ ID NO: 79 - DNA, 102 bp, Artificial Sequence
  Full sequence of sgRNA targeting atpA (E. coli)
gatcagttcgctgatttcgggttttagagctagaaatagcaagttaaaataaggctagtccgttatcaacttgaaaaagtggcaccgagtcggtgctttttt

SEQ ID NO: 80 - DNA, 102 bp, Artificial Sequence
  Full sequence of sgRNA targeting atpH (E. coli)
caaaagctgctttggcgtaggttttagagctagaaatagcaagttaaaataaggctagtccgttatcaacttgaaaaagtggcaccgagtcggtgctttttt

SEQ ID NO: 81 - DNA, 102 bp, Artificial Sequence
  Full sequence of sgRNA targeting atpC (E. coli)
gctctgcgctgacgacgtccgttttagagctagaaatagcaagttaaaataaggctagtccgttatcaacttgaaaaagtggcaccgagtcggtgctttttt

SEQ ID NO: 82 - DNA, 20 bp, Escherichia coli
aatggaaagcgttccggttc

SEQ ID NO: 83 - DNA, 20 bp, Escherichia coli
aggagaattcgtaacagcgc

SEQ ID NO: 84 - DNA, 20 bp, Escherichia coli
ctgccaaagcgaaagtgaca

SEQ ID NO: 85 - DNA, 20 bp, Escherichia coli
gatcattaactgtgaatgat

SEQ ID NO: 86 - DNA, 102 bp, Artificial Sequence; identical to SEQ ID NO: 66
  Full sequence of sgRNA targeting thyA (E. coli)

SEQ ID NO: 87 - DNA, 102 bp, Artificial Sequence
  Full sequence of sgRNA targeting pyrF (E. coli)
aggagaattcgtaacagcgcgttttagagctagaaatagcaagttaaaataaggctagtccgttatcaacttgaaaaagtggcaccgagtcggtgctttttt

SEQ ID NO: 88 - DNA, 102 bp, Artificial Sequence
  Full sequence of sgRNA targeting dnaA (E. coli)
ctgccaaagcgaaagtgacagttttagagctagaaatagcaagttaaaataaggctagtccgttatcaacttgaaaaagtggcaccgagtcggtgctttttt

SEQ ID NO: 89 - DNA, 102 bp, Artificial Sequence
  Full sequence of sgRNA targeting oriC (E. coli)
gatcattaactgtgaatgatgttttagagctagaaatagcaagttaaaataaggctagtccgttatcaacttgaaaaagtggcaccgagtcggtgctttttt

SEQ ID NO: 90 - DNA, 82 bp, Artificial Sequence; identical to SEQ ID NO: 31
  nc (blank)

SEQ ID NO: 91 - DNA, 55 bp, Artificial Sequence
  Primer
catctaattcaacaagaattgttttagagctagaaatagcaagttaaaataaggc

SEQ ID NO: 92 - DNA, 49 bp, Artificial Sequence
  Primer
aattcttgttgaattagatgactagtattatacctaggactgagctagc

SEQ ID NO: 93 - DNA, 55 bp, Artificial Sequence
  Primer
aatggaaagcgttccggttcgttttagagctagaaatagcaagttaaaataaggc

SEQ ID NO: 94 - DNA, 49 bp, Artificial Sequence
  Primer
gaaccggaacgctttccattactagtattatacctaggactgagctagc

SEQ ID NO: 95 - DNA, 55 bp, Artificial Sequence
  Primer
aggagaattcgtaacagcgcgttttagagctagaaatagcaagttaaaataaggc

SEQ ID NO: 96 - DNA, 49 bp, Artificial Sequence
  Primer
gcgctgttacgaattctcctactagtattatacctaggactgagctagc

SEQ ID NO: 97 - DNA, 35 bp, Artificial Sequence
  Primer
gttttagagctagaaatagcaagttaaaataaggc

SEQ ID NO: 98 - DNA, 49 bp, Artificial Sequence
  Primer
gctatttctagctctaaaacactagtattatacctaggactgagctagc

SEQ ID NO: 99 - DNA, 49 bp, Artificial Sequence
  Primer
gctatttctagctctaaaacactagtcttttctctatcactgataggga

SEQ ID NO: 100 - DNA, 44 bp, Artificial Sequence
  Primer
actttaataaggagatatacggttacggttgagtaataaatgga

SEQ ID NO: 101 - DNA, 41 bp, Artificial Sequence
  Primer
tggcagcagcctaggttaatcgaaccgaacaggcttatgtc

SEQ ID NO: 102 - DNA, 18 bp, Artificial Sequence
  Primer
agatgcauggcgcctaac

SEQ ID NO: 103 - DNA, 24 bp, Artificial Sequence
  Primer
agccctcuagaggatccccgggta

SEQ ID NO: 104 - DNA, 28 bp, Artificial Sequence
  Primer
agagggcucaacgtctcattttcgccag

SEQ ID NO: 105 - DNA, 30 bp, Artificial Sequence
  Primer
atgcatcuatccttactcgagttagtcacc

SEQ ID NO: 106 - DNA, 20 bp, Artificial Sequence
  Primer
agtcggugctttttttgaag

SEQ ID NO: 107 - DNA, 29 bp, Artificial Sequence
  Primer
accgacuactagtcttttctctatcactg

SEQ ID NO: 108 - DNA, 27 bp, Artificial Sequence
  Primer
ctccctatcagtgatagagaaaagact

SEQ ID NO: 109 - DNA, 60 bp, Artificial Sequence
  Primer
cgggcccaagcttcaaaaaaagcaccgactcggtgccactttttcaagttgataacggac

SEQ ID NO: 110 - DNA, 20 bp, Artificial Sequence
  Primer
agtcggtgctttttttgaag

SEQ ID NO: 111 - DNA, 30 bp, Artificial Sequence
  Primer
actagtcttttctctatcactgatagggag

SEQ ID NO: 112 - DNA, 55 bp, Artificial Sequence
  Primer
acttcaattaacaccagcgggttttagagctagaaatagcaagttaaaataaggc

SEQ ID NO: 113 - DNA, 49 bp, Artificial Sequence
  Primer
ccgctggtgttaattgaagtactagtcttttctctatcactgataggga

SEQ ID NO: 114 - DNA, 55 bp, Artificial Sequence
  Primer
agacgcgttagtgtcttatcgttttagagctagaaatagcaagttaaaataaggc

SEQ ID NO: 115 - DNA, 49 bp, Artificial Sequence
  Primer
gataagacactaacgcgtctactagtcttttctctatcactgataggga

SEQ ID NO: 116 - DNA, 55 bp, Artificial Sequence
  Primer
agctcttcctcaatgttgacgttttagagctagaaatagcaagttaaaataaggc

SEQ ID NO: 117 - DNA, 49 bp, Artificial Sequence
  Primer
gtcaacattgaggaagagctactagtcttttctctatcactgataggga

SEQ ID NO: 118 - DNA, 55 bp, Artificial Sequence
  Primer
attaccttttgtgaaggcaggttttagagctagaaatagcaagttaaaataaggc

SEQ ID NO: 119 - DNA, 49 bp, Artificial Sequence
  Primer
ctgccttcacaaaaggtaatactagtcttttctctatcactgataggga

SEQ ID NO: 120 - DNA, 55 bp, Artificial Sequence
  Primer
cgtcagggtgactttcttgcgttttagagctagaaatagcaagttaaaataaggc

SEQ ID NO: 121 - DNA, 49 bp, Artificial Sequence
  Primer
gcaagaaagtcaccctgacgactagtcttttctctatcactgataggga

SEQ ID NO: 122 - DNA, 55 bp, Artificial Sequence
  Primer
cgtcgcggttcagacatgaagttttagagctagaaatagcaagttaaaataaggc

SEQ ID NO: 123 - DNA, 49 bp, Artificial Sequence
  Primer
ttcatgtctgaaccgcgacgactagtcttttctctatcactgataggga

SEQ ID NO: 124 - DNA, 55 bp, Artificial Sequence
  Primer
gagaagcagatgacttccgggttttagagctagaaatagcaagttaaaataaggc

SEQ ID NO: 125 - DNA, 49 bp, Artificial Sequence
  Primer
ccggaagtcatctgcttctcactagtcttttctctatcactgataggga

SEQ ID NO: 126 - DNA, 55 bp, Artificial Sequence
  Primer
gttgaatgtccagaacgggtgttttagagctagaaatagcaagttaaaataaggc

SEQ ID NO: 127 - DNA, 49 bp, Artificial Sequence
  Primer
acccgttctggacattcaacactagtcttttctctatcactgataggga

SEQ ID NO: 128 - DNA, 55 bp, Artificial Sequence
  Primer
taaactgtggcggataggatgttttagagctagaaatagcaagttaaaataaggc

SEQ ID NO: 129 - DNA, 49 bp, Artificial Sequence
  Primer
atcctatccgccacagtttaactagtcttttctctatcactgataggga

SEQ ID NO: 130 - DNA, 55 bp, Artificial Sequence
  Primer
tactaagactaccagggcgggttttagagctagaaatagcaagttaaaataaggc

SEQ ID NO: 131 - DNA, 49 bp, Artificial Sequence
  Primer
ccgccctggtagtcttagtaactagtcttttctctatcactgataggga

SEQ ID NO: 132 - DNA, 55 bp, Artificial Sequence
  Primer
aatcctgcgcctgacaggccgttttagagctagaaatagcaagttaaaataaggc

SEQ ID NO: 133 - DNA, 49 bp, Artificial Sequence
  Primer
ggcctgtcaggcgcaggattactagtcttttctctatcactgataggga

SEQ ID NO: 134 - DNA, 55 bp, Artificial Sequence
  Primer
ataggtaaatttctgggtccgttttagagctagaaatagcaagttaaaataaggc

SEQ ID NO: 135 - DNA, 49 bp, Artificial Sequence
  Primer
ggacccagaaatttacctatactagtcttttctctatcactgataggga

SEQ ID NO: 136 - DNA, 55 bp, Artificial Sequence
  Primer
cggcatatacatttgggtccgttttagagctagaaatagcaagttaaaataaggc

SEQ ID NO: 137 - DNA, 49 bp, Artificial Sequence
  Primer
ggacccaaatgtatatgccgactagtcttttctctatcactgataggga

SEQ ID NO: 138 - DNA, 55 bp, Artificial Sequence
  Primer
cttcggttattgccgggtccgttttagagctagaaatagcaagttaaaataaggc

SEQ ID NO: 139 - DNA, 49 bp, Artificial Sequence
  Primer
ggacccggcaataaccgaagactagtcttttctctatcactgataggga

SEQ ID NO: 140 - DNA, 55 bp, Artificial Sequence
  Primer
tgtttaacaaatgggggcacgttttagagctagaaatagcaagttaaaataaggc

SEQ ID NO: 141 - DNA, 49 bp, Artificial Sequence
  Primer
gtgcccccatttgttaaacaactagtcttttctctatcactgataggga

SEQ ID NO: 142 - DNA, 59 bp, Artificial Sequence
  Primer
tcgtcggcagcgtcagatgtgtataagagacagcactccctatcagtgatagagaaaag

SEQ ID NO: 143 - DNA, 58 bp, Artificial Sequence
  Primer
gtctcgtgggctcggagatgtgtataagagacagattcagatcctcttctgagatgag

SEQ ID NO: 144 - DNA, 20 bp, Artificial Sequence
  Primer
acctgtttcgcgccacttcc

SEQ ID NO: 145 - DNA, 20 bp, Artificial Sequence
  Primer
gtagaccagacggctgttgg

SEQ ID NO: 146 - DNA, 54 bp, Artificial Sequence
  Primer
agctgaaauagctgcgcttttttgtgtcataactaataacgtaacgtgactggc

SEQ ID NO: 147 - DNA, 83 bp, Artificial Sequence
  Primer
aatcttttcuccctgataatttaacacactttcaaaagagtgtcaacgtgtattgacgcagtcgaacgaaaatcgccattcgc

SEQ ID NO: 148 - DNA, 25 bp, Artificial Sequence
  Primer
aaacatgagtaaaggcgaagagctg

SEQ ID NO: 149 - DNA, 47 bp, Artificial Sequence
  Primer
atttcagcugcgctttttttatttgtacagttcatccataccatgcg

SEQ ID NO: 150 - DNA, 20 bp, Artificial Sequence
  Primer
gtgtagaagggaacggttgg

SEQ ID NO: 151 - DNA, 21 bp, Artificial Sequence
  Primer
ttgtattccagcttatggccc

SEQ ID NO: 152 - DNA, 23 bp, Artificial Sequence
  Primer
cgtgcggaagtgaaatttgaagg

SEQ ID NO: 153 - DNA, 20 bp, Escherichia coli
tttaccttcgtcaccccatt

SEQ ID NO: 154 - DNA, 20 bp, Escherichia coli
tccatcgacaggggaaacgg

SEQ ID NO: 155 - DNA, 20 bp, Escherichia coli
cgggttttccgtgctgtata

SEQ ID NO: 156 - DNA, 20 bp, Escherichia coli
cggcgactgggccgctttcc

SEQ ID NO: 157 - DNA, 20 bp, Escherichia coli
gacacgcgccggattattgc

SEQ ID NO: 158 - DNA, 20 bp, Escherichia coli
tcataaatcgactggttaac

SEQ ID NO: 159 - DNA, 20 bp, Escherichia coli
aaacactgagcagagcgcgg

SEQ ID NO: 160 - DNA, 20 bp, Escherichia coli
ggcctaactgcccgttaccg

SEQ ID NO: 161 - DNA, 20 bp, Escherichia coli
attcggaatgccgacagtgc

SEQ ID NO: 162 - DNA, 20 bp, Escherichia coli
ggcatctttgtagctaagag

SEQ ID NO: 163 - DNA, 20 bp, Escherichia coli
ctgtaaattacttccgttgc

SEQ ID NO: 164 - DNA, 20 bp, Escherichia coli
gagaaccgaagtccagaatg

SEQ ID NO: 165 - DNA, 20 bp, Escherichia coli
cggtagagtgagcaggaacg

SEQ ID NO: 166 - DNA, 20 bp, Escherichia coli
atgatatgtttctgatatag

SEQ ID NO: 167 - DNA, 20 bp, Escherichia coli
aaaagggctttacgaacgaa

SEQ ID NO: 168 - DNA, 20 bp, Escherichia coli
taagcgcaaattcaataaac

SEQ ID NO: 169 - DNA, 20 bp, Escherichia coli
aggagaattcgtaacagcgc

SEQ ID NO: 170 - DNA, 20 bp, Escherichia coli
ggcaatgcctttacccagag

SEQ ID NO: 171 - DNA, 20 bp, Escherichia coli
tttatagacgggttttgcat

SEQ ID NO: 172 - DNA, 20 bp, Escherichia coli
ggccatcaatggtaataacc

SEQ ID NO: 173 - DNA, 20 bp, Escherichia coli
acgttttttgctaccgcgtt

SEQ ID NO: 174 - DNA, 102 bp, Artificial Sequence; identical to SEQ ID NO: 42
  Full sequence of sgRNA targeting purA (E. coli)

SEQ ID NO: 175 - DNA, 102 bp, Artificial Sequence; identical to SEQ ID NO: 40
  Full sequence of sgRNA targeting purB (E. coli)

SEQ ID NO: 176 - DNA, 102 bp, Artificial Sequence
  Full sequence of sgRNA targeting purC (E. coli)
cgggttttccgtgctgtatagttttagagctagaaatagcaagttaaaataaggctagtccgttatcaacttgaaaaagtggcaccgagtcggtgctttttt

SEQ ID NO: 177 - DNA, 102 bp, Artificial Sequence; identical to SEQ ID NO: 33
  Full sequence of sgRNA targeting purD (E. coli)

SEQ ID NO: 178 - DNA, 102 bp, Artificial Sequence; identical to SEQ ID NO: 38
  Full sequence of sgRNA targeting purE (E. coli)

SEQ ID NO: 179 - DNA, 102 bp, Artificial Sequence; identical to SEQ ID NO: 32
  Full sequence of sgRNA targeting purF (E. coli)

SEQ ID NO: 180 - DNA, 102 bp, Artificial Sequence; identical to SEQ ID NO: 41
  Full sequence of sgRNA targeting purH (E. coli)

SEQ ID NO: 181 - DNA, 102 bp, Artificial Sequence; identical to SEQ ID NO: 37
  Full sequence of sgRNA targeting purK (E. coli)

SEQ ID NO: 182 - DNA, 102 bp, Artificial Sequence; identical to SEQ ID NO: 35
  Full sequence of sgRNA targeting purL (E. coli)

SEQ ID NO: 183 - DNA, 102 bp, Artificial Sequence; identical to SEQ ID NO: 36
  Full sequence of sgRNA targeting purM (E. coli)

SEQ ID NO: 184 - DNA, 102 bp, Artificial Sequence; identical to SEQ ID NO: 34
  Full sequence of sgRNA targeting purN (E. coli)

SEQ ID NO: 185 - DNA, 102 bp, Artificial Sequence; identical to SEQ ID NO: 43
  Full sequence of sgRNA targeting guaA (E. coli)

SEQ ID NO: 186 - DNA, 102 bp, Artificial Sequence; identical to SEQ ID NO: 44
  Full sequence of sgRNA targeting guaB (E. coli)

SEQ ID NO: 187 - DNA, 102 bp, Artificial Sequence; identical to SEQ ID NO: 52
  Full sequence of sgRNA targeting pyrB (E. coli)

SEQ ID NO: 188 - DNA, 102 bp, Artificial Sequence; identical to SEQ ID NO: 54
  Full sequence of sgRNA targeting pyrD (E. coli)

SEQ ID NO: 189 - DNA, 102 bp, Artificial Sequence; identical to SEQ ID NO: 55
  Full sequence of sgRNA targeting pyrE (E. coli)

SEQ ID NO: 190 - DNA, 102 bp, Artificial Sequence; identical to SEQ ID NO: 87
  Full sequence of sgRNA targeting pyrF (E. coli)

SEQ ID NO: 191 - DNA, 102 bp, Artificial Sequence; identical to SEQ ID NO: 57
  Full sequence of sgRNA targeting pyrG (E. coli)

SEQ ID NO: 192 - DNA, 102 bp, Artificial Sequence; identical to SEQ ID NO: 58
  Full sequence of sgRNA targeting pyrH (E. coli)

SEQ ID NO: 193 - DNA, 102 bp, Artificial Sequence; identical to SEQ ID NO: 60
  Full sequence of sgRNA targeting cmk (E. coli)

SEQ ID NO: 194 - DNA, 102 bp, Artificial Sequence; identical to SEQ ID NO: 59
  Full sequence of sgRNA targeting ndk (E. coli)

SEQ ID NO: 195 - DNA, 55 bp, Artificial Sequence
  Primer
tttaccttcgtcaccccattgttttagagctagaaatagcaagttaaaataaggc

SEQ ID NO: 196 - DNA, 49 bp, Artificial Sequence
  Primer
aatggggtgacgaaggtaaaactagtcttttctctatcactgataggga

SEQ ID NO: 197 - DNA, 55 bp, Artificial Sequence
  Primer
tccatcgacaggggaaacgggttttagagctagaaatagcaagttaaaataaggc

SEQ ID NO: 198 - DNA, 49 bp, Artificial Sequence
  Primer
ccgtttcccctgtcgatggaactagtcttttctctatcactgataggga

SEQ ID NO: 199 - DNA, 55 bp, Artificial Sequence
  Primer
cgggttttccgtgctgtatagttttagagctagaaatagcaagttaaaataaggc

SEQ ID NO: 200 - DNA, 49 bp, Artificial Sequence
  Primer
tatacagcacggaaaacccgactagtcttttctctatcactgataggga

SEQ ID NO: 201 - DNA, 55 bp, Artificial Sequence
  Primer
cggcgactgggccgctttccgttttagagctagaaatagcaagttaaaataaggc

SEQ ID NO: 202 - DNA, 49 bp, Artificial Sequence
  Primer
ggaaagcggcccagtcgccgactagtcttttctctatcactgataggga

SEQ ID NO: 203 - DNA, 55 bp, Artificial Sequence
  Primer
gacacgcgccggattattgcgttttagagctagaaatagcaagttaaaataaggc

SEQ ID NO: 204 - DNA, 49 bp, Artificial Sequence
  Primer
gcaataatccggcgcgtgtcactagtcttttctctatcactgataggga

SEQ ID NO: 205 - DNA, 55 bp, Artificial Sequence
  Primer
tcataaatcgactggttaacgttttagagctagaaatagcaagttaaaataaggc

SEQ ID NO: 206 - DNA, 49 bp, Artificial Sequence
  Primer
gttaaccagtcgatttatgaactagtcttttctctatcactgataggga

SEQ ID NO: 207 - DNA, 55 bp, Artificial Sequence
  Primer
aaacactgagcagagcgcgggttttagagctagaaatagcaagttaaaataaggc

SEQ ID NO: 208 - DNA, 49 bp, Artificial Sequence
  Primer
ccgcgctctgctcagtgtttactagtcttttctctatcactgataggga

SEQ ID NO: 209 - DNA, 55 bp, Artificial Sequence
  Primer
ggcctaactgcccgttaccggttttagagctagaaatagcaagttaaaataaggc

SEQ ID NO: 210 - DNA, 49 bp, Artificial Sequence
  Primer
cggtaacgggcagttaggccactagtcttttctctatcactgataggga

SEQ ID NO: 211 - DNA, 55 bp, Artificial Sequence
  Primer
attcggaatgccgacagtgcgttttagagctagaaatagcaagttaaaataaggc

SEQ ID NO: 212 - DNA, 49 bp, Artificial Sequence
  Primer
gcactgtcggcattccgaatactagtcttttctctatcactgataggga

SEQ ID NO: 213 - DNA, 55 bp, Artificial Sequence
  Primer
ggcatctttgtagctaagaggttttagagctagaaatagcaagttaaaataaggc

SEQ ID NO: 214 - DNA, 49 bp, Artificial Sequence
  Primer
ctcttagctacaaagatgccactagtcttttctctatcactgataggga

SEQ ID NO: 215 - DNA, 55 bp, Artificial Sequence
  Primer
ctgtaaattacttccgttgcgttttagagctagaaatagcaagttaaaataaggc

SEQ ID NO: 216 - DNA, 49 bp, Artificial Sequence
  Primer
gcaacggaagtaatttacagactagtcttttctctatcactgataggga

SEQ ID NO: 217 - DNA, 55 bp, Artificial Sequence
  Primer
gagaaccgaagtccagaatggttttagagctagaaatagcaagttaaaataaggc

SEQ ID NO: 218 - DNA, 49 bp, Artificial Sequence
  Primer
cattctggacttcggttctcactagtcttttctctatcactgataggga

SEQ ID NO: 219 - DNA, 55 bp, Artificial Sequence
  Primer
cggtagagtgagcaggaacggttttagagctagaaatagcaagttaaaataaggc

SEQ ID NO: 220 - DNA, 49 bp, Artificial Sequence
  Primer
cgttcctgctcactctaccgactagtcttttctctatcactgataggga

SEQ ID NO: 221 - DNA, 55 bp, Artificial Sequence
  Primer
atgatatgtttctgatataggttttagagctagaaatagcaagttaaaataaggc

SEQ ID NO: 222 - DNA, 49 bp, Artificial Sequence
  Primer
ctatatcagaaacatatcatactagtcttttctctatcactgataggga

SEQ ID NO: 223 - DNA, 55 bp, Artificial Sequence
  Primer
aaaagggctttacgaacgaagttttagagctagaaatagcaagttaaaataaggc

SEQ ID NO: 224 - DNA, 49 bp, Artificial Sequence
  Primer
ttcgttcgtaaagcccttttactagtcttttctctatcactgataggga

SEQ ID NO: 225 - DNA, 55 bp, Artificial Sequence
  Primer
taagcgcaaattcaataaacgttttagagctagaaatagcaagttaaaataaggc

SEQ ID NO: 226 - DNA, 49 bp, Artificial Sequence
  Primer
gtttattgaatttgcgcttaactagtcttttctctatcactgataggga

SEQ ID NO: 227 - DNA, 55 bp, Artificial Sequence; identical to SEQ ID NO: 95
  Primer

SEQ ID NO: 228 - DNA, 49 bp, Artificial Sequence
  Primer
gcgctgttacgaattctcctactagtcttttctctatcactgataggga

SEQ ID NO: 229 - DNA, 55 bp, Artificial Sequence
  Primer
ggcaatgcctttacccagaggttttagagctagaaatagcaagttaaaataaggc

SEQ ID NO: 230 - DNA, 49 bp, Artificial Sequence
  Primer
ctctgggtaaaggcattgccactagtcttttctctatcactgataggga

SEQ ID NO: 231 - DNA, 55 bp, Artificial Sequence
  Primer
tttatagacgggttttgcatgttttagagctagaaatagcaagttaaaataaggc

SEQ ID NO: 232 - DNA, 49 bp, Artificial Sequence
  Primer
atgcaaaacccgtctataaaactagtcttttctctatcactgataggga

SEQ ID NO: 233 - DNA, 55 bp, Artificial Sequence
  Primer
ggccatcaatggtaataaccgttttagagctagaaatagcaagttaaaataaggc

SEQ ID NO: 234 - DNA, 49 bp, Artificial Sequence
  Primer
ggttattaccattgatggccactagtcttttctctatcactgataggga

SEQ ID NO: 235 - DNA, 55 bp, Artificial Sequence
  Primer
acgttttttgctaccgcgttgttttagagctagaaatagcaagttaaaataaggc

SEQ ID NO: 236 - DNA, 49 bp, Artificial Sequence
  Primer
aacgcggtagcaaaaaacgtactagtcttttctctatcactgataggga

SEQ ID NO: 237 - DNA, 24 bp, Artificial Sequence
  Primer
accgcctutgagtgagctgatacc

SEQ ID NO: 238 - DNA, 26 bp, Artificial Sequence
  Primer
aaggcgguaaacgaccgggtcatcgt

SEQ ID NO: 239 - DNA, 26 bp, Artificial Sequence
  Primer
aaccgtucaagatctttaagacccac

SEQ ID NO: 240 - DNA, 28 bp, Artificial Sequence
  Primer
aacggtucagggcagggtcgttaaatag

(19) United States
(12) Patent Application Publication
(10) Pub. No.: US 2019/0367930 A1
(43) Pub. Date: Dec. 5, 2019

(54) METHODS FOR DECOUPLING CELL GROWTH FROM PRODUCTION OF BIOCHEMICALS AND RECOMBINANT POLYPEPTIDES

(71) Applicant: Danmarks Tekniske Universitet, Kgs. Lyngby (DK)

(72) Inventors: Songyuan Li, Copenhagen Ø (DK); Christian Bille Jendresen, Copenhagen Ø (DK); Lasse Ebdrup Pedersen, Copenhagen N (DK); Jenny Landberg, Frederiksberg (DK); Kristoffer Bach Falkenberg, Kgs. Lyngby (DK); Hemanshu Mundhada, Nivå (DK); Alex Toftgaard Nielsen, Rungsted Kyst (DK)

(21) Appl. No.: 16/321,131

(22) PCT Filed: Jul. 28, 2017
(86) PCT No.: PCT/EP2017/069197

§ 371 Date: Jan. 28, 2019

(30) Foreign Application Priority Data
Jul. 29, 2016 (EP) 16182046.9

(51) Int. Cl. C12N 15/63; C12N 9/88; C12P 13/22; C12P 21/02; C12P 7/42; C12N 15/52; C12N 15/113

(52) U.S. Cl. CPC C12N 15/63; C12N 9/88; C12P 13/22; C12P 21/02; C12Y 208/02001; C12N 15/52; C12N 15/113; C12Y 403/01023; C12P 7/42

(57) ABSTRACT

The present invention generally relates to industrial microbiology, and specifically to the production of biochemical compounds, such as L-serine, L-tyrosine, mevalonate and their derivatives, and recombinant polypeptides using genetically modified microorganisms. More particularly, the present invention pertains to the decoupling of cell growth from production of biochemical compounds, such as L-serine, L-tyrosine, mevalonate and their derivatives, in a microorganism by down regulating the nucleotide biosynthesis in said microorganism.

### TECHNICAL FIELD OF THE INVENTION

The present invention generally relates to industrial microbiology, and specifically to the production of biochemical compounds, such as L-tyrosine, mevalonate and their derivatives, and recombinant polypeptides using genetically modified microorganisms. More particularly, the present invention pertains to the decoupling of cell growth from production of biochemical compounds, such as L-tyrosine, mevalonate and their derivatives, in a microorganism by down regulating the nucleotide biosynthesis in said microorganism.

### BACKGROUND OF THE INVENTION

Biotechnological production of chemicals and fuels through cell factories provide an alternative approach compared to the current fossil based production employed by the petrochemical industry, and it promises to do so in a sustainable way with a smaller environmental impact. E. coli is one of the most studied bacterial model organisms for metabolic engineering, and it has been employed successfully as a cell factory for production of a range of biochemicals (Lee et al., 2012). Although various compounds have been successfully produced in E. coli, improved production yields are required for most compounds to achieve industrial attractive production. Different strategies of strain engineering have been employed for this purpose: (1) Increased carbon flux through the target pathway leading to the biochemical of interest, (2) reduction of side product formation, and (3) enhancement of the availability of energy equivalents (ATP) or adjustment of the redox balance (such as improving NADP+/NADPH and NAD+/NADH ratios). However, the cell's potential has not been fully explored by focusing locally on the production pathway. In an industrial fermentation process, the dry cell weight can easily reach 10-30 g/L (Luli and Strohl, 1990), and a large portion of feedstock will therefore be used for producing biomass. If biomass formation can be reduced during the fermentation process, the yield of target biochemical compounds may be enhanced consequently.

Different techniques and strategies have been employed for the purpose of enhancing biochemical production by controlling cell growth. E. coli limited for various nutrients while having excess glucose was investigated for its metabolic activity, and a high glucose uptake rate was observed for magnesium limitation (Chubukov and Sauer, 2014). The toxin-antitoxin systems also provide a method for controlling cell growth. A single protein production system was for example developed for enriching target proteins in cells, in which a toxin protein MazF was overexpressed to arrest the cell growth (Suzuki et al., 2007). A growth arresting system, which is the result of overexpression of a toxin protein HipA, has also been shown to render the cells more resistant to antibiotics. It was therefore employed as a candidate system for antibiotics production (US2015/0353939). The production of myo-inositol was enhanced by switching on the degradation of the enzyme phosphofructokinase, which results in a reduced flux through glycolysis thus reducing cell growth (Brockman and Prather, 2015). A synthetic toggle switch has also been designed to control cell growth by conditionally turning off the TCA cycle, by which the production of isopropanol was enhanced (Soma et al., 2014). The success of previous efforts together proves that systems for growth arrest can be desirable for the production of biochemicals.

The previously developed systems for controlling growth typically involve identifying suitable toxin proteins, constructing complex synthetic pathways and engineering essential genes, which make the systems challenging to establish and maintain.

### SUMMARY OF THE INVENTION

The objective of the present invention is to provide means allowing a more efficient production of biochemical compounds, such as L-tyrosine, mevalonate and their derivatives. Particularly, it is an objective of the present invention to provide means allowing the production of biochemical compounds, such as L-tyrosine, mevalonate and their derivatives, at higher nominal yield and/or improved mass yield.

A further objective of the present invention is to provide means allowing a more efficient production of a recombinant polypeptide. Particularly, it is a further objective of the present invention to provide means allowing the production of a recombinant polypeptide at higher nominal yield and/or improved mass yield.

These objectives are addressed by the present invention which is based on the surprising finding that fermentative production of biochemical compounds, notably L-tyrosine and mevalonate, as well as the recombinant production of polypeptides by a microorganism can be enhanced by decoupling the production from cell growth through the down regulation of the biosynthesis of at least one type of nucleotide in the producing microorganism. Particularly, the present inventors have demonstrated that growth of a microorganism, exemplified by the bacterium Escherichia coli, can be controlled by inhibiting the DNA replication machinery by down regulating nucleotide biosynthesis. This way, total production of GFP as an example of a recombinant polypeptide was shown to be increased by up to 2.2-fold. Decoupling of growth from production of, e.g., mevalonate, a precursor for isoprenoid compounds, resulted in an increase in mass yield of 41% from glucose.

The present invention thus provides in a first aspect a method for decoupling cell growth from production of a biochemical compound, such as L-tyrosine or a derivative thereof, in a microorganism, especially a microorganism having an ability to produce said biochemical compound, the method comprises down regulating the biosynthesis of at least one type of nucleotide in the microorganism.

The present invention provides in a further aspect a method for decoupling cell growth from production of a recombinant polypeptide in a microorganism, the method comprises down regulating the biosynthesis of at least one type of nucleotide in the microorganism.

The present invention provides in a further aspect a method for the production of a biochemical compound, such as L-tyrosine or a derivative thereof, the method comprises:

a) growing a microorganism, especially a microorganism having the ability to produce said biochemical compound, in a culture medium; and

b) reducing the growth of the microorganism by down regulating (e.g. inhibiting) the biosynthesis of at least one type of nucleotide in the microorganism.

The present invention provides in a further aspect a method for the production of a recombinant polypeptide, the method comprises:

a) growing a microorganism, especially a microorganism having the ability to produce said recombinant polypeptide, in a culture medium; and

b) reducing the growth of the microorganism by down regulating (e.g. inhibiting) the biosynthesis of at least one type of nucleotide in the microorganism.

The present invention provides in a further aspect a genetically modified microorganism, wherein the microorganism has been modified to have a down regulated biosynthesis of at least one type of nucleotide compared to an otherwise identical microorganism that does not carry said modification. More particularly, the present invention provides a genetically modified microorganism comprising (e.g., expressing) a heterologous polypeptide having tyrosine ammonia lyase activity and/or a heterologous polypeptide having an aryl sulfotransferase activity, wherein the microorganism has been modified to have a down regulated biosynthesis of at least one type of nucleotide compared to an otherwise identical microorganism that does not carry said modification.

The present invention may be further summarized by the following items:

1. A method for decoupling cell growth from production of a biochemical compound in a microorganism, especially a microorganism having the ability to produce said biochemical compound, the method comprises inhibiting the expression and/or activity of at least one enzyme involved in the biosynthesis of at least one type of nucleotide.

2. A method for the production of a biochemical compound, the method comprises:

a) growing a microorganism, especially a microorganism having an ability to produce said biochemical compound, in a culture medium; and

b) reducing the growth of the microorganism by inhibiting the expression and/or activity of at least one enzyme involved in the biosynthesis of at least one type of nucleotide in the microorganism.

3. The method according to item 1 or 2, wherein the biochemical compound is L-tyrosine or a derivative thereof.

4. The method according to item 3, wherein the derivative is a hydroxycinnamic acid or derivative thereof.

5. The method according to item 4, wherein the hydroxycinnamic acid is p-coumaric acid.

6. The method according to item 4 or 5, wherein the hydroxycinnamic acid derivative is zosteric acid.

7. The method according to any one of items 4 to 6, wherein the microorganism comprises (e.g. expresses) a heterologous polypeptide having tyrosine ammonia lyase activity.

8. The method according to any one of items 4 to 7, wherein the microorganism comprises (e.g. expresses) a heterologous polypeptide having an aryl sulfotransferase activity.

9. The method according to any one of items 1 or 2, wherein the biochemical compound is mevalonate or a derivative thereof.

10. A method for decoupling cell growth from production of a recombinant polypeptide in a microorganism, especially a microorganism having the ability to produce said recombinant polypeptide, the method comprises inhibiting the expression and/or activity of at least one enzyme involved in the biosynthesis of at least one type of nucleotide in the microorganism.

11. A method for the production of a recombinant polypeptide, the method comprises:

a) growing a microorganism, especially a microorganism having the ability to produce a recombinant polypeptide, in a culture medium; and

b) reducing the growth of the microorganism by inhibiting the expression and/or activity of at least one enzyme involved in the biosynthesis of at least one type of nucleotide in the microorganism.

12. The method according to any one of items 1 to 11, wherein the method comprises inhibiting the expression and/or activity of at least one enzyme involved in the biosynthesis of a pyrimidine nucleotide.

13. The method according to any one of items 1 to 12, wherein the method comprises inhibiting the expression and/or activity of at least one enzyme involved in the biosynthesis of a pyrimidine nucleotide selected from the group consisting of an enzyme having orotidine-5′-phosphate decarboxylase activity, an enzyme having carbamoyl phosphate synthase activity, an enzyme having aspartate carbamoyltransferase activity, an enzyme having dihydroorotase activity, an enzyme having dihydroorotate dehydrogenase activity, an enzyme having orotate phosphoribosyltransferase activity, an enzyme having UMP kinase activity, an enzyme having nucleoside diphosphate kinase activity and an enzyme having CTP synthase activity.

14. The method according to any one of items 1 to 13, wherein the method comprises inhibiting the expression and/or activity of an enzyme having orotidine-5′-phosphate decarboxylase activity.

15. The method according to any one of items 1 to 14, wherein the method comprises inhibiting the expression and/or activity of at least one enzyme involved in the biosynthesis of a purine nucleotide.

16. The method according to any one of items 1 to 15, wherein the method comprises inhibiting the expression and/or activity of at least one enzyme involved in the biosynthesis of a purine nucleotide selected from the group consisting of an enzyme having amidophosphoribosyltransferase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N⁵-carboxyaminoimidazole ribonucleotide synthetase activity, an enzyme having N⁵-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, an enzyme having IMP cyclohydolase activity, an enzyme having adenylosuccinate synthase activity, an enzyme having adenylate kinase activity, an enzyme having ATP synthase activity, an enzyme having IMP dehydrogenase activity, an enzyme having GMP synthase activity, an enzyme having guanylate kinase activity, and an enzyme having nucleoside-diphosphate kinase activity.

17. The method according to any one of items 1 to 16, wherein the expression of the at least one enzyme is inhibited by transcriptional and/or translational repression of gene encoding said enzyme.

18. The method according to any one of items 1 to 17, wherein the expression of the at least one enzyme is inhibited by introducing or expressing in the microorganism an inhibitory nucleic acid molecule that specifically hybridizes (e.g. binds) under cellular conditions with cellular mRNA and/or genomic DNA encoding said enzyme.

19. The method according to item 18, wherein the inhibitory nucleic acid molecule is an antisense oligonucleotide, ribozyme or interfering RNA (RNAi) molecule.

20. The method according to item 19, wherein the interfering RNA molecule is a micro RNA (miRNA), small interfering RNA (siRNA) or short hairpin RNA (shRNA).

21. The method according to any one of items 18 to 20, wherein the expression of said inhibitory nucleic acid molecule is under the control of an inducible promoter, such as a temperature-inducible promoter.

22. The method according to any one of items 14 to 20, wherein the expression of the at least one enzyme is inhibited by introducing or expressing in the microorganism a catalytically inactive RNA-guided endonuclease, such as a catalytically inactive Cas9 protein, and a single guide RNA (sgRNA) specifically hybridizing (e.g. binding) under cellular conditions with the genomic DNA encoding said enzyme.

23. The method according to item 22, wherein the expression of the catalytically inactive RNA-guided endonuclease, such as the catalytically inactive Cas9 protein, and the single guide RNA (sgRNA) is under the control of an inducible promoter, such as a temperature-inducible promoter.

24. The method according to any one of items 1 to 16, wherein the expression of the at least one enzyme is under the control of a repressible promoter.

25. The method according to any one of items 1 to 17, wherein the at least one enzyme is encoded by a gene the regulatory sequence of which comprises a repressible promoter.

26. The method according to any one of items 1 to 17, wherein the at least one enzyme is encoded by a gene the regulatory sequence of which comprises an operator located between the promoter and the open reading frame encoding said enzyme.

27. The method according to item 26, wherein the expression of the at least one enzyme is inhibited by introducing or expressing in the microorganism a repressor that is capable of binding to the operator.

28. The method according to item 27, wherein the expression of the repressor is under the control of an inducible promoter, such as an temperature inducible promoter.

29. The method according to any one of items 1 to 16, wherein the activity of the at least one enzyme is inhibited by exposing the microorganism to an inhibitor of the enzyme.

30. The method according to any one of items 1 to 29, wherein the microorganism is a bacterium.

31. The method according to item 30, wherein the bacterium is a bacterium of the genus Escherichia, Bacillus, Lactococcus, Lactobacillus, Clostridium, Corynebacterium, Geobacillus, Thermoanaerobacterium, Streptococcus, Pediococcus, Moorella, Pseudomonas, Streptomyces, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus, or Yersinia.

32. The method according to item 30, wherein the bacterium is a bacterium of the genus Bacillus.

33. The method according to item 32, wherein the bacterium is Bacillus subtilis.

34. The method according to item 30, wherein the bacterium is a bacterium of the genus Lactococcus.

35. The method according to item 34, wherein the bacterium is Lactococcus lactis.

36. The method according to item 30, wherein the bacterium is a bacterium of the genus Pseudomonas.

37. The method according to item 36, wherein the bacterium is Pseudomonas putida.

38. The method according to item 30, wherein the bacterium is a bacterium of the genus Corynebacterium.

39. The method according to item 38, wherein the bacterium is Corynebacterium glutamicum.

40. The method according to item 30, wherein the bacterium is a bacterium of the genus Escherichia.

41. The method according to item 40, wherein the bacterium is Escherichia coli.

42. The method according to any one of item 1 to 29, wherein the microorganism is a yeast.

43. The method according to item 42, wherein the yeast is of the genus Saccharomyces, Pichia, Schizosacharomyces, Zygosaccharomyces, Hansenula, Pachyosolen, Kluyveromyces, Debaryomyces, Yarrowia, Candida, Cryptococcus, Komagataella, Lipomyces, Rhodospiridium, Rhodotorula, or Trichosporon.

44. The method according to item 43, wherein the yeast is of the genus Saccharomyces.

45. The method according to item 44, wherein the yeast is Saccharomyces cerevisiae.

46. A genetically modified microorganism which comprises one or more of the following modifications a) to l):

a) an exogenous nucleic acid molecule comprising a nucleotide sequence encoding an inhibitory nucleic acid molecule that specifically hybridizes (e.g. binds) under cellular conditions with cellular mRNA and/or genomic DNA encoding an enzyme involved in the biosynthesis of a pyrimidine nucleotide;

b) an exogenous nucleic acid molecule comprising a nucleotide sequence encoding an inhibitory nucleic acid molecule that specifically hybridizes (e.g. binds) under cellular conditions with cellular mRNA and/or genomic DNA encoding an enzyme involved in the biosynthesis of a purine nucleotide;

c) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a pyrimidine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a pyrimidine nucleotide;

d) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a purine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a purine nucleotide;

e) a gene encoding an enzyme involved in the biosynthesis of a pyrimidine nucleotide, the regulatory sequence of said gene comprises a repressible promoter;

f) a gene encoding an enzyme involved in the biosynthesis of a pyrimidine nucleotide, the regulatory sequence of said gene comprises an operator; wherein the genetically modified microorganism further comprises an exogenous nucleic acid molecule comprising a nucleotide sequence encoding a repressor that is capable of binding to the operator;

g) a gene encoding an enzyme involved in the biosynthesis of a purine nucleotide, the regulatory sequence of said gene comprises a repressible promoter;

h) a gene encoding an enzyme involved in the biosynthesis of a purine nucleotide, the regulatory sequence of said gene comprises an operator; wherein the genetically modified microorganism further comprises an exogenous nucleic acid molecule comprising a nucleotide sequence encoding a repressor that is capable of binding to the operator; and

i) an inactivated gene encoding an enzyme involved in the biosynthesis of a pyrimidine nucleotide;

j) an inactivated gene encoding an enzyme involved in the biosynthesis of a purine nucleotide;

k) a gene encoding an enzyme involved in the biosynthesis of a pyrimidine nucleotide, wherein the gene comprises within the region encoding an UTR, such as a 5′-UTR, a nucleotide sequence encoding a riboswitch;

l) a gene encoding an enzyme involved in the biosynthesis of a purine nucleotide, wherein the gene comprises within the region encoding an UTR, such as a 5′-UTR, a nucleotide sequence encoding a riboswitch.

47. A genetically modified microorganism which comprises an exogenous nucleic acid molecule comprising a nucleotide sequence encoding an inhibitory nucleic acid molecule that specifically hybridizes (e.g. binds) under cellular conditions with an mRNA and/or gene encoding an enzyme involved in the biosynthesis of a pyrimidine nucleotide.

48. A genetically modified microorganism which comprises an exogenous nucleic acid molecule comprising a nucleotide sequence encoding an inhibitory nucleic acid molecule that specifically hybridizes (e.g. binds) under cellular conditions with an mRNA and/or gene encoding an enzyme involved in the biosynthesis of a purine nucleotide.

49. A genetically modified microorganism which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with a gene encoding an enzyme involved in the biosynthesis of a pyrimidine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with a gene encoding an enzyme involved in the biosynthesis of a pyrimidine nucleotide.

50. The genetically modified microorganism according to item 49 which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a pyrimidine nucleotide.

51. The genetically modified microorganism according to item 49, which comprises an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a pyrimidine nucleotide.

52. A genetically modified microorganism which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with a gene encoding an enzyme involved in the biosynthesis of a purine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a purine nucleotide.

53. The genetically modified microorganism according to item 52 which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a purine nucleotide.

54. The genetically modified microorganism according to item 52, which comprises an exogenous nucleic acid molecule comprising a nucleotide sequence encoding a catalytically inactive RNA-guided endonuclease, a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a purine nucleotide.

55. A genetically modified microorganism which comprises a gene encoding an enzyme involved in the biosynthesis of a pyrimidine nucleotide, the regulatory sequence of said gene comprises a repressible promoter.

56. A genetically modified microorganism which comprises a gene encoding an enzyme involved in the biosynthesis of a pyrimidine nucleotide, the regulatory sequence of said gene comprises an operator; wherein the genetically modified microorganism further comprises an exogenous nucleic acid molecule comprising a nucleotide sequence encoding a repressor that is capable of binding to the operator.

57. A genetically modified microorganism which comprises a gene encoding an enzyme involved in the biosynthesis of a purine nucleotide, the regulatory sequence of said gene comprises a repressible promoter.

58. A genetically modified microorganism which comprises a gene encoding an enzyme involved in the biosynthesis of a purine nucleotide, the regulatory sequence of said gene comprises an operator; wherein the genetically modified microorganism further comprises an exogenous nucleic acid molecule comprising a nucleotide sequence encoding a repressor that is capable of binding to the operator.

59. A genetically modified microorganism which comprises an inactivated gene encoding an enzyme involved in the biosynthesis of a pyrimidine nucleotide.

60. A genetically modified microorganism which comprises an inactivated gene encoding an enzyme involved in the biosynthesis of a purine nucleotide.

61. The genetically modified microorganism according to any one of items 46 to 60, which has been modified to have a down regulated biosynthesis of a pyrimidine or purine nucleotide compared to an otherwise identical microorganism that does not carry said modification.

62. The genetically modified microorganism according to any one of items 46 to 61, wherein the enzyme involved in the biosynthesis of a pyrimidine nucleotide is selected from the group consisting of an enzyme having orotidine-5′-phosphate decarboxylase activity, an enzyme having carbamoyl phosphate synthase activity, an enzyme having aspartate carbamoyltransferase activity, an enzyme having dihydroorotase activity, an enzyme having dihydroorotate dehydrogenase activity, an enzyme having orotate phosphoribosyltransferase activity, an enzyme having UMP kinase activity, an enzyme having nucleoside diphosphate kinase activity and an enzyme having CTP synthase activity.

63. The genetically modified microorganism according to any one of items 46 to 62, wherein the enzyme involved in the biosynthesis of a pyrimidine nucleotide is an enzyme having orotidine-5′-phosphate decarboxylase activity.

64. The genetically modified microorganisms according to any one of items 46 to 61, wherein the enzyme involved in the biosynthesis of a purine nucleotide is selected from the group consisting of an enzyme having amidophosphoribosyltransferase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N5-carboxyaminoimidazole ribonucleotide synthetase activity, an enzyme having N5-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, an enzyme having IMP cyclohydolase activity, an enzyme having adenylosuccinate synthase activity, an enzyme having adenylate kinase activity, an enzyme having ATP synthase activity, an enzyme having IMP dehydrogenase activity, an enzyme having GMP synthase activity, an enzyme having guanylate kinase activity, and an enzyme having nucleoside-diphosphate kinase activity.

65. The genetically modified microorganism according to any one of items 46 to 64, which further comprises (e.g., expresses) a heterologous polypeptide having tyrosine ammonia lyase activity.

66. The genetically modified microorganism according to any one of items 46 to 65, which further comprises (e.g., expresses) a heterologous polypeptide having an aryl sulfotransferase activity.

67. The genetically modified microorganism according to any one of items 46 to 66, which is a bacterium.

68. The genetically modified microorganism according to item 67, wherein the bacterium is a bacterium of the genus Escherichia, Bacillus, Lactococcus, Lactobacillus, Clostridium, Corynebacterium, Geobacillus, Streptococcus, Pediococcus, Moorella, Pseudomonas, Streptomyces, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus, or Yersinia.

69. The genetically modified microorganism according to item 67, wherein the bacterium is a bacterium of the genus Bacillus.

70. The genetically modified microorganism according to item 69, wherein the bacterium is Bacillus subtilis.

71. The genetically modified microorganism according to item 67, wherein the bacterium is a bacterium of the genus Lactococcus.

72. The genetically modified microorganism according to item 71, wherein the bacterium is Lactococcus lactis.

73. The genetically modified microorganism according to item 67, wherein the bacterium is a bacterium of the genus Pseudomonas.

74. The genetically modified microorganism according to item 73, wherein the bacterium is Pseudomonas putida.

75. The genetically modified microorganism according to item 67, wherein the bacterium is a bacterium of the genus Corynebacterium.

76. The genetically modified microorganism according to item 75, wherein the bacterium is Corynebacterium glutamicum.

77. The genetically modified microorganism according to item 67, wherein the bacterium is a bacterium of the genus Escherichia.

78. The genetically modified microorganism according to item 77, wherein the bacterium is Escherichia coli.

79. The genetically modified microorganism according to any one of items 46 to 66, which is a yeast.

80. The genetically modified microorganism according to item 79, wherein the yeast is of the genus Saccharomyces, Pichia, Schizosacharomyces, Zygosaccharomyces, Hansenula, Pachyosolen, Kluyveromyces, Debaryomyces, Yarrowia, Candida, Cryptococcus, Komagataella, Lipomyces, Rhodospiridium, Rhodotorula, or Trichosporon.

81. The genetically modified microorganism according to item 80, wherein the yeast is of the genus Saccharomyces.

82. The genetically modified microorganism according to item 81, wherein the yeast is Saccharomyces cerevisiae.

83. A method for decoupling cell growth from production of a biochemical compound in a microorganism, especially a microorganism having the ability to produce said biochemical compound, the method comprises inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

84. A method for the production of a biochemical compound, the method comprises:

a) growing a microorganism, especially a microorganism having an ability to produce said biochemical compound, in a culture medium; and

b) reducing the growth of the microorganism by inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

85. The method according to items 83 or 84, wherein the expression of a polypeptide encoded by the gene yheV is inhibited.

86. The method according to any one of items 83 to 85, wherein biochemical compound is L-tyrosine or a derivative thereof.

87. The method according to item 86, wherein the derivative is a hydroxycinnamic acid or derivative thereof.

88. The method according to item 87, wherein the hydroxycinnamic acid is p-coumaric acid.

89. The method according to item 87 or 88, wherein the hydroxycinnamic acid derivative is zosteric acid.

90. The method according to any one of items 87 to 89, wherein the microorganism comprises (e.g. expresses) a heterologous polypeptide having tyrosine ammonia lyase activity.

91. The method according to any one of items 87 to 90, wherein the microorganism comprises (e.g. expresses) a heterologous polypeptide having an aryl sulfotransferase activity.

92. The method according to any one of items 83 to 85, wherein the biochemical compound is mevalonate or a derivative thereof.

93. A method for the production of a recombinant polypeptide, the method comprises:

a) growing a microorganism, especially a microorganism having the ability to produce said recombinant polypeptide, in a culture medium; and

b) reducing the growth of the microorganism by inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

94. A method for decoupling cell growth from production of a recombinant polypeptide in a microorganism, especially a microorganism having the ability to produce said recombinant polypeptide, the method comprises inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

95. The method according to items 93 or 94, wherein the expression of a polypeptide encoded by the gene yheV is inhibited.

96. The method according to any one of items 83 to 95, wherein the expression is inhibited by transcriptional and/or translational repression of the gene encoding said polypeptide.

97. The method according to any one of items 83 to 96, wherein the expression is inhibited by introducing or expressing in the microorganism an inhibitory nucleic acid molecule that specifically hybridizes (e.g. binds) under cellular conditions with cellular mRNA and/or genomic DNA encoding said polypeptide.

98. The method according to item 97, wherein the inhibitory nucleic acid molecule is an antisense oligonucleotide, ribozyme or interfering RNA (RNAi) molecule.

99. The method according to item 98, wherein the interfering RNA molecule is a micro RNA (miRNA), small interfering RNA (siRNA) or short hairpin RNA (shRNA).

100. The method according to any one of items 97 to 99, wherein the expression of said inhibitory nucleic acid molecule is under the control of an inducible promoter, such as a temperature-inducible promoter.

101. The method according to any one of items 83 to 96, wherein the expression is inhibited by introducing or expressing in the microorganism a catalytically inactive RNA-guided endonuclease, such as a catalytically inactive Cas9 protein, and a single guide RNA (sgRNA) specifically hybridizing (e.g. binding) under cellular conditions with the genomic DNA encoding said polypeptide.

102. The method according to item 101, wherein the expression of the catalytically inactive RNA-guided endonuclease, such as the catalytically inactive Cas9 protein, and the single guide RNA (sgRNA) is under the control of an inducible promoter, such as a temperature-inducible promoter.

103. The method according to any one of items 83 to 96, wherein the expression of the at least one polypeptide is under the control of a repressible promoter.

104. The method according to any one of items 83 to 96, wherein the at least one polypeptide is encoded by a gene the regulatory sequence of which comprises a repressible promoter.

105. The method according to any one of items 83 to 96, wherein the at least one polypeptide is encoded by a gene the regulatory sequence of which comprises an operator located between the promoter and the open reading frame encoding said enzyme.

106. The method according to item 105, wherein the expression is inhibited by introducing or expressing in the microorganism a repressor that is capable of binding to the operator.

107. The method according to item 107, wherein the expression of the repressor is under the control of an inducible promoter, such as a temperature inducible promoter.

108. A method for decoupling cell growth from production a biochemical compound in a microorganism, especially a microorganism having the ability to produce said biochemical compound, the method comprises inhibiting the expression of SibB (small RNA antisense regulator of toxic lbsB protein) and/or increasing the expression of lbsB or a variant thereof.

109. A method for the production of a biochemical compound, the method comprises:

a) growing a microorganism, especially a microorganism having an ability to produce said biochemical compound, in a culture medium; and

b) reducing the growth of the microorganism by inhibiting the expression of SibB (small RNA antisense regulator of toxic lbsB protein) and/or increasing the expression of lbsB of a variant thereof.

110. The method according to item 108 or 109, wherein biochemical compound is L-tyrosine or a derivative thereof.

111. The method according to item 110, wherein the derivative is a hydroxycinnamic acid or derivative thereof.

112. The method according to item 111, wherein the hydroxycinnamic acid is p-coumaric acid.

113. The method according to item 111 or 112, wherein the hydroxycinnamic acid derivative is zosteric acid.

114. The method according to any one of items 111 to 113, wherein the microorganism comprises (e.g. expresses) a heterologous polypeptide having tyrosine ammonia lyase activity.

115. The method according to any one of items 111 to 114, wherein the microorganism comprises (e.g. expresses) a heterologous polypeptide having an aryl sulfotransferase activity.

116. The method according to item 108 or 109, wherein the biochemical compound is mevalonate or a derivative thereof.

117. A method for decoupling cell growth from production of a recombinant polypeptide in a microorganism, especially a microorganism having the ability to produce said recombinant polypeptide, the method comprises inhibiting the expression of SibB and/or increasing the expression of lbsB or a variant thereof.

118. A method for the production of a recombinant polypeptide, the method comprises:

a) growing a microorganism, especially a microorganism having the ability to produce said recombinant polypeptide, in a culture medium; and

b) reducing the growth of the microorganism by inhibiting the expression SibB and/or increasing the expression of lbsB or a variant thereof.

119. The method according to any one of items 108 to 118, wherein the expression of SibB is inhibited by transcriptional and/or translational repression of gene encoding SibB.

120. The method according to any one of items 108 to 119, wherein the expression of SibB is inhibited by introducing or expressing in the microorganism an inhibitory nucleic acid molecule that specifically hybridizes (e.g. binds) under cellular conditions with SibB or genomic DNA encoding SibB.

121. The method according to item 120, wherein the inhibitory nucleic acid molecule is an antisense oligonucleotide, ribozyme or interfering RNA (RNAi) molecule. 122. The method according to item 121, wherein the interfering RNA molecule is a micro RNA (miRNA), small interfering RNA (siRNA) or short hairpin RNA (shRNA).

123. The method according to any one of items 120 to 122, wherein the expression of said inhibitory nucleic acid molecule is under the control of an inducible promoter, such as a temperature-inducible promoter.

124. The method according to any one of items 108 to 119, wherein the expression of SibB is inhibited by introducing or expressing in the microorganism a catalytically inactive RNA-guided endonuclease, a catalytically inactive Cas9 protein, and a single guide RNA (sgRNA) specifically hybridizing (e.g. binding) under cellular conditions with genomic DNA encoding SibB.

125. The method according to item 124, wherein the expression of the catalytically inactive RNA-guided endonuclease, such as the catalytically inactive Cas9 protein, and the single guide RNA (sgRNA) is under the control of an inducible promoter, such as a temperature-inducible promoter.

126. The method according to any one of items 108 to 118, wherein the expression of SibB is under the control of a repressible promoter.

127. The method according to any one of items 108 to 118, wherein SibB is encoded by a gene the regulatory sequence of which comprises a repressible promoter.

128. The method according to any one of items 108 to 118, wherein SibB is encoded by a gene the regulatory sequence of which comprises an operator located between the promoter and the open reading frame encoding SibB.

129. The method according to item 128, wherein the expression of SibB is inhibited by introducing or expressing in the microorganism a repressor that is capable of binding to the operator.

130. The method according to item 129, wherein the expression of the repressor is under the control of an inducible promoter, such as a temperature inducible promoter.

131. The method according to any one of items 83 to 130, wherein the microorganism is a bacterium.

132. The method according to item 131, wherein the bacterium is a bacterium of the genus Escherichia, Bacillus, Lactococcus, Lactobacillus, Clostridium, Corynebacterium, Geobacillus, Thermoanaerobacterium, Streptococcus, Pediococcus, Moorella, Pseudomonas, Streptomyces, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus, or Yersinia.

133. The method according to item 131, wherein the bacterium is a bacterium of the genus Bacillus.

134. The method according to item 133, wherein the bacterium is Bacillus subtilis.

135. The method according to item 131, wherein the bacterium is a bacterium of the genus Lactococcus.

136. The method according to item 135, wherein the bacterium is Lactococcus lactis.

137. The method according to item 131, wherein the bacterium is a bacterium of the genus Pseudomonas.

138. The method according to item 137, wherein the bacterium is Pseudomonas putida.

139. The method according to item 131, wherein the bacterium is a bacterium of the genus Corynebacterium.

140. The method according to item 139, wherein the bacterium is Corynebacterium glutamicum.

141. The method according to item 131, wherein the bacterium is a bacterium of the genus Escherichia.

142. The method according to item 141, wherein the bacterium is Escherichia coli.

143. The method according to any one of item 83 to 130, wherein the microorganism is a yeast.

144. The method according to item 143, wherein the yeast is of the genus Saccharomyces, Pichia, Schizosacharomyces, Zygosaccharomyces, Hansenula, Pachyosolen, Kluyveromyces, Debaryomyces, Yarrowia, Candida, Cryptococcus, Komagataella, Lipomyces, Rhodospiridium, Rhodotorula, or Trichosporon.

147. The method according to item 143, wherein the yeast is of the genus Saccharomyces.

148. The method according to item 147, wherein the yeast is Saccharomyces cerevisiae.

149. A genetically modified microorganism which comprises one or more of the following modifications A-1) to F-1):

A-1) an exogenous nucleic acid molecule comprising a nucleotide sequence encoding an inhibitory nucleic acid molecule that specifically hybridizes (e.g. binds) under cellular conditions with cellular mRNA and/or genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes;

B-1) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes;

C-1) a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises a repressible promoter;

D-1) a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises an operator; wherein the genetically modified microorganism further comprises an exogenous nucleic acid molecule comprising a nucleotide sequence encoding a repressor that is capable of binding to the operator;

E-1) an inactivated gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes;

F-1) a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes; wherein the gene comprises within the region encoding an UTR, such as a 5′-UTR, a nucleotide sequence encoding a riboswitch.

150. A genetically modified microorganism which comprises an exogenous nucleic acid molecule comprising a nucleotide sequence encoding an inhibitory nucleic acid molecule that specifically hybridizes (e.g. binds) under cellular conditions with cellular mRNA and/or genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

151. A genetically modified microorganism which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

152. A genetically modified microorganism which comprises a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises a repressible promoter.

153. A genetically modified microorganism which comprises a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises an operator; wherein the genetically modified microorganism further comprises an exogenous nucleic acid molecule comprising a nucleotide sequence encoding a repressor that is capable of binding to the operator.

154. A genetically modified microorganism which comprises an inactivated gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

155. The genetically modified microorganism according to any one of items 149 to 154, which has a reduced expression of the polypeptide compared to an otherwise identical microorganism that does not carry said modification.

156. A genetically modified microorganism which comprises one or more of the following modifications A-2) to G-2):

A-2) an exogenous nucleic acid molecule comprising a nucleotide sequence encoding an inhibitory nucleic acid molecule that specifically hybridizes (e.g. binds) under cellular conditions with SibB and/or genomic DNA encoding SibB;

B-2) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding SibB; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding SibB;

C-2) a gene encoding SibB, the regulatory sequence of said gene comprises a repressible promoter;

D-2) a gene encoding SibB, the regulatory sequence of said gene comprises an operator; wherein the genetically modified microorganism further comprises an exogenous nucleic acid molecule comprising a nucleotide sequence encoding a repressor that is capable of binding to the operator;

E-2) an inactivated gene encoding SibB;

F-2) an exogenous nucleic acid molecule comprising a nucleotide sequence encoding a polypeptide comprising an amino acid sequence set forth in SEQ ID NO: 6, wherein the exogenous nucleic acid optionally comprises an inducible promoter that is functional in the microorganism to cause the production of an mRNA molecule the translation of which results in said polypeptide and that is operably linked to the nucleotide sequence encoding said polypeptide;

G-2) an exogenous nucleic acid molecule comprising a nucleotide sequence encoding a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 6, wherein the exogenous nucleic acid optionally comprises an inducible promoter that is functional in the microorganism to cause the production of an mRNA molecule the translation of which results in said polypeptide and that is operably linked to the nucleotide sequence encoding said polypeptide.

157. A genetically modified microorganism which comprises an exogenous nucleic acid molecule comprising a nucleotide sequence encoding an inhibitory nucleic acid molecule that specifically hybridizes (e.g. binds) under cellular conditions with SibB and/or genomic DNA encoding SibB.

158. A genetically modified microorganism which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding SibB.

159. A genetically modified microorganism which comprises an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding SibB.

160. A genetically modified microorganism which comprises a gene encoding SibB, the regulatory sequence of said gene comprises a repressible promoter.

161. A genetically modified microorganism which comprises a gene encoding SibB, the regulatory sequence of said gene comprises an operator; wherein the genetically modified microorganism further comprises an exogenous nucleic acid molecule comprising a nucleotide sequence encoding a repressor that is capable of binding to the operator.

162. A genetically modified microorganism which comprises an inactivated gene encoding SibB.

163. The genetically modified microorganism according to any one of items 149 to 162, which further comprises (e.g., expresses) a heterologous polypeptide having tyrosine ammonia lyase activity.

164. The genetically modified microorganism according to any one of items 149 to 163, which further comprises (e.g., expresses) a heterologous polypeptide having an aryl sulfotransferase activity.

165. The genetically modified microorganism according to any one of items 149 to 164, which is a bacterium.

166. The genetically modified microorganism according to item 165, wherein the bacterium is a bacterium of the genus Escherichia, Bacillus, Lactococcus, Lactobacillus, Clostridium, Corynebacterium, Geobacillus, Streptococcus, Pediococcus, Moorella, Pseudomonas, Streptomyces, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus, or Yersinia.

167. The genetically modified microorganism according to item 165, wherein the bacterium is a bacterium of the genus Bacillus.

168. The genetically modified microorganism according to item 167, wherein the bacterium is Bacillus subtilis.

169. The genetically modified microorganism according to item 165, wherein the bacterium is a bacterium of the genus Lactococcus.

170. The genetically modified microorganism according to item 169, wherein the bacterium is Lactococcus lactis.

171. The genetically modified microorganism according to item 165, wherein the bacterium is a bacterium of the genus Pseudomonas.

172. The genetically modified microorganism according to item 171, wherein the bacterium is Pseudomonas putida.

173. The genetically modified microorganism according to item 165, wherein the bacterium is a bacterium of the genus Corynebacterium.

174. The genetically modified microorganism according to item 173, wherein the bacterium is Corynebacterium glutamicum.

175. The genetically modified microorganism according to item 165, wherein the bacterium is a bacterium of the genus Escherichia.

176. The genetically modified microorganism according to item 175, wherein the bacterium is Escherichia coli.

177. The genetically modified microorganism according to any one of items 149 to 164, which is a yeast.

178. The genetically modified microorganism according to item 177, wherein the yeast is of the genus Saccharomyces, Pichia, Schizosacharomyces, Zygosaccharomyces, Hansenula, Pachyosolen, Kluyveromyces, Debaryomyces, Yarrowia, Candida, Cryptococcus, Komagataella, Lipomyces, Rhodospiridium, Rhodotorula, or Trichosporon.

179. The genetically modified microorganism according to item 177, wherein the yeast is of the genus Saccharomyces.

180. The genetically modified microorganism according to item 179, wherein the yeast is Saccharomyces cerevisiae.

### BRIEF DESCRIPTION OF THE DRAWINGS

FIG. 1: The effect on growth (dark grey) and expression (light grey) of recombinant protein (GFP) as a function of repression of certain genes. The values represent the ratio between induced and non-induced samples, where the CRISPRi system is used to repress the expression of selected genes.

FIG. 2: Growth profiling of strains carrying different growth switches. (A) The cell growth was measured as the optical density (OD) of cell cultures at 630 nm. The highest OD reached during growth phase is shown for each strain with or without induction. Error bars indicate standard deviations (n=3). A paired t-test was performed for the significance analysis where * and ** indicate p<0.05 or p<0.01, respectively. (B-F) Growth curves for each strain with or without induction, shown with standard deviations for each time point (n=3), reveal different patterns of inhibition.

FIG. 3: GFP production in bacterial strains expressing different growth switches. (A-E) The specific fluorescence measured for strains with or without induction of the CRISPRi systems. (F) The ratio of fluorescence, OD and specific fluorescence after 24 hours of incubation of induced and uninduced cultures. (G) Average fluorescence intensity measured from flow cytometry of different strains after 24 hours. (H) Average forward scatter measured from flow cytometry of different strains. Error bars indicate standard deviations (n=3), while * and ** indicate p<0.05 or p<0.01 in paired t-tests.

FIG. 4: Mevalonate production in strains with or without inhibition of cell growth. Specific mevalonate production (A) and mevalonate yield (B) of strains containing different growth switches. Parent represents the original MG1655 strain with pMevT encoding the mevalonate pathway (SoT17). Blank represent the control strain with pSLQ1236-blank (SoT96). Numbers are shown as mean values with standard deviations (n=2) (C-E) Cell density (OD600), mevalonate concentration and glucose concentration changes in induced cultures during cultivation. Data are shown as mean values with standard deviation (n=3).

FIG. 5: Characterization of production yield, cell density and specific production by applying 5-FU. The effect of 5-FU on production of mevalonate (left) and tyrosine (right). Data are shown as mean values and standard deviations (n=4 for mevalonate and n=3 for tyrosine), normalized to the values obtained in the control cultures, where no growth inhibitors were added in normal M9 media.

FIG. 6: Map of plasmid pSLQ1236 (pSon33)

FIG. 7: Map of plasmid pSLQ1236-dnaA (pSon37)

FIG. 8: Map of plasmid pSLQ1236-oriC (pSon38)

FIG. 9: Map of plasmid pSLQ1236-pyrF (pSon39)

FIG. 10: Map of plasmid pSLQ1236-thyA (pSon40)

FIG. 11: Map of plasmid pSLQ1236-nc (pSon44)

FIG. 12: Map of plasmid pSLQ1236-blank (pSon49)

FIG. 13: Map of plasmid CDP-GFP (pSon31)

FIG. 14: Growth profile (A) and fluorescence intensity over time (B) of the strains analyzed in the experiment. C represents both values recorded for B. subtilis 168 lacA::pJMP1 amyE::pJMP222 thrC::pDG1731-PS1-sfGFP after 32 h. Shaded areas on the growth profile and fluorescence measurements, and the error bars on the bar chart represents the standard deviations (n=3 biological replicates). Paired t-tests were performed for values shown in C, where * and ** indicates p<0.05 and p<0.01, respectively.

FIG. 15: Enzymes involved in purine and pyrimidine de novo biosynthesis in E. coli.

FIG. 16: GFP fluorescence (FITC-A) and growth (OD) for the induced/uninduced strains.

FIG. 17: Map of pCDF-Duet1-serAmut-serC-gRNA-pyrF under control of a tetracycline inducible promoter.

FIG. 18. Growth curves of strains as a function of time. Induction of dcas9 and pGRNA was performed 1.5 h after inoculation, while serine production was induced at O.D 0.6. The error bars indicate variations from duplicate biological replicates.

FIG. 19. Serine production (g/L) by the control strain and variants expressing gRNAs targeting different sites in the genome. The error bars indicate variation from duplicate biological replicates.

FIG. 20. Specific serine production (g/g dry cell weight) by the control strain and variants containing gRNAs targeting different sites in the genome. The error bars indicate variation from the duplicate biological replicates.

### DETAILED DESCRIPTION OF THE INVENTION

Unless specifically defined herein, all technical and scientific terms used have the same meaning as commonly understood by a skilled artisan in the fields of microbiology, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, and recombinant DNA, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); B. Perbal, A Practical Guide To Molecular Cloning (1984); and the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.).

Methods of the Invention

As indicated above, the present invention is inter alio based on the surprising finding that that fermentative production of biochemcial compounds, such as L-tyrosine and mevalonate, as well as the recombinant production of polypeptides by a microorganism can be enhanced by decoupling the production from cell growth through the down regulation of the biosynthesis of at least one type of nucleotide in the producing microorganism.

Accordingly, the present invention provides a method for decoupling cell growth from production of a biochemical compound in a microorganism, especially a microorganism having the ability to produce said biochemical compound, the method comprises inhibiting the expression and/or activity of at least one enzyme involved in the biosynthesis of at least one type of nucleotide.

The present invention also provides a method for the production of a biochemical compound, the method comprises:

a) growing a microorganism, especially a microorganism having an ability to produce said biochemical compound, in a culture medium; and

b) reducing the growth of the microorganism by inhibiting the expression and/or activity of at least one enzyme involved in the biosynthesis of at least one type of nucleotide in the microorganism.

The present invention also provides a method for decoupling cell growth from production of a recombinant polypeptide in a microorganism, especially a microorganism having the ability to produce said recombinant polypeptide, the method comprises inhibiting the expression and/or activity of at least one enzyme involved in the biosynthesis of at least one type of nucleotide.

The present invention also provides a method for the production of a recombinant polypeptide, the method comprises:

a) growing a microorganism, especially a microorganism having the ability to produce said recombinant polypeptide, in a culture medium; and

b) reducing the growth of the microorganism by inhibiting the expression and/or activity of at least one enzyme involved in the biosynthesis of at least one type of nucleotide.

The recombinant polypeptide may be any polypeptide one wishes to produce (e.g., express) by the microorganism. Suitably, the microorganism has been modified using, e.g., DNA recombination techniques, to comprise an exogenous nucleic acid molecule comprising a nucleotide sequence encoding said polypeptide operably linked to a promoter that is functional in the microorganism to cause the production of an mRNA molecule the translation of which results in said polypeptide.

According to certain embodiments, a method as detailed above comprises inhibiting the expression of at least one (such as at least two) enzyme involved in the biosynthesis of at least one type of nucleotide.

According to certain embodiments, a method as detailed above comprises inhibiting the activity of at least one enzyme (such as at least two) involved in the biosynthesis of at least one type of nucleotide.

According to certain embodiments, a method as detailed above comprises inhibiting the expression of at least one (such as at least two) enzyme involved in the biosynthesis of a pyrimidine nucleotide.

According to certain embodiments, a method as detailed above comprises inhibiting the expression of at least one (such as at least two) enzyme involved in the UMP biosynthesis pathway.

According to certain embodiments, a method as detailed above comprises inhibiting the activity of at least one (such as at least two) enzyme involved in the biosynthesis of a pyrimidine nucleotide.

According to certain embodiments, a method as detailed above comprises inhibiting the activity of at least one (such as at least two) enzyme involved in the UMP biosynthesis pathway.

According to certain embodiments, a method as detailed above comprises inhibiting the expression of at least one (such as at least two) enzyme involved in the biosynthesis of a purine nucleotide.

According to certain embodiments, a method as detailed above comprises inhibiting the expression of at least one (such as at least two) enzyme involved in the IMP biosynthesis pathway.

According to certain embodiments, a method as detailed above comprises inhibiting the activity of at least one (such as at least two) enzyme involved in the biosynthesis of a purine nucleotide.

According to certain embodiments, a method as detailed above comprises inhibiting the activity of at least one (such as at least two) enzyme involved in the IMP biosynthesis pathway.

The at least one enzyme involved in the biosynthesis of at least one type of nucleotide (such as a pyrimidine or purine nucleotide) which expression and/or activity is inhibited may be an enzyme selected from the group consisting of: an enzyme having orotidine-5′-phosphate decarboxylase activity, an enzyme having carbamoyl phosphate synthase activity, an enzyme having aspartate carbamoyltransferase activity, an enzyme having dihydroorotase activity, an enzyme having dihydroorotate dehydrogenase activity, an enzyme having orotate phosphoribosyltransferase activity, an enzyme having UMP kinase activity, an enzyme having nucleoside diphosphate kinase activity, an enzyme having cytidylate kinase activity, an enzyme having CTP synthase activity, an enzyme having amidophosphoribosyltransferase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N5-carboxyaminoimidazole ribonucleotide synthetase activity, an enzyme having N5-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, an enzyme having IMP cyclohydolase activity, an enzyme having adenylosuccinate synthase activity, an enzyme having adenylate kinase activity, an enzyme having ATP synthase activity, an enzyme having IMP dehydrogenase activity, an enzyme having GMP synthase activity, an enzyme having guanylate kinase activity, an enzyme having nucleoside-diphosphate kinase activity, an enzyme having pyruvate kinase II activity, an enzyme having GMP reductase activity, an enzyme having deoxyguanosine triphosphate triphosphohydrolase activity, an enzyme having ribonucleoside-diphosphate reductase activity, an enzyme having ribonucleoside-triphosphate reductase activity, an enzyme having dTMP kinase activity, and an enzyme having deoxyuridine triphosphatase activity, an enzyme having thymidylate synthase activity.

According to particular embodiments, the at least one enzyme involved in the biosynthesis of a pyrimidine nucleotide is selected from the group consisting of: an enzyme having orotidine-5′-phosphate decarboxylase activity, an enzyme having carbamoyl phosphate synthase activity, an enzyme having aspartate carbamoyltransferase activity, an enzyme having dihydroorotase activity, an enzyme having dihydroorotate dehydrogenase activity, an enzyme having orotate phosphoribosyltransferase activity, an enzyme having UMP kinase activity, an enzyme having nucleoside diphosphate kinase activity, an enzyme having cytidylate kinase activity and an enzyme having CTP synthase activity.

According to particular embodiments, the at least one enzyme involved in the biosynthesis of a pyrimidine nucleotide is selected from the group consisting of: an enzyme having orotidine-5′-phosphate decarboxylase activity, an enzyme having carbamoyl phosphate synthase activity, an enzyme having aspartate carbamoyltransferase activity, an enzyme having dihydroorotase activity, an enzyme having dihydroorotate dehydrogenase activity, an enzyme having orotate phosphoribosyltransferase activity, an enzyme having UMP kinase activity, an enzyme having nucleoside diphosphate kinase activity and an enzyme having CTP synthase activity.

According to particular embodiments, the at least one enzyme involved in the UMP biosynthesis pathway is selected from the group consisting of: an enzyme having orotidine-5′-phosphate decarboxylase activity, an enzyme having carbamoyl phosphate synthase activity, an enzyme having aspartate carbamoyltransferase activity, an enzyme having dihydroorotase activity, an enzyme having dihydroorotate dehydrogenase activity, and an enzyme having orotate phosphoribosyltransferase activity.

According to particular embodiments, the at least one enzyme involved in the biosynthesis of a pyrimidine nucleotide is an enzyme having orotidine-5′-phosphate decarboxylase activity.

According to particular embodiments, the at least one enzyme involved in the biosynthesis of a pyrimidine nucleotide is an enzyme having carbamoyl phosphate synthase activity.

According to particular embodiments, the at least one enzyme involved in the biosynthesis of a pyrimidine nucleotide is an enzyme having aspartate carbamoyltransferase activity.

According to particular embodiments, the at least one enzyme involved in the biosynthesis of a pyrimidine nucleotide is an enzyme having dihydroorotase activity.

According to particular embodiments, the at least one enzyme involved in the biosynthesis of a pyrimidine nucleotide is an enzyme having dihydroorotate dehydrogenase activity.

According to particular embodiments, the at least one enzyme involved in the biosynthesis of a pyrimidine nucleotide is an enzyme having orotate phosphoribosyltransferase activity.

According to particular embodiments, the at least one enzyme involved in the biosynthesis of a pyrimidine nucleotide is an enzyme having UMP kinase activity.

According to particular embodiments, the at least one enzyme involved in the biosynthesis of a pyrimidine nucleotide is an enzyme having nucleoside diphosphate kinase activity.

According to particular embodiments, the at least one enzyme involved in the biosynthesis of a pyrimidine nucleotide is an enzyme having CTP synthase activity.

According to particular embodiments, the at least one enzyme involved in the biosynthesis of a pyrimidine nucleotide is an enzyme having cytidylate kinase activity.

According to particular embodiments, the at least one enzyme involved in the biosynthesis of a purine nucleotide is selected from the group consisting of: an enzyme having amidophosphoribosyltransferase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N5-carboxyaminoimidazole ribonucleotide synthetase activity, an enzyme having N5-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, an enzyme having IMP cyclohydolase activity, an enzyme having adenylosuccinate synthase activity, an enzyme having adenylate kinase activity, an enzyme having ATP synthase activity, an enzyme having IMP dehydrogenase activity, an enzyme having GMP synthase activity, an enzyme having guanylate kinase activity, and an enzyme having nucleoside-diphosphate kinase activity.

According to particular embodiments, the at least one enzyme involved in the IMP biosynthesis pathway is selected from the group consisting of: an enzyme having amidophosphoribosyltransferase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N5-carboxyaminoimidazole ribonucleotide synthetase activity, an enzyme having N5-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, and an enzyme having IMP cyclohydolase activity.

According to particular embodiments, the at least one enzyme involved in the biosynthesis of a purine nucleotide is an enzyme having amidophosphoribosyltransferase activity.

According to particular embodiments, the at least one enzyme involved in the biosynthesis of a purine nucleotide is an enzyme having phosphoribosylamine-glycine ligase activity.

According to particular embodiments, the at least one enzyme involved in the biosynthesis of a purine nucleotide is an enzyme having phosphoribosylglycineamide formyltransferase activity.

According to particular embodiments, the at least one enzyme involved in the biosynthesis of a purine nucleotide is an enzyme having phosphoribosylformylglycinamidine synthase activity.

According to particular embodiments, the at least one enzyme involved in the biosynthesis of a purine nucleotide is an enzyme having phosphoribosylformylglycineamidine cyclo-ligase activity.

According to particular embodiments, the at least one enzyme involved in the biosynthesis of a purine nucleotide is an enzyme having N5-carboxyaminoimidazole ribonucleotide synthetase activity.

According to particular embodiments, the at least one enzyme involved in the biosynthesis of a purine nucleotide is an enzyme having N5-carboxyaminoimidazole ribonucleotide mutase activity.

According to particular embodiments, the at least one enzyme involved in the biosynthesis of a purine nucleotide is an enzyme having phosphoribosylaminoimidazolesuccino-carboxamide synthase activity.

According to particular embodiments, the at least one enzyme involved in the biosynthesis of a purine nucleotide is an enzyme having adenylosuccinate lyase activity.

According to particular embodiments, the at least one enzyme involved in the biosynthesis of a purine nucleotide is an enzyme having phosphoribosylaminoimidazole-carboxamide formyltransferase activity.

According to particular embodiments, the at least one enzyme involved in the biosynthesis of a purine nucleotide is an enzyme having IMP cyclohydolase activity.

According to particular embodiments, the at least one enzyme involved in the biosynthesis of a purine nucleotide is an enzyme having adenylosuccinate synthase activity.

According to particular embodiments, the at least one enzyme involved in the biosynthesis of a purine nucleotide is an enzyme having adenylate kinase activity.

According to particular embodiments, the at least one enzyme involved in the biosynthesis of a purine nucleotide is an enzyme having ATP synthase activity.

According to particular embodiments, the at least one enzyme involved in the biosynthesis of a purine nucleotide is an enzyme having IMP dehydrogenase activity.

According to particular embodiments, the at least one enzyme involved in the biosynthesis of a purine nucleotide is an enzyme having GMP synthase activity.

According to particular embodiments, the at least one enzyme involved in the biosynthesis of a purine nucleotide is an enzyme having guanylate kinase activity.

According to particular embodiments, the at least one enzyme involved in the biosynthesis of a purine nucleotide is an enzyme having nucleoside-diphosphate kinase activity.

In addition to genes and respectively encoded enzymes which are involved in the nucleotide biosynthesis, the present inventors have also identified other genes which when repressed lead to a decoupling of growth from production, exemplified by the recombinant production of GFP in Escherichia coli. As shown in Example 1, the repression of certain genes can be used to repress or inhibit the growth of a production microorganism, and at the same time increase the production of recombinant proteins (exemplified by the expression of GFP). In particular, lpxC, yaiY(p), ydiB, sibB, yheV, ygaQ, glcA, yjeN and malZ were found to reduce growth while significantly increasing recombinant protein expression in the cell. Moreover, the inhibiting the expression of SibB (small RNA antisense regulator of toxic lbsB protein) of the toxin/anti-toxin system sibB/ibsB provides a significant 5-fold increase in GFP production as indicated by an increased fluorescence per cell.

Therefore, the present invention also provides a method for decoupling cell growth from production of a recombinant polypeptide in a microorganism, especially a microorganism having the ability to produce said recombinant polypeptide, the method comprises inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

The present invention also provides a method for the production of a recombinant polypeptide, the method comprises:

a) growing a microorganism, especially a microorganism having the ability to produce said recombinant polypeptide, in a culture medium; and

b) reducing the growth of the microorganism by inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

The recombinant polypeptide may be any polypeptide one wishes to produce (e.g., express) by the microorganism. Suitably, the microorganism has been modified using, e.g., DNA recombination techniques, to comprise an exogenous nucleic acid molecule comprising a nucleotide sequence encoding said polypeptide operably linked to a promoter that is functional in the microorganism to cause the production of an mRNA molecule the translation of which results in said polypeptide.

The present invention also provides a method for decoupling cell growth from production of a biochemical compound, such as L-tyrosine or a derivative thereof, in a microorganism, especially a microorganism having the ability to produce said biochemical compound, the method comprises inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

The present invention also provides a method for the production of a biochemical compound, such as L-tyrosine or a derivative thereof, the method comprises:

a) growing a microorganism, especially a microorganism having an ability to produce said biochemical compound, in a culture medium; and

b) reducing the growth of the microorganism by inhibiting the expression of at least one polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

According to certain embodiments, the expression of a polypeptide encoded by the gene lpxC or an ortholog thereof is inhibited. Further information regarding lpxC of, e.g., Escherichia coli is available at EcoCyc (www.biocyc.org) or EcoGene (www.ecogene.org) under Accession number EG10265. See also NCBI Reference Sequence: NP_414638.1 for the amino acid sequence (E. coli).

According to certain embodiments, the expression of a polypeptide encoded by the gene yaiY or an ortholog thereof is inhibited. Further information regarding yaiY of, e.g., Escherichia coli is available at EcoCyc (www.biocyc.org) or EcoGene (www.ecogene.org) under Accession number EG14279. See also NCBI Reference Sequence: NP_414913.1 for the amino acid sequence (E. coli).

According to certain embodiments, the expression of a polypeptide encoded by the gene ydiB or an ortholog thereof is inhibited. Further information regarding ydiB of, e.g., Escherichia coli is available at EcoCyc (www.biocyc.org) or EcoGene (www.ecogene.org) under Accession number EG11234. See also NCBI Reference Sequence: NP_416207.1 for the amino acid sequence (E. coli).

According to certain embodiments, the expression of a polypeptide encoded by the gene yheV or an ortholog thereof is inhibited. Further information regarding yheV of, e.g., Escherichia coli is available at EcoCyc (www.biocyc.org) or EcoGene (www.ecogene.org) under Accession number EG14364. See also NCBI Reference Sequence: YP_588468.1 for the amino acid sequence (E. coli). A representative nucleotide sequence of the E. coli yheV gene is set forth in SEQ ID NO: 3.

According to certain embodiments, the expression of a polypeptide encoded by the gene ygaQ or an ortholog thereof is inhibited. Further information regarding ygaQ of, e.g., Escherichia coli is available at EcoCyc (www.biocyc.org) or EcoGene (www.ecogene.org) under Accession number EG13520. See also NCBI Reference Sequence: NP_417140.1 for the amino acid sequence (E. coli).

According to certain embodiments, the expression of a polypeptide encoded by the gene glcA or an ortholog thereof is inhibited. Further information regarding glcA of, e.g., Escherichia coli is available at EcoCyc (www.biocyc.org) or EcoGene (www.ecogene.org) under Accession number EG12995. See also NCBI Reference Sequence: NP_417449.1 for the amino acid sequence (E. coli).

According to certain embodiments, the expression of a polypeptide encoded by the gene yjeN or an ortholog thereof is inhibited. Further information regarding yjeN of, e.g., Escherichia coli is available at EcoCyc (www.biocyc.org) or EcoGene (www.ecogene.org) under Accession number EG12476. See also NCBI Reference Sequence: NP_418581.1 for the amino acid sequence (E. coli).

According to certain embodiments, the expression of a polypeptide encoded by the gene malZ or an ortholog thereof is inhibited. Further information regarding malZ of, e.g., Escherichia coli is available at EcoCyc (www.biocyc.org) or EcoGene (www.ecogene.org) under Accession number EG10565. See also NCBI Reference Sequence: NP_414937.1 for the amino acid sequence (E. coli).

The present invention also provides a method for decoupling cell growth from production of a recombinant polypeptide in a microorganism, especially a microorganism having the ability to produce said recombinant polypeptide, the method comprises inhibiting the expression of SibB (small RNA antisense regulator of toxic lbsB protein) and/or increasing the expression of lbsB or a variant thereof.

According to certain embodiments, the present invention provides a method for decoupling cell growth from production of a recombinant polypeptide in a microorganism, especially a microorganism having the ability to produce said recombinant polypeptide, the method comprises inhibiting the expression of SibB (small RNA antisense regulator of toxic lbsB protein).

According to certain embodiments, the present invention provides a method for decoupling cell growth from production of a recombinant polypeptide in a microorganism, especially a microorganism having the ability to produce said recombinant polypeptide, the method comprises increasing the expression lbsB or a variant thereof.

The present invention also provides a method for the production of a recombinant polypeptide, the method comprises:

a) growing a microorganism, especially a microorganism having the ability to produce said recombinant polypeptide, in a culture medium; and

b) reducing the growth of the microorganism by inhibiting the expression of SibB and/or increasing the expression of lbsB or a variant thereof.

According to certain embodiments, the present invention provides a method for the production of a recombinant polypeptide, the method comprises:

a) growing a microorganism, especially a microorganism having the ability to produce said recombinant polypeptide, in a culture medium; and

b) reducing the growth of the microorganism by inhibiting the expression of SibB.

According to certain embodiments, the present invention provides a method for the production of a recombinant polypeptide, the method comprises:

a) growing a microorganism, especially a microorganism having the ability to produce said recombinant polypeptide, in a culture medium; and

b) reducing the growth of the microorganism by increasing the expression of lbsB or a variant thereof.

The recombinant polypeptide may be any polypeptide one wishes to produce (e.g., express) by the microorganism. Suitably, the microorganism has been modified using, e.g., DNA recombination techniques, to comprise an exogenous nucleic acid molecule comprising a nucleotide sequence encoding said polypeptide operably linked to a promoter that is functional in the microorganism to cause the production of an mRNA molecule the translation of which results in said polypeptide.

The present invention also provides a method for decoupling cell growth from production of a biochemical compound, such as L-tyrosine or a derivative thereof, in a microorganism, especially a microorganism having the ability to produce L-tyrosine or a derivative thereof, the method comprises inhibiting the expression of SibB (small RNA antisense regulator of toxic lbsB protein) and/or increasing the expression of lbsB or a variant thereof.

According to certain embodiments, the present invention also provides a method for decoupling cell growth from production of a biochemical compound, such as L-tyrosine or a derivative thereof, in a microorganism, especially a microorganism having the ability to produce L-tyrosine or a derivative thereof, the method comprises inhibiting the expression of SibB (small RNA antisense regulator of toxic lbsB protein).

According to certain embodiments, the present invention also provides a method for decoupling cell growth from production of a biochemical compound, such as L-tyrosine or a derivative thereof, in a microorganism, especially a microorganism having the ability to produce L-tyrosine or a derivative thereof, the method comprises increasing the expression of lbsB.

The present invention also provides a method for the production of a biochemical compound, such as L-tyrosine or a derivative thereof, the method comprises:

a) growing a microorganism, especially a microorganism having an ability to produce said biochemical compound, in a culture medium; and

b) reducing the growth of the microorganism by inhibiting the expression of SibB (small RNA antisense regulator of toxic lbsB protein) and/or increasing the expression of lbsB or a variant thereof.

The present invention also provides a method for the production of a biochemical compound, such as L-tyrosine or a derivative thereof, the method comprises:

a) growing a microorganism, especially a microorganism having an ability to produce said biochemical compound, in a culture medium; and

b) reducing the growth of the microorganism by inhibiting the expression of SibB (small RNA antisense regulator of toxic lbsB protein).

The present invention also provides a method for the production of a biochemical compound, such as L-tyrosine or a derivative thereof, the method comprises:

a) growing a microorganism, especially a microorganism having an ability to produce said biochemical compound, in a culture medium; and

b) reducing the growth of the microorganism by increasing the expression of lbsB or a variant thereof.

Further information regarding sibB of, e.g., Escherichia coli is available at EcoCyc (www.biocyc.org) or EcoGene (www.ecogene.org) under Accession number EG31152. A representative nucleotide sequence of the E. coli sibB gene is set forth in SEQ ID NO: 4. A representative RNA sequence of the E. coli SibB is set forth in SEQ ID NO: 5.

Further information regarding lbsB of, e.g., Escherichia coli is available at EcoCyc (www.biocyc.org) or EcoGene (www.ecogene.org) under Accession number EG14473. A representative amino acid sequence of the E. coli lbsB is set forth in SEQ ID NO: 6.

A variant of lbsB is a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 6. Preferably, the variant of lbsB is toxic. With “toxic” it is meant that the variant of lbsB reduces the growth of the producing microorganism. Suitably, the toxicity of the variant of lbsB is similar to that of a polypeptide comprising the amino acid sequence set forth in SEQ ID NO: 6. With “similar” toxicity it is meant that the variant of lbsB has at least about 10%, such as at least about 20%, at least about 30%, at least about 40%, at least about 50%, at least about 60, at least about 75%, at least about 80%, at least about 90%, at least about 95%, at least about 100%, at least about 200%, at least about 200%, at least about 400% or at least about 800%, of the toxicity of the reference polypeptide (e.g., SEQ ID NO: 6).

According to certain embodiments, the genetically modified microorganism comprises an exogenous nucleic acid molecule comprising a nucleotide sequence encoding a polypeptide comprising an amino acid sequence set forth in SEQ ID NO: 6 or a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 6. Suitably, the exogenous nucleic acid molecule comprising an inducible promoter that is functional in the microorganism to cause the production of an mRNA molecule the translation of which results in said polypeptide and that is operably linked to the nucleotide sequence encoding said polypeptide.

According to certain embodiments, the genetically modified microorganism comprises an exogenous nucleic acid molecule comprising a nucleotide sequence encoding a polypeptide comprising an amino acid sequence set forth in SEQ ID NO: 6.

According to certain embodiments, the genetically modified microorganism comprises an exogenous nucleic acid molecule comprising a nucleotide sequence encoding a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 6. Preferably, the polypeptide is toxic. Suitably, the toxicity of the polypeptide is similar to that of the polypeptide comprising an amino acid sequence set forth in SEQ ID NO: 6.

In order to obtain a high nominal yield and/or mass yield of product, accumulation of a certain cell biomass concentration is desirable before cell growth is decoupled from production. Therefore, according to certain embodiments, the microorganism is grown in step a) to a desired cell density before step b) is initiated. The desired cell density may be any cell density one considered being sufficient for production. A desirable cell density range for production of biochemical compounds and recombinant polypeptide could for example be from about 1×10⁸ to about 1×10¹¹ cells/ml of culture.

According to certain embodiments, the microorganism is grown to a cell density of at least about 1×10⁸ cells/ml of culture. According to certain embodiments, the microorganism is grown to a cell density of at least about 5×10⁸ cells/ml of culture. According to certain embodiments, the microorganism is grown to a cell density of at least about 8×10⁸ cells/ml of culture. According to certain embodiments, the microorganism is grown to a cell density of at least about 1×10⁹ cells/ml of culture. According to certain embodiments, the microorganism is grown to a cell density of at least about 5×10⁹ cells/ml of culture. According to certain embodiments, the microorganism is grown to a cell density of at least about 8×10⁹ cells/ml of culture. According to certain embodiments, the microorganism is grown to a cell density of at least about 1×10¹⁰ cells/ml of culture. According to certain embodiments, the microorganism is grown to a cell density of at least about 5×10¹⁰ cells/ml of culture. According to certain embodiments, the microorganism is grown to a cell density of at least about 8×10¹⁰ cells/ml of culture.

According to certain embodiments, the microorganism is grown to a cell density in the range from about 1×10⁸ to about 1×10¹¹ cells/ml of culture. According to certain embodiments, the microorganism is grown to a cell density in the range from about 5×10⁸ to about 1×10¹¹ cells/ml of culture. According to certain embodiments, the microorganism is grown to a cell density in the range from about 1×10⁹ to about 1×10¹¹ cells/ml of culture. According to certain embodiments, the microorganism is grown to a cell density in the range from about 5×10⁹ to about 1×10¹¹ cells/ml of culture. According to certain embodiments, the microorganism is grown to a cell density in the range from about 1×10¹⁰ to about 1×10¹¹ cells/ml of culture. According to certain embodiments, the microorganism is grown to a cell density in the range from about 1×10⁸ to about 1×10¹⁰ cells/ml of culture. According to certain embodiments, the microorganism is grown to a cell density in the range from about 5×10⁸ to about 1×10¹⁰ cells/ml of culture. According to certain embodiments, the microorganism is grown to a cell density in the range from about 1×10⁹ to about 1×10¹⁰ cells/ml of culture. According to certain embodiments, the microorganism is grown to a cell density in the range from about 5×10⁹ to about 1×10¹⁰ cells/ml of culture. According to certain embodiments, the microorganism is grown to a cell density in the range from about 1×10⁸ to about 5×10⁹ cells/ml of culture. According to certain embodiments, the microorganism is grown to a cell density in the range from about 5×10⁸ to about 1×10⁹ cells/ml of culture.

According to certain embodiments, the microorganism is grown to a cell density OD600 of at least about 1. According to certain embodiments, the microorganism is grown to a cell density OD600 of at least about 2.5. According to certain embodiments, the microorganism is grown to a cell density OD600 of at least about 5. According to certain embodiments, the microorganism is grown to a cell density OD600 of at least about 10. According to certain embodiments, the microorganism is grown to a cell density OD600 of at least about 20. According to certain embodiments, the microorganism is grown to a cell density OD600 of at least about 50.

According to certain embodiments, the microorganism is grown to a cell density OD600 in the range from about 1 to about 150. According to certain embodiments, the microorganism is grown to a cell density OD600 in the range from about 2.5 to about 150. According to certain embodiments, the microorganism is grown to a cell density OD600 in the range from about 5 to about 150. According to certain embodiments, the microorganism is grown to a cell density OD600 in the range from about 10 to about 150. According to certain embodiments, the microorganism is grown to a cell density OD600 in the range from about 1 to about 100. According to certain embodiments, the microorganism is grown to a cell density OD600 in the range from about 2.5 to about 100. According to certain embodiments, the microorganism is grown to a cell density OD600 in the range from about 5 to about 100. According to certain embodiments, the microorganism is grown to a cell density OD600 in the range from about 10 to about 100. According to certain embodiments, the microorganism is grown to a cell density OD600 in the range from about 20 to about 150. According to certain embodiments, the microorganism is grown to a cell density OD600 in the range from about 20 to about 100. According to certain embodiments, the microorganism is grown to a cell density OD600 in the range from about 50 to about 100. According to certain embodiments, the microorganism is grown to a cell density OD600 in the range from about 20 to about 80.

Optical density (OD) can be measured using a spectrophotometer. For measuring optical density of a culture, the sample is diluted to an appropriate concentration as needed, and the absorbance of the sample is measured with a spectrophotometer (for example VWR model UV-1600PC) at 600 nm with a 1 cm cuvette filled with 1 mL of sample. The spectrophotometer is first blanked on the original fermentation medium prior to measuring the absorbance of the sample. The accuracy of the method is the highest when the absorbance is between 0.1 and 0.5. The optical density of the culture can be calculated from the measurements taking the dilution factor into account.

The cell biomass concentration during fermentation could also be measured as Dry Cell Weight (DCW) and is often measured in g/L. A desirable DCW range for production of biochemical compounds and recombinant polypeptides may be for example from 1.5 g/L to 60 g/L of culture.

According to certain embodiments, the microorganism is grown to a cell density of at least about 1.5 g/L (g dry cell weight/L of culture). According to certain embodiments, the microorganism is grown to a cell density of at least about 1.75 g/L (g dry cell weight/L of culture). According to certain embodiments, the microorganism is grown to a cell density of at least about 2.5 g/L (g dry cell weight/L of culture). According to certain embodiments, the microorganism is grown to a cell density of at least about 3.5 g/L (g dry cell weight/L of culture). According to certain embodiments, the microorganism is grown to a cell density of at least about 5 g/L (g dry cell weight/L of culture). According to certain embodiments, the microorganism is grown to a cell density of at least about 7.5 g/L (g dry cell weight/L of culture). According to certain embodiments, the microorganism is grown to a cell density of at least about 10 g/L (g dry cell weight/L of culture). According to certain embodiments, the microorganism is grown to a cell density of at least about 15 g/L (g dry cell weight/L of culture). According to certain embodiments, the microorganism is grown to a cell density of at least about 15 g/L (g dry cell weight/L of culture). According to certain embodiments, the microorganism is grown to a cell density of at least about 15 g/L (g dry cell weight/L of culture).

According to certain embodiments, the microorganism is grown to a cell density in the range from about 1.5 to about 60 g/L (g dry cell weight/L of culture). According to certain embodiments, the microorganism is grown to a cell density in the range from about 1.75 to about 60 g/L (g dry cell weight/L of culture). According to certain embodiments, the microorganism is grown to a cell density in the range from about 2.5 to about 60 g/L (g dry cell weight/L of culture). According to certain embodiments, the microorganism is grown to a cell density in the range from about 3.5 to about 60 g/L (g dry cell weight/L of culture). According to certain embodiments, the microorganism is grown to a cell density in the range from about 5 to about 60 g/L (g dry cell weight/L of culture). According to certain embodiments, the microorganism is grown to a cell density in the range from about 7.5 to about 60 g/L (g dry cell weight/L of culture). According to certain embodiments, the microorganism is grown to a cell density in the range from about 10 to about 60 g/L (g dry cell weight/L of culture). According to certain embodiments, the microorganism is grown to a cell density in the range from about 15 to about 60 g/L (g dry cell weight/L of culture). According to certain embodiments, the microorganism is grown to a cell density in the range from about 1.5 to about 30 g/L (g dry cell weight/L of culture). According to certain embodiments, the microorganism is grown to a cell density in the range from about 1.75 to about 30 g/L (g dry cell weight/L of culture). According to certain embodiments, the microorganism is grown to a cell density in the range from about 2.5 to about 30 g/L (g dry cell weight/L of culture). According to certain embodiments, the microorganism is grown to a cell density in the range from about 3.5 to about 30 g/L (g dry cell weight/L of culture). According to certain embodiments, the microorganism is grown to a cell density in the range from about 5 to about 30 g/L (g dry cell weight/L of culture). According to certain embodiments, the microorganism is grown to a cell density in the range from about 7.5 to about 30 g/L (g dry cell weight/L of culture). According to certain embodiments, the microorganism is grown to a cell density in the range from about 10 to about 30 g/L (g dry cell weight/L of culture). According to certain embodiments, the microorganism is grown to a cell density in the range from about 15 to about 30 g/L (g dry cell weight/L of culture).

Well described methods are available for determining the DCW of a fermentation sample.

The culture medium employed may be any conventional medium suitable for culturing the microorganism in question, and may be composed according to the principles of the prior art. The medium will usually contain all nutrients necessary for the growth and survival of the respective microorganism, such as carbon and nitrogen sources and other inorganic salts. Suitable media, e.g. minimal or complex media, are available from commercial suppliers, or may be prepared according to published receipts, e.g. the American Type Culture Collection (ATCC) Catalogue of strains. Non-limiting standard medium well known to the skilled person include Luria Bertani (LB) broth, Sabouraud Dextrose (SD) broth, MS broth, Yeast Peptone Dextrose, BMMY, GMMY, or Yeast Malt Extract (YM) broth, which are all commercially available. A non-limiting example of suitable media for culturing bacterial cells, such as B. subtilis, L. lactis or E. coli cells, including minimal media and rich media such as Luria Broth (LB), M9 media, M17 media, SA media, MOPS media, Terrific Broth, YT and others. Suitable media for culturing eukaryotic cells, such as yeast cells, are RPMI 1640, MEM, DMEM, all of which may be supplemented with serum and/or growth factors as required by the particular host cell being cultured. The medium for culturing eukaryotic cells may also be any kind of minimal media such as Yeast minimal media.

The fermentable carbon substrate may be any suitable carbon substrate known in the art, and in particularly any carbon substrate commonly used in the cultivation of microorganisms and/or fermentation. Non-limiting examples of suitable fermentable carbon substrates include carbohydrates (e.g., C5 sugars such as arabinose or xylose, or C6 sugars such as glucose), glycerol, glycerine, acetate, dihydroxyacetone, one-carbon source, methanol, methane, oils, animal fats, animal oils, plant oils, fatty acids, lipids, phospholipids, glycerolipids, monoglycerides, diglycerides, triglycerides, renewable carbon sources, polypeptides (e.g., a microbial or plant protein or peptide), yeast extract, component from a yeast extract, peptone, casaminoacids or any combination of two or more of the foregoing.

According to certain embodiments, the carbon substrate is selected from the group consisting of C5 sugars (such as arabinose or xylose), C6 sugars (such as glucose or fructose), lactose, sucrose, glycerol, glycerine, acetate, Corn steep liquor, yeast extract, component from a yeast extract, peptone, casaminoacids or combinations thereof.

According to certain embodiments, the medium comprises glucose. According to certain embodiments, the medium comprises glycerol. According to certain embodiments, the medium comprises acetate.

It is also contemplated to use starch as a carbon substrate. Depending on the microorganism used, the metabolization of starch may require the supplementation of beta-glucosidase, such as the beta-glucosidase from Neurospora crassa, to the medium. Alternatively, a microorganism may be further genetically modified to comprise (e.g., express) a beta-glucosidase, such as the beta-glucosidase from Neurospora crassa.

When a fermentable carbon substrate is employed it is thus possible that the microorganism produces the biochemical compound, such as L-tyrosine or mevalonate, directly from such primary carbon substrate.

Suitably, the microorganism is cultivated under suitable conditions for the production of the desired product. Suitable conditions for culturing the respective microorganism are well known to the skilled person. Typically, a microorganism is cultured at a temperature ranging from about 23 to about 60° C., such as from about 25 to about 40° C., such as at about 37° C. The pH of the culture medium may range from pH 1.0 to pH 14.0, such as from about pH 1 to about pH 2, from about pH 4 to about pH 11, from about pH 5 to about pH 10, from about pH 6 to about pH 10, or from about pH 7 to about pH 9.5, e.g. at pH 6.0, pH 7.0, pH 7.5, pH 8.0, pH 8.5, pH 9.0, pH 9.5, pH 10.0, pH 10.5 or pH 11.0. Preferably, the pH of the culture medium is in the range from about pH 7 to about pH 9.5, such as at about pH 7.5.

The production methods of the present invention may further comprise the step of recovering the produced biochemical compound (such as L-tyrosine or a derivative thereof) or recombinant polypeptide. The produced biochemical compound or recombinant polypeptide may be recovered by conventional method for isolation and purification from a medium. Well-known purification procedures include centrifugation or filtration, precipitation, and chromatographic methods such as e.g. ion exchange chromatography, gel filtration chromatography, etc.

Means for Inhibiting Expression According to the Invention

Inhibition of the expression of a polypeptide (such as an enzyme as described herein, such as an enzyme having orotidine-5′-phosphate decarboxylase activity) may be achieved by any suitable means known in the art. For example, the expression may be inhibited by gene silencing techniques involving the use of inhibitory nucleic acid molecules, such as antisense oligonucleotides, ribozymes or interfering RNA (RNAi) molecules, such as microRNA (miRNA), small interfering RNA (siRNA) or short hairpin RNA (shRNA). Also contemplated by the present invention is the use of the CRISPRi system. These techniques may also be employed to inhibit the expression of SibB, and the details provided below apply mutatis mutandis.

According to certain embodiments, the expression is inhibited by introducing or expressing in the microorganism an inhibitory nucleic acid molecule. For example, the inhibitory nucleic acid molecule may be introduced by way of an exogenous nucleic acid molecule comprising a nucleotide sequence encoding said inhibitory nucleic acid molecule operably linked to a promoter, such as an inducible promoter, that is functional in the microorganism to cause the production of said inhibitory nucleic acid molecule. Suitably, the inhibitory nucleic acid molecule is one that specifically hybridizes (e.g. binds) under cellular conditions with cellular mRNA and/or genomic DNA encoding the polypeptide or enzyme of interest (such as an enzyme having orotidine-5′-phosphate decarboxylase activity). Depending on the target, transcription of the encoding genomic DNA and/or translation of the encoding mRNA is/are inhibited. In case of SibB, the inhibitory nucleic acid molecule is one that specifically hybridizes (e.g. binds) under cellular conditions with genomic DNA encoding SibB.

According to certain embodiments, the inhibitory nucleic acid molecule is an antisense oligonucleotide, ribozyme or interfering RNA (RNAi) molecule. Preferably, such nucleic acid molecule comprises at least 10 consecutive nucleotides of the complement of the cellular mRNA and/or genomic DNA encoding the polypeptide or enzyme of interest (e.g., the cellular mRNA and/or genomic DNA encoding an enzyme having orotidine-5′-phosphate decarboxylase activity).

By way of example, if the expression of an enzyme having orotidine-5′-phosphate decarboxylase activity is to be inhibited in Escherichia coli, such inhibitory nucleic acid molecule may comprise at least 10 consecutive nucleotides of the complement of SEQ ID NO: 1. Similarly, if the expression of an enzyme having orotidine-5′-phosphate decarboxylase activity is to be inhibited in Saccharomyces cerevisiae, such inhibitory nucleic acid molecule may comprise at least 10 consecutive nucleotides of the complement of SEQ ID NO: 2.

By way of further example, if the expression of the polypeptide encoded by the gene yheV is to be inhibited in Escherichia coli, such inhibitory nucleic acid molecule may comprise at least 10 consecutive nucleotides of the complement of SEQ ID NO: 3. Likewise, if the expression of SibB is to be inhibited in Escherichia coli, such inhibitory nucleic acid molecule may comprise at least 10 consecutive nucleotides of the complement of SEQ ID NO: 4 or SEQ ID NO: 5.

According to certain embodiments, the inhibitory nucleic acid is an antisense oligonucleotide. Such antisense oligonucleotide is a nucleic acid molecule (either DNA or RNA) which specifically hybridizes (e.g. binds) under cellular conditions with the cellular mRNA and/or genomic DNA encoding the polypeptide or enzyme of interest (e.g., the mRNA encoding an enzyme having orotidine-5′-phosphate decarboxylase activity). The binding may be by conventional base pair complementarity. Alternatively, the binding may be, for example, in case of binding to DNA duplexes, through specific interactions in the major groove of the double helix. Absolute complementarity, although preferred, is not required.

Antisense oligonucleotides employed according to the invention may be DNA or RNA or chimeric mixtures or derivatives or modified versions thereof, and may be single-stranded or double stranded. Thus, according to certain embodiment, the antisense oligonucleotide is a single-stranded or double-stranded DNA molecule, preferably a double-stranded DNA molecule. According to other certain embodiments, the antisense oligonucleotide is a single-stranded or double-stranded RNA molecule, preferably a single-stranded RNA molecule.

According to certain embodiments, the antisense oligonucleotide is a modified oligonucleotide which is resistant to endogenous nucleases, e.g., exonucleases and/or endonucleases, and is therefore stable in vivo and in vitro.

The antisense oligonucleotide may be modified at the base moiety, sugar moiety, or phosphate backbone, for example, to improve stability of the molecule. The antisense oligonucleotide may include other appended groups such as peptides (e.g., for targeting host cell receptors), or agents facilitating transport across the cell membrane. Hence, the antisense oligonucleotide may be conjugated to another molecule such as a peptide or transport agent.

According to certain embodiments, the antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w and 2,6-diaminopurine.

According to certain embodiments, the antisense oligonucleotide comprises at least one modified sugar moiety selected from the group including, but not limited to, arabinose, 2-fluoroarabinose, xylulose and hexose.

According to certain embodiments, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

An antisense oligonucleotide may be delivered into the microorganism, for example, in form of an expression vector, such as a plasmid or viral vector, which, when transcribed in the microorganism, produces RNA which is complementary to at least a unique portion of the cellular mRNA encoding the polypeptide or enzyme of interest. Alternatively, the antisense oligonucleotide may be generated ex vivo and introduced into the microorganism by any known means in the art. The antisense oligonucleotide may be synthesized ex vivo by standard method known in the art, e.g., by use of an automated DNA synthesizer (such as automated DNA synthesizer are commercially available from, e.g., Applied Biosystems). A number of methods have been developed for delivering antisense DNA or RNA to cells, e.g. by direct injection or through modification designed to target the desired microorganism (e.g., using antisense oligonucleotides linked to peptides or antibodies that specifically bind receptors or antigens expressed on the surface of the target microorganism.

According to certain embodiments, a recombinant DNA vector is used in which a nucleotide sequence coding for an antisense oligonucleotide inhibiting the expression of polypeptide or enzyme of interest (such as an enzyme having orotidine-5′-phosphate decarboxylase activity) is placed under the control of a promoter, preferably under the control of an inducible promoter, such as a temperature-inducible promoter. The use of such a construct to transfect a target microorganism, such as a bacterium, will result in the transcription of a sufficient amount of single-stranded RNA that will form complementary base pairs with the endogenous transcript and thereby prevent translation of the mRNA encoding the polypeptide or enzyme of interest (such as an enzyme having orotidine-5′-phosphate decarboxylase activity). In accordance with these embodiments, a DNA vector comprising the nucleotide sequence encoding the antisense oligonucleotide is introduced into the microorganism where the transcription of an antisense RNA occurs. Such vector can remain episomal or be chromosomally integrated, as long as it can be transcribed to produce the antisense RNA. The expression of the sequence encoding the antisense RNA can be under the control of a promoter known in the art to act in a microorganism, such as a bacterium. Preferably, such promoter is an inducible promoter, such as a temperature-inducible promoter. An inducible promoter allows the expression of the sequence encoding the antisense RNA to occur at the desired time point if a physical or chemical stimulus is present, such as a change in temperature or the presence of a chemical substance (“chemical inducer”).

Alternatively, antisense cDNA constructs that synthesize antisense RNA, either constitutively or inducibly, although inducibly is preferred, can be introduced into the microorganism.

By way of example, if the expression of an enzyme having orotidine-5′-phosphate decarboxylase activity is to be inhibited in Escherichia coli, the antisense oligonucleotide may comprise at least 10 consecutive nucleotides of the complement of SEQ ID NO: 1. In case of a double stranded molecule, such double-stranded antisense oligonucleotide comprises a first strand comprising at least 10 consecutive nucleotide of SEQ ID NO: 1, and a second strand complementary to said first strand. In case of a single-stranded molecule, such single-stranded oligonucleotide comprises at least 10 consecutive nucleotides of the complement of SEQ ID NO: 1.

Similarly, if the expression of an enzyme having orotidine-5′-phosphate decarboxylase activity is to be inhibited in Saccharomyces cerevisiae, the antisense oligonucleotide may comprise at least 10 consecutive nucleotides of the complement of SEQ ID NO: 2. In case of a double stranded molecule, such double-stranded antisense oligonucleotide comprises a first strand comprising at least 10 consecutive nucleotide of SEQ ID NO: 2, and a second strand complementary to said first strand. In case of a single-stranded molecule, such single-stranded oligonucleotide comprises at least 10 consecutive nucleotides of the complement of SEQ ID NO: 2.

By way of further example, if the expression of the polypeptide encoded by the gene yheV is to be inhibited in Escherichia coli, the antisense oligonucleotide may comprise at least 10 consecutive nucleotides of the complement of SEQ ID NO: 3. In case of a double stranded molecule, such double-stranded antisense oligonucleotide comprises a first strand comprising at least 10 consecutive nucleotide of SEQ ID NO: 3, and a second strand complementary to said first strand. In case of a single-stranded molecule, such single-stranded oligonucleotide comprises at least 10 consecutive nucleotides of the complement of SEQ ID NO: 3.

Likewise, if the expression of SibB is to be inhibited in Escherichia coli, the antisense oligonucleotide may comprise at least 10 consecutive nucleotides of the complement of SEQ ID NO: 4. In case of a double stranded molecule, such double-stranded antisense oligonucleotide comprises a first strand comprising at least 10 consecutive nucleotide of SEQ ID NO: 4, and a second strand complementary to said first strand. In case of a single-stranded molecule, such single-stranded oligonucleotide comprises at least 10 consecutive nucleotides of the complement of SEQ ID NO: 4 or SEQ ID NO: 5.

The antisense oligonucleotide may comprise a nucleotide sequence complementary to a non-coding or a coding region of the mRNA encoding the polypeptide or enzyme of interest. According to certain embodiments, the antisense oligonucleotide comprises a nucleotide sequence complementary to the 5′ end of the mRNA, e.g., the 5′ untranslated sequence up to and including the AUG initiation codon. According to other embodiments, the antisense oligonucleotide comprises a nucleotide sequence complementary to the 3′ untranslated sequence of the mRNA. According to other embodiments, the antisense oligonucleotide comprises a nucleotide sequence complementary to the coding region of the mRNA. Whether designed to hybridize to the 5′, 3′ or coding region of the mRNA, an antisense oligonucleotide should be at least six nucleotides in length, preferably at least 10 nucleotides in length, and is preferably less than about 100, and more preferably less than about 50, 25, 20, 15 or 10 nucleotides in length. According to particular embodiments, the antisense oligonucleotide is 6 to 25, such as 10 to 25 nucleotides in length.

In accordance with certain embodiments, the inhibitory nucleic acid molecule is a ribozyme. A ribozyme molecule is designed to catalytically cleave the mRNA transcript to prevent translation of the polypeptide or enzyme of interest.

According to certain embodiments, the ribozyme is a hammerhead ribozyme. Hammerhead ribozymes cleave mRNAs at locations dictated by flanking regions that form complementary base pairs with the target mRNA, e.g. the mRNA encoding an enzyme having orotidine-5′-phosphate decarboxylase activity. The sole requirement is that the target mRNA has the following sequence of two bases: 5′-UG-3′. The constructions and production of hammerhead ribozymes is well known in the art and is described in more detail in Haseloff and Gerlach (1988). In accordance with certain embodiments, the ribozyme is engineered such that the cleavage recognition site is located near the 5′ end of the target mRNA, e.g. the mRNA encoding an enzyme having orotidine-5′-phosphate decarboxylase activity. This increases the efficiency and minimizes the intracellular accumulation of non-functional mRNA transcripts.

Like with antisense oligonucleotides, a ribozyme used in accordance with the invention may be composed of modified oligonucleotides to, e.g., improve stability. The ribozyme may be introduced into the microorganism by any means known in the art. The ribozyme may be introduced into the microorganism in form of an expression vector, such as a plasmid or viral vector, which, when transcribed in the microorganism, produces the ribozyme. According to certain embodiments, a recombinant DNA vector is used in which a nucleotide sequence coding for the ribozyme is placed under the control of a promoter, preferably under the control of an inducible promoter, such as a temperature-inducible promoter, so that a transformed or transfected microorganism will produce sufficient amounts of the ribozyme to destroy endogenous mRNA and inhibit translation. Because ribozymes, unlike antisense oligonucleotides, are catalytic, a lower intracellular concentration is required for efficiency.

In accordance with certain embodiments, the inhibitory nucleic acid molecule is an interfering RNA (RNAi) molecule. RNA interference is a biological process in which RNA molecules inhibit gene expression, typically causing the destruction of specific mRNA. Exemplary types of RNAi molecules include microRNA (miRNA), small interfering RNA (siRNA) and short hairpin RNA (shRNA). According to particular embodiments, the RNAi molecule is a miRNA. According to other embodiments, the RNAi molecule is a siRNA. According to yet other embodiments, the RNAi molecule is a shRNA. The production of RNAi molecules in vivo and in vitro and their methods of use are described in, e.g., U.S. Pat. No. 6,506,559, WO 01/36646, WO 00/44895, US2002/01621126, US2002/0086356, US2003/0108923, WO 02/44321, WO 02/055693, WO 02/055692 and WO 03/006477.

By way of example, if the expression of an enzyme having orotidine-5′-phosphate decarboxylase activity is to be inhibited in Escherichia coli, the RNAi molecule may be an interfering RNA complementary to SEQ ID NO: 1. The RNAi molecule may be a ribonucleic acid molecule comprising at least 10 consecutive nucleotides of the complement of SEQ ID NO: 1. The RNAi molecule may be a double-stranded ribonucleic acid molecule comprising a first strand identical to 20 to 25, such as 21 to 23, consecutive nucleotides of SEQ ID NO: 1, and a second strand complementary to said first strand.

Similarly, if the expression of an enzyme having orotidine-5′-phosphate decarboxylase activity is to be inhibited in Saccharomyces cerevisiae, the RNAi molecule may be an interfering RNA complementary to SEQ ID NO: 2. The RNAi molecule may be a ribonucleic acid molecule comprising at least 10 consecutive nucleotides of the complement of SEQ ID NO: 1. The RNAi molecule may be a double-stranded ribonucleic acid molecule comprising a first strand identical to 20 to 25, such as 21 to 23, consecutive nucleotides of SEQ ID NO: 2, and a second strand complementary to said first strand.

By way of further example, if the expression of the polypeptide encoded by the gene yheV is to be inhibited in Escherichia coli, the RNAi molecule may be an interfering RNA complementary to SEQ ID NO: 3. The RNAi molecule may be a ribonucleic acid molecule comprising at least 10 consecutive nucleotides of the complement of SEQ ID NO: 3. The RNAi molecule may be a double-stranded ribonucleic acid molecule comprising a first strand identical to 20 to 25, such as 21 to 23, consecutive nucleotides of SEQ ID NO: 3, and a second strand complementary to said first strand.

Likewise, if the expression of SibB is to be inhibited in Escherichia coli, the RNAi molecule may be an interfering RNA complementary to SEQ ID NO: 4 or SEQ ID NO: 5. The RNAi molecule may be a ribonucleic acid molecule comprising at least 10 consecutive nucleotides of the complement of SEQ ID NO: 4 or SEQ ID NO: 5. The RNAi molecule may be a double-stranded ribonucleic acid molecule comprising a first strand identical to 20 to 25, such as 21 to 23, consecutive nucleotides of SEQ ID NO: 4 or SEQ ID NO: 5, and a second strand complementary to said first strand.

According to certain embodiments, the expression is inhibited using the CRISPRi system.

The CRISPRi system was developed as a tool for targeted repression of gene expression or for blocking targeted locations on the genome (Qi et al., 2013). The CRISPRi system consists of the catalytically inactive, “dead” Cas9 protein (dCas9) and a guide RNA that defines the binding site for the dCas9 to DNA. Cas9 is the effector protein of the type II clustered regularly interspaced short palindromic repeat (CRISPR) immune system of Streptococcus pyogenes and functions as a RNA-guided endonuclease (Carroll, 2012; Jinek et al., 2012). In the CRISPRi system, the wild-type S. pyogenes cas9 nuclease has been made catalytically inactive through mutations (for example D10A and H841A) that inactivate the RuvC1 and HNH nuclease domains (Jinek et al., 2012). By changing the target sequence of the guide RNA, the dCas9 can be guided to any location on the genome for which a guide RNA can be designed. In principle, any Cas9 protein could be engineered and used in similar ways.

The specificity of the native CRISPR system comes from two noncoding RNAs called CRISPR-RNA (crRNA) and trans-activating crRNA (tracrRNA). The specificity is brought about by the crRNA that base pairs to the target DNA. The target site must be adjacent to a protospacer adjacent motif (PAM) consisting of a random nucleotide and two guanines (NGG) (Jinek et al., 2012; Mali et al., 2013). The tracrRNA molecule together with crRNA functions as a scaffold onto which the Cas9 protein binds. A chimeric RNA that combines the crRNA and tracrRNA termed single guide RNA (sgRNA) has been applied (see for example DiCarlo et al., 2013). In the case of S. pyogenes nuclease, the sgRNA scaffold can be programmed for a specific site by including 20 bp of the target locus at the 5′ position of the double guanine PAM motif (NGG) (20N-NGG), where N designates the specific target sequence. It is also possible to reprogram Cas9 by using tracrRNA and a synthetic array containing 30 bp of the target (5′ of NGG) embedded between two repeat regions that will be subsequently be processed in the mature crRNA (Deltcheva et al., 2011). In these cases, the PAM motif is not included in the target sequence used for the sgRNA or crRNA array.

According to certain embodiments, the expression is inhibited by introducing or expressing in the microorganism a catalytically inactive RNA-guided endonuclease and a single guide RNA (sgRNA) specifically hybridizing (e.g. binding) under cellular conditions with the genomic DNA encoding a polypeptide or enzyme of interest (such as an enzyme having orotidine-5′-phosphate decarboxylase activity.

For example, the catalytically inactive RNA-guided endonuclease and the single guide RNA (sgRNA) may be introduced by way of an exogenous nucleic acid molecule comprising a nucleotide sequence encoding the catalytically inactive RNA-guided endonuclease and a nucleotide sequence encoding the single guide RNA (sgRNA); or by introducing an exogenous nucleic acid molecule comprising a nucleotide sequence encoding the catalytically inactive RNA-guided endonuclease and an exogenous nucleic acid molecule comprising a nucleotide sequence encoding the single guide RNA (sgRNA). To drive transcription of the catalytically inactive RNA-guided endonuclease and single guide RNA (sgRNA) the nucleotide sequences are operably linked to a promoter, such as an inducible promoter, that is functional in the microorganism to cause the production of the catalytically inactive RNA-guided endonuclease and single guide RNA (sgRNA).

According to certain embodiments, the expression is inhibited by introducing or expressing in the microorganism a catalytically inactive Cas9 protein and a single guide RNA (sgRNA) specifically hybridizing (e.g. binding) under cellular conditions with the genomic DNA encoding a polypeptide or enzyme of interest (such as an enzyme having orotidine-5′-phosphate decarboxylase activity.

For example, the catalytically inactive Cas9 protein and a single guide RNA (sgRNA) may be introduced by way of an exogenous nucleic acid molecule comprising a nucleotide sequence encoding the catalytically inactive Cas9 protein and a nucleotide sequence encoding the single guide RNA (sgRNA); or by introducing an exogenous nucleic acid molecule comprising a nucleotide sequence encoding the catalytically inactive Cas9 protein and an exogenous nucleic acid molecule comprising a nucleotide sequence encoding the single guide RNA (sgRNA). To drive transcription of the catalytically inactive Cas9 protein and single guide RNA (sgRNA) the nucleotide sequences are operably linked to a promoter, such as an inducible promoter, that is functional in the microorganism to cause the production of the catalytically inactive Cas9 protein and single guide RNA (sgRNA).

By way of example, if the expression of an enzyme having orotidine-5′-phosphate decarboxylase activity is to be inhibited in Escherichia coli, the single guide RNA (sgRNA) may comprise at least 20 consecutive nucleotides of SEQ ID NO: 1 or its complement.

Similarly, if the expression of an enzyme having orotidine-5′-phosphate decarboxylase activity is to be inhibited in Saccharomyces cerevisiae, the single guide RNA (sgRNA) may comprise at least 20 consecutive nucleotides of SEQ ID NO: 2 or its complement.

Non-limiting examples of single guide RNAs (sgRNA) targeting genes encoding for enzymes involved in the biosynthesis of nucleotides in, e.g., Escherichia coli are provided in Table 5 below.

By way of further example, if the expression of the polypeptide encoded by the gene yheV is to be inhibited in Escherichia coli, the single guide RNA (sgRNA) may comprise at least 20 consecutive nucleotides of SEQ ID NO: 3 or its complement.

Likewise, if the expression of SibB is to be inhibited in Escherichia coli, the single guide RNA (sgRNA) may comprise at least 20 consecutive nucleotides of SEQ ID NO: 4 or its complement.

An alternative approach in inhibiting expression is by modifying the microorganism to render the endogenous promoter of the gene of interest (such as gene encoding an enzyme as described herein, such as a gene encoding an enzyme having orotidine-5′-phosphate decarboxylase activity) regulatable, and more specifically repressible. In this respect, the microorganism may be modified by replacing the endogenous promoter of the gene of interest (such as gene encoding an enzyme as described herein, such as a gene encoding an enzyme having orotidine-5′-phosphate decarboxylase activity) by an exogenous regulatable promoter, and more particularly by a repressible promoter. Promoter replacements are frequently used to regulate the expression of genes in a specific manner such as for their conditional expression. Chromosomal integration of a regulatable promoter, such as a repressible promoter, upstream of an open reading frame (ORF) by, e.g., homologous recombination using PCR-based gene targeting is well known.

The term “repressible” used in the context of a promoter means that the transcriptional activity is decrease or inhibited if a repressing agent (“repressor”), such as a repressor protein, is present. Suitable repressible promoter systems functional in microorganisms are well known in the art and may be employed in accordance with the present invention. Non-limiting examples of repressible promoters include TetR-repressible promoters, Lacl-repressible promoters, LuxR-repressible promoter, which have been shown to regulate expression in bacteria. Other non-limiting examples of repressible promoters are the pL and/or pR λ phage promoters which are regulated by the thermolabile cl857 repressor.

In certain embodiments, the repressible promoter is a TetR-repressible promoter and is regulated by a Tet repressor. The TetR-repressible promoter may comprise at least one tetO sequence. In certain embodiments, the repressible promoter is a Lacl-repressible promoter and is regulated by the Lacl repressor.

Other non-limiting examples of repressible promoters are those from the gene of ANB1, HEM 13, ERG 11, OLE 1, GAL1, GAL10, ADH2, or TETR, which have been shown to regulate expression in yeast.

Suitably, the expression of the repressor protein in the microorganism is itself under the control of an inducible promoter. This way the repression of the repressible promoter by the repressor protein can be timely controlled. Suitable inducible promoter systems are well known in the art and are described in more detail below. Thus, according to certain embodiments, the microorganism comprise an exogenous nucleic acid molecule comprising a nucleotide sequence encoding the repressor protein operably linked to an inducible promoter that is functional in the microorganism to cause the production of the repressor.

The repressible promoter may also be a chemically-repressible promoter. Chemically-repressible promoters are promoters whose transcriptional activity is decrease or inhibited by the presence a chemical substance (“chemical inducer”), such as metal or other compounds.

Alternatively to replacing the endogenous promoter of the gene of interest (such as gene encoding an enzyme as described herein, such as a gene encoding an enzyme having orotidine-5′-phosphate decarboxylase activity) by a exogenous regulatable promoter, and more particularly by an exogenous repressible promoter, the endogenous promoter itself may be rendered regulatable, respectively repressible, by introducing an operator between the endogenous promoter and the open reading frame encoding the polypeptide of interest (such as an enzyme as described herein, such as an enzyme having orotidine-5′-phosphate decarboxylase activity). The expression of the polypeptide of interest may then be inhibited by introducing or expressing in the microorganism a repressor that is capable of binding to the operator. If the repressor itself is a protein, the microorganism may further be modified to comprise an exogenous nucleic acid molecule comprising a nucleotide sequence encoding the repressor operably linked to an inducible promoter that is functional in the microorganism to cause the production of the repressor.

Also contemplated for inhibiting the expression is the use of a riboswitch which is located in the UTR, such as the 5′-UTR, of an mRNA molecule encoding for a polypeptide of interest (such as an enzyme as described herein, such as an enzyme having orotidine-5′-phosphate decarboxylase activity). A riboswitch is a regulatory segment of a mRNA molecule that binds a small molecule, resulting in a change in expression of the polypeptide encoded by the mRNA. Thus, a mRNA that contains a riboswitch is directly involved in regulating its own activity, in response to its effector molecule. Following small molecule binding, expression is inhibited by transcription termination, translation inhibition or mRNA degradation processes. Suitable riboswitches which may be employed in accordance of the invention are well known in the art. See, e.g., Aghdam et al. (2016) for a detailed review. Non-limiting examples include Cobalamin riboswitch (also B12-element) which binds adenosylcobalamin, SAH riboswitches which bind S-adenosylhomocysteine, cyclic di-GMP riboswitches which bind cyclic di-GMP, FMN riboswitch (also RFN-element) which binds flavin mononucleotide (FMN), glmS riboswitch which is a ribozyme that cleaves itself when there is a sufficient concentration of glucosamine-6-phosphate, PreQ1 riboswitches which bind pre-queuosine1, SAH riboswitches which bind S-adenosylhomocysteine, SAM riboswitches which bind S-adenosyl methionine (SAM), SAM-SAH riboswitches which bind both SAM and SAH with similar affinities, and tetrahydrofolate riboswitches which bind tetrahydrofolate and TPP riboswitches (also THI-box) which binds thiamin pyrophosphate (TPP). It is also possible to use novel engineered riboswitches that have been derived from aptamer sequences. Well described methods, such as for example SELEX, are available for developing such aptamer sequences having specificity towards desired ligands.

Thus, according to certain embodiments, the microorganism comprises a gene encoding for a polypeptide of interest (such as an enzyme as described herein, such as an enzyme having orotidine-5′-phosphate decarboxylase activity; wherein said gene comprises in the region which encodes an UTR, such as a 5′-UTR, a nucleotide sequence encoding a riboswitch. The expression of the polypeptide of interest may then be inhibited by exposing the microorganism to the respective small molecule which binds to the riboswitch leading to transcription termination, translation inhibition or mRNA degradation.

Means for Inhibiting Activity According to the Invention

Inhibition of the activity of an enzyme as described herein (such as an enzyme having orotidine-5′-phosphate decarboxylase activity) may be achieved by any suitable means known in the art. For example, the activity may be inhibited by exposing the microorganism to an inhibitor of the enzyme. Suitable inhibitors for each enzyme are well known in the art.

By way of example, if the activity of an enzyme having orotidine-5′-phosphate decarboxylase activity is to be inhibited, the inhibitor may be, but is not limited to, 5-Fluoroorotic acid (5-FOA), 6-Azauridine-5′-monophosphate (6-Aza-UMP), 1-ribosylallopurinol-5′-phosphate or 6-iodouridine-5′-monophosphate (6-iodo-UMP) among others.

Biochemical Compounds Produced According to the Invention

A biochemical compound to be produced by any of the methods of the invention, or which production is decoupled from the growth of the producing microorganism in accordance of the present invention, may be any carbon-containing compound which can be produced by a living microorganism.

According to certain embodiments, the biochemical compound is an amino acid or a derivative thereof.

According to certain embodiments, the biochemical compound is an L-amino acid or a derivative thereof.

According to certain embodiments, the biochemical compound is a L-amino acid selected from the group consisting of: L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine and L-valine.

According to certain embodiments, the biochemical compound is L-tyrosine or a derivative thereof.

According to certain embodiments, the L-tyrosine derivative is a hydroxycinnamic acid or derivative thereof.

According to certain embodiments, the hydroxycinnamic acid is selected from the group consisting of: p-coumaric acid, caffeic acid and ferulic acid.

According to certain embodiments, the L-tyrosine derivative is a compound selected from the group consisting of: p-coumaric acid, caffeic acid, ferulic acid, vanillin, vanillic acid, cinnamic acid, resveratrol, naringenin, fisetin, curcumin and morphine.

In order to convert L-tyrosine into the desired derivative, such as p-coumaric acid, the microorganism suitably comprises (e.g., expresses) one or more enzymes catalyzing the chemical reaction(s) leading to the desired derivative. Table 1 below provides an overview of L-tyrosine derivatives and the enzymes involved in the conversion of L-tyrosine into the respective derivative. The microorganism may inherently express the one or more enzymes or may be modified to express the one or more enzymes by using, e.g., DNA recombination techniques.

TABLE 1
L-tyrosine derivatives and the enzyme(s) involved in the conversion of L-tyrosine
into said derivatives.

| Derivative | Enzyme(s) |
|---|---|
| p-coumaric acid | Tyrosine ammonia-lyase (EC 4.3.1.23) |
| (1 enzymatic step) | |
| zosteric acid | Tyrosine ammonia-lyase (EC 4.3.1.23); Aryl |
| (2 enzymatic steps) | sulfotransferase (EC: 2.8.2.1) |
| Caffeic acid | tyrosine ammonia lyase (EC 4.3.1.23); p-coumarate 3- |
| (2 enzymatic steps) | hydroxylase (EC1.14.13.) |
| Ferulic acid | tyrosine ammonia lyase (EC 4.3.1.23); p-coumarate 3- |
| (3 enzymatic steps) | hydroxylase (EC1.14.13.); caffeic acid 3-O- |
| | methyltransferase (EC 2.1.1.68) |
| Vanillin | tyrosine ammonia lyase (EC 4.3.1.23); p-coumarate 3- |
| (6 enzymatic steps) | hydroxylase (EC1.14.13.); caffeic acid 3-O- |
| | methyltransferase (EC 2.1.1.68); trans-feruloyl-CoA |
| | synthase (EC 6.2.1.34); trans-feruloyl-CoA hydratase (EC |
| | 4.2.1.101); vanillin synthase (4.1.2.41) |
| Vanillic acid | tyrosine ammonia lyase (EC 4.3.1.23); p-coumarate 3- |
| (7 enzymatic steps) | hydroxylase (EC1.14.13.); caffeic acid 3-O- |
| | methyltransferase (EC 2.1.1.68); trans-feruloyl-CoA |
| | synthase (EC 6.2.1.34); trans-feruloyl-CoA hydratase (EC |
| | 4.2.1.101); vanillin synthase (4.1.2.41); vanillin dehydrogenase |
| | (EC 1.2.1.67) |
| Cinnamic acid | tyrosine ammonia lyase (EC 4.3.1.23); trans-cinnamate 4- |
| (2 enzymatic steps) | monooxygenase (EC 1.14.13.11) |
| Resveratrol | tyrosine ammonia lyase (EC 4.3.1.23); 4-coumaroyl-CoA |
| (3 enzymatic steps) | synthetase (EC 6.2.1.12); resveratrol synthase (2.3.1.95) |
| Naringenin | tyrosine ammonia lyase (EC 4.3.1.23); 4-coumaroyl-CoA |
| (4 enzymatic steps) | synthetase (EC 6.2.1.12); chalcone synthase (EC 2.3.1.74); |
| | chalcone isomerase (EC 5.5.1.6) |
| Fisetin | tyrosine ammonia lyase (EC 4.3.1.23); 4-coumaroyl-CoA |
| (8 enzymatic steps) | synthetase (EC 6.2.1.12); chalcone synthase (EC 2.3.1.74); |
| | chalcone reductase (EC 2.3.1.170); chalcone isomerase (EC |
| | 5.5.1.6); flavanone 3-hydroxylase (EC 1.14.11.9); flavonol |
| | synthase (EC 1.14.11.23); flavonoid 3′-monooxygenase (EC |
| | 1.14.13.88/EC 1.14.13.21) |
| Curcumin | tyrosine ammonia lyase(EC 4.3.1.23); p-coumarate 3- |
| (6 enzymatic steps) | hydroxylase (EC 1.14.13); caffeic acid 3-O- |
| | methyltransferase (EC 2.1.1.68); trans-feruloyl-CoA |
| | synthase (EC 6.2.1.34); diketide-CoA synthase (EC |
| | 2.3.1.218); curcumin synthase (EC 2.3.1.217) |

According to certain embodiments, the hydroxycinnamic acid is p-coumaric acid. In order to convert L-tyrosine into p-coumaric acid the microorganism suitably comprises (e.g. expresses) a heterologous polypeptide having tyrosine ammonia lyase activity. Tyrosine ammonia-lyases (EC 4.3.1.23) have been described in the patent and non-patent literature. Non-limiting examples of polypeptides having tyrosine ammonia lyase activity which can be employed according to the present invention are disclosed, for example, in International patent application PCT/EP2015/066067 (published as WO2016/008886), which is hereby incorporated by reference. Details on specific polypeptides having tyrosine ammonia lyase activity which can be employed according to the present invention are given below.

According to certain embodiments, the polypeptide having tyrosine ammonia lyase activity is a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 7, 8, 9, 10, 11, 12, 13, 14, 15, 16 or 17 (preferably, SEQ ID NO: 7). Suitably, the polypeptide has a tyrosine ammonia lyase activity similar to that of the polypeptide comprising an amino acid sequence set forth in SEQ ID NO: 7, 8, 9, 10, 11, 12, 13, 14, 15, 16 or 17 (preferably, SEQ ID NO: 7). With “similar” tyrosine ammonia lyase activity it is meant that the polypeptide has at least about 10%, such as at least about 20%, at least about 30%, at least about 40%, at least about 50%, at least about 60, at least about 75%, at least about 80%, at least about 90%, at least about 95%, at least about 100%, at least about 200%, at least about 200%, at least about 400% or at least about 800%, of the ammonia lyase activity of the reference polypeptide (e.g., SEQ ID NO: 7). The tyrosine ammonia lyase activity may for instance be determined in accordance with the method described in WO2016/008886 at page 9, line 29 to page 10, line 2.

According to certain embodiments, the polypeptide having tyrosine ammonia lyase activity is a polypeptide comprising an amino acid sequence set forth in SEQ ID NO: 7, 8, 9, 10, 11, 12, 13, 14, 15, 16 or 17 (preferably, SEQ ID NO: 7).

According to certain embodiments, the hydroxycinnamic acid derivative is zosteric acid. In order to convert p-coumaric acid into zosteric acid the microorganism suitably comprises (e.g. expresses) a heterologous polypeptide having an aryl sulfotransferase activity. Aryl sulfotransferases (EC: 2.8.2.1) have been described in the patent and non-patent literature. Non-limiting examples of polypeptides having aryl sulfotransferase activity which can be employed according to the present invention are disclosed, for example, in International patent application PCT/EP2015/069298 (published as WO2016/026979), which is hereby incorporated by reference. Details on specific polypeptides having aryl sulfotransferase activity which can be employed according to the present invention are given below.

According to certain embodiments, the polypeptide having aryl sulfotransferase activity is a mammalian aryl sulfotransferase, such as a mammalian sulfotransferase 1A1 enzyme.

According to certain embodiments, the polypeptide having aryl sulfotransferase activity is an aryl sulfotransferase from Rattus norvegicus or a variant thereof. Such variant may have at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of the aryl sulfotransferase from Rattus norvegicus.

According to certain embodiments, the polypeptide having aryl sulfotransferase activity is a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 18, 19, 20, 21, 22, 23, 24, 25, 26, 27, 28, 29 or 30 (preferably, SEQ ID NO: 18). Suitably, the polypeptide has a aryl sulfotransferase activity similar to that of the polypeptide comprising an amino acid sequence set forth in SEQ ID NO: 18, 19, 20, 21, 22, 23, 24, 25, 26, 27, 28, 29 or 30 (preferably, SEQ ID NO: 18). With “similar” aryl sulfotransferase activity it is meant that the polypeptide has at least about 10%, such as at least about 20%, at least about 30%, at least about 40%, at least about 50%, at least about 60, at least about 75%, at least about 80%, at least about 90%, at least about 95%, at least about 100%, at least about 200%, at least about 200%, at least about 400% or at least about 800%, of the aryl sulfotransferase activity of the reference polypeptide (e.g., SEQ ID NO: 18). The aryl sulfotransferase activity may for instance be determined in accordance with the method described in WO2016/026979 at page 12, line 22 to page 13, line 2.

According to certain embodiments, the polypeptide having aryl sulfotransferase activity is a polypeptide comprising an amino acid sequence set forth in SEQ ID NO: 18, 19, 20, 21, 22, 23, 24, 25, 26, 27, 28, 29 or 30 (preferably, SEQ ID NO: 18).

According to certain embodiments, the biochemical compound is L-serine or a derivative thereof.

According to certain embodiment, the biochemical compound is a biochemical compound derived from Acetyl-CoA. Acetyl-CoA derivatives and their respective biosynthetic pathways are well known.

Non-limiting examples of Acetyl-CoA derived biochemical compounds are mevalonate, PHA (polyhydroxyalkanoates), PHB (poly-3-hydroxybutanoate), acetone, isopropanol, 1-butanol, fatty acids, and polyketides such as Lovastatin.

In order to convert Acetyl-CoA into the desired derivative, the microorganism suitably comprises (e.g., expresses) one or more enzymes catalyzing the chemical reaction(s) leading to the desired derivative. Table 2 below provides an overview of Acetyl-CoA derived biochemical compounds and the enzyme(s) involved in the conversion of Acetyl-CoA into the respective derivative. The microorganism may inherently express the one or more enzymes or may be modified to express the one or more enzymes by using, e.g., DNA recombination techniques.

TABLE 2
Acetyl-CoA derivatives and the enzyme(s) involved in the conversion of Acetyl-CoA
into said derivatives.

| Acetly-CoA derivative | Enzyme(s) |
|---|---|
| Mevalonate | acetyl-CoA acetyltransferase (EC 2.3.1.9); 3-hydroxy-3- |
| | methylglutaryl-CoA (HMG-CoA) synthase (EC 2.3.3.10); N- |
| | terminally truncated HMG-CoA reductase (tHMGR). |
| PHA | acetyl-CoA acetyltransferase (EC 2.3.1.9); acetoacetyl-CoA |
| (polyhydroxyalkanoates) | reductase (1.1.1.36); PHA synthase |
| PHB (poly-3- | acetyl-CoA acetyltransferase (EC 2.3.1.9); acetoacetyl-CoA |
| hydroxybutanoate) | reductase (EC 1.1.1.36); poly-β-hydroxybutyrate |
| | polymerase (EC 2.3.1.—) |
| Acetone | acetyl-CoA acetyltransferase (EC 2.3.1.9); butyrate- |
| | acetoacetate CoA-transferase (EC 3.1.2.11); Acetoacetate |
| | carboxy-lyase (EC 4.1.1.4) |
| Isopropanol | acetyl-CoA acetyltransferase (EC 2.3.1.9); butyrate- |
| | acetoacetate CoA-transferase (EC 3.1.2.11); Acetoacetate |
| | carboxy-lyase (EC 4.1.1.4); secondary alcohol |
| | dehydrogenase (EC 1.1.1.80) |
| 1-butanol | acetyl-CoA acetyltransferase (EC 2.3.1.9); 3-hydroxyacyl- |
| | CoA dehydrogenase (EC 1.1.1.35); 3-hydroxybutyryl-CoA |
| | dehydratase (EC 4.2.1.150); trans-2-enoyl-CoA reductase |
| | (EC 1.3.1.44); butanal dehydrogenase (EC 1.2.1.57); |
| | butanol dehydrogenase (EC 1.1.1.—) |
| Fatty acids | acetyl-CoA carboxylase (EC 6.4.1.2); malonyl-CoA-ACP |
| (biosynthetic pathway in | transacylase (EC 2.3.1.39); β-ketoacyl-ACP synthase III/β- |
| bacteria) | ketoacyl-ACP synthase I/β-ketoacyl-ACP synthase II (EC |
| | 2.3.1.180/2.3.1.41/2.3.1.179); β-ketoacyl-ACP reductase |
| | (EC 1.1.1.100); β-hydroxy acyl-ACP dehydrase (EC |
| | 4.2.1.59); enol acyl-ACP reductase (EC1.3.1.9); fatty acyl- |
| | ACP thioesterase (EC 3.1.2.14/3.1.2.21) |
| Fatty acids | acetyl-CoA carboxylase (EC 6.4.1.2); fatty acid synthase (EC |
| (biosynthetic pathway in | 2.3.1.86/2.3.1.85); fatty acyl-ACP thioesterase (EC |
| eukaryotes such as yeast) | 3.1.2.14/3.1.2.21) |
| Lovastatin | acetyl-CoA carboxylase (EC 6.4.1.2); lovastatin nonaketide |
| | synthase (EC 2.3.1.161); dihydromonacolin L-[lovastatin |
| | nonaketide synthase] thioesterase (EC 3.1.2.31); |
| | dihydromonacolin L hydroxylase (EC 1.4.13.197); |
| | monacolin L hydroxylase (EC 1.14.13.198); 2- |
| | methylbutanoate polyketide synthase (EC 2.3.1.244); |
| | monacolin J acid methylbutanoate transferase (EC |
| | 2.3.1.238) |

According to certain embodiments, the biochemical compound is a polyhydroxyalkanoate (PHA). According to certain embodiments, the biochemical compound is poly-3-hydroxybutanoate (PHB). According to certain embodiments, the biochemical compound is acetone. According to certain embodiments, the biochemical compound is isopropanol. According to certain embodiments, the biochemical compound is 1-butanol. According to certain embodiments, the biochemical compound is a fatty acid. According to certain embodiments, the biochemical compound is Lovastatin.

According to certain embodiments, the biochemical compound is mevalonate or a derivative thereof. According to certain embodiments, the biochemical compound is mevalonate. According to certain embodiments, the biochemical compound is mevalonate derivative. Mevalonate derivatives and their respective biosynthetic pathways are well known.

According to certain embodiments, the mevalonate derivative is an isoprenoid.

According to certain embodiments, the mevalonate derivative is a terpenoid.

According to certain embodiments, the mevalonate derivative is selected from the group consisting of: Mev-P, Mev-PP, IPP, GPP, GGPP, FPP, GGPP, DMAPP, isoprene, (4S)-limonene, (R)-limonene, phytoene, lycopene, beta-carotene, astaxanthin, amorphadiene, taxadiene, alpha-farnesene, beta-farnesene, and (2E,6E)-farnesol.

In order to convert mevalonate into the desired derivative, the microorganism suitably comprises (e.g., expresses) one or more enzymes catalyzing the chemical reaction(s) leading to the desired derivative. Table 3 below provides an overview of mevalonate derivatives and the enzyme(s) involved in the conversion of mevalonate into the respective derivative. The microorganism may inherently express the one or more enzymes or may be modified to express the one or more enzymes by using, e.g., DNA recombination techniques.

TABLE 3
Mevalonate derivatives and the enzyme(s) involved in
the conversion of mevalonate into said derivatives.

| Derivative | Enzyme(s) |
|---|---|
| Mev-P | ERG12 (mevalonate kinase) (EC 2.7.1.36) |
| (1 enzymatic step) | |
| Mev-PP | ERG12 (mevalonate kinase) (EC 2.7.1.36); ERG8 |
| (2 enzymatic steps) | (phosphomevalonate kinase) (EC 2.7.4.2) |
| IPP | ERG12 (mevalonate kinase) (EC 2.7.1.36); ERG8 |
| (3 enzymatic steps) | (phosphomevalonate kinase) (EC 2.7.4.2); MVD1 |
| | (mevalonate pyrophosphate decarboxylase) (EC 4.1.1.33) |
| DMAPP | ERG12 (mevalonate kinase) (EC 2.7.1.36); ERG8 |
| (4 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2) |
| GPP | ERG12 (mevalonate kinase) (EC 2.7.1.36); ERG8 |
| (5 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1) |
| FPP | ERG12 (mevalonate kinase) (EC 2.7.1.36); ERG8 |
| (6 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10) |
| GGPP | ERG12 (mevalonate kinase) (EC 2.7.1.36); ERG8 |
| (7 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); geranylgeranyl diphosphate |
| | synthase (2.5.1.29) |
| Isoprene | ERG12 (mevalonate kinase) (EC 2.7.1.36); ERG8 |
| (4 enzymatic steps) | (phosphomevalonate kinase) (EC 2.7.4.2); MVD1 |
| | (mevalonate pyrophosphate decarboxylase) (EC 4.1.1.33); |
| | Isopentenylpyrophosphate isomerase (EC 4.2.3.27) |
| (4S)-limonene | ERG12 (mevalonate kinase) (EC 2.7.1.36); ERG8 |
| (6 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); (4S)-limonene synthase |
| | (EC 4.2.3.16) |
| (R)-limonene | ERG12 (mevalonate kinase) (EC 2.7.1.36); ERG8 |
| (6 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); (R)-limonene synthase |
| | (EC 4.2.3.20) |
| Phytoene | ERG12 (mevalonate kinase) (EC 2.7.1.36); ERG8 |
| (8 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); geranylgeranyl diphosphate |
| | synthase (2.5.1.29); phytoene synthase (EC 2.5.1.32) |
| Lycopene | ERG12 (mevalonate kinase) (EC 2.7.1.36); ERG8 |
| (9 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); geranylgeranyl diphosphate |
| | synthase (2.5.1.29); phytoene synthase (EC 2.5.1.32); |
| | phytoene desaturase (EC 1.3.99.31) |
| beta-carotene | ERG12 (mevalonate kinase) (EC 2.7.1.36); ERG8 |
| (10 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); geranylgeranyl diphosphate |
| | synthase (2.5.1.29); phytoene synthase (EC 2.5.1.32); |
| | phytoene desaturase (EC 1.3.99.31); lycopene beta-cyclase |
| | (EC 5.5.1.19) |
| Astaxanthin | ERG12 (mevalonate kinase) (EC 2.7.1.36); ERG8 |
| (12 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); geranylgeranyl diphosphate |
| | synthase (2.5.1.29); phytoene synthase (EC 2.5.1.32); |
| | phytoene desaturase (EC 1.3.99.31); lycopene beta-cyclase |
| | (EC 5.5.1.19); beta-carotene hydroxylase (EC 1.14.13.129); |
| | beta-carotene ketolase (crtW) |
| Amorphadiene | ERG12 (mevalonate kinase) (EC 2.7.1.36); ERG8 |
| (7 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); amorphadiene synthase (EC |
| | 4.2.3.24) |
| Taxadiene | ERG12 (mevalonate kinase) (EC 2.7.1.36); ERG8 |
| (8 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); geranylgeranyl diphosphate |
| | synthase (2.5.1.29); taxadiene synthase (EC 4.2.3.17) |
| alpha-farnesene | ERG12 (mevalonate kinase) (EC 2.7.1.36); ERG8 |
| (7 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); alpha-farnesene synthase (EC |
| | 4.2.3.46) |
| beta-farnesene | ERG12 (mevalonate kinase) (EC 2.7.1.36); ERG8 |
| (7 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); beta-farnesene synthase (4.2.3.47) |
| (2E,6E)-farnesol | ERG12 (mevalonate kinase) (EC 2.7.1.36); ERG8 |
| (7 enzymatic steps) | (phosphomevalonate kinase) (EC 2.7.4.2); MVD1 |
| | (mevalonate pyrophosphate decarboxylase) (EC 4.1.1.33); |
| | isopentenylpyrophosphate isomerase (EC 5.3.3.2); geranyl |
| | diphosphate synthase (EC 2.5.1.1); Farnesyl-diphosphate |
| | synthase (EC 2.5.1.10); farnesyl diphosphatase (EC 3.1.7.6) |

Microorganism of the Invention

As indicated above, the present invention employs microorganisms which may comprise certain modification to achieve the present invention, and thus form part of the present invention. The respective details given above apply mutatis mutandis.

The present invention thus provides a genetically modified microorganism which comprises one or more of the following modifications a) to l):

a) an exogenous nucleic acid molecule comprising a nucleotide sequence encoding an inhibitory nucleic acid molecule that specifically hybridizes (e.g. binds) under cellular conditions with cellular mRNA and/or genomic DNA encoding an enzyme involved in the biosynthesis of a pyrimidine nucleotide;

b) an exogenous nucleic acid molecule comprising a nucleotide sequence encoding an inhibitory nucleic acid molecule that specifically hybridizes (e.g. binds) under cellular conditions with cellular mRNA and/or genomic DNA encoding an enzyme involved in the biosynthesis of a purine nucleotide;

c) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a pyrimidine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a pyrimidine nucleotide;

d) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a purine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a purine nucleotide;

e) a gene encoding an enzyme involved in the biosynthesis of a pyrimidine nucleotide, the regulatory sequence of said gene comprises a repressible promoter;

f) a gene encoding an enzyme involved in the biosynthesis of a pyrimidine nucleotide, the regulatory sequence of said gene comprises an operator; and an exogenous nucleic acid molecule comprising a nucleotide sequence encoding a repressor that is capable of binding to the operator;

g) a gene encoding an enzyme involved in the biosynthesis of a purine nucleotide, the regulatory sequence of said gene comprises a repressible promoter;

h) a gene encoding an enzyme involved in the biosynthesis of a purine nucleotide, the regulatory sequence of said gene comprises an operator; wherein the genetically modified microorganism further comprises an exogenous nucleic acid molecule comprising a nucleotide sequence encoding a repressor that is capable of binding to the operator; and

i) an inactivated gene encoding an enzyme involved in the biosynthesis of a pyrimidine nucleotide;

j) an inactivated gene encoding an enzyme involved in the biosynthesis of a purine nucleotide;

k) a gene encoding an enzyme involved in the biosynthesis of a pyrimidine nucleotide, wherein the gene comprises within the region encoding an UTR, such as a 5′-UTR, a nucleotide sequence encoding a riboswitch;

l) a gene encoding an enzyme involved in the biosynthesis of a purine nucleotide, wherein the gene comprises within the region encoding an UTR, such as a 5′-UTR, a nucleotide sequence encoding a riboswitch.

According to certain embodiments, a genetically modified microorganism is provided which comprises an exogenous nucleic acid molecule comprising a nucleotide sequence encoding an inhibitory nucleic acid molecule that specifically hybridizes (e.g. binds) under cellular conditions with an mRNA and/or gene encoding an enzyme involved in the biosynthesis of a pyrimidine nucleotide.

According to certain embodiments, a genetically modified microorganism is provided which comprises an exogenous nucleic acid molecule comprising a nucleotide sequence encoding an inhibitory nucleic acid molecule that specifically hybridizes (e.g. binds) under cellular conditions with an mRNA and/or gene encoding an enzyme having orotidine-5′-phosphate decarboxylase activity.

According to certain embodiments, a genetically modified microorganism is provided which comprises an exogenous nucleic acid molecule comprising a nucleotide sequence encoding an inhibitory nucleic acid molecule that specifically hybridizes (e.g. binds) under cellular conditions with an mRNA and/or gene encoding an enzyme involved in the biosynthesis of a purine nucleotide.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with a gene encoding an enzyme involved in the biosynthesis of a pyrimidine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with a gene encoding an enzyme involved in the biosynthesis of a pyrimidine nucleotide.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with a gene encoding an enzyme having orotidine-5′-phosphate decarboxylase activity; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with a gene encoding an enzyme having orotidine-5′-phosphate decarboxylase activity.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a pyrimidine nucleotide.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme having orotidine-5′-phosphate decarboxylase activity.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a pyrimidine nucleotide.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme having orotidine-5′-phosphate decarboxylase activity.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with a gene encoding an enzyme involved in the biosynthesis of a purine nucleotide; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a purine nucleotide.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding an enzyme involved in the biosynthesis of a purine nucleotide.

According to certain embodiments, a genetically modified microorganism is provided which comprises an exogenous nucleic acid molecule comprising a nucleotide sequence encoding a catalytically inactive RNA-guided endonuclease, a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding an enzyme involved in the biosynthesis of a purine nucleotide.

According to certain embodiments, a genetically modified microorganism is provided which comprises a gene encoding an enzyme involved in the biosynthesis of a pyrimidine nucleotide, the regulatory sequence of said gene comprises a repressible promoter.

According to certain embodiments, a genetically modified microorganism is provided which comprises a gene encoding an enzyme having orotidine-5′-phosphate decarboxylase activity, the regulatory sequence of said gene comprises a repressible promoter.

According to certain embodiments, a genetically modified microorganism is provided which comprises a gene encoding an enzyme involved in the biosynthesis of a pyrimidine nucleotide, the regulatory sequence of said gene comprises an operator; wherein the genetically modified microorganism further comprises an exogenous nucleic acid molecule comprising a nucleotide sequence encoding a repressor that is capable of binding to the operator. According to certain embodiments, the exogenous nucleic acid molecule comprises an inducible promoter, such as a temperature inducible promoter, that is functional in the microorganism to cause the production of said repressor and that is operably linked to the nucleotide sequence encoding said repressor.

According to certain embodiments, a genetically modified microorganism is provided which comprises a gene encoding an enzyme having orotidine-5′-phosphate decarboxylase activity, the regulatory sequence of said gene comprises an operator; wherein the genetically modified microorganism further comprises an exogenous nucleic acid molecule comprising a nucleotide sequence encoding a repressor that is capable of binding to the operator. According to certain embodiments, the exogenous nucleic acid molecule comprises an inducible promoter, such as a temperature inducible promoter, that is functional in the microorganism to cause the production of said repressor and that is operably linked to the nucleotide sequence encoding said repressor.

According to certain embodiments, a genetically modified microorganism is provided which comprises a gene encoding an enzyme involved in the biosynthesis of a purine nucleotide, the regulatory sequence of said gene comprises a repressible promoter.

According to certain embodiments, a genetically modified microorganism is provided which comprises a gene encoding an enzyme involved in the biosynthesis of a purine nucleotide, the regulatory sequence of said gene comprises an operator; wherein the genetically modified microorganism further comprises an exogenous nucleic acid molecule comprising a nucleotide sequence encoding a repressor that is capable of binding to the operator.

According to certain embodiments, a genetically modified microorganism is provided which comprises an inactivated gene encoding an enzyme involved in the biosynthesis of a pyrimidine nucleotide.

According to certain embodiments, a genetically modified microorganism is provided which comprises an inactivated gene encoding an enzyme having orotidine-5′-phosphate decarboxylase activity.

According to certain embodiments, a genetically modified microorganism is provided which comprises an inactivated gene encoding an enzyme involved in the biosynthesis of a purine nucleotide.

According to certain embodiments, a genetically modified microorganism is provided which comprises a gene encoding an enzyme involved in the biosynthesis of a pyrimidine nucleotide, wherein the gene comprises within the region encoding an UTR, such as a 5′-UTR, a nucleotide sequence encoding a riboswitch.

According to certain embodiments, a genetically modified microorganism is provided which comprises a gene encoding an enzyme having orotidine-5′-phosphate decarboxylase activity, wherein the gene comprises within the region encoding an UTR, such as a 5′-UTR, a nucleotide sequence encoding a riboswitch.

According to certain embodiments, a genetically modified microorganism is provided which comprises a gene encoding an enzyme involved in the biosynthesis of a purine nucleotide, wherein the gene comprises within the region encoding an UTR, such as a 5′-UTR, a nucleotide sequence encoding a riboswitch.

According to certain embodiments, the genetically modified microorganism as detailed above has been modified to have a down regulated biosynthesis of a pyrimidine or purine nucleotide compared to an otherwise identical microorganism that does not carry said modification.

Further provided is a genetically modified microorganism which comprises one or more of the following modifications A-1) to F-1):

A-1) an exogenous nucleic acid molecule comprising a nucleotide sequence encoding an inhibitory nucleic acid molecule that specifically hybridizes (e.g. binds) under cellular conditions with cellular mRNA and/or genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes;

B-1) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes;

C-1) a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises a repressible promoter;

D-1) a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises an operator; wherein the genetically modified microorganism further comprises an exogenous nucleic acid molecule comprising a nucleotide sequence encoding a repressor that is capable of binding to the operator;

E-1) an inactivated gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes;

F-1) a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes; wherein the gene comprises within the region encoding an UTR, such as a 5′-UTR, a nucleotide sequence encoding a riboswitch.

According to certain embodiments, a genetically modified microorganism is provided which comprises an exogenous nucleic acid molecule comprising a nucleotide sequence encoding an inhibitory nucleic acid molecule that specifically hybridizes (e.g. binds) under cellular conditions with cellular mRNA and/or genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

According to certain embodiments, a genetically modified microorganism is provided which comprises an exogenous nucleic acid molecule comprising a nucleotide sequence encoding an inhibitory nucleic acid molecule that specifically hybridizes (e.g. binds) under cellular conditions with cellular mRNA and/or genomic DNA encoding a polypeptide encoded by the gene yheV or an ortholog thereof.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

According to certain embodiments, a genetically modified microorganism which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding a polypeptide encoded by the gene yheV or an ortholog thereof.

According to certain embodiments, a genetically modified microorganism is provided which comprises a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises a repressible promoter.

According to certain embodiments, a genetically modified microorganism is provided which comprises the gene yheV or an ortholog thereof, wherein the regulatory sequence of said gene comprises a repressible promoter.

According to certain embodiments, a genetically modified microorganism is provided which comprises a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes, the regulatory sequence of said gene comprises an operator; wherein the genetically modified microorganism further comprises an exogenous nucleic acid molecule comprising a nucleotide sequence encoding a repressor that is capable of binding to the operator.

According to certain embodiments, the exogenous nucleic acid molecule comprises an inducible promoter, such as a temperature inducible promoter, that is functional in the microorganism to cause the production of said repressor and that is operably linked to the nucleotide sequence encoding said repressor.

According to certain embodiments, a genetically modified microorganism is provided which comprises the gene yheV or an ortholog thereof, the regulatory sequence of said gene comprises an operator; wherein the genetically modified microorganism further comprises an exogenous nucleic acid molecule comprising a nucleotide sequence encoding a repressor that is capable of binding to the operator. According to certain embodiments, the exogenous nucleic acid molecule comprises an inducible promoter, such as a temperature inducible promoter, that is functional in the microorganism to cause the production of said repressor and that is operably linked to the nucleotide sequence encoding said repressor.

According to certain embodiments, a genetically modified microorganism is provided which comprises an inactivated gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes.

According to certain embodiments, a genetically modified microorganism is provided which comprises an inactivated yheV gene or ortholog thereof.

According to certain embodiments, a genetically modified microorganism is provided which comprises a gene encoding a polypeptide selected from the group consisting of: a polypeptide encoded by the gene lpxC, a polypeptide encoded by the gene yaiY, a polypeptide encoded by the gene ydiB, a polypeptide encoded by the gene yheV, a polypeptide encoded by the gene ygaQ, a polypeptide encoded by the gene glcA, a polypeptide encoded by the gene yjeN, a polypeptide encoded by the gene malZ, and a polypeptide encoded by an ortholog of any one of the aforementioned genes; wherein the gene comprises within the region encoding an UTR, such as a 5′-UTR, a nucleotide sequence encoding a riboswitch.

According to certain embodiments, a genetically modified microorganism is provided which comprises a yheV gene or an ortholog thereof; wherein the gene comprises within the region encoding an UTR, such as a 5′-UTR, a nucleotide sequence encoding a riboswitch.

The genetically modified microorganism as detailed above has been modified to have a reduced expression of the polypeptide compared to an otherwise identical microorganism that does not carry said modification.

Further provided is a genetically modified microorganism which comprises one or more of the following modifications A-2) to G-2):

A-2) an exogenous nucleic acid molecule comprising a nucleotide sequence encoding an inhibitory nucleic acid molecule that specifically hybridizes (e.g. binds) under cellular conditions with SibB and/or genomic DNA encoding SibB;

B-2) an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding SibB; or an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding SibB;

C-2) a gene encoding SibB, the regulatory sequence of said gene comprises a repressible promoter;

D-2) a gene encoding SibB, the regulatory sequence of said gene comprises an operator; wherein the genetically modified microorganism further comprises an exogenous nucleic acid molecule comprising a nucleotide sequence encoding a repressor that is capable of binding to the operator;

E-2) an inactivated gene encoding SibB;

F-2) an exogenous nucleic acid molecule comprising a nucleotide sequence encoding a polypeptide comprising an amino acid sequence set forth in SEQ ID NO: 6, wherein the exogenous nucleic acid optionally comprises an inducible promoter that is functional in the microorganism to cause the production of an mRNA molecule the translation of which results in said polypeptide and that is operably linked to the nucleotide sequence encoding said polypeptide;

G-2) an exogenous nucleic acid molecule comprising a nucleotide sequence encoding a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 6, wherein the exogenous nucleic acid optionally comprises an inducible promoter that is functional in the microorganism to cause the production of an mRNA molecule the translation of which results in said polypeptide and that is operably linked to the nucleotide sequence encoding said polypeptide.

According to certain embodiments, a genetically modified microorganism is provided which comprises an exogenous nucleic acid molecule comprising a nucleotide sequence encoding an inhibitory nucleic acid molecule that specifically hybridizes (e.g. binds) under cellular conditions with SibB and/or genomic DNA encoding SibB.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and a nucleotide sequence encoding a single guide RNA (sgRNA) which specifically hybridizes (e.g. binds) under cellular conditions with genomic DNA encoding SibB.

According to certain embodiments, a genetically modified microorganism is provided which comprises an exogenous nucleic acid molecule comprising a nucleotide sequence encoding a catalytically inactive RNA-guided endonuclease, such as a catalytically inactive Cas9 protein, and an exogenous nucleic acid molecule comprising a nucleotide sequence encoding a single guide RNA (sgRNA) which specifically hybridizes (e.g. binds) under cellular conditions with genomic DNA encoding SibB.

According to certain embodiments, a genetically modified microorganism is provided which comprises a gene encoding SibB, the regulatory sequence of said gene comprises a repressible promoter.

According to certain embodiments, a genetically modified microorganism is provided which comprises a gene encoding SibB, the regulatory sequence of said gene comprises an operator; wherein the genetically modified microorganism further comprises an exogenous nucleic acid molecule comprising a nucleotide sequence encoding a repressor that is capable of binding to the operator.

According to certain embodiments, a genetically modified microorganism is provided which comprises an inactivated gene encoding SibB.

According to certain embodiments, a genetically modified microorganism is provided which comprises an exogenous nucleic acid molecule comprising a nucleotide sequence encoding a polypeptide comprising an amino acid sequence set forth in SEQ ID NO: 6, wherein the exogenous nucleic acid optionally comprises an inducible promoter that is functional in the microorganism to cause the production of an mRNA molecule the translation of which results in said polypeptide and that is operably linked to the nucleotide sequence encoding said polypeptide.

According to certain embodiments, a genetically modified microorganism is provided which comprises an exogenous nucleic acid molecule comprising a nucleotide sequence encoding a polypeptide comprising an amino acid sequence which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence set forth in SEQ ID NO: 6, wherein the exogenous nucleic acid optionally comprises an inducible promoter that is functional in the microorganism to cause the production of an mRNA molecule the translation of which results in said polypeptide and that is operably linked to the nucleotide sequence encoding said polypeptide.

The genetically modified microorganism as described above may be further modified to, e.g., comprise (e.g., express) a heterologous polypeptide having tyrosine ammonia lyase activity and/or a heterologous polypeptide having an aryl sulfotransferase activity. Further details on polypeptides having tyrosine ammonia lyase activity and polypeptides having an aryl sulfotransferase activity are given above.

General Matter

Generally, a microorganism as referred to herein may be any suitable microorganism, including single-celled or multicellular microorganisms such as bacteria, yeast, fungi and algae.

Bacterial microorganisms may be Gram-positive or Gram-negative bacteria. Non-limiting examples for Gram-negative bacteria include species from the genera Escherichia, Erwinia, Klebsiella and Citrobacter. Non-limiting examples of Gram-positive bacteria include species from the genera Bacillus, Lactococcus, Lactobacillus, Geobacillus, Pediococcus, Moorella, Clostridium, Corynebacterium, Streptomyces, Streptococcus, and Cellulomonas.

According to certain embodiments, the microorganism is a bacterium, which may be a bacterium of the genus Bacillus, Lactococcus, Lactobacillus, Clostridium, Corynebacterium, Geobacillus, Streptococcus, Pediococcus, Moorella, Pseudomonas, Streptomyces, Escherichia, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus, or Yersinia.

According to certain embodiments, the microorganism is a bacterium of the genus Escherichia. A non-limiting example of a bacterium of the genus Escherichia is Escherichia coli. According to certain embodiments, the microorganism is Escherichia coli.

According to certain embodiments, the microorganism is a bacterium of the genus Bacillus. Non-limiting examples of a bacterium of the genus Bacillus are Bacillus subtitlis, Bacillus amyloliquefaciens, Bacillus licheniformis, and Bacillus mojavensis. According to certain embodiments, the microorganism is Bacillus subtitlis. According to certain embodiments, the microorganism is Bacillus licheniformis.

According to certain embodiments, the microorganism is a bacterium of the genus Lactococcus. A non-limiting example of a bacterium of the genus Lactococcus is Lactococcus lactis. According to certain embodiments, the microorganism is Lactococcus lactis.

According to certain embodiments, the microorganism is a bacterium of the genus Lactobacillus. A non-limiting example of a bacterium of the genus Lactococcus is Lactobacillus reuteri. According to certain embodiments, the microorganism is Lactobacillus reuteri.

According to certain embodiments, the microorganism is a bacterium of the genus Corynebacterium. A non-limiting example of a bacterium of the genus Corynebacterium is Corynebacterium glutamicum. According to certain embodiments, the microorganism is Corynebacterium glutamicum.

According to certain embodiments, the microorganism is a bacterium of the genus Geobacillus. Non-limiting examples of a bacterium of the genus Geobacillus are Geobacillus thermoglucosidasius and Geobacillus sp. GHH. According to certain embodiments, the microorganism is Geobacillus thermoglucosidasius. According to certain embodiments, the microorganism is Geobacillus sp. GHH.

According to certain embodiments, the microorganism is a bacterium of the genus Streptomyces. Non-limiting examples of a bacterium of the genus Streptomyces are Streptomyces lividans, Streptomyces coelicolor, or Streptomyces griseus. According to certain embodiments, the microorganism is Streptomyces lividans. According to other embodiments, the microorganism is Streptomyces coelicolor. According to other embodiments, the microorganism is Streptomyces griseus.

According to certain embodiments, the microorganism is a bacterium of the genus Pseudomonas. A non-limiting example of a bacterium of the genus Pseudomonas is Pseudomonas putida. According to certain embodiments, the microorganism is Pseudomonas putida.

According to certain embodiments, the microorganism is a bacterium of the genus Pediococcus. A non-limiting example of a bacterium of the genus Pediococcus is Pediococcus acidilactici. According to certain embodiments, the microorganism is Pediococcus acidilactici.

According to certain embodiments, the microorganism is a bacterium of the genus Moorella. A non-limiting example of a bacterium of the genus Moorella is Moorella thermoacetica. According to certain embodiments, the microorganism is Moorella thermoacetica.

Yeast cells may be derived from e.g., Saccharomyces, Pichia, Schizosacharomyces, Zygosaccharomyces, Hansenula, Pachyosolen, Kluyveromyces, Debaryomyces, Yarrowia, Candida, Cryptococcus, Komagataella, Lipomyces, Rhodospiridium, Rhodotorula, or Trichosporon.

According to certain embodiments, the microorganism is a yeast, which may be a yeast is of the genus Saccharomyces, Pichia, Schizosacharomyces, Zygosaccharomyces, Hansenula, Pachyosolen, Kluyveromyces, Debaryomyces, Yarrowia, Candida, Cryptococcus, Komagataella, Lipomyces, Rhodospiridium, Rhodotorula, or Trichosporon.

According to certain embodiments, the microorganism is a yeast of the genus Saccharomyces. A non-limiting example of a yeast of the genus Saccharomyces is Saccharomyces cerevisiae. According to certain embodiments, the microorganism is Saccharomyces cerevisiae.

According to certain embodiments, the microorganism is a yeast of the genus Pichia. Non-limiting example of a yeast of the genus Pichia are Pichia pastoris and pichia kudriavzevii. According to certain embodiments, the microorganism is Pichia pastoris. According to certain embodiments, the microorganism is pichia kudriavzevii.

Fungi cells may be derived from, e.g., Aspergillus.

According to certain embodiments, the microorganism is a fungus, such as a fungi of the genus Aspergillus. Non-limiting examples of a fungus of the genus Aspergillus are Aspergillus Oryzae, Aspergillus niger or Aspergillus awamsii. According to certain embodiments, the microorganism is Aspergillus Oryzae. According to other embodiments, the microorganism is Aspergillus niger. According to other embodiments, the microorganism is Aspergillus awamsii.

Algae cells may be derived from, e.g., Chlamydomonas, Haematococcus, Phaedactylum, Volvox or Dunaliella.

According to certain embodiments, the microorganism is an alga, which may be an alga of the genus Chlamydomonas, Haematococcus, Phaedactylum, Volvox or Dunaliella.

According to certain embodiments, the microorganism is an alga of the genus Chlamydomonas. A non-limiting example of an alga of the genus Chlamydomonas is Chlamydomonas reinhardtii.

According to certain embodiments, the microorganism is an alga of the genus Haematococcus. A non-limiting example of an alga of the genus Haematococcus is Haematococcus pluvialis.

According to certain embodiments, the microorganism is an alga of the genus Phaedactylum. A non-limiting example of an alga of the genus Phaedactylum is Phaedactylum tricornatum.

Generally, a microorganism (employed) according to the invention may be genetically modified to express a nucleic acid molecule (such as an inhibitor nucleic acid molecule) or polypeptide as detailed herein, which means that an exogenous nucleic acid molecule, such as a DNA molecule, which comprises a nucleotide sequence encoding said nucleic acid molecule or polypeptide has been introduced in the microorganism. Techniques for introducing exogenous nucleic acid molecule, such as a DNA molecule, into the various host cells are well-known to those of skill in the art, and include transformation (e.g., heat shock or natural transformation), transfection, conjugation, electroporation and microinjection.

Accordingly, a microorganism (employed) according to the invention may comprise an exogenous nucleic acid molecule comprising a nucleotide sequence encoding a nucleic acid molecule or polypeptide as detailed herein.

In order to facilitate expression of the nucleic acid molecule or polypeptide in the microorganism, the exogenous nucleic acid molecule may comprise suitable regulatory elements such as a promoter that is functional in the microorganism to cause the production of said encoded nucleic acid molecule or an mRNA molecule the translation of which results in said polypeptide and that is operably linked to the nucleotide sequence encoding said nucleic acid molecule or polypeptide.

Promoters useful in accordance with the invention are any known promoters that are functional in a given microorganism. Many such promoters are known to the skilled person. Such promoters include promoters normally associated with other genes, and/or promoters isolated from any bacteria, yeast, fungi, alga or plant cell. The use of promoters for protein expression is generally known to those of skilled in the art of molecular biology, for example, see Sambrook et al., Molecular cloning: A Laboratory Manual, Cold Spring Harbor Laboratory, Cold Spring Harbor, N.Y., 1989. The promoter employed may be inducible. A great number of inducible promoters have been described in the patent and non-patent literature. The term “inducible” used in the context of a promoter means that the promoter only directs transcription of an operably linked nucleotide sequence if a chemical or physical stimulus is present. Chemically-inducible promoters are promoters whose transcriptional activity is induced by the presence a chemical substance (“chemical inducer”), such as alcohol, tetracycline, steroids, metal or other compounds. As used herein, “chemical induction” refers to the physical application of an exogenous or endogenous substance (incl. macromolecules, e.g., proteins or nucleic acids) to a microorganism. This has the effect of causing the target promoter present in the microorganism to increase the rate of transcription.

Physically-inducible promoters are promoters whose transcriptional activity is induced by the presence a physical factor, such as light or low or high temperatures. Temperature induction systems work, for example, by employing promoters that are repressed by thermolabile repressors. These repressors are active at lower temperatures for example at 30° C., while unable to fold correctly at 37° C. and are therefore inactive. Such circuits therefore can be used to directly regulate the genes of interest (St-Pierre et al. 2013) also by genome integration of the genes along with the repressors. Non-limiting examples of temperature inducible promoter systems are based on the pL and/or pR λ phage promoters which are regulated by the thermolabile cl857 repressor. The repressor is temperature-sensitive and is functional at lower temperatures but denatures at temperatures higher than 37.5° C. Hence, induction of expression is achieved by shifting the temperature above 37.5° C. Conversely, inhibition of expression is achieved by shifting the temperature below 37.5° C. Similar to the genome integrated DE3 system, the expression of the T7 RNA polymerase gene may also be controlled using a temperature controlled promoter system (Mertens et al. 1995), while the expression of the gene of interest can be controlled using a T7 promoter. Another example of a temperature inducible promoter is the cspA promoter. While this promoter is only weakly induced by a change in temperature, a 159 nucleotide long untranslated region at the 5′ end of cspA driven mRNA transcripts makes them highly unstable at 37° C. and significantly increases their stability at low temperatures (below 20° C.).

Where desired, the promoter employed may be constitutive. The term “constitutive” used in the context of a promoter means that the promoter is capable of directing transcription of an operably linked nucleotide sequence in the absence of stimulus (such as heat shock, chemicals etc.).

Non-limiting examples of promoters functional in bacteria, such as Bacillus subtilis, Lactococcus lactis or Escherichia coli, include both constitutive and inducible promoters such as T7 promoter, the beta-lactamase and lactose promoter systems; alkaline phosphatase (phoA) promoter, a tryptophan (trp) promoter system, tetracycline promoter, lambda-phage promoter, ribosomal protein promoters; and hybrid promoters such as the tac promoter. Other bacterial and synthetic promoters are also suitable.

Non-limiting examples of promoters functional in yeast, such as Saccharomyces cerevisiae, include xylose promoter, GAL1 and GAL10 promoters, TEF1 promoter, and pgk1 promoter.

Non-limiting examples of promoters functional in fungi, such as Aspergillus Oryzae or Aspergillus niger, include promotors derived from the gene encoding Aspergillus oryzae TAKA amylase, Aspergillus niger neutral α-amylase, Aspergillus niger acid stable α-amylase, Aspergillus niger or Aspergillus awamsii glucoamylase (gluA), Aspergillus niger acetamidase, Aspergillus oryzae alkaline protease, Aspergillus oryzae triose phosphatase isomerase, Rhizopus meihei aspartic proteinase, and Rhizopus meihei lipase.

Non-limiting examples of promoters functional in alga, such as Haematococcus pluvialis, include the CaMV35S promoter, the SV40 promoter, and promoter of the Chlamydomonas reinhardtii RBCS2 gene and the promoter of the Volvox carteri ARS gene.

Besides a promoter, the exogenous nucleic acid molecule may further comprise at least one regulatory element selected from a 5′ untranslated region (5′UTR) and 3′ untranslated region (3′ UTR). Many such 5′ UTRs and 3′ UTRs derived from prokaryotes and eukaryotes are well known to the skilled person. Such regulatory elements include 5′ UTRs and 3′ UTRs normally associated with other genes, and/or 5′ UTRs and 3′ UTRs isolated from any bacteria, yeast, fungi or alga.

If the microorganism is a prokaryotic organism, the 5′ UTR usually contains a ribosome binding site (RBS), also known as the Shine Dalgarno sequence which is usually 3-10 base pairs upstream from the initiation codon. Meanwhile, if the host cell is an eukaryotic organism the 5′UTR usually contains the Kozak consensus sequence. A eukaryotic 5′

UTR may also contain cis-acting regulatory elements.

An exogenous nucleic acid molecule may be a vector or part of a vector, such as an expression vector. Normally, such a vector remains extrachromosomal within the host cell which means that it is found outside of the nucleus or nucleoid region of the host cell.

It is also contemplated by the present invention that an exogenous nucleic acid molecule is stably integrated into the genome of the microorganism. Means for stable integration into the genome of a microorganism, e.g., by homologous recombination, are well known to the skilled person.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the invention.

### Certain Definitions

The phrase “decoupling cell growth from production” as used herein means that the growth of a microorganism is reduced while still allowing for continued production.

The phrase “microorganism having the ability to produce a biochemical compound” or “microorganism having the ability to produce said biochemical compound” as used herein means a microorganism, such as a bacterium, which is able to produce, excrete or secrete, and/or cause accumulation of a biochemical compound of interest in a culture medium or in the microorganism when the microorganism is cultured in the medium. The phrase can mean that the microorganism is able to cause accumulation of the biochemical compound of interest in an amount not less than 0.05 g/L, when cultured in minimal M9 media supplemented with 2 g/L glucose at 37° C. with adequate aeration for 40 hours. A microorganism may be considered as having the ability to produce the biochemical compound of interest if it expresses all enzymes involved in the biosynthetic pathway resulting in the biochemical compound. The microorganism may inherently have the ability to produce the biochemical compound of interest or may be modified to have the ability to produce the biochemical compound of interest by using, e.g., DNA recombination techniques.

As used herein, a “biochemical compound” means any carbon-based compound that is produced by a living organism.

The phrase “microorganism having the ability to produce L-tyrosine or a derivative thereof” as used herein means a microorganism, such as a bacterium, which is able to produce, excrete or secrete, and/or cause accumulation of L-tyrosine or a derivative thereof in a culture medium or in the microorganism when the microorganism is cultured in the medium. The phrase can mean that the microorganism is able to cause accumulation of L-tyrosine or a derivative thereof in an amount not less than 0.05 g/L, when cultured in minimal M9 media supplemented with 2 g/L glucose, at 37° C. with adequate aeration for 40 hours. A microorganism may be considered as having the ability to produce L-tyrosine if it expresses all enzymes involved in the biosynthetic pathway resulting in L-tyrosine. For example, a microorganism may be considered as having the ability to produce L-tyrosine if it expresses the following enzymes: transketolase I (EC 2.2.1.1; encoded by the gene tktA or ortholog thereof); 2-dehydro-3-deoxyphosphoheptonate aldolase (EC 2.5.1.54; encoded by the gene aroG or ortholog thereof); 3-dehydroquinate synthase (EC 4.2.3.4; encoded by the gene aroB or ortholog thereof); 3-dehydroquinate dehydratase (EC 4.2.1.10; encoded by the gene aroD or ortholog thereof); shikimate dehydrogenase (EC 1.1.1.25; encoded by the gene aroE or ortholog thereof); shikimate kinase I (EC 2.7.1.71; encoded by the gene aroK or ortholog thereof); shikimate kinase II (EC 2.7.1.71; encoded by the gene aroL or ortholog thereof); 3-phosphoshikimate-1-carboxyvinyltransferase (EC 2.5.1.19; encoded by the gene aroA or ortholog thereof); chorismate synthase (EC4.2.3.5; encoded by the gene aroC or ortholog thereof); chorismate mutase/prephenate dehydrogenase (EC 5.4.99.5/EC 1.3.1.12; encoded by the gene tyrA or ortholog thereof); and tyrosine aminotransferase (EC 2.6.1.5; encoded by the gene tyrB or ortholog thereof). The microorganism may inherently have the ability to produce L-tyrosine or a derivative thereof or may be modified to have the ability to produce L-tyrosine or a derivative thereof by using, e.g., DNA recombination techniques.

The phrase “microorganism having the ability to produce L-serine or a derivative thereof” as used herein means a microorganism, such as a bacterium, which is able to produce, excrete or secrete, and/or cause accumulation of L-serine or a derivative thereof in a culture medium or in the microorganism when the microorganism is cultured in the medium. The phrase can mean that the microorganism is able to cause accumulation of L-serine or a derivative thereof in an amount not less than 0.05 g/L, when cultured in minimal M9 media supplemented with 2 g/L glucose, at 37° C. with adequate aeration for 40 hours. A microorganism may be considered as having the ability to produce L-serine if it expresses all enzymes involved in the biosynthetic pathway resulting in L-serine. For example, a microorganism may be considered as having the ability to produce L-serine if it expresses the following enzymes: phosphoglycerate dehydrogenase (EC 1.1.1.95; encoded by the gene serA or an ortholog thereof); phosphoserine/phosphohydroxythreonine aminotransferase (EC 2.6.1.52; encoded by the gene serC or an ortholog thereof); and phosphoserine phosphatase (EC 3.1.3.3; encoded by the gene serB or an ortholog thereof). The microorganism may inherently have the ability to produce L-serine or a derivative thereof or may be modified to have the ability to produce L-serine or a derivative thereof by using, e.g., DNA recombination techniques.

The phrase “microorganism having the ability to produce mevalonate or a derivative thereof” as used herein means a microorganism, such as a bacterium, which is able to produce, excrete or secrete, and/or cause accumulation of mevalonate or a derivative thereof in a culture medium or in the microorganism when the microorganism is cultured in the medium. The phrase can mean that the microorganism is able to cause accumulation of mevalonate or a derivative thereof in an amount not less than 0.05 g/L, when cultured in minimal M9 media supplemented with 2 g/L glucose, at 37° C. with adequate aeration for 40 hours. A microorganism may be considered as having the ability to produce mevalonate if it expresses all enzymes involved in the biosynthetic pathway resulting in mevalonate. For example, a microorganism may be considered as having the ability to produce mevalonate if it expresses the following enzymes: acetyl-CoA acetyltransferase (EC 2.3.1.9; encoded by the gene atoB or an ortholog thereof); 3-hydroxy-3-methylglutaryl-CoA (HMG-CoA) synthase (EC 2.3.3.10; encoded by the gene HMGS or an ortholog thereof); N-terminally truncated HMG-CoA reductase (encoded by the gene tHMGR or ortholog thereof). The microorganism may inherently have the ability to produce mevalonate or a derivative thereof or may be modified to have the ability to produce mevalonate or a derivative thereof by using, e.g., DNA recombination techniques.

The phrase “microorganism having the ability to produce a recombinant polypeptide” as used herein means a microorganism, such as a bacterium, which is able to produce, excrete or secrete, and/or cause accumulation of a recombinant polypeptide of interest. Suitably, the microorganism has been modified using, e.g., DNA recombination techniques, to comprise an exogenous nucleic acid molecule comprising a nucleotide sequence encoding said polypeptide operably linked to a promoter that is functional in the microorganism to cause the production of an mRNA molecule the translation of which results in said polypeptide.

As used herein, an “enzyme having orotidine-5′-phosphate decarboxylase activity” means an enzyme that catalyzes the reaction: Orotidine 5′-phosphate<=>UMP+CO₂ (EC 4.1.1.23). An enzyme having orotidine-5′-phosphate decarboxylase activity is, for example, encoded by the bacterial gene pyrF or an ortholog thereof. Further information regarding pyrF of, e.g., Escherichia coli is available at EcoCyc (www.biocyc.org) or EcoGene (www.ecogene.org) under Accession number EG10809. A representative nucleotide sequence of the E. coli pyrF gene is set forth in SEQ ID NO: 1. See also NCBI Reference Sequence: NP_415797.1 for the amino acid sequence (E. coli). In yeast, such as Saccharomyces cerevisiae, an enzyme having orotidine-5′-phosphate decarboxylase activity is encoded by the pyrF ortholog URA3. Further information regarding URA3 of Saccharomyces cerevisiae is available at YeastCyc (http://yeast.biocyc.org/) under Accession number YEL021W. A representative nucleotide sequence of the S. cerevisiae gene URA3 gene is set forth in SEQ ID NO: 2.

As used herein, an “enzyme having carbamoyl phosphate synthase activity” means an enzyme that catalyzes the reaction: 2 ATP+L-glutamine+HCO₃⁻+H₂O<=>2 ADP+phosphate+L-glutamate+carbamoyl phosphate (EC 6.3.5.5). An enzyme having carbamoyl phosphate synthase activity is, for example, encoded by the bacterial genes carA (encoding the alpha chain) and carB (encoding the beta chain) or orthologs thereof. Further information regarding carA and carB of, e.g., Escherichia coli is available at EcoCyc (www.biocyc.org) or EcoGene (www.ecogene.org) under Accession numbers EG10134 and EG10135, respectively. See also NCBI Reference Sequences: NP_414573.1 and NP_414574.1 for the respective amino acid sequences (E. coli). In yeast, such as Saccharomyces cerevisiae, an enzyme having carbamoyl phosphate synthase activity is encoded by the gene URA2. Further information regarding URA2 of Saccharomyces cerevisiae is available at YeastCyc (http://yeast.biocyc.org/) under Accession number YJL130C.

As used herein, an “enzyme having aspartate carbamoyltransferase activity” means an enzyme that catalyzes the reaction: Carbamoyl phosphate+L-aspartate<=>phosphate+N-carbamoyl-L-aspartate (EC 2.1.3.2). An enzyme having aspartate carbamoyltransferase activity is, for example, encoded by the bacterial gene pyrB or an ortholog thereof. Further information regarding pyrB of, e.g., Escherichia coli is available at EcoCyc (www.biocyc.org) or EcoGene (www.ecogene.org) under Accession number EG10805. See also NCBI Reference Sequences: NP_418666.1 for the amino acid sequence (E. coli). In yeast, such as Saccharomyces cerevisiae, an enzyme having aspartate carbamoyltransferase activity is encoded by the gene URA2. Further information regarding URA2 of Saccharomyces cerevisiae is available at YeastCyc (http://yeast.biocyc.org/) under Accession number YJL130C.

As used herein, an “enzyme having dihydroorotase activity” means an enzyme that catalyzes the reaction: (S)-dihydroorotate+H₂O<=>N-carbamoyl-L-aspartate (EC 3.5.2.3). An enzyme having dihydroorotase activity is, for example, encoded by the bacterial gene pyrC or an ortholog thereof. Further information regarding pyrC of, e.g., Escherichia coli is available at EcoCyc (www.biocyc.org) or EcoGene (www.ecogene.org) under Accession number EG10806. See also NCBI Reference Sequence: NP_415580.1 for the amino acid sequence (E. coli). In yeast, such as Saccharomyces cerevisiae, an enzyme having dihydroorotase activity is encoded by the pyrC ortholog URA4. Further information regarding URA4 of Saccharomyces cerevisiae is available at YeastCyc (http://yeast.biocyc.org/) under Accession number YLR420W.

As used herein, an “enzyme having dihydroorotate dehydrogenase activity” means an enzyme that catalyzes the reaction: (S)-dihydroorotate+a quinone<=>orotate+a quinol (EC 1.3.5.2); or catalyzes the reaction: (S)-dihydroorotate+NAD⁺<=>orotate+NADH (EC 1.3.1.14); or catalyzes the reaction: (S)-dihydroorotate+NADP⁺<=>orotate+NADPH (EC 1.3.1.15); or catalyzes the reaction: (S)-dihydroorotate+fumarate<=>orotate+succinate (EC 1.3.98.1). An enzyme having dihydroorotate dehydrogenase activity is, for example, encoded by the bacterial gene pyrD or an ortholog thereof. Further information regarding pyrD of, e.g., Escherichia coli is available at EcoCyc (www.biocyc.org) or EcoGene (www.ecogene.org) under Accession number EG10807. See also NCBI Reference Sequence: NP_415465.1 for the amino acid sequence (E. coli). In yeast, such as Saccharomyces cerevisiae, an enzyme having dihydroorotase activity is encoded by the pyrD ortholog URA1. Further information regarding URA1 of Saccharomyces cerevisiae is available at YeastCyc (http://yeast.biocyc.org/) under Accession number YKL216W.

As used herein, an “enzyme having orotate phosphoribosyltransferase activity” means an enzyme that catalyzes the reaction: Orotidine 5′-phosphate+diphosphate<=>orotate+5-phospho-alpha-D-ribose 1-diphosphate (EC 2.4.2.10). An enzyme having orotate phosphoribosyltransferase activity is, for example, encoded by the bacterial gene pyrE or an ortholog thereof. Further information regarding pyrE of, e.g., Escherichia coli is available at EcoCyc (www.biocyc.org) or EcoGene (www.ecogene.org) under Accession number EG10808. See also NCBI Reference Sequence: NP_418099.1 for the amino acid sequence (E. coli). In yeast, such as Saccharomyces cerevisiae, an enzyme having orotate phosphoribosyltransferase is encoded by the pyrE orthologs URA5 (main isoform) and URA10 (minor isoform). Further information regarding URA5 and URA10 of Saccharomyces cerevisiae is available at YeastCyc (http://yeast.biocyc.org/) under Accession numbers YML106W and URA10, respectively.

As used herein, an “enzyme having UMP kinase activity” means an enzyme that catalyzes the reaction: ATP+UMP<=>ADP+UDP (EC 2.7.4.22). An enzyme having UMP kinase activity is, for example, encoded by the bacterial gene pyrH or an ortholog thereof. Further information regarding pyrH of, e.g., Escherichia coli is available at EcoCyc (www.biocyc.org) or EcoGene (www.ecogene.org) under Accession number EG11539. See also NCBI Reference Sequence: NP_414713.1 for the amino acid sequence (E. coli). In yeast, such as Saccharomyces cerevisiae, an enzyme having UMP kinase activity is encoded by the pyrH ortholog URA6. Further information regarding URA6 of Saccharomyces cerevisiae is available at YeastCyc (http://yeast.biocyc.org/) under Accession number YKL024C.

As used herein, an “enzyme having nucleoside diphosphate kinase activity” means an enzyme that catalyzes at least the reaction: ATP+UDP<=>ADP+UTP (EC 2.7.4.6). An enzyme having nucleoside diphosphate kinase activity is, for example, encoded by the bacterial gene ndk or an ortholog thereof. Further information regarding ndk of, e.g., Escherichia coli is available at EcoCyc (www.biocyc.org) or EcoGene (www.ecogene.org) under Accession number EG10650. See also NCBI Reference Sequence: NP_417013.1 for the amino acid sequence (E. coli). In yeast, such as Saccharomyces cerevisiae, an enzyme having nucleoside diphosphate kinase activity is encoded by the ndk ortholog YNK1. Further information regarding YNK1 of Saccharomyces cerevisiae is available at YeastCyc (http://yeast.biocyc.org/) under Accession number YKL067W.

As used herein, an “enzyme having cytidylate kinase activity” means an enzyme that catalyzes at least the reaction: ATP+CMP<=>ADP+CDP (EC 2.7.4.25). An enzyme having cytidylate kinase activity is, for example, encoded by the bacterial gene cmk or an ortholog thereof. Further information regarding cmk of, e.g., Escherichia coli is available at EcoCyc (www.biocyc.org) or EcoGene (www.ecogene.org) under Accession number EG11265. See also NCBI Reference Sequence: NP_415430.1 for the amino acid sequence (E. coli). In yeast, such as Saccharomyces cerevisiae, an enzyme having cytidylate kinase activity is encoded by the cmk ortholog URA6. Further information regarding URA6 of Saccharomyces cerevisiae is available at YeastCyc (http://yeast.biocyc.org/) under Accession number YKL024C.

As used herein, an “enzyme having CTP synthase activity” means an enzyme that catalyzes the reaction: ATP+UTP+L-glutamine<=>ADP+phosphate+CTP+L-glutamate (EC 6.3.4.2). An enzyme having CTP synthase activity is, for example, encoded by the bacterial gene pyrG or an ortholog thereof. Further information regarding pyrG of, e.g., Escherichia coli is available at EcoCyc (www.biocyc.org) or EcoGene (www.ecogene.org) under Accession number EG10810. See also NCBI Reference Sequence: NP_417260.1 for the amino acid sequence (E. coli). In yeast, such as Saccharomyces cerevisiae, an enzyme having CTP synthase activity is encoded by the pyrG ortholog URA8. Further information regarding URA8 of Saccharomyces cerevisiae is available at YeastCyc (http://yeast.biocyc.org/) under Accession number YJR103W.

As used herein, an “enzyme having amidophosphoribosyltransferase activity” means an enzyme that catalyzes the reaction: 5-phospho-beta-D-ribosylamine+diphosphate+L-glutamate<=>L-glutamine+5-phospho-alpha-D-ribose 1-diphosphate+H₂O (EC 2.4.2.14). An enzyme having amidophosphoribosyltransferase activity is, for example, encoded by the bacterial gene purF or an ortholog thereof. Further information regarding purF of, e.g., Escherichia coli is available at EcoCyc (www.biocyc.org) or EcoGene (www.ecogene.org) under Accession number EG10794. See also NCBI Reference Sequence: NP_416815.1 for the amino acid sequence (E. coli). In yeast, such as Saccharomyces cerevisiae, an enzyme having amidophosphoribosyltransferase activity is encoded by the purF ortholog ADE4. Further information regarding ADE4 of Saccharomyces cerevisiae is available at YeastCyc (http://yeast.biocyc.org/) under Accession number YMR300C.

As used herein, an “enzyme having phosphoribosylamine-glycine ligase activity” means an enzyme that catalyzes the reaction: ATP+5-phospho-beta-D-ribosylamine+glycine<=>ADP+phosphate+N¹-(5-phospho-beta-D-ribosyl)glycinamide (EC 6.3.4.13). An enzyme having phosphoribosylamine-glycine ligase activity is, for example, encoded by the bacterial gene purD or an ortholog thereof. Further information regarding purD of, e.g., Escherichia coli is available at EcoCyc (www.biocyc.org) or EcoGene (www.ecogene.org) under Accession number EG10792. See also NCBI Reference Sequence: NP_418433.1 for the amino acid sequence (E. coli). In yeast, such as Saccharomyces cerevisiae, an enzyme having phosphoribosylamine-glycine ligase activity is encoded by the purD ortholog ADE5,7 (encoding a bifunctional protein). Further information regarding ADE5,7 of Saccharomyces cerevisiae is available at YeastCyc (http://yeast.biocyc.org/) under Accession number YGL234W.

As used herein, an “enzyme having phosphoribosylglycineamide formyltransferase activity” means an enzyme that catalyzes the reaction: 10-formyltetrahydrofolate+N¹-(5-phospho-beta-D-ribosyl)glycinamide<=>tetrahydrofolate+N²-formyl-N¹-(5-phospho-beta-D-ribosyl)glycinamide (EC 2.1.2.2). An enzyme having phosphoribosylglycineamide formyltransferase activity is, for example, encoded by the bacterial gene purT or an ortholog thereof. Further information regarding purT of, e.g., Escherichia coli is available at EcoCyc (www.biocyc.org) or EcoGene (www.ecogene.org) under Accession number EG11809. See also NCBI Reference Sequence: NP_416363.1 for the amino acid sequence (E. coli). In yeast, such as Saccharomyces cerevisiae, an enzyme having phosphoribosylglycineamide formyltransferase activity is encoded by the purT ortholog ADE8. Further information regarding ADE8 of Saccharomyces cerevisiae is available at YeastCyc (http://yeast.biocyc.org/) under Accession number YDR408C.

As used herein, an “enzyme having phosphoribosylformylglycinamidine synthase activity” means an enzyme that catalyzes the reaction: ATP+N²-formyl-N¹-(5-phospho-beta-D-ribosyl)glycinamide+L-glutamine+H₂O<=>ADP+phosphate+2-(formamido)-N¹-(5-phospho-beta-D-ribosyl)acetamidine+L-glutamate (EC 6.3.5.3). An enzyme having phosphoribosylformylglycinamidine synthase activity is, for example, encoded by the bacterial gene purL or an ortholog thereof. Further information regarding purL of, e.g., Escherichia coli is available at EcoCyc (www.biocyc.org) or EcoGene (www.ecogene.org) under Accession number EG10797. See also NCBI Reference Sequence: YP_026170.1 for the amino acid sequence (E. coli). In yeast, such as Saccharomyces cerevisiae, an enzyme having phosphoribosylformylglycinamidine synthase activity is encoded by the purL ortholog ADE6. Further information regarding ADE6 of Saccharomyces cerevisiae is available at YeastCyc (http://yeast.biocyc.org/) under Accession number YGR061C.

As used herein, an “enzyme having phosphoribosylformylglycineamidine cyclo-ligase activity” means an enzyme that catalyzes the reaction: ATP+2-(formamido)-N¹-(5-phospho-beta-D-ribosyl)acetamidine<=>ADP+phosphate+5-amino-1-(5-phospho-beta-D-ribosyl)imidazole (EC 6.3.3.1). An enzyme having phosphoribosylformylglycineamidine cyclo-ligase activity is, for example, encoded by the bacterial gene purM or an ortholog thereof. Further information regarding purM of, e.g., Escherichia coli is available at EcoCyc (www.biocyc.org) or EcoGene (www.ecogene.org) under Accession number EG10798. See also NCBI Reference Sequence: NP_416994.1 for the amino acid sequence (E. coli). In yeast, such as Saccharomyces cerevisiae, an enzyme having phosphoribosylformylglycineamidine cyclo-ligase activity is encoded by the purM ortholog ADE5,7 (encoding a bifunctional protein). Further information regarding ADE5,7 of Saccharomyces cerevisiae is available at YeastCyc (http://yeast.biocyc.org/) under Accession number YGL234W.

As used herein, an “enzyme having N5-carboxyaminoimidazole ribonucleotide synthetase activity” means an enzyme that catalyzes the reaction: ATP+5-amino-1-(5-phospho-D-ribosyl)imidazole+HCO₃⁻<=>ADP+phosphate+5-carboxyamino-1-(5-phospho-D-ribosyl)imidazole (EC 6.3.4.18). An enzyme having N5-carboxyaminoimidazole ribonucleotide synthetase activity is, for example, encoded by the bacterial gene purK or an ortholog thereof. Further information regarding purK of, e.g., Escherichia coli is available at EcoCyc (www.biocyc.org) or EcoGene (www.ecogene.org) under Accession number EG10796. See also NCBI Reference Sequence: NP_415055.1 for the amino acid sequence (E. coli).

As used herein, an “enzyme having N5-carboxyaminoimidazole ribonucleotide mutase activity” means an enzyme that catalyzes the reaction: 5-carboxyamino-1-(5-phospho-D-ribosyl)imidazole<=>5-amino-1-(5-phospho-D-ribosyl)imidazole-4-carboxylate (EC 5.4.99.18). An enzyme having N5-carboxyaminoimidazole ribonucleotide mutase activity is, for example, encoded by the bacterial gene purE or an ortholog thereof. Further information regarding purE of, e.g., Escherichia coli is available at EcoCyc (www.biocyc.org) or EcoGene (www.ecogene.org) under Accession number EG10793. See also NCBI Reference Sequence: NP_415056.1 for the amino acid sequence (E. coli).

As used herein, an “enzyme having phosphoribosylaminoimidazolesuccinocarboxamide synthase activity” means an enzyme that catalyzes the reaction: ATP+5-amino-1-(5-phospho-D-ribosyl)imidazole-4-carboxylate+L-aspartate<=>ADP+phosphate+(S)-2-(5-amino-1-(5-phospho-D-ribosyl)imidazole-4-carboxamido)succinate (EC 6.3.2.6). An enzyme having phosphoribosylaminoimidazolesuccinocarboxamide synthase activity is, for example, encoded by the bacterial gene purC or an ortholog thereof. Further information regarding purC of, e.g., Escherichia coli is available at EcoCyc (www.biocyc.org) or EcoGene (www.ecogene.org) under Accession number EG10791. See also NCBI Reference Sequence: NP_416971.1 for the amino acid sequence (E. coli). In yeast, such as Saccharomyces cerevisiae, an enzyme having phosphoribosylaminoimidazolesuccinocarboxamide synthase activity is encoded by the purC ortholog ADE1. Further information regarding ADE1 of Saccharomyces cerevisiae is available at YeastCyc (http://yeast.biocyc.org/) under Accession number YAR015W.

As used herein, an “enzyme having adenylosuccinate lyase activity” means an enzyme that catalyzes the reaction: (S)-2-(5-amino-1-(5-phospho-D-ribosyl)imidazole-4-carboxamido)succinate<=>fumarate+5-amino-1-(5-phospho-D-ribosyl)imidazole-4-carboxamide (EC 4.3.2.2). An enzyme having adenylosuccinate lyase activity is, for example, encoded by the bacterial gene purB or an ortholog thereof. Further information regarding purB of, e.g., Escherichia coli is available at EcoCyc (www.biocyc.org) or EcoGene (www.ecogene.org) under Accession number EG11314. See also NCBI Reference Sequence: NP_415649.1 for the amino acid sequence (E. coli). In yeast, such as Saccharomyces cerevisiae, an enzyme having adenylosuccinate lyase activity is encoded by the purB ortholog ADE13. Further information regarding ADE13 of Saccharomyces cerevisiae is available at YeastCyc (http://yeast.biocyc.org/) under Accession number YLR359W.

As used herein, an “enzyme having phosphoribosylaminoimidazole-carboxamide formyltransferase activity” means an enzyme that catalyzes the reaction: 10-formyltetrahydrofolate+5-amino-1-(5-phospho-D-ribosyl)imidazole-4-carboxamide<=>tetrahydrofolate+5-formamido-1-(5-phospho-D-ribosyl)imidazole-4-carboxamide (EC 2.1.2.3). An enzyme having phosphoribosylaminoimidazole-carboxamide formyltransferase activity is, for example, encoded by the bacterial gene purH or an ortholog thereof. Further information regarding purH of, e.g., Escherichia coli is available at EcoCyc (www.biocyc.org) or EcoGene (www.ecogene.org) under Accession number EG10795. See also NCBI Reference Sequence: NP_418434.1 for the amino acid sequence (E. coli). In yeast, such as Saccharomyces cerevisiae, an enzyme having phosphoribosylaminoimidazole-carboxamide formyltransferase activity is encoded by the purH orthologs ADE16 and ADE17. Further information regarding ADE16 and ADE17 of Saccharomyces cerevisiae is available at YeastCyc (http://yeast.biocyc.org/) under Accession numbers YLR028C and YMR120C, respectively.

As used herein, an “enzyme having IMP cyclohydolase activity” means an enzyme that catalyzes the reaction: IMP+H₂O<=>5-formamido-1-(5-phospho-D-ribosyl)imidazole-4-carboxamide (EC 3.5.4.10). An enzyme having IMP cyclohydolase activity is, for example, encoded by the bacterial gene purH or an ortholog thereof. Further information regarding purH of, e.g., Escherichia coli is available at EcoCyc (www.biocyc.org) or EcoGene (www.ecogene.org) under Accession number EG10795. See also NCBI Reference Sequence: NP_418434.1 for the amino acid sequence (E. coli). In yeast, such as Saccharomyces cerevisiae, an enzyme having IMP cyclohydolase activity is encoded by the purH orthologs ADE16 and ADE17. Further information regarding ADE16 and ADE17 of Saccharomyces cerevisiae is available at YeastCyc (http://yeast.biocyc.org/) under Accession numbers YLR028C and YMR120C, respectively.

As used herein, an “enzyme having adenylosuccinate synthase activity” means an enzyme that catalyzes the reaction: GTP+IMP+L-aspartate<=>GDP+phosphate+N⁶-(1,2-dicarboxyethyl)-AMP (EC 6.3.4.4). An enzyme having adenylosuccinate synthase activity is, for example, encoded by the bacterial gene purA or an ortholog thereof. Further information regarding purA of, e.g., Escherichia coli is available at EcoCyc (www.biocyc.org) or EcoGene (www.ecogene.org) under Accession number EG10790. See also NCBI Reference Sequence: NP_418598.1 for the amino acid sequence (E. coli). In yeast, such as Saccharomyces cerevisiae, an enzyme having adenylosuccinate synthase activity is encoded by the purA ortholog ADE12. Further information regarding ADE12 of Saccharomyces cerevisiae is available at YeastCyc (http://yeast.biocyc.org/) under Accession number YNL220W.

As used herein, an “enzyme having adenylate kinase activity” means an enzyme that catalyzes the reaction: ATP+AMP<=>2 ADP (EC 2.7.4.3). An enzyme having adenylate kinase activity is, for example, encoded by the bacterial gene adk or an ortholog thereof. Further information regarding adk of, e.g., Escherichia coli is available at EcoCyc (www.biocyc.org) or EcoGene (www.ecogene.org) under Accession number EG10032. See also NCBI Reference Sequence: NP_415007.1 for the amino acid sequence (E. coli). In yeast, such as Saccharomyces cerevisiae, an enzyme having adenylate kinase activity is encoded by the adk ortholog ADK1. Further information regarding ADK1 of Saccharomyces cerevisiae is available at YeastCyc (http://yeast.biocyc.org/) under Accession number YDR226W.

As used herein, an “enzyme having ATP synthase activity” means an enzyme that catalyzes the reaction: ATP+H₂O+H⁺ (cytosol)<=>ADP+phosphate+H+(periplasm) (EC 3.6.3.14). An enzyme having ATP synthase activity is, for example, the ATP synthase F₀ or F₁ complexe encoded by the bacterial atp operon (including the genes atpB, atpF, atpE, atpD, atpG, atpA, atpH and atpC) or orthologs thereof. Further information regarding atpB, atpF, atpE, atpD, atpG, atpA, atpH and atpC of, e.g., Escherichia coli is available at EcoCyc (www.biocyc.org) or EcoGene (www.ecogene.org) under Accession numbers EG10099, EG10103, EG10102, EG10101, EG10104, EG10098, EG10105 and EG10100, respectively. See also NCBI Reference Sequence: NP_418194, NP_418192, NP_418193, NP_418188, NP_418189, NP_418190, NP_418191 and NP_418187 for the respective amino acid sequence (E. coli). In yeast, such as Saccharomyces cerevisiae, ATP synthase complexes are encoded by the genes ATP1, ATP2, ATP3, ATP4, ATP5, ATP6, ATP7, ATP8, ATP10, ATP11, ATP12, ATP14, ATP15, ATP16, ATP17, ATP19 and ATP20. Further information regarding ATP1, ATP2, ATP3, ATP4, ATP5, ATP6, ATP7, ATP8, ATP10, ATP11, ATP12, ATP14, ATP15, ATP16, ATP17, ATP19 and ATP20. of Saccharomyces cerevisiae is available at YeastCyc (http://yeast.biocyc.org/) under Accession numbers YBL099W, YJR121W, YBR039W, YPL078C, YDR298C, Q0085, YKL016C, Q0080, YLR393W, YNL315C, YJL180C, YLR295C, YPL271W, YDL004W, YDR377W, YOL077W-A and YPR020W, respectively.

As used herein, an “enzyme having IMP dehydrogenase activity” means an enzyme that catalyzes the reaction: Inosine 5′-phosphate+NAD⁺+H₂O<=>xanthosine 5′-phosphate+NADH (EC 1.1.1.205). An enzyme having IMP dehydrogenase activity is, for example, encoded by the bacterial gene guaB or an ortholog thereof. Further information regarding guaB of, e.g., Escherichia coli is available at EcoCyc (www.biocyc.org) or EcoGene (www.ecogene.org) under Accession number EG10421. See also NCBI Reference Sequence: NP_417003.1 for the amino acid sequence (E. coli). In yeast, such as Saccharomyces cerevisiae, an enzyme having IMP dehydrogenase activity is encoded by the guaB orthologs IMD2, IMD3 and IMD4. Further information regarding IMD2, IMD3 and IMD4 of Saccharomyces cerevisiae is available at YeastCyc (http://yeast.biocyc.org/) under Accession numbers YHR216W, YLR432W and YML056C, respectively.

As used herein, an “enzyme having GMP synthase activity” means an enzyme that catalyzes the reaction: ATP+XMP+L-glutamine+H₂O<=>AMP+diphosphate+GMP+L-glutamate (EC 6.3.5.2). An enzyme having GMP synthase activity is, for example, encoded by the bacterial gene guaA or an ortholog thereof. Further information regarding guaA of, e.g., Escherichia coli is available at EcoCyc (www.biocyc.org) or EcoGene (www.ecogene.org) under Accession number EG10420. See also NCBI Reference Sequence: NP_417002.1 for the amino acid sequence (E. coli). In yeast, such as Saccharomyces cerevisiae, an enzyme having GMP synthase activity is encoded by the guaA ortholog GUA1. Further information regarding GUA1 of Saccharomyces cerevisiae is available at YeastCyc (http://yeast.biocyc.org/) under Accession number YMR217W.

As used herein, an “enzyme having guanylate kinase activity” means an enzyme that catalyzes the reaction: ATP+GMP<=>ADP+GDP (EC 2.7.4.8). An enzyme having guanylate kinase activity is, for example, encoded by the bacterial gene gmk or an ortholog thereof. Further information regarding gmk of, e.g., Escherichia coli is available at EcoCyc (www.biocyc.org) or EcoGene (www.ecogene.org) under Accession number EG10965. See also NCBI Reference Sequence: NP_418105.1 for the amino acid sequence (E. coli). In yeast, such as Saccharomyces cerevisiae, an enzyme having guanylate kinase activity is encoded by the gmk ortholog GUK1. Further information regarding GUK1 of Saccharomyces cerevisiae is available at YeastCyc (http://yeast.biocyc.org/) under Accession number YDR454C.

As used herein “an enzyme having pyruvate kinase II activity” means an enzyme that catalyzes the reaction: ATP+pyruvate<=>ADP+phosphoenolpyruvate (EC 2.7.1.40). An enzyme having pyruvate kinase II activity is, for example, encoded by the bacterial gene pykA or an ortholog thereof. Further information regarding pykA of, e.g., Escherichia coli is available at EcoCyc (www.biocyc.org) or EcoGene (www.ecogene.org) under Accession number EG10803. See also NCBI Reference Sequence: NP_416368.1 for the amino acid sequence (E. coli). In yeast, such as Saccharomyces cerevisiae, an enzyme having pyruvate kinase II activity is encoded by the pykA orthologs PYK1 and PYK2. Further information regarding PYK1 and PYK2 of Saccharomyces cerevisiae is available at YeastCyc (http://yeast.biocyc.org/) under Accession numbers YAL038W and YOR347C.

As used herein, “an enzyme having GMP reductase activity” means an enzyme that catalyzes the reaction: Inosine 5′-phosphate+NH₃+NADP⁺<=>guanosine 5′-phosphate+NADPH (EC 1.7.1.7). An enzyme having GMP reductase activity is, for example, encoded by the bacterial gene guaC or an ortholog thereof. Further information regarding guaC of, e.g., Escherichia coli is available at EcoCyc (www.biocyc.org) or EcoGene (www.ecogene.org) under Accession number EG10422. See also NCBI Reference Sequence: NP_414646.1 for the amino acid sequence (E. coli).

As used herein, an “enzyme having deoxyguanosine triphosphate triphosphohydrolase activity” means an enzyme that catalyzes the reaction: dGTP+H₂O<=>deoxyguanosine+triphosphate (EC 3.1.5.1). An enzyme having deoxyguanosine triphosphate triphosphohydrolase activity is, for example, encoded by the bacterial gene dgt or an ortholog thereof. Further information regarding dgt of, e.g., Escherichia coli is available at EcoCyc (www.biocyc.org) or EcoGene (www.ecogene.org) under Accession number EG10225. See also NCBI Reference Sequence: NP_414702.1 for the amino acid sequence (E. coli).

As used herein, an “enzyme having ribonucleoside-diphosphate reductase activity” means an enzyme that catalyzes the reaction: 2′-deoxyribonucleoside diphosphate+thioredoxin disulfide+H₂O<=>ribonucleoside diphosphate+thioredoxin (EC 1.17.4.1). An enzyme having ribonucleoside-diphosphate reductase activity is, for example, encoded by the bacterial genes nrdA (encoding the alpha subunit) and nrdB (encoding the beta subunit) or orthologs thereof. Further information regarding nrdA and nrdB of, e.g., Escherichia coli is available at EcoCyc (www.biocyc.org) or EcoGene (www.ecogene.org) under Accession numbers EG10660 and EG10661, respectively. See also NCBI Reference Sequences: NP_416737.1 and NP_416738 for the respective amino acid sequence (E. coli). In yeast, such as Saccharomyces cerevisiae, the ribonucleoside-diphosphate reductase is encoded by the genes RNR1, RNR2, RNR3 and RNR4 (encoding the small and large subunits for the dimeric complexes forming the tetramer). Further information regarding RNR1, RNR2, RNR3 and RNR4 of Saccharomyces cerevisiae is available at YeastCyc (http://yeast.biocyc.org/) under Accession numbers YER070W, YJL026W, YIL066C and YGR180C, respectively.

As used herein, an “enzyme having ribonucleoside-triphosphate reductase activity” means an enzyme that catalyzes the reaction: 2′-deoxyribonucleoside triphosphate+thioredoxin disulfide+H₂O<=>ribonucleoside triphosphate+thioredoxin (EC 1.17.4.2). An enzyme ribonucleoside-triphosphate reductase activity is, for example, encoded by the bacterial gene nrdD or an ortholog thereof. Further information regarding nrdD of, e.g., Escherichia coli is available at EcoCyc (www.biocyc.org) or EcoGene (www.ecogene.org) under Accession number EG11417. See also NCBI Reference Sequence: NP_418659.1 for the amino acid sequence (E. coli).

As used herein, an “enzyme having dTMP kinase activity” means an enzyme that catalyzes the reaction: ATP+dTMP<=>ADP+dTDP (EC 2.7.4.9). An enzyme having dTMP kinase activity is, for example, encoded by the bacterial gene tmk or an ortholog thereof. Further information regarding tmk of, e.g., Escherichia coli is available at EcoCyc (www.biocyc.org) or EcoGene (www.ecogene.org) under Accession number EG12302. See also NCBI Reference Sequence: NP_415616.1 for the amino acid sequence (E. coli). In yeast, such as Saccharomyces cerevisiae, an enzyme having dTMP kinase activity is encoded by the tmk ortholog CDC8. Further information regarding CDC8 of Saccharomyces cerevisiae is available at YeastCyc (http://yeast.biocyc.org/) under Accession number YJR057W.

As used herein, an “enzyme having deoxyuridine triphosphatase activity” means an enzyme that catalyzes the reaction: dUTP+H(2)O<=>dUMP+diphosphate (EC 3.6.1.23). An enzyme having deoxyuridine triphosphatase activity is, for example, encoded by the bacterial gene dut or an ortholog thereof. Further information regarding dut of, e.g., Escherichia coli is available at EcoCyc (www.biocyc.org) or EcoGene (www.ecogene.org) under Accession number EG10251. See also NCBI Reference Sequence: NP_418097.1 for the amino acid sequence (E. coli). In yeast, such as Saccharomyces cerevisiae, an enzyme having deoxyuridine triphosphatase activity is encoded by the dut ortholog DUT1. Further information regarding DUT1 of Saccharomyces cerevisiae is available at YeastCyc (http://yeast.biocyc.org/) under Accession number YBR252W.

As used herein, an “enzyme having thymidylate synthase activity” means an enzyme that catalyzes the reaction: 5,10-methylenetetrahydrofolate+dUMP<=>dihydrofolate+dTMP (EC 2.1.1.45). An enzyme having thymidylate synthase activity is, for example, encoded by the bacterial gene thyA or an ortholog thereof. Further information regarding thyA of, e.g., Escherichia coli is available at EcoCyc (www.biocyc.org) or EcoGene (www.ecogene.org) under Accession number EG11002. See also NCBI Reference Sequence: NP_417304.1 for the amino acid sequence (E. coli). In yeast, such as Saccharomyces cerevisiae, an enzyme having thymidylate synthase activity is encoded by the thyA ortholog CDC21. Further information regarding CDC21 of Saccharomyces cerevisiae is available at YeastCyc (http://yeast.biocyc.org/) under Accession number YOR074C.

As used herein, an “enzyme having dCTP deaminase activity” means an enzyme that catalyzes the reaction: dCTP+H₂O<=>dUTP+NH₃ (EC 3.5.4.13). An enzyme having dCTP deaminase activity is, for example, encoded by the bacterial gene dcd or an ortholog thereof. Further information regarding dcd of, e.g., Escherichia coli is available at EcoCyc (www.biocyc.org) or EcoGene (www.ecogene.org) under Accession number EG11418. See also NCBI Reference Sequence: NP_416569.1 for the amino acid sequence (E. coli). In yeast, such as Saccharomyces cerevisiae, an enzyme having dCTP deaminase activity is encoded by the dcd ortholog DCD1. Further information regarding DCD1 of Saccharomyces cerevisiae is available at YeastCyc (http://yeast.biocyc.org/) under Accession number YHR144C.

As used herein, an “enzyme having cytidine deaminase activity” means an enzyme that catalyzes the reaction: Cytidine+H₂O<=>uridine+NH₃ (EC 3.5.4.5). An enzyme having cytidine deaminase activity is, for example, encoded by the bacterial gene cdd or an ortholog thereof. Further information regarding cdd of, e.g., Escherichia coli is available at EcoCyc (www.biocyc.org) or EcoGene (www.ecogene.org) under Accession number EG10137. See also NCBI Reference Sequence: NP_416648.1 for the amino acid sequence (E. coli). In yeast, such as Saccharomyces cerevisiae, an enzyme having cytidine deaminase activity is encoded by the cdd ortholog CDD1. Further information regarding CDD1 of Saccharomyces cerevisiae is available at YeastCyc (http://yeast.biocyc.org/) under Accession number YLR245C.

As used herein, an “enzyme having cytosine deaminase activity” means an enzyme that catalyzes the reaction: Cytosine+H₂O<=>uracil+NH₃ (EC 3.5.4.1). An enzyme having cytosine deaminase activity is, for example, encoded by the bacterial gene codA or an ortholog thereof. Further information regarding codA of, e.g., Escherichia coli is available at EcoCyc (www.biocyc.org) or EcoGene (www.ecogene.org) under Accession number EG11326. See also NCBI Reference Sequence: NP_414871.1 for the amino acid sequence (E. coli). In yeast, such as Saccharomyces cerevisiae, an enzyme having cytosine deaminase activity is encoded by the codA ortholog FCY1. Further information regarding FCY1 of Saccharomyces cerevisiae is available at YeastCyc (http://yeast.biocyc.org/) under Accession number YPR062W.

As used herein, an “enzyme having uridine kinase activity” means an enzyme that catalyzes the reaction: ATP+uridine<=>ADP+UMP (EC 2.7.1.48). An enzyme having uridine kinase activity is, for example, encoded by the bacterial gene udk or an ortholog thereof. Further information regarding udk of, e.g., Escherichia coli is available at EcoCyc (www.biocyc.org) or EcoGene (www.ecogene.org) under Accession number EG11701. See also NCBI Reference Sequence: NP_416570.1 for the amino acid sequence (E. coli). In yeast, such as Saccharomyces cerevisiae, an enzyme having uridine kinase activity is encoded by the udk ortholog URK1. Further information regarding URK1 of Saccharomyces cerevisiae is available at YeastCyc (http://yeast.biocyc.org/) under Accession number YNR012W.

As used herein, an “enzyme having thymidine kinase activity” means an enzyme that catalyzes the reaction: ATP+thymidine<=>ADP+thymidine 5′-phosphate (EC 2.7.1.21). An enzyme having thymidine kinase activity is, for example, encoded by the bacterial gene tdk or an ortholog thereof. Further information regarding tdk of, e.g., Escherichia coli is available at EcoCyc (www.biocyc.org) or EcoGene (www.ecogene.org) under Accession number EG10994. See also NCBI Reference Sequence: NP_415754.1 for the amino acid sequence (E. coli).

As used herein, an “enzyme having uridine phosphorylase activity” means an enzyme that catalyzes the reaction: Uridine+phosphate<=>uracil+alpha-D-ribose 1-phosphate (EC 2.4.2.3). An enzyme having uridine phosphorylase activity is, for example, encoded by the bacterial gene udp or an ortholog thereof. Further information regarding udp of, e.g., Escherichia coli is available at EcoCyc (www.biocyc.org) or EcoGene (www.ecogene.org) under Accession number EG11045. See also NCBI Reference Sequence: NP_418275.1 for the amino acid sequence (E. coli).

As used herein, an “enzyme having thymidine phosphorylase activity” means an enzyme that catalyzes the reaction: Thymidine+phosphate<=>thymine+2-deoxy-alpha-D-ribose 1-phosphate (EC 2.4.2.4). An enzyme having thymidine phosphorylase activity is, for example, encoded by the bacterial gene deoA or an ortholog thereof. Further information regarding deoA of, e.g., Escherichia coli is available at EcoCyc (www.biocyc.org) or EcoGene (www.ecogene.org) under Accession number EG10219. See also NCBI Reference Sequence: NP_418799.1 for the amino acid sequence (E. coli).

“Aryl sulfotransferase activity” as used herein refers to the ability of a polypeptide to catalyze the transfer of a sulfate group from a donor molecule to an aryl acceptor molecule.

“Tyrosine ammonia lyase activity” as used herein refers to the ability of a polypeptide to catalyzed the conversion of L-tyrosine into p-coumaric acid.

As used herein, “polypeptide” or “protein” are used interchangeably and denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

As used herein, “nucleic acid” or “polynucleotide” are used interchangeably and denote a polymer of at least two nucleic acid monomer units or bases (e.g., adenine, cytosine, guanine, thymine) covalently linked by a phosphodiester bond, regardless of length or base modification.

“Recombinant” or “non-naturally occurring”, when used with reference to, e.g., a host cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant host cells expressing genes that are not found within the native (non-recombinant) form of the cell or express native genes that are otherwise expressed at a different level. Particularly, a “recombinant polypeptide” signifies a polypeptide produced with molecular biological techniques based on the natural DNA of the original genome or the natural DNA modified with a heterogeneous DNA sequence and with which it can be combined, e.g. with plasmids, and can be replicated and expressed in a suitable host cell.

As used herein, “reducing”, “reduction of” or “reduced” growth of a microorganism, which has been modified or subjected to treatment (e.g., exposure to an inhibitor of an enzyme”), means that the rate of cell biomass formation of said microorganism is reduced compared to the rate of cell biomass formation of an unmodified or untreated microorganism of the same type (control) when grown under otherwise identical conditions. The rate of cell biomass formation of the modified or treated microorganism may be reduced so that the cell biomass concentration is less than about 95%, such as less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15% or less than about 10%, or any percentage, in whole integers between about 95% and about 10% (e.g., 94%, 93%, 92%, etc.), compared to the cell biomass concentration of an unmodified or untreated microorganism of the same type (control) when grown under otherwise identical conditions for at least 48 hours after inducing the growth reduction (e.g. initiating step b)). The rate of cell biomass formation of the modified or treated microorganism may be reduced so that the final biomass concentration is in the range of about 10% to about 95%, such as about 20% to about 95%, about 30% to about 95%, about 40% to about 95%, about 50% to about 95%, about 10% to about 80%, about 20% to about 80%, about 30% to about 80%, about 10% to about 70%, about 20% to about 70%, about 30% to about 70%, about 10% to about 60%, about 20% to about 60%, about 30% to about 60%, about 10% to about 50%, about 20% to about 50% or about 30% to about 50%, of the final biomass concentration of the unmodified or untreated microorganism of the same type (control) when grown under otherwise identical conditions for at least 48 hours after inducing the growth reduction (e.g. initiating step b)). The cell biomass concentration of a microorganism can be measured using standard methods including, but not limited to, measuring optical density or determining dry cell weight of the culture. The rate of biomass formation or growth rate may be determined directly from these measurements.

As used herein, “inhibiting” or “inhibition of” the expression of a polypeptide (such as an enzyme as described herein, such as an enzyme having orotidine-5′-phosphate decarboxylase activity) means that the expression of said polypeptide in a modified microorganism is reduced compared to the expression of said polypeptide in an unmodified microorganism of the same type (control). The expression of polypeptide in a modified microorganism may be reduced by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the expression of said polypeptide in an unmodified microorganism of the same type (control). More particularly, “inhibiting”, “inhibition of” or “inhibit” expression of a polypeptide (such as an enzyme as described herein, such as an enzyme having orotidine-5′-phosphate decarboxylase activity) means that the amount of the polypeptide in the microorganism is reduced by at least about 10%, and preferably by at least about 20%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the amount of said polypeptide in an unmodified microorganism of the same type (control). The expression or amount of a polypeptide (such as an enzyme as described herein, such as an enzyme having orotidine-5′-phosphate decarboxylase activity) in a microorganism can be determined by any suitable means know in the art, including techniques such as ELISA, Immunohistochemistry, Western Blotting or Flow Cytometry.

As used herein, “inhibiting” or “inhibition of” the expression of SibB means that the expression of SibB in a modified microorganism is reduced compared to the expression of SibB in an unmodified microorganism of the same type (control). The expression of SibB in a modified microorganism may be reduced by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the expression of SibB in an unmodified microorganism of the same type (control). More particularly, “inhibiting”, “inhibition of” or “inhibit” expression of SibB means that the amount of SibB in the microorganism is reduced by at least about 10%, and preferably by at least about 20%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the amount of SibB in an unmodified microorganism of the same type (control). The expression or amount of SibB in a microorganism can be determined by any suitable means know in the art, including techniques such as Northern blotting, quantitative RT-PCR, and the like.

As used herein, “increasing” or “increase of” the expression of lbsB or a variant thereof means that the expression of lbsB or a variant thereof in a modified microorganism is increased compared to the expression of lbsB or a variant thereof in an unmodified microorganism of the same type (control). The expression of lbsB or a variant thereof in a modified microorganism may be increased by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 200%, at least about 300%, at least about 400%, at least about 500%, at least about 600%, at least about 700%, at least about 800%, at least about 900% or at least about 1000% compared to the expression of lbsB or a variant thereof in an unmodified microorganism of the same type (control). More particularly, “increasing”, “increase of” or “increase” the expression of lbsB or a variant thereof means that the amount of lbsB or a variant thereof in the microorganism is increased by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 200%, at least about 300%, at least about 400%, at least about 500%, at least about 600%, at least about 700%, at least about 800%, at least about 900% or at least about 1000% compared to the amount of lbsB or a variant thereof in an unmodified microorganism of the same type (control). The expression or amount of lbsB or a variant thereof in a microorganism can be determined by any suitable means know in the art, including techniques such as ELISA, Immunohistochemistry, Western Blotting or Flow Cytometry.

“Inactivating”, “inactivation” and “inactivated”, when used in the context of a gene, generally means that the gene of interest (e.g. a gene encoding an enzyme as described herein, such as a gene encoding an enzyme having orotidine-5′-phosphate decarboxylase activity) is not expressed in a functional protein form. The phrases can mean that the modified gene encodes a completely non-functional protein. It is also possible that the modified DNA region is unable to naturally express the gene due to the deletion of a part of or the entire gene sequence, the shifting of the reading frame of the gene, the introduction of missense/nonsense mutation(s), or the modification of an adjacent region of the gene, including sequences controlling gene expression, such as a promoter, enhancer, attenuator, ribosome-binding site, etc. Techniques for inactivating a gene are well-known to those of skill in the art, and include random mutagenesis, site specific mutagenesis, recombination, integration and others. Preferably, a gene of interest is inactivated by deletion of a part of or the entire gene sequence, such as by gene replacement. Gene replacement using homologous recombination can be conducted by employing a linear DNA, which is known as “lambda-red mediated gene replacement” (Datsenko and Wanner, 2000), or by employing a plasmid containing a temperature-sensitive replication origin (U.S. Pat. No. 6,303,383 or JP 05-007491 A).

The presence or absence of a gene on the chromosome of a microorganism can be detected by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the protein encoded by the gene can be measured by well-known methods, including techniques such as ELISA, Immunohistochemistry, Western Blotting or Flow Cytometry.

As used herein, “heterologous”, “foreign” and “exogenous” nucleic acid molecule are used interchangeably and refer to a DNA or RNA molecule that does not occur naturally as part of the genome of the microorganism in which it is present or which is found in a location or locations in the genome that differ from that in which it occurs in nature. Thus, a “heterologous”, “foreign” or “exogenous” nucleic acid molecule is a DNA or RNA molecule that is not normally found in the host genome in an identical context. It is a DNA or RNA molecule that is not endogenous to the microorganism and has been exogenously introduced into the microorganism. In one aspect, a “heterologous” DNA molecule may be the same as the host DNA but modified by methods known in the art, where the modification(s) includes, but are not limited to, insertion in a vector, linked to a foreign promoter and/or other regulatory elements, or repeated at multiple copies. In another aspect, a “heterologous” DNA molecule may be from a different organism, a different species, a different genus or a different kingdom, as the host DNA.

As used herein, the phrase “inhibitor of the enzyme” refers to any chemical compound, natural or synthetic, that inhibits the catalytic activity of the enzyme. An inhibitor of the enzyme does not necessarily need to achieve 100% or complete inhibition. In this regard, an inhibitor of the enzyme can induce any level of inhibition. Desirably, an inhibitor of the enzyme can inhibit at least about 10% of the catalytic activity of the enzyme in the absence of any inhibitors of the enzyme. It is more preferred that an inhibitor of the enzyme achieve at least about 50% inhibition. Most preferably, an inhibitor of the enzyme inhibits at least about 90% of the catalytic activity of the enzyme in the absence of any inhibitors of the enzyme. Non-limiting examples of, e.g., inhibitors of an enzyme having orotidine-5′-phosphate decarboxylase activity include 5-Fluoroorotic acid (5-FOA), 6-Azauridine-5′-monophosphate (6-Aza-UMP), 1-ribosylallopurinol-5′-phosphate and 6-iodouridine-5′-monophosphate (6-iodo-UMP) among others.

As used herein, the term “ortholog” or “orthologs” refers to genes, nucleic acid molecules encoded thereby, i.e., mRNA, or proteins encoded thereby that are derived from a common ancestor gene but are present in different species.

As used herein, “heterologous” polypeptide means that a polypeptide is normally not found in or made (i.e. expressed) by the host microorganism, but derived from a different organism, a different species, a different genus or a different kingdom.

As used herein, “host cell” as used herein refers to a microorganism that is capable of reproducing its genetic material and along with it recombinant genetic material that has been introduced into it—e.g., via heterologous transformation.

As used herein, “expression” includes any step involved in the production of a polypeptide (e.g., encoded enzyme) including, but not limited to, transcription, post-transcriptional modification, translation, post-translational modification, and secretion.

As used herein, “vector” refers to a nucleic acid molecule capable of transporting another nucleic acid molecule to which it has been linked. One type of vector is a “plasmid”, which refers to a circular double stranded nucleic acid loop into which additional nucleic acid segments can be ligated. Certain vectors are capable of directing the expression of genes to which they are operatively linked. Such vectors are referred to herein as “expression vectors”. Certain other vectors are capable of facilitating the insertion of a exogenous nucleic acid molecule into a genome of a host cell. Such vectors are referred to herein as “transformation vectors”. In general, vectors of utility in recombinant nucleic acid techniques are often in the form of plasmids. In the present specification, “plasmid” and “vector” can be used interchangeably as the plasmid is the most commonly used form of a vector. Large numbers of suitable vectors are known to those of skill in the art and commercially available.

As used herein, “regulatory elements” refers nucleic acid sequences that affect the expression of a coding sequence. Regulatory elements are known in the art and include, but are not limited to, promoters, enhancers, transcription terminators, polyadenylation sites, matrix attachment regions and/or other elements that regulate expression of a coding sequence.

As used herein, “promoter” refers to a sequence of DNA, usually upstream (5′) of the coding region of a structural gene, which controls the expression of the coding region by providing recognition and binding sites for RNA polymerase and other factors which may be required for initiation of transcription. The selection of the promoter will depend upon the nucleic acid sequence of interest. A “promoter functional in a host cell” refers to a “promoter” which is capable of supporting the initiation of transcription in said cell, causing the production of an mRNA molecule.

The term “inducible” used in the context of a promoter means that the promoter only directs transcription of an operably linked nucleotide sequence if a chemical or physical stimulus is present, such as the presence of a chemical substance (“chemical inducer”) or a change in temperature.

A temperature inducible promoter as referred to herein is a promoter which directs transcription only below or above a certain temperature. Non-limiting examples of temperature inducible promoters include the pL and pR λ phage promoters and the cspA promoter (all functional in bacterial cells).

As used herein, “chemical induction” according to the present invention refers to the physical application of a exogenous or endogenous substance (incl. macromolecules, e.g., proteins or nucleic acids) to a microorganism.

As used herein, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. A promoter sequence is “operably-linked” to a gene when it is in sufficient proximity to the transcription start site of a gene to regulate transcription of the gene.

The term “expression cassette” as used herein refers to a nucleotide sequence which is capable of affecting expression of a structural gene (i.e., a protein coding sequence) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers.

As used herein, an “operon” is a functioning unit of DNA containing a cluster of genes under the control of a single promoter.

“Percentage of sequence identity,” “% sequence identity” and “percent identity” are used herein to refer to comparisons between an amino acid sequence and a reference amino acid sequence. The “% sequence identify”, as used herein, is calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence.

“Reference sequence” or “reference amino acid sequence” refers to a defined sequence to which another sequence is compared. In the context of the present invention a reference amino acid sequence may be any amino acid sequence set forth in SEQ ID NO: 6 to 30.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### EXAMPLES

### Summary of Examples

As demonstrated in the following examples, decoupling of growth from production can significantly increase specific production and production yield of both proteins (GFP used as an example) and biochemical compounds (tyrosine and mevalonate used as examples). Using a library screening approach, we have identified a number of target genes that can be used to inhibit growth and increase production. Some toxin-anti toxin systems can for example be used to significantly increase protein production. In addition, we show that inhibition of nucleotide biosynthesis is an effective method for limiting growth while still allowing for continued production. This resulted in a 2.6-fold increased GFP expression per cell (or 2.2-fold total GFP production) as well as a 41% increase in mevalonate yield from glucose. We also demonstrate that the CRISPRi system can be used to create effective and long lasting inducible growth switches. By adding 5-fluorouracil (5-FU), an inhibitor of nucleotide biosynthesis, we obtained a higher specific production of both mevalonate and tyrosine compared to control. By controlling the growth of pyrF knock-out strain through supplementation with uracil, higher yields of both mevalonate and tyrosine were achieved. Mevalonate is a precursor for all isoprenoid compounds while tyrosine is a precursor for most aromatic compounds in nature, and the developed method is therefore applicable to a wide range of biochemical compounds.

### Example 1—Library Screening for Targets with Growth Inhibition and Protein Enrichment Effects

In order to identify targets for inhibiting growth from production, an experiment was designed to conduct a genome wide screening. A CRISPRi library was designed to target all genes as well as some non-coding regions across the E. coli MG1655 genome, in order to identify genes or locations that turn down cell growth while maintain protein production when repressed or blocked. 12238 sgRNAs were designed to target locations across the genome, with 2 sgRNAs for each gene coding sequence and 3497 sgRNAs distributing evenly in the non-coding regions. SgRNAs targeting gene coding sequences were designed to bind non-template strand near start codon region. A custom sgRNA-design software Crispy++ was used to estimate off-target efficiency of each sgRNAs, and sgRNAs with low off-target efficiency (scores <5000) were preferred (Qi et al., 2013). Designed oligonucleotides were ordered as a pooled library (CustomArray Inc), and amplified using primers SON172 and SON173 (Table S3). Plasmid pSLQ1236 (obtained as a gift from Professor Stanley Qi) was amplified using primers SON178 and SON179 (Table S3) and assembled with the amplified library (Larson et al., 2013). 100 μg/mL carbenicillin was used to select cells with correct constructs. 150× coverage of colonies were obtained to ensure sufficient representation. Plasmids carrying the sgRNAs library were transformed into E. coli Sij17, a strain with GFP constitutive expression cassette in genome (Bonde et al., 2016), with plasmid pdCas9-bacteria (Addgene; Plasmid #44249), and 60× coverage of transformants were obtained and used as the cell library for screening. 5 mL culture of the cell library was sampled for plasmids extraction, and extracts were used for next generation sequencing. 100 μg/mL carbenicillin and 25 μg/mL chloramphenicol was used to select correct transformants as well as maintain transformed cells in following experiments.

The prepared cell library was grown overnight in M9 media with 0.5% (w/v) glucose and 0.02% (w/v) yeast extract (M9G0.5YE) used as pre-culture. Pre-culture was then diluted 100 times in fresh M9 media with 0.5% (w/v) glucose (M9G0.5) for following experiments. In order to identify targets affecting cell growth, cultures with and without the expression of CRISPRi system were compared, and effective targets were expected to reduce in cultures expressing CRISPRi system. Cell cultures were prepared in 20 mL volumes with or without induction, grown for 24 hours, and 5 mL were sampled from each culture for plasmids extraction, respectively. The induction of CRISPRi system was performed by adding 200 ng/mL anhydrotetracycline (aTc) one hour after inoculation. Prepared plasmids were used for next generation sequencing. Triplicate experiments were performed. In order to identify targets increasing GFP production, the induced culture was analyzed and sorted on FACS Aria (Becton Dickinson, San Jose, USA), and top 1% of cells with fluorescence (FITC) higher than 2800 were collected. Sorted cells were recovered in 1 mL SOC for 2 hours and then transferred into M9G0.5YE for overnight growth. Overnight culture was used as pre-culture for next round sorting, and 5 mL sample was taken for plasmids extraction and sequencing analysis. Three rounds of sorting were performed using the same setting. 33.000 cells were collected in the first and second round, and 50.000 cells were collected in the third round. Triplicate experiments were performed. Forward-scatter and side-scatter was detected as small- and large-angle scatters of the 488 nm laser, respectively. GFP fluorescence was detected with a 488 nm long-pass and a 530/30 nm band-pass filter set. 37° C. and 250 rpm were used as cultivation conditions through the whole experiments.

The target regions of prepared plasmid extracts were amplified through two rounds of PCR and used for next generation sequencing. In the first round of PCR, 20 μL reaction system was used with Phusion Hot Start II DNA Polymerase (Thermo Fisher Scientific), and around 40 ng DNA were added for each sample and amplified with primers SON233 and SON234 (98° C. for 5 min and then 25 cycles of 98° C. for 30 s, 65° C. for 30 s, and 72° C. for 30 s, with a final elongation step at 72° C. for 7 min). Each PCR product was purified using AMPure XP beads (Beckman Coulter, Calif.), and diluted in 50 μL Tris (pH=8.5). In the second round of PCR, 20 μL reaction system was used with Phusion High-Fidelity PCR Master Mix with HF Buffer (Thermo Fisher Scientific), and around 5 μL purified products were added for each sample and amplified with Nextera XT Index primers (Illumina no. FC-131-1001) (98° C. for 5 min and then 25 cycles of 98° C. for 30 s, 65° C. for 30 s, and 72° C. for 30 s, with a final elongation step at 72° C. for 7 min). Each PCR product was again purified using AMPure XP beads (Beckman Coulter, Calif.). Each prepared sample was verified and quantified by 2100 Bioanalyzer (Agilent) and Qubit® 2.0 Fluorometer (Thermo Fisher Scientific) respectively, diluted to appropriate concentration, and analysed by next generation sequencing. Sequencing was performed on a NextSeq 500 desktop sequencer (Illumina, San Diego, Calif.) using 75 bp single read.

Counts of sgRNAs in each sample were extracted from sequencing files and normalized by Tag Count Comparison (TCC) method (Sun et al., 2013). SgRNAs with reduced frequency in induced cultures compared to uninduced cultures were estimated by TCC method, and suggested as targets with the effect of growth inhibition. SgRNAs with increased frequency in sorted cultures, especially in the cultures after 3 rounds of sortings, were suggested to increase the production of GFP.

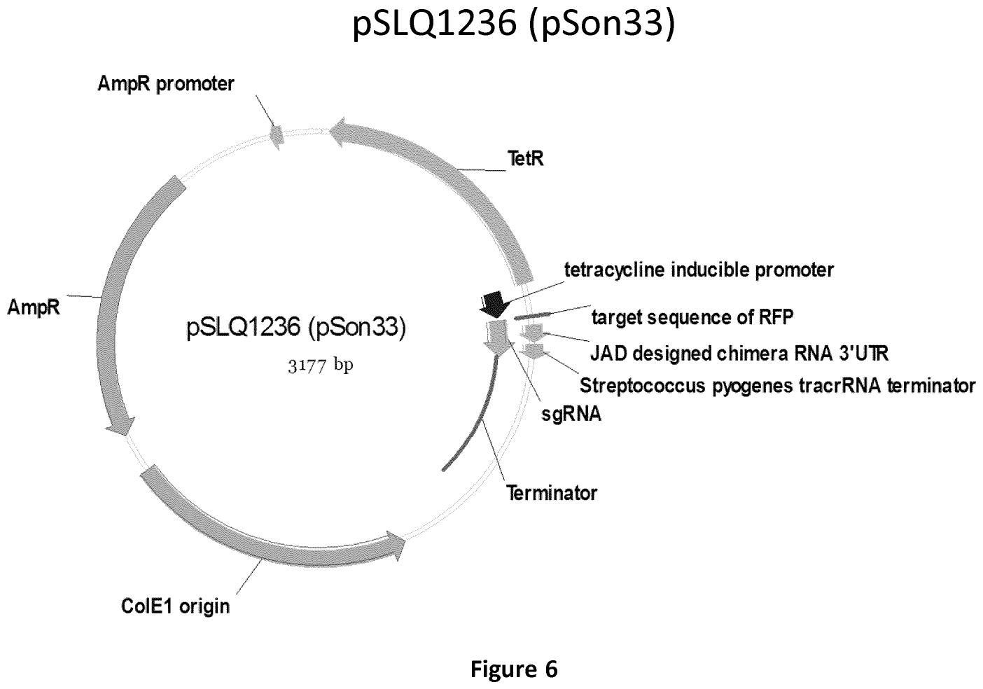

Desired CRISPRi systems should be able to repress cell growth and maintain protein production while activated. Therefore, sgRNAs with decreased frequency in induced cultures and increased frequency in sorted cultures, comparing to uninduced cultures, were considered as promising candidates. According to this rule, top 15 sgRNAs were selected for further testing. Candidate sgRNAs were assembled into plasmid pSLQ1236 (FIG. 6) using primers SON203-SON232 (Table S3). A control plasmid without sgRNA expression was assembled using pSLQ1236 as template with primers SON176 and SON177 (Table S3). Standard reagents and methods described above were used for this assembly. Different derivatives of pSLQ1236 were transformed into Sii17 with pdCas9-bacteria for further testing. Candidate strains as well as the control was inoculated in M9G0.5YE for overnight growth, then diluted 100 times in 800 μL fresh M9G0.5, and transferred into 96 deep well plate. For each strain, cultures were prepared in triplicates with or without induction, cultivated for 24 hours at 37° C. and 300 rpm, and sampled for optical density (OD) and fluorescence measurement. The induction was performed as described above. OD was measured at 630 nm using Synergy Mx plate reader (BioTek, USA) and fluorescence was measured on FACS Aria (Becton Dickinson, San Jose, USA) with the same setting.

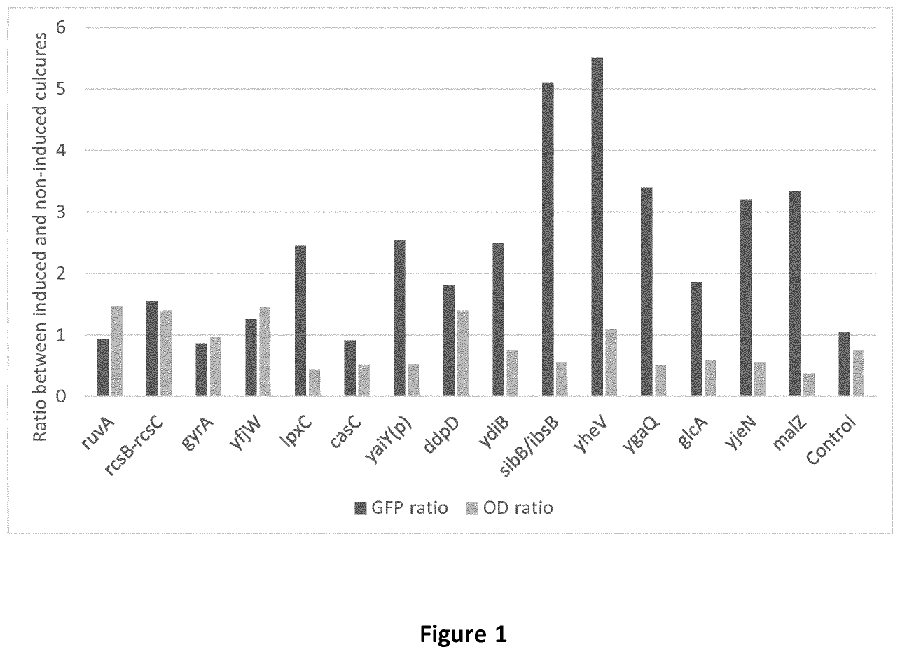

In FIG. 1, the effect of repression of a selection of candidate genes is shown for growth (OD) and protein production (GFP). In particular, inhibition of sibB of the toxin/anti-toxin system sib/ibsB provides more than a 5-fold increase in GFP fluorescence per cell. Repression of other selected targets also increases GFP production per cell.

Nucleotide biosynthesis is essential for cell growth, and we were therefore interested if genes involved in these pathways could be used to repress growth. Almost all genes involved in nucleotide biosynthesis were consistently found to repress growth of E. coli as shown in the Table 4. The majority of these genes were also found amongst cells sorted for having increased production of heterologous proteins.

TABLE 4
Inhibition of genes involved in nucleotide biosynthesis effectively inhibits growth
of E. coli. A library of sgRNA was used to direct dCas9 to inhibit gene expression
of selected target genes. The occurrence (frequency) of the different sgRNA sequences
in the library grown with and without induction of the CRISPRi system was used
to determine the growth inhibition caused by inhibition of each of the genes.
The remaining fraction of cells after induction was calculated by dividing the
frequency of a specific sgRNA in the induced cultures by that in the uninduced
cultures (column 3). Cells repressing genes involved in nucleotide biosynthesis
were also found amongst cells sorted for having increased production of heterologous
protein (GFP) after several rounds of sorting (column 4-6).

| | | Remaining | | | |
|---|---|---|---|---|---|
| | | fraction of | Normalized | Normalized | Normalized |
| | | cells after | reads in 1st | reads in | reads in |
| Gene | pathway | induction | sorting | 2nd sorting | 3rd sorting |
| purF | purine de novo | 0.08 | 0.00 | 0.00 | 0.00 |
| purD | purine de novo | 0.29 | 0.26 | 0.00 | 0.00 |
| purN | purine de novo | 0.68 | 133.78 | 0.00 | 0.00 |
| purL | purine de novo | 0.44 | 266.03 | 0.00 | 0.00 |
| purM | purine de novo | 0.21 | 181.10 | 0.00 | 0.00 |
| purK | purine de novo | 0.43 | 3703.61 | 4682.37 | 1241.36 |
| purE | purine de novo | 0.15 | 70.35 | 0.00 | 0.00 |
| purC | purine de novo | 0.41 | 796.54 | 423.57 | 280.00 |
| purB | purine de novo | 0.34 | 121.05 | 181.02 | 0.00 |
| purH | purine de novo | 0.56 | 0.00 | 0.00 | 0.00 |
| purA | purine de novo | 0.63 | 0.00 | 0.00 | 0.00 |
| guaA | purine de novo | 0.33 | 187.49 | 128.15 | 2.80 |
| guaB | purine de novo | 0.27 | 1128.71 | 692.55 | 16.79 |
| adk | NMP kinase | 0.29 | 0.00 | 0.00 | 0.00 |
| gmk | NMP kinase | 0.40 | 20.88 | 97.53 | 52.01 |
| pykA | NDP kinase | 0.85 | 0.00 | 0.00 | 0.00 |
| guaC | purine | 0.90 | 266.52 | 38.46 | 0.00 |
| | interconversions | | | | |
| dgt | purine | 0.41 | 28.77 | 0.00 | 0.00 |
| | interconversions | | | | |
| carA | Arg and pyrimidine | 0.41 | 92.84 | 53.53 | 0.00 |
| | de novo | | | | |
| carB | Arg and pyrimidine | 0.41 | 11.99 | 0.00 | 0.00 |
| | de novo | | | | |
| pyrB | pyrimidine de novo | 0.17 | 0.00 | 0.00 | 0.00 |
| pyrC | pyrimidine de novo | 0.84 | 120.64 | 16.00 | 28.00 |
| pyrD | pyrimidine de novo | 0.25 | 0.00 | 0.00 | 0.00 |
| pyrE | pyrimidine de novo | 0.59 | 93.58 | 0.00 | 0.00 |
| pyrF | pyrimidine de novo | 0.40 | 2651.24 | 1523.18 | 186.87 |
| pyrG | pyrimidine de novo | 0.22 | 70.89 | 1.17 | 0.00 |
| pyrH | NMP kinase | 0.39 | 54.33 | 0.00 | 0.00 |
| ndk | NDP kinase | 0.88 | 4.11 | 0.00 | 0.00 |
| cmk | NMP kinase | 0.49 | 270.77 | 0.00 | 0.00 |
| nrdA | deoxy synth | 0.03 | 0.00 | 0.00 | 0.00 |
| nrdB | deoxy synth | 0.06 | 0.00 | 0.00 | 0.00 |
| nrdD | deoxy synth | 0.80 | 793.01 | 196.23 | 48.01 |
| tmk | pyr deoxy NMP kinase | 0.17 | 0.00 | 0.00 | 0.00 |
| dut | pyr deoxy | 0.18 | 0.00 | 0.00 | 0.00 |
| | interconversion | | | | |
| thyA | pyr deoxy | 0.17 | 0.00 | 0.00 | 0.00 |
| | interconversion | | | | |
| dcd | pyr deoxy | 0.17 | 0.26 | 0.00 | 0.00 |
| | interconversion | | | | |
| cdd | pyrimidine | 0.69 | 205.93 | 47.51 | 0.00 |
| | interconversions | | | | |
| codA | pyrimidine | 0.89 | 1150.52 | 189.85 | 6.40 |
| | interconversions | | | | |
| udk | pyrimidine | 0.77 | 0.00 | 0.00 | 0.00 |
| | interconversions | | | | |
| tdk | pyrimidine | 0.82 | 1707.59 | 1616.36 | 223.12 |
| | interconversions | | | | |
| dcd | pyrimidine | 0.17 | 0.26 | 0.00 | 0.00 |
| | interconversions | | | | |
| udk | pyrimidine salvage | 0.77 | 0.00 | 0.00 | 0.00 |
| udp | pyrimidine salvage | 0.88 | 333.06 | 0.00 | 0.00 |
| deoA | pyrimidine salvage | 0.61 | 632.12 | 32.77 | 0.00 |
| atpB | ATP synthase | 0.33 | 212.77 | 0.00 | 0.00 |
| atpF | ATP synthase | 0.57 | 66.32 | 0.39 | 0.00 |
| atpE | ATP synthase | 0.51 | 642.92 | 75.12 | 44.62 |
| atpD | ATP synthase | 0.37 | 0.00 | 0.00 | 0.00 |
| atpG | ATP synthase | 0.62 | 232.98 | 266.98 | 29.85 |
| atpA | ATP synthase | 0.61 | 217.76 | 321.67 | 131.50 |
| atpH | ATP synthase | 0.34 | 192.02 | 110.22 | 0.00 |
| atpC | ATP synthase | 0.37 | 2663.77 | 1930.37 | 325.06 |

TABLE 5
Full sequence of sgRNA for each target
gene. The sgRNA is composed of the target
sequence and the sgRNA body sequence (sgRNA
body sequence: GTTTTAGAGCTAGAAATAGCAAGTTAA
AATAAGGCTAGTCCGTTATCAACTTGAAAAAGTGGCACCGAGT
CGGTGCTTTTTT).

| Gene | Full sequence of sgRNA |
|---|---|
| purF | TCATAAATCGACTGGTTAACGTTTTAGAGCTAGAAATAGCAAGT |
| | TAAAATAAGGCTAGTCCGTTATCAACTTGAAAAAGTGGCACCGA |
| | GTCGGTGCTTTTTT |
| purD | CGGCGACTGGGCCGCTTTCCGTTTTAGAGCTAGAAATAGCAAGT |
| | TAAAATAAGGCTAGTCCGTTATCAACTTGAAAAAGTGGCACCGA |
| | GTCGGTGCTTTTTT |
| purN | CTGTAAATTACTTCCGTTGCGTTTTAGAGCTAGAAATAGCAAGT |
| | TAAAATAAGGCTAGTCCGTTATCAACTTGAAAAAGTGGCACCGA |
| | GTCGGTGCTTTTTT |
| purL | ATTCGGAATGCCGACAGTGCGTTTTAGAGCTAGAAATAGCAAGT |
| | TAAAATAAGGCTAGTCCGTTATCAACTTGAAAAAGTGGCACCGA |
| | GTCGGTGCTTTTTT |
| purM | GGCATCTTTGTAGCTAAGAGGTTTTAGAGCTAGAAATAGCAAGT |
| | TAAAATAAGGCTAGTCCGTTATCAACTTGAAAAAGTGGCACCGA |
| | GTCGGTGCTTTTTT |
| purK | GGCCTAACTGCCCGTTACCGGTTTTAGAGCTAGAAATAGCAAGT |
| | TAAAATAAGGCTAGTCCGTTATCAACTTGAAAAAGTGGCACCGA |
| | GTCGGTGCTTTTTT |
| purE | GACACGCGCCGGATTATTGCGTTTTAGAGCTAGAAATAGCAAGT |
| | TAAAATAAGGCTAGTCCGTTATCAACTTGAAAAAGTGGCACCGA |
| | GTCGGTGCTTTTTT |
| purC | ATTCGAGCACCAACAGGTCCGTTTTAGAGCTAGAAATAGCAAGT |
| | TAAAATAAGGCTAGTCCGTTATCAACTTGAAAAAGTGGCACCGA |
| | GTCGGTGCTTTTTT |
| purB | TCCATCGACAGGGGAAACGGGTTTTAGAGCTAGAAATAGCAAGT |
| | TAAAATAAGGCTAGTCCGTTATCAACTTGAAAAAGTGGCACCGA |
| | GTCGGTGCTTTTTT |
| purH | AAACACTGAGCAGAGCGCGGGTTTTAGAGCTAGAAATAGCAAGT |
| | TAAAATAAGGCTAGTCCGTTATCAACTTGAAAAAGTGGCACCGA |
| | GTCGGTGCTTTTTT |
| purA | TTTACCTTCGTCACCCCATTGTTTTAGAGCTAGAAATAGCAAGT |
| | TAAAATAAGGCTAGTCCGTTATCAACTTGAAAAAGTGGCACCGA |
| | GTCGGTGCTTTTTT |
| guaA | GAGAACCGAAGTCCAGAATGGTTTTAGAGCTAGAAATAGCAAGT |
| | TAAAATAAGGCTAGTCCGTTATCAACTTGAAAAAGTGGCACCGA |
| | GTCGGTGCTTTTTT |
| guaB | CGGTAGAGTGAGCAGGAACGGTTTTAGAGCTAGAAATAGCAAGT |
| | TAAAATAAGGCTAGTCCGTTATCAACTTGAAAAAGTGGCACCGA |
| | GTCGGTGCTTTTTT |
| adk | TGAGTCCCTTTCCCCGCGCCGTTTTAGAGCTAGAAATAGCAAGT |
| | TAAAATAAGGCTAGTCCGTTATCAACTTGAAAAAGTGGCACCGA |
| | GTCGGTGCTTTTTT |
| gmk | TGGATTTACCCGCGCCACTGGTTTTAGAGCTAGAAATAGCAAGT |
| | TAAAATAAGGCTAGTCCGTTATCAACTTGAAAAAGTGGCACCGA |
| | GTCGGTGCTTTTTT |
| pykA | ATCTGTTGCTGGGCCTAACGGTTTTAGAGCTAGAAATAGCAAGT |
| | TAAAATAAGGCTAGTCCGTTATCAACTTGAAAAAGTGGCACCGA |
| | GTCGGTGCTTTTTT |
| guaC | TAAGAGTGGAGCGTTTAGGGGTTTTAGAGCTAGAAATAGCAAGT |
| | TAAAATAAGGCTAGTCCGTTATCAACTTGAAAAAGTGGCACCGA |
| | GTCGGTGCTTTTTT |
| dgt | TTTTAACGCCCTGCGGTGAAGTTTTAGAGCTAGAAATAGCAAGT |
| | TAAAATAAGGCTAGTCCGTTATCAACTTGAAAAAGTGGCACCGA |
| | GTCGGTGCTTTTTT |
| carA | TATGGCCCGACCGTGAAACTGTTTTAGAGCTAGAAATAGCAAGT |
| | TAAAATAAGGCTAGTCCGTTATCAACTTGAAAAAGTGGCACCGA |
| | GTCGGTGCTTTTTT |
| carB | TCGGGCCCGCACCCAGAATCGTTTTAGAGCTAGAAATAGCAAGT |
| | TAAAATAAGGCTAGTCCGTTATCAACTTGAAAAAGTGGCACCGA |
| | GTCGGTGCTTTTTT |
| pyrB | ATGATATGTTTCTGATATAGGTTTTAGAGCTAGAAATAGCAAGT |
| | TAAAATAAGGCTAGTCCGTTATCAACTTGAAAAAGTGGCACCGA |
| | GTCGGTGCTTTTTT |
| pyrC | CGGATCTTTAATACCTGGGAGTTTTAGAGCTAGAAATAGCAAGT |
| | TAAAATAAGGCTAGTCCGTTATCAACTTGAAAAAGTGGCACCGA |
| | GTCGGTGCTTTTTT |
| pyrD | AAAAGGGCTTTACGAACGAAGTTTTAGAGCTAGAAATAGCAAGT |
| | TAAAATAAGGCTAGTCCGTTATCAACTTGAAAAAGTGGCACCGA |
| | GTCGGTGCTTTTTT |
| pyrE | TAAGCGCAAATTCAATAAACGTTTTAGAGCTAGAAATAGCAAGT |
| | TAAAATAAGGCTAGTCCGTTATCAACTTGAAAAAGTGGCACCGA |
| | GTCGGTGCTTTTTT |
| pyrF | CAGGAGAATTCGTAACAGCGGTTTTAGAGCTAGAAATAGCAAGT |
| | TAAAATAAGGCTAGTCCGTTATCAACTTGAAAAAGTGGCACCGA |
| | GTCGGTGCTTTTTT |
| pyrG | GGCAATGCCTTTACCCAGAGGTTTTAGAGCTAGAAATAGCAAGT |
| | TAAAATAAGGCTAGTCCGTTATCAACTTGAAAAAGTGGCACCGA |
| | GTCGGTGCTTTTTT |
| pyrH | TTTATAGACGGGTTTTGCATGTTTTAGAGCTAGAAATAGCAAGT |
| | TAAAATAAGGCTAGTCCGTTATCAACTTGAAAAAGTGGCACCGA |
| | GTCGGTGCTTTTTT |
| ndk | ACGTTTTTTGCTACCGCGTTGTTTTAGAGCTAGAAATAGCAAGT |
| | TAAAATAAGGCTAGTCCGTTATCAACTTGAAAAAGTGGCACCGA |
| | GTCGGTGCTTTTTT |
| cmk | GGCCATCAATGGTAATAACCGTTTTAGAGCTAGAAATAGCAAGT |
| | TAAAATAAGGCTAGTCCGTTATCAACTTGAAAAAGTGGCACCGA |
| | GTCGGTGCTTTTTT |
| nrdA | GTGGGAGCGCAGCTCGACCTGTTTTAGAGCTAGAAATAGCAAGT |
| | TAAAATAAGGCTAGTCCGTTATCAACTTGAAAAAGTGGCACCGA |
| | GTCGGTGCTTTTTT |
| nrdB | ATTTTTCGTCTGTGAAAAGGGTTTTAGAGCTAGAAATAGCAAGT |
| | TAAAATAAGGCTAGTCCGTTATCAACTTGAAAAAGTGGCACCGA |
| | GTCGGTGCTTTTTT |
| nrdD | CCGTCTCGTTTCATCACATGGTTTTAGAGCTAGAAATAGCAAGT |
| | TAAAATAAGGCTAGTCCGTTATCAACTTGAAAAAGTGGCACCGA |
| | GTCGGTGCTTTTTT |
| tmk | CTCAACCACCACATTACGCGGTTTTAGAGCTAGAAATAGCAAGT |
| | TAAAATAAGGCTAGTCCGTTATCAACTTGAAAAAGTGGCACCGA |
| | GTCGGTGCTTTTTT |
| dut | GAAATTCCTTCCCAACGCGCGTTTTAGAGCTAGAAATAGCAAGT |
| | TAAAATAAGGCTAGTCCGTTATCAACTTGAAAAAGTGGCACCGA |
| | GTCGGTGCTTTTTT |
| thyA | AATGGAAAGCGTTCCGGTTCGTTTTAGAGCTAGAAATAGCAAGT |
| | TAAAATAAGGCTAGTCCGTTATCAACTTGAAAAAGTGGCACCGA |
| | GTCGGTGCTTTTTT |
| dcd | CAAGCCAGGCTTCAATATCTGTTTTAGAGCTAGAAATAGCAAGT |
| | TAAAATAAGGCTAGTCCGTTATCAACTTGAAAAAGTGGCACCGA |
| | GTCGGTGCTTTTTT |
| cdd | ATCCGCAAGTTGGGCAAAAGGTTTTAGAGCTAGAAATAGCAAGT |
| | TAAAATAAGGCTAGTCCGTTATCAACTTGAAAAAGTGGCACCGA |
| | GTCGGTGCTTTTTT |
| codA | CCCCTCTTCGCCTGGTAACCGTTTTAGAGCTAGAAATAGCAAGT |
| | TAAAATAAGGCTAGTCCGTTATCAACTTGAAAAAGTGGCACCGA |
| | GTCGGTGCTTTTTT |
| udk | CAATTCACGATAAAGGGTACGTTTTAGAGCTAGAAATAGCAAGT |
| | TAAAATAAGGCTAGTCCGTTATCAACTTGAAAAAGTGGCACCGA |
| | GTCGGTGCTTTTTT |
| tdk | CAGTGCGCATGCCGCGTTCCGTTTTAGAGCTAGAAATAGCAAGT |
| | TAAAATAAGGCTAGTCCGTTATCAACTTGAAAAAGTGGCACCGA |
| | GTCGGTGCTTTTTT |
| udp | GAGATGAAAAACATCAGACTGTTTTAGAGCTAGAAATAGCAAGT |
| | TAAAATAAGGCTAGTCCGTTATCAACTTGAAAAAGTGGCACCGA |
| | GTCGGTGCTTTTTT |
| deoA | GAAAATGGTCATCGCGAGGGGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpB | TGGTGTCCTATGTAATCCTGGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpF | ACAGGACAAACGCGATGGCCGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpE | TGTACAGCAGATCCATATTCGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpD | AGGGAATTCGACGTCAACTAGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpG | TAGTGATCTTTTGCGTGTTCGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpA | GATCAGTTCGCTGATTTCGGGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpH | CAAAAGCTGCTTTGGCGTAGGTTTTAGAGCTAGAAATAGCAAGT |
| | TAAAATAAGGCTAGTCCGTTATCAACTTGAAAAAGTGGCACCGA |
| | GTCGGTGCTTTTTT |
| atpC | GCTCTGCGCTGACGACGTCCGTTTTAGAGCTAGAAATAGCAAGT |
| | TAAAATAAGGCTAGTCCGTTATCAACTTGAAAAAGTGGCACCGA |
| | GTCGGTGCTTTTTT |

In conclusion, it was found that repression of certain genes can be used to repress or inhibit the growth of a production organism, and at the same time increase the production of a recombinant polypeptide (exemplified by the expression of GFP). In particular, lpxC, yaiY(p), ydiB, sibB, yheV, ygaQ, glcA, yjeN and malZ were found to repress growth while significantly increasing recombinant polypeptide expression in the cell. In addition, it was found that genes involved in nucleotide biosynthesis are excellent candidates for repressing growth of the production organism, and that such targets may generally lead to increased expression of heterologous proteins.

### Example 2—Growth Arrested by Inhibiting Nucleotide Synthesis or DNA Replication

In order to further investigate the potential of reduction of growth by inhibiting the expression of certain genes for increasing the production of proteins and biochemical compounds, four specific CRISPRi systems were designed to inhibit cell growth by inhibiting nucleotide synthesis or DNA replication.

Materials and Methods

Culture Media, Strains and Plasmids

Escherichia coli strains and plasmids used in this study are listed in Table S1 and S2. Primer sequences are listed in Table S3.

NEB 5-alpha (New England Biolabs) was used for all cloning work in this study. LB media and agar plates with corresponding antibiotics were used for cultivation and selection for cloning. Kanamycin, carbenicillin, ampicillin, spectinomycin and chloramphenicol were used in this study with working concentrations of 50 μg/mL, 100 μg/mL, 100 μg/mL, 50 μg/mL and 25 μg/mL respectively.

MG1655 was used as the background strain for growth profiling experiments. Different growth switches as well as a negative control system were transformed into MG1655 in order to create test strains SoT53, 54, 55, 56 and 65. All the growth profiling experiments were performed in M9 medium with 0.5% (w/v) glucose and 0.02% (w/v) yeast extract (M9G0.5YE).

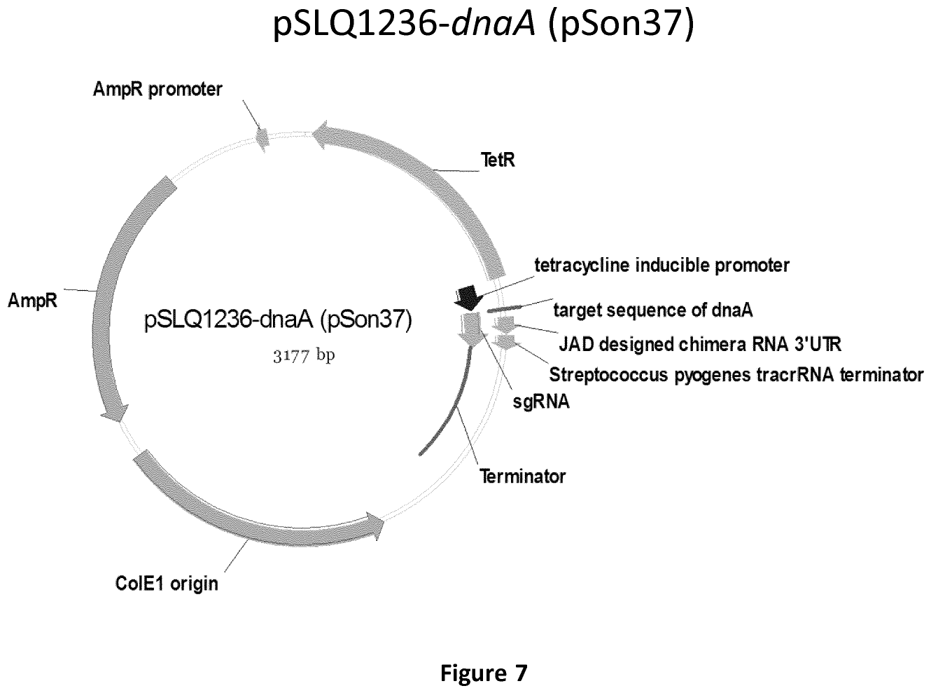

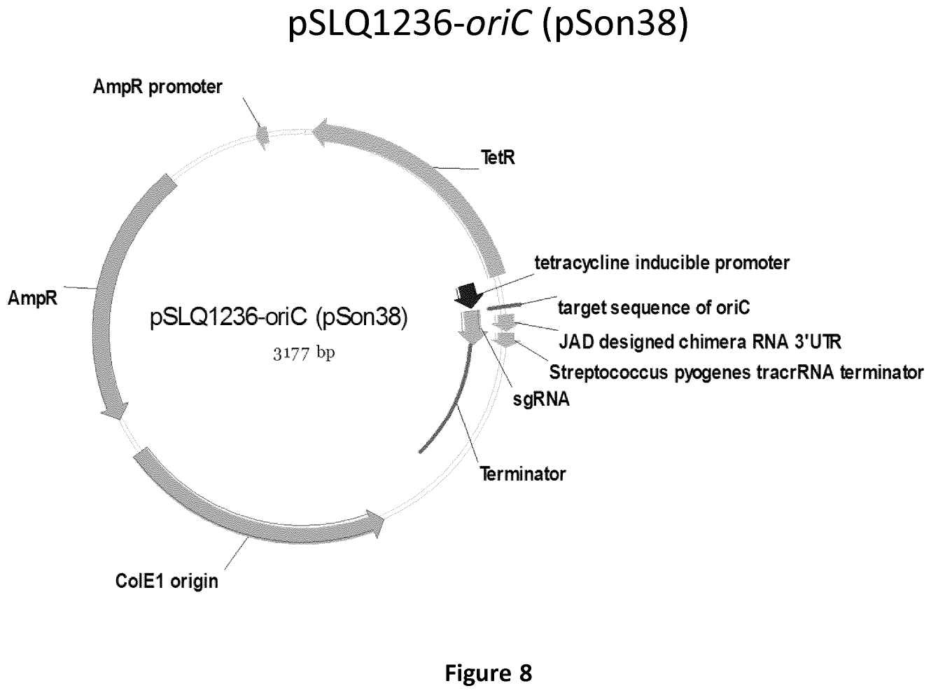

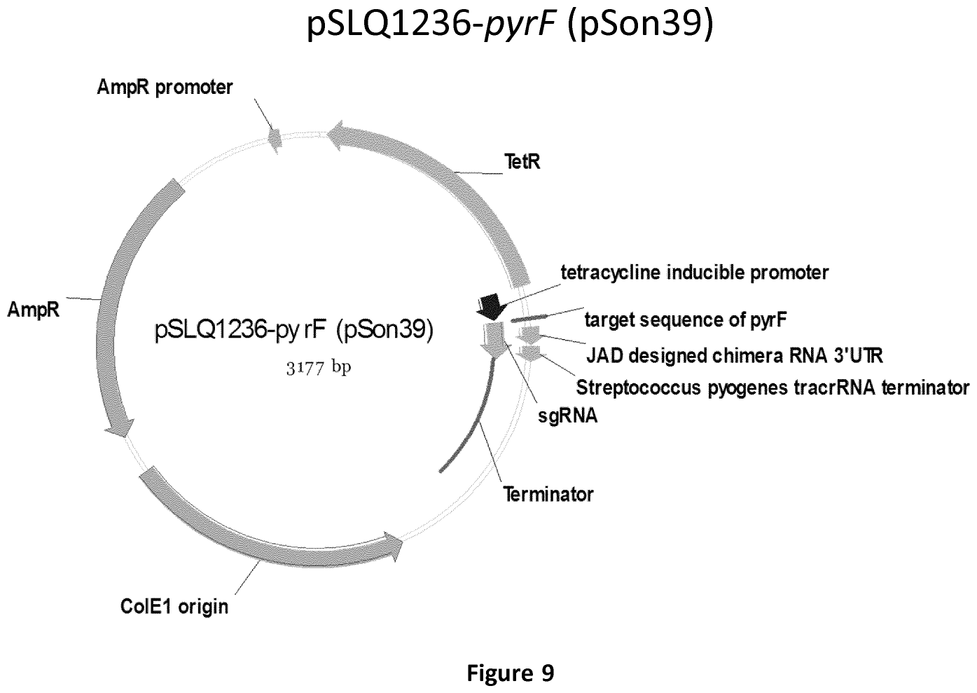

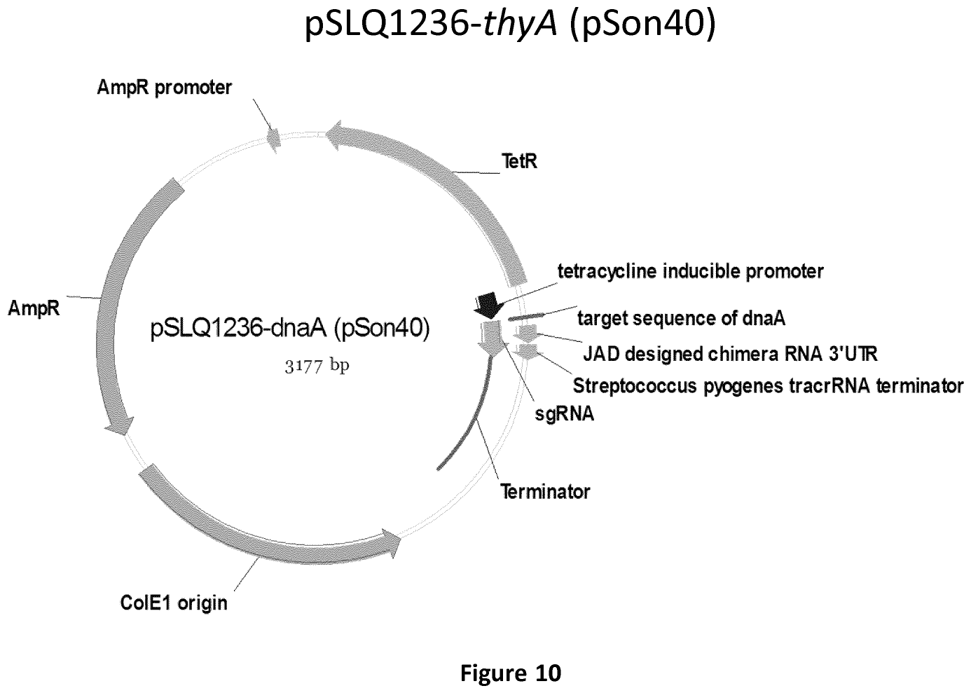

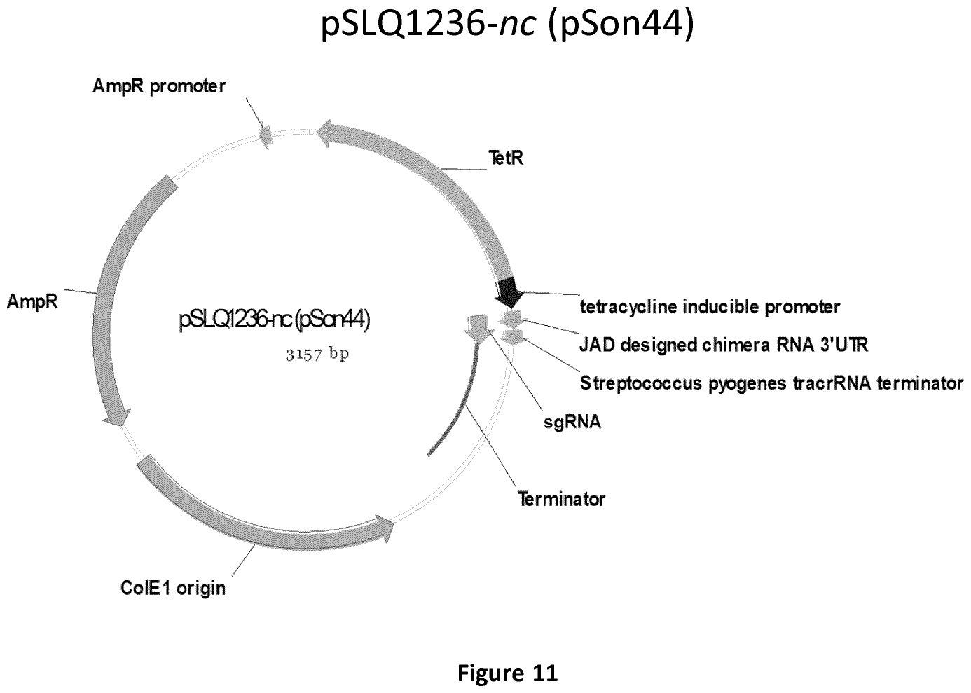

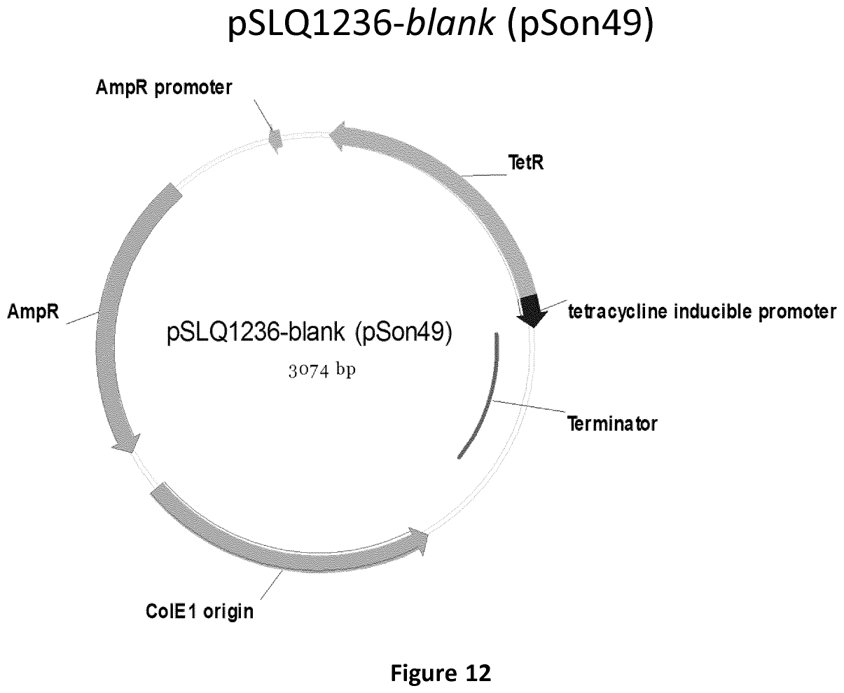

The growth switches as well as the control switches consist of a pdCas9-bacteria plasmid (Addgene; Plasmid #44249) and one derivative of the pSLQ1236 plasmid (obtained as a gift from Professor Stanley Qi) (Larson et al., 2013). Derivatives of pSLQ1236 were obtained by modifying the original plasmid to target different locations (pSLQ1236-GFP, dnaA, oriC, pyrF and thyA) (Table 6A, Table 6B, and FIGS. 7-10). The pSLQ1236-nc (FIG. 11) was constructed by deleting the 20 bp target sequence, while the pSLQ1236-blank (FIG. 12) was constructed by deleting the whole sgRNA sequence. Standard protocols for digestion-ligation (SpeI-HindIII), Gibson assembly and USER cloning were used for cloning. The sequence of sgRNAs targeting dnaA and oriC were initially synthesized by Integrated DNA Technologies, Inc (Leuven, Belgium).

TABLE 6A
Target sequence of sgRNAs for
selected genes or locations.

| Target gene or | |
|---|---|
| location | Sequence |
| thyA | AATGGAAAGCGTTCCGGTTC |
| pyrF | AGGAGAATTCGTAACAGCGC |
| dnaA | CTGCCAAAGCGAAAGTGACA |
| oriC | GATCATTAACTGTGAATGAT |
| nc (blank) | none |

TABLE 6B
Complete sequence of selected sgRNAs

| Target gene | |
|---|---|
| or location | Sequence |
| thyA | AATGGAAAGCGTTCCGGTTCGTTTTAGAGCTAGAAAT |
| | AGCAAGTTAAAATAAGGCTAGTCCGTTATCAACTTGA |
| | AAAAGTGGCACCGAGTCGGTGCTTTTTT |
| pyrF | AGGAGAATTCGTAACAGCGCGTTTTAGAGCTAGAAAT |
| | AGCAAGTTAAAATAAGGCTAGTCCGTTATCAACTTGA |
| | AAAAGTGGCACCGAGTCGGTGCTTTTTT |
| dnaA | CTGCCAAAGCGAAAGTGACAGTTTTAGAGCTAGAAAT |
| | AGCAAGTTAAAATAAGGCTAGTCCGTTATCAACTTGA |
| | AAAAGTGGCACCGAGTCGGTGCTTTTTT |
| oriC | GATCATTAACTGTGAATGATGTTTTAGAGCTAGAAAT |
| | AGCAAGTTAAAATAAGGCTAGTCCGTTATCAACTTGA |
| | AAAAGTGGCACCGAGTCGGTGCTTTTTT |
| nc (blank) | GTTTTAGAGCTAGAAATAGCAAGTTAAAATAAGGCTA |
| | GTCCGTTATCAACTTGAAAAAGTGGCACCGAGTCGGT |
| | GCTTTTTT |

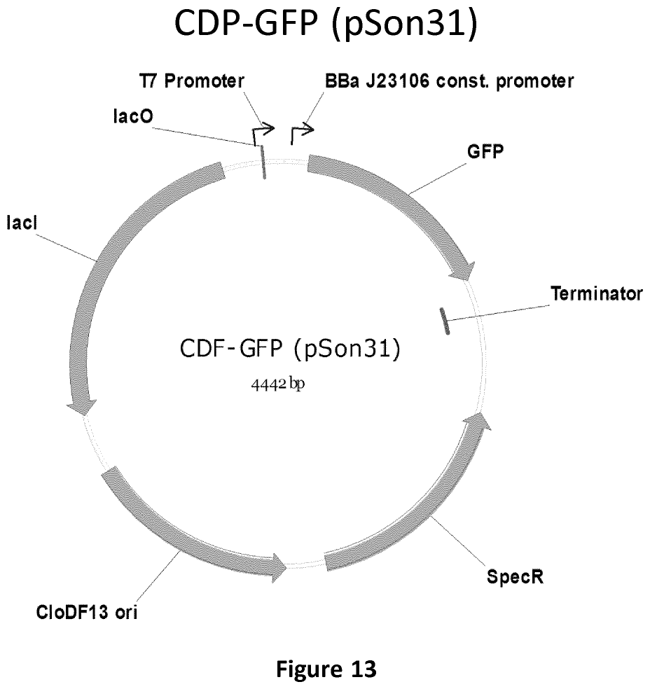

The CDF-GFP plasmid (FIG. 13) was cloned by Gibson assembly. The gfp variant used was reported in previous research (Bonde et al., 2016) and its expression was controlled by a constitutive promoter cloned from the biobrick BBa_J23106 (Registry of Standard Biological Parts) in combination with a strong SD sequence. The backbone was obtained from pCDFduet-1 (Novagen) digested with NcoI and PacI. In order to test the effect of growth decoupling on GFP production, the plasmid CDF-GFP was transformed into MG1655 together with different growth switches as well as the negative controls (SoT58, 59, 60, 61, 62, 66). GFP production and cell growth were monitored in M9G0.5YE media.

For mevalonate production, the inducible dCas9 expression cassette was introduced into the genome to create strain E. coli TCR. The expression cassette of dCas9 was amplified and cloned into pOSIP (St-Pierre et al., 2013) by USER cloning and was integrated into the phage 186 attachment site (the primary 0 site) in the genome. The kanamycin marker was subsequently looped out using pE-FLP according to the published protocol (St-Pierre et al., 2013).

In order to test mevalonate production, pMevT (Martin et al., 2003) was transformed into the TCR strain with or without different derivatives of pSLQ1236 (pSLQ1236-GFP, dnaA, oriC, pyrF, thyA and blank) to create testing strains (SoT80, 81, 82, 83, 84 and 96). The same M9G0.5YE media was used in this experiment except glucose was supplemented to a concentration of 1% (w/v) in this defined media in the time course experiments.

Growth Profiling Experiments

A single colony was inoculated into M9G0.5YE with appropriate antibiotics for overnight growth at 37° C. and 250 rpm. The overnight culture was diluted 100-fold into fresh M9G0.5YE media with corresponding antibiotics. For each strain, six 150 μL parallel cultures were prepared and transferred into 96-well microtiter plates for growth profiling. Plates were cultivated in an ELx808 plate reader (BioTek, USA) at 37° C. with medium shaking, and the optical density (OD) of each culture was measured at 630 nm for every 10 minutes for the following 24 hours. One hour after inoculation, 200 ng/mL anhydrotetracycline (aTc) was added to half of the cultures of each strain for induction.

GFP Production Experiments

The pre-culture was prepared as described above, and the overnight culture was diluted 100 times in fresh M9G0.5YE with corresponding antibiotics. Six 3-mL parallel cultures were prepared for each strain and transferred into 24-well plates for cultivation at 37° C. and 250 rpm. One hour after inoculation, half of the cultures for each strain were induced by the addition of 200 ng/mL aTc. At different time points for the following 24 hours, 20 μL of each culture were diluted 10 times and transferred into a 96-well plate for OD and fluorescence measurement. A Synergy Mx plate reader (BioTek, USA) was used for this measurement. The GFP fluorescence was measured with excitation at 485 nm and emission at 535 nm with a gain set to 80, and the OD was measured at 630 nm. If the OD was higher than 0.3, an appropriate dilution was made.

Flow Cytometry

After 24 hours, samples were taken from each culture and diluted in FACS Flow for flow cytometry analysis on FACS Aria (Becton Dickinson, San Jose, USA) in order to assess the concentration of heterologous GFP protein per cell. For each sample 50,000 events were counted. Forward-scatter and side-scatter were detected as small- and large-angle scatters of the 488 nm laser, respectively. GFP fluorescence was detected with a 488 nm long-pass and a 530/30 nm band-pass filter set. Data were analyzed using Flowjo (Tree Star Inc., Asland, Oreg.).

Mevalonate Production Experiments

Pre-cultures were prepared as described above. The overnight culture was diluted 1000 times in fresh M9G0.5YE with corresponding antibiotics. Four parallel cultures of 25 mL were prepared for each strain and cultivated in 250 mL shake flasks. All the cultures were induced by the addition of 500 μM IPTG for mevalonate pathway expression and cultivated at 37° C. and 250 rpm. Half of the cultures for each strain were induced with 200 ng/mL aTc one hour after induction. After 24 hours of cultivation, samples were taken for OD and high performance liquid chromatography (HPLC) analysis. The cell dry weight was estimated from OD by the factors determined in previous studies (Mundhada et al., 2015; von Stockar and Liu, 1999).

For time course experiments, three parallel cultures of 50 mL were prepared for each strain, and cultivated under the same conditions. All of the cultures were induced as described above. During 48 hours, samples were taken regularly for OD and HPLC analysis.

HPLC Analysis

Samples were first filtered through AcroPrep™ 96-well Filter Plates with 0.2 μm Supor® (Pall Corporation, USA). Filtered cultures were diluted two-fold in 125 μl milli-Q water, and then 19 μL of 20% (v/v) sulfuric acid was added. The mixture was incubated on ice for five minutes and then transferred for HPLC analysis. Mevalonate and glucose were quantified using HPLC (Thermo) equipped with a Bio-RAD Aminex HPX-87H ion exclusion column (catalog #125-0140) incubated at 50° C. Samples were run at a flow rate of 0.6 mL/min in 0.01 N sulfuric acid running buffer as described before (Beck et al., 2012). Mevalonate and glucose were detected by refractive index detector, and their concentrations were determined by comparison to a standard curve. Mevalonolactone and glucose were purchased from Sigma-Aldrich for standard preparation.

Results

Inhibition of Cell Growth by Blocking DNA Replication Machinery and Nucleotide Synthesis

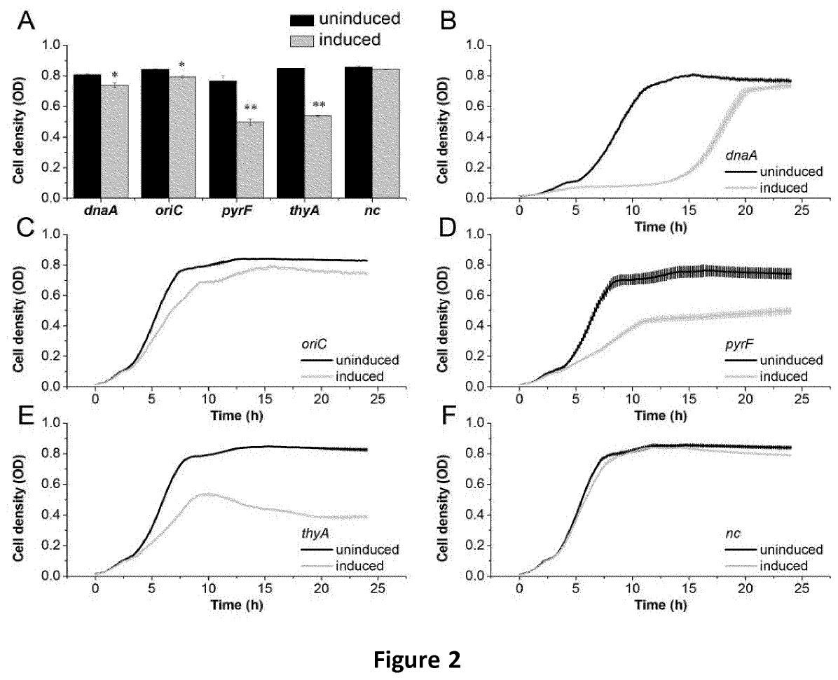

SoT 53, 54, 55, 56 and 65 were used to test the function of growth inhibition by targeting different genes or locations. As shown in FIG. 2, four of the designed growth switches were effective for controlling cell growth.

Characterization of Protein Expression During Growth Inhibition

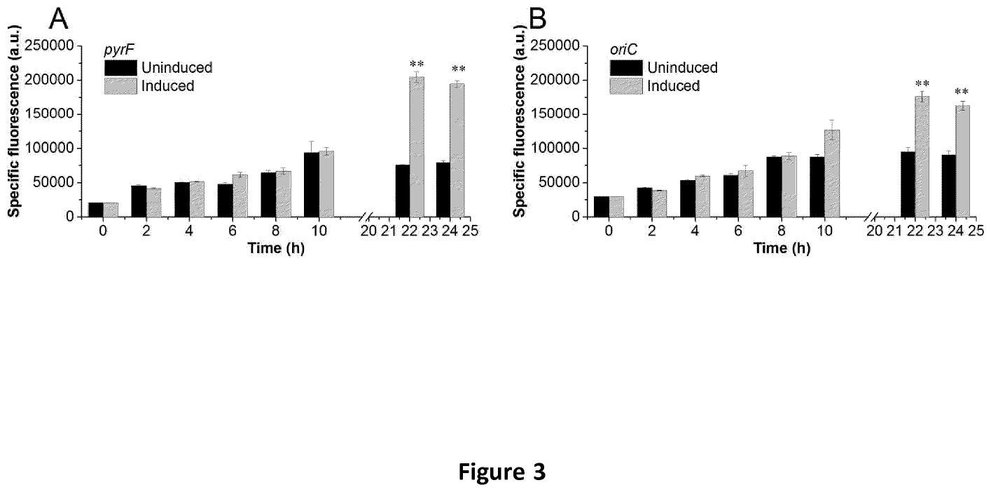

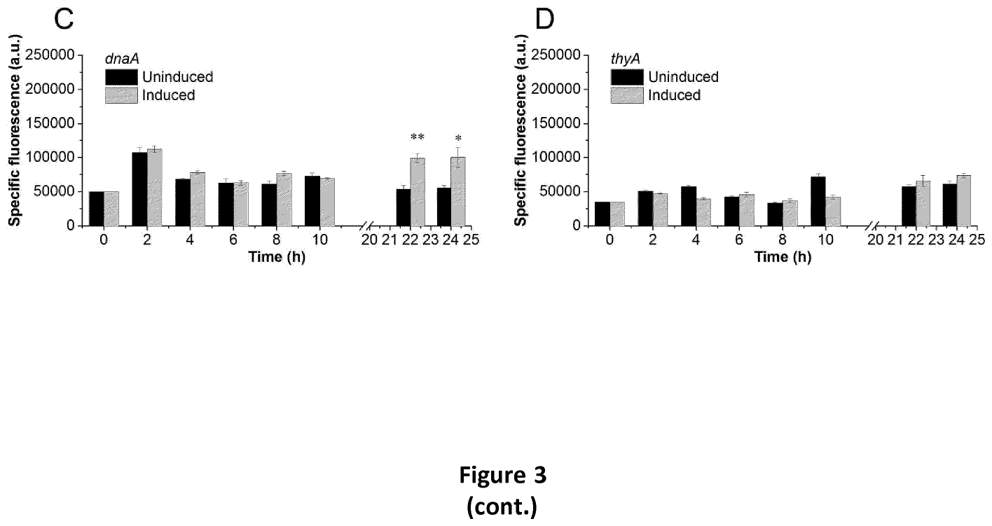

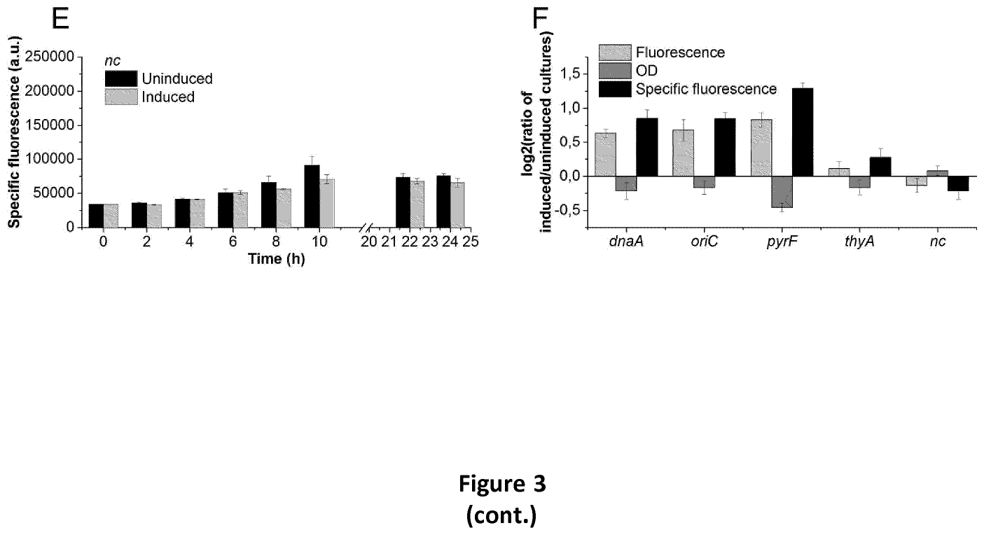

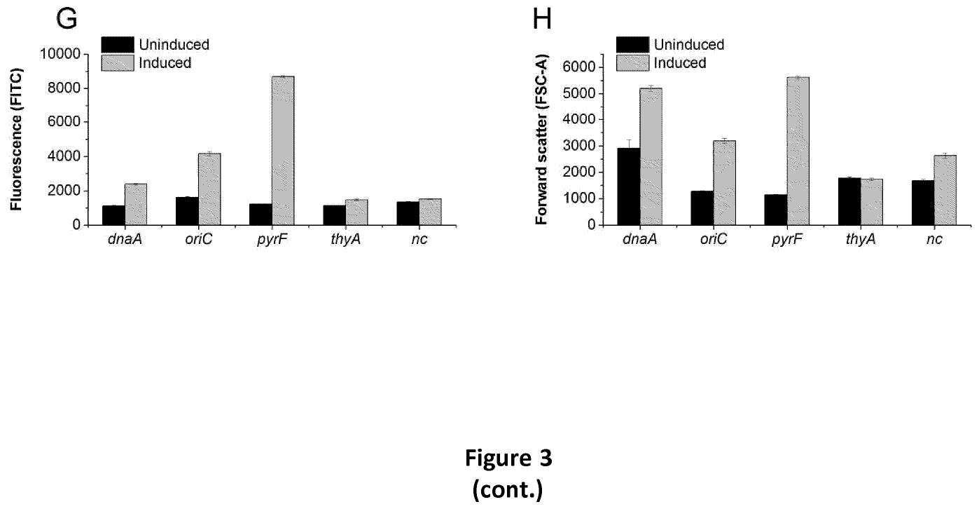

SoT 59, 60, 61, 62 and 66 were used to test the effect of different growth switches on the production of proteins. As demonstrated in FIG. 3, three of our designed growth switches, targeting pyrF, oriC and dnaA, showed to significantly increase the specific fluorescence in cells. Furthermore, an increased total fluorescence in the cultures was observed by inhibiting these designed targets. Among all of them, the CRISPRi system targeting pyrF increased the specific fluorescence and total fluorescence about 2.6 and 2.2 fold, respectively.

Growth Inhibition Results in Increased Production of Mevalonate

SoT 81, 82, 83, 84 and 96 were used to test the effect of different growth switches on the production of mevalonate. As demonstrated in FIG. 4, all the designed growth switches were shown to increase the specific mevalonate production. Furthermore, three of them, the ones targeting pryF, oriC and dnaA, increased the yield of mevalonate. Among all the tested targets, the inhibition of pyrF expression resulted in the highest production yield of mevalonate.

In another experiment, the dynamic process of mevalonate production was monitored in the growth arrested cells. In the strain expressing CRISPRi-pyrF system, the production of mevalonate continues for more than 20 hours after the stop of cell growth.

Conclusion

In conclusion, it was found that inhibition of nucleotide biosynthesis through inhibition of pyrF expression, in particular, resulted in efficient growth inhibition while at the same time significantly increasing the yield of mevalonate production as well as heterologous protein production.

### Example 3—Growth Arrested by Inhibitors Targeting Nucleotide Synthesis

In order to further test the effect of inhibiting cell growth by disrupting nucleotide synthesis, an inhibitor of nucleotide synthesis, 5-fluorouracil (5-FU), was applied to control cell growth. The production of both mevalonate and tyrosine was investigated when growth was repressed due to the addition of 5-FU.

Materials and Methods

Strains, Medium and Plasmids

Escherichia coli strains and plasmids used in this study are listed in Table S1 and S2. E. coli MG1655 was used as the parental strain for the production of mevalonate and tyrosine. The mevalonate producing strain was generated by transforming the plasmid pMevT into MG1655 (Martin et al., 2003), while the tyrosine producing strain was made by transforming the plasmids pS3 and pY3 into MG1655 (Juminaga et al., 2012), using standard methods (Sambrook and Russell, 2001). LB medium and LB agar plates with corresponding antibiotics were used for transformation and selection. Carbenicillin, ampicillin, and chloramphenicol were used to select for maintenance of plasmids in concentrations of 100 μg/mL, 100 μg/mL and 25 μg/mL, respectively. Minimal M9 medium was used as the base medium in this study.

Production Characterization

A single colony of each strain was used to inoculate M9 medium supplemented with 1% (w/v) glucose and 0.02% yeast extract (M9G1YE). The pre-cultures were diluted 100 times in fresh M9 medium with 0.2% (w/v) glucose and 0.02% (w/v) yeast extract. 500 μM and 50 μM IPTG were added to induce the production pathway in the mevalonate and tyrosine producing strains, respectively. Corresponding antibiotics were also added to the cultures. Twelve 3-ml cultures were aliquoted into 24 well plates for cultivation at 37° C. and 250 rpm. Four selected concentrations of 5-FU were added to corresponding cultures when cells entered early log phase. The exact time point for the addition of inhibitors to the mevalonate and tyrosine producing strains was 2 hours and 5 hours after inoculation, respectively. After 24 hours, 20-μl samples from each culture were diluted 10 times into 96-well plates for OD measurement. Another sample was taken from each culture for HPLC analysis. Details for HPLC sample preparation are described below.

HPLC Analysis

For the mevalonate producing strain, samples were first filtered through AcroPrep™ 96-well Filter Plates with 0.2 μm Supor® (Pall Corporation, USA). Filtered cultures were diluted with an appropriate amount of milli-Q water to 250 μl, and then 19 μL of 20% (v/v) sulfuric acid was added. The mixture was incubated on ice for five minutes and then transferred for HPLC analysis. Mevalonate and glucose were quantified using HPLC (Thermo) equipped with a Bio-RAD Aminex HPX-87H ion exclusion column (catalog #125-0140) incubated at 50° C. and with 0.01 N sulfuric acid as a mobile phase running at 0.6 ml/min as described before (Beck et al., 2012). Mevalonate and glucose were detected by refractive index, and their concentrations were determined by comparison to a standard curve.

For the tyrosine producing strain, samples were first centrifuged, and supernatants were collected and diluted with appropriate milli-Q water. Afterwards the prepared samples were divided into two portions for tyrosine and glucose quantification, separately. Tyrosine was quantified by HPLC similar to a previous method used for measurement of p-coumaric acid (Jendresen et al., 2015). The supernatant (20 μL) was separated on a Discovery HS F5 (5 μm) column (30° C.) in a Thermo HPLC setup, using a gradient elution with two solvents: 10 mM ammonium formate adjusted to pH 3.0 with formic acid (A) and acetonitrile (B) running at 1.5 ml/min, starting at 5% B. The fraction of B increased linearly from 5% to 60% from 1.5 min to 7 min after injection. Then the fraction of B decreased back to 5% between 9.5 and 9.6 min, and remained there until 12 min. Tyrosine eluting at 2.7 minutes was quantified by absorbance at 277 nm. Glucose was quantified as described above, except that the column was incubated at 37° C. Mevalonolactone, L-tyrosine and glucose standards were purchased from Sigma-Aldrich.

Results

Enhanced Production of Mevalonate and Tyrosine Per OD Achieved Through Growth Limitation

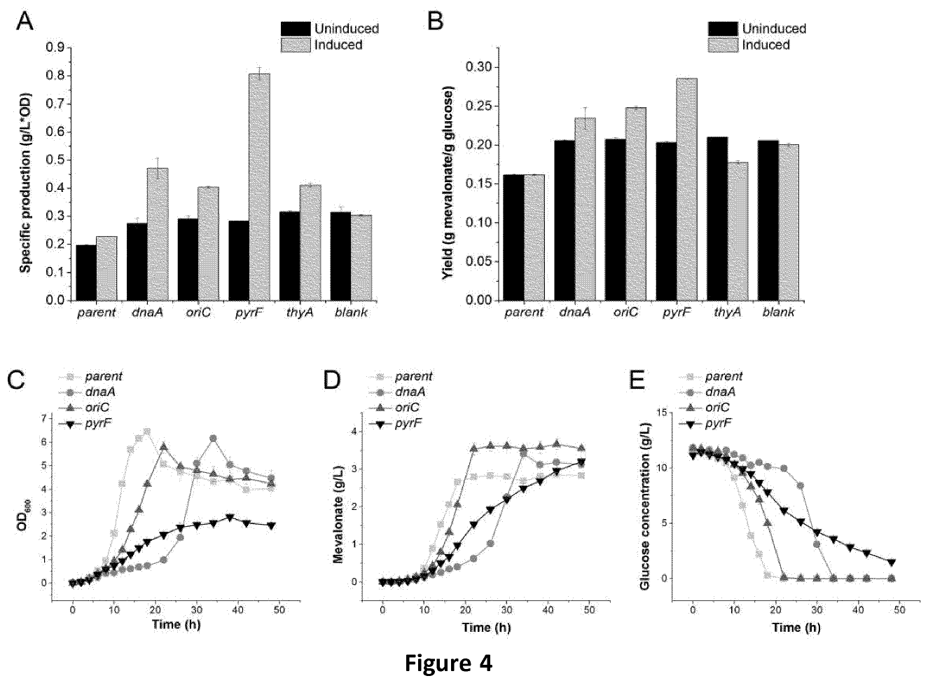

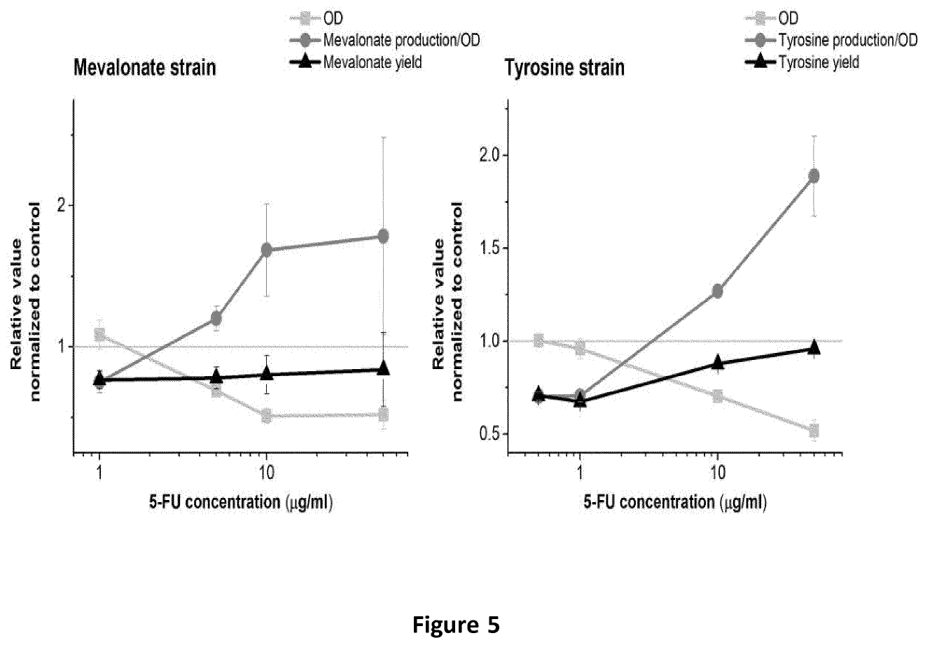

As shown in FIG. 5, the production of either mevalonate or tyrosine per OD was increased, compared to the cultures without treatment, when selected concentrations of 5-FU were added to the cultures. The yield of both compounds were shown to increase while the growth inhibition was increased by adding more 5-FU to the cultures.

Conclusion

In conclusion, the experiment shows that inhibition of nucleotide biosynthesis can be used limit the growth of the production host and to increase the yield and specific productivity of both tyrosine and mevalonate.

### Example 4—Growth Controlled by Nucleotide Supplementation to a pyrF Deletion Strain

The idea of repressing cell growth by limiting nucleotide synthesis was further tested in the pyrF knock-out strain of MG1655, in which the cell growth was controlled by the supplementation with uracil. This strain requires uracil supplementation for growth, and by supplying a limiting concentration of uracil to the growth medium, the growth can be limited at a given cell density.

SoT30, a pyrF knock-out strain of MG1655, was obtained by inserting the kanamycin cassette from pyrF knock-out strain of KEIO collections (Baba et al., 2006) into MG1655, with the assist of pKD46. The kanamycin cassette was amplified using primers SON63 and SON64 (Table S3). Standard protocols were used for the deletion and plasmids curing process (Baba et al., 2006). Plasmid pMevT was transformed into SoT30 to obtain the mevalonate producing strain SoT32. Plasmids pS4 and pY3 were transformed into SoT30 and MG1655 to obtain tyrosine producing strain SoT102 and SoT101 (Table S3), respectively. Selection of correct transformants were carried out using the protocols described above.

In order to test the effect of growth control on the production of biochemicals, pre-cultures were first prepared for SoT30, SoT17, SoT102 and SoT101 by cultivating each strain overnight in M9 media with 1% glucose, 0.02% yeast extract and for knock-out strain also 200 mg/L uracil. Pre-cultures were diluted 50 times in M9 media with 0.02% yeast extract and 0.2% glucose (SoT17 and SoT30) or 0.5% glucose (SoT101 and SoT102). 0.5 mM and 0.05 mM IPTG was added to the cultures of the mevalonate producing strains (SoT17 and SoT30) and tyrosine producing strains (SoT101 and SoT102), respectively. Different concentrations of uracil were supplemented to pyrF knock-out strains (SoT30 and SoT102) in order to enable cell growth to different levels, after which uracil becomes limiting for growth. Cultures were cultivated at 37° C. and 250 rpm. For SoT17 and SoT30, cultures were sampled after 25 hours of cultivation in 3 mL volume, and used for OD, glucose and mevalonate analysis. For SoT101 and SoT102, cultures were sampled after 48 hours of cultivation in 25 mL volume, and used for OD, glucose and tyrosine analysis. The analysis was carried out as described in example 3. Duplicates experiments were performed.

As it shown in Table 7, by adjusting the concentration of uracil, the growth of pyrF knock-out strain was successfully controlled. The yield of both mevalonate and tyrosine could be increased by reducing cell growth. The specific production of both compounds could also be enhanced in this way.

Conclusion

In conclusion, it was found that repression of growth through the starvation of nucleotides results in a surprisingly high increase in the production yield and specific productivity of both mevalonate and tyrosine. The supplementation of the pyrF-deletion strain with nucleotides in amounts that become limiting for growth, corresponds directly to the repression of nucleotide biosynthesis through other means. It is therefore clear that repression of nucleotide biosynthesis is effective for increasing production of recombinant proteins and biochemicals, including for example tyrosine and mevalonate, as well as their derivatives.

TABLE 7
Effect of repressing growth of a pyrF-deletion strain on the
production of both tyrosine and mevalonate. A pyrF-deletion strain
requires supplementation with uracil, and by supplementing the
growth medium with low concentrations of uracil, the growth of
the production organism can therefore be repressed at certain
cell densities or biomass concentrations. The cell density, yield
and specific production of mevalonate and tyrosine. Data are
shown as mean values and standard deviation.
Mevalonate production

| Concentration of uracil | | Mevalonate | Mevalonate |
|---|---|---|---|
| (mg/L) | OD | yield | production/OD |
| 2 | 0.4 ± 0.01 | 1.03 ± 0.06 | 2.66 ± 0.25 |
| 5 | 0.49 ± 0.01 | 1.17 ± 0.07 | 2.45 ± 0.09 |
| 10 | 0.83 ± 0.08 | 0.72 ± 0.01 | 0.89 ± 0.07 |
| 20 | 0.93 ± 0 | 0.52 ± 0.02 | 0.57 ± 0.02 |
| 200 | 0.83 ± 0.01 | 0.39 ± 0 | 0.48 ± 0 |

Tyrosine production

| Concentration of uracil | | Tyrosine | Tyrosine |
|---|---|---|---|
| (mg/L) | OD | yield | production/OD |
| 20 | 0.57 ± 0 | 4.15 ± 0.03 | 7.33 ± 0.15 |
| 200 | 0.94 ± 0 | 2.67 ± 0.05 | 2.77 ± 0.05 |

TABLE S1
Strains used for the experiments

| No. | Strains | Description | Source |
|---|---|---|---|
| | E. coli NEB 5-alpha | fhuA2 Δ(argF-lacZ)U169 phoA | NEB Bio Inc |
| | | glnV44 Φ80 Δ(lacZ)M15 | |
| | | gyrA96 recA1 relA1 endA1 thi- | |
| | | 1 hsdR17 (cloning strain) | |
| | E. coli MG1655 | F- lambda- ilvG- rfb-50 rph-1 | |
| | E. coli TCR | MG1655 attB- | This work |
| | | 186(O): tetracycline inducible | |
| | | dCas9 expression cassette | |
| | E. coli Sii17 | E. coli MG1655 | This work |
| | | Tn7::BBJ23100-GFP::KanR | |
| SoT30 | E. coli MG1655[ΔpyrF] | E. coli MG1655 ΔpyrF::KanR | This work |
| SoT53 | E. coli MG1655[Tcrispri-DnaA] | MG1655 with pdCas9-bacteria | This work |
| | | and pSLQ1236-dnaA | |
| SoT54 | E. coli MG1655[Tcrispri-OriC] | MG1655 with pdCas9-bacteria | This work |
| | | and pSLQ1236-oriC | |
| SoT55 | E. coli MG1655[Tcrispri-pyrF] | MG1655 with pdCas9-bacteria | This work |
| | | and pSLQ1236-pyrF | |
| SoT56 | E. coli MG1655[Tcrispri-thyA] | MG1655 with pdCas9-bacteria | This work |
| | | and pSLQ1236-thyA | |
| SoT65 | E. coli MG1655[Tcrispri-NON] | MG1655 with pdCas9-bacteria | This work |
| | | and pSLQ1236-nc | |
| SoT58 | E. coli MG1655[GFP + Tcrispri-GFP] | MG1655 with CDF-GFP, | This work |
| | | pdCas9-bacteria and | |
| | | pSLQ1236-GFP | |
| SoT59 | E. coli MG1655[GFP + Tcrispri-DnaA] | MG1655 with CDF-GFP, | This work |
| | | pdCas9-bacteria and | |
| | | pSLQ1236-dnaA | |
| SoT60 | E. coli MG1655[GFP + Tcrispri-OriC] | MG1655 with CDF-GFP, | This work |
| | | pdCas9-bacteria and | |
| | | pSLQ1236-oriC | |
| S0T61 | E. coli MG1655[GFP + Tcrispri-pyrF] | MG1655 with CDF-GFP, | This work |
| | | pdCas9-bacteria and | |
| | | pSLQ1236-pyrF | |
| SoT62 | E. coli MG1655[GFP + Tcrispri-thyA] | MG1655 with CDF-GFP, | This work |
| | | pdCas9-bacteria and | |
| | | pSLQ1236-thyA | |
| S0T66 | E. coli MG1655[GFP + Tcrispri-NON] | MG1655 with CDF-GFP, | This work |
| | | pdCas9-bacteria and | |
| | | pSLQ1236-nc | |
| SoT79 | E. coli TCR[GFP-6NON] | TCR with CDF-GFP and | This work |
| | | pSLQ1236-nc | |
| S0T80 | E. coli TCR[GFP-6GFP] | TCR with CDF-GFP and | This work |
| | | pSLQ1236-GFP | |
| S0T81 | E. coli TCR[MevT-6DnaA] | TCR with pMevT and | This work |
| | | pSLQ1236-dnaA | |
| SoT82 | E. coli TCR[MevT-6OriC] | TCR with pMevT and | This work |
| | | pSLQ1236-oriC | |
| SoT83 | E. coli TCR[MevT-6pyrF] | TCR with pMevT and | This work |
| | | pSLQ1236-pyrF | |
| SoT84 | E. coli TCR[MevT-6thyA] | TCR with pMevT and | This work |
| | | pSLQ1236-thyA | |
| SoT96 | E. coli TCR[MevT-6empty] | TCR with pMevT and | This work |
| | | pSLQ1236-blank | |
| SoT17 | E. coli MG1655[MevT] | MG1655 with pMevT | This work |
| SoT64 | E. coli MG1655[pY3 + pS3] | MG1655 with pS3 and pY3 | This work |
| SoT100 | E. coli Sii17[dCas9-6blank] | Sii17 with pdCas9-bacteria and | This work |
| | | pSLQ1236-blank | |
| SoT32 | MG1655[ΔpyrF][Mev] | SoT30 with pMevT | This work |
| SoT101 | MG1655[pS4 + pY3] | MG1655 with pS4 and pY3 | This work |
| SoT102 | MG1655[ΔpyrF][pS4 + pY3] | SoT30 with pS4 and pY3 | This work |

TABLE S2
Plasmids used for the experiments.

| No. | Plasmids | Description | Reference/source | Antibiotics |
|---|---|---|---|---|
| | pCDFDuet-1 | | Novagen | SpeR |
| pSon25 | pdCas9-bacteria | dCas9 expression | Addgene Bio Inc | CamR |
| | | plasmid, dCas9 was | | |
| | | expressed under | | |
| | | tetracycline inducible | | |
| | | promoter | | |
| pSon33 | pSLQ1236 | sgRNA expression | (Larson et al., | AmpR |
| | | plasmid, sgRNA was | 2013) | |
| | | expressed under | | |
| | | tetracycline inducible | | |
| | | promoter | | |
| pSon17 | pMevT | Plasmid for mevaloante | (Martin et al., | CamR |
| | | production, pLac33( Low- | 2003) | |
| | | copy broad-host | | |
| | | expression plasmid; CmR) | | |
| | | derivative containing the | | |
| | | atoB, HMGS and tHMGR | | |
| | | genes; CmR | | |
| | pOSIP | one step cloning- | (St-Pierre et al., | KanR |
| | | integration plasmid, | 2013) | |
| | | integration site at phage | | |
| | | 186 sites | | |
| | pE-FLP | looping out kanR cassette | (St-Pierre et al., | AmpR |
| | | for integration | 2013) | |
| pSon31 | CDF-GFP | GFP reporter plasmids, | This work | SpeR |
| | | GFP expression were | | |
| | | controlled by | | |
| | | constitutively promoter | | |
| pSon36 | pSLQ1236-GFP | pSLQ1236 with sgRNA | This work | AmpR |
| | | targeting GFP | | |
| pSon37 | pSLQ1236-dnaA | pSLQ1236 with sgRNA | This work | AmpR |
| | | targeting DnaA | | |
| pSon38 | pSLQ1236-oriC | pSLQ1236 with sgRNA | This work | AmpR |
| | | targeting OriC | | |
| pSon39 | pSLQ1236-pyrF | pSLQ1236 with sgRNA | This work | AmpR |
| | | targeting pyrF | | |
| pSon40 | pSLQ1236-thyA | pSLQ1236 with sgRNA | This work | AmpR |
| | | targeting thyA | | |
| pSon49 | pSLQ1236-blank | pSLQ1236 without sgRNA | This work | AmpR |
| | | sequence | | |
| pSon44 | pSLQ1236-nc | pSLQ1236 with sgRNA | This work | AmpR |
| | | without targeting | | |
| | | sequence | | |
| pSon50 | pOSIP-dCas9 | pOSIP carry dCas9 | This work | KanR |
| | | expression cassette for | | |
| | | integration, dCas9 was | | |
| | | expressed under | | |
| | | tetracycline inducible | | |
| | | promoter | | |
| pSon24 | psgRNA- | constitutively express | Addgene Bio Inc | AmpR |
| | bacteria | sgRFP | | |
| pSon47 | pY3 | Plasmid for tyrosine | (Juminaga et al., | AmpR |
| | | production | 2012) | |
| | | (downstream), | | |
| | | pBbA5a::tyrB-tyrA*-aroC | | |
| | | T1-Ptrc-aroA-aroL | | |
| pSon23 | pS3 | Plasmid for tyrosine | (Juminaga et al., | CamR |
| | | production (upstream), | 2012) | |
| | | pBbB5c::aroE-aroD- | | |
| | | aroBop-aroG*-ppsA-tktA | | |
| pSon18 | pKD46 | red recombinase systems | Datsenko and | AmpR |
| | | | Wanner | |
| | | | (2000) | |
| pSon51 | pS4 | | (Juminaga et al., | CamR |
| | | | 2012) | |

TABLE S3
Primer sequences used in the experiments.

| No. | Primers | Sequence(5′-3′) |
|---|---|---|
| SON112 | Gib_sg_GFP_F | CATCTAATTCAACAAGAATTGTTTTA |
| | | GAGCTAGAAATAGCAAGTTAAAATAA |
| | | GGC |
| SON113 | Gib_sg_GFP_R | AATTCTTGTTGAATTAGATGACTAGT |
| | | ATTATACCTAGGACTGAGCTAGC |
| SON114 | Gib_sg_thyA_F | AATGGAAAGCGTTCCGGTTCGTTTTA |
| | | GAGCTAGAAATAGCAAGTTAAAATAA |
| | | GGC |
| SON115 | Gib_sg_thyA_R | GAACCGGAACGCTTTCCATTACTAGT |
| | | ATTATACCTAGGACTGAGCTAGC |
| SON116 | Gib_sg_pyrF_F | AGGAGAATTCGTAACAGCGCGTTTTA |
| | | GAGCTAGAAATAGCAAGTTAAAATAA |
| | | GGC |
| SON117 | Gib_sg_pyrF_R | GCGCTGTTACGAATTCTCCTACTAGT |
| | | ATTATACCTAGGACTGAGCTAGC |
| SON138 | sgBlank_F | GTTTTAGAGCTAGAAATAGCAAGTTA |
| | | AAATAAGGC |
| SON139 | sgBlank_R | GCTATTTCTAGCTCTAAAACACTAGT |
| | | ATTATACCTAGGACTGAGCTAGC |
| SON144 | 1236Gib_sg_ | GCTATTTCTAGCTCTAAAACACTAGT |
| | Blank_R | CTTTTCTCTATCACTGATAGGGA |
| SON108 | Gib1110_GFP_F | ACTTTAATAAGGAGATATACGGTTAC |
| | | GGTTGAGTAATAAATGGA |
| SON109 | Gib1110_GFP_R | TGGCAGCAGCCTAGGTTAATCGAACC |
| | | GAACAGGCTTATGTC |
| SON235 | pOSIP_ufwd | AGATGCAUGGCGCCTAAC |
| | (1297) [se] | |
| SON236 | pOSIP_urev | AGCCCTCUAGAGGATCCCCGGGTA |
| | (1298) [se] | |
| SON168 | Cas_F_U | AGAGGGCUCAACGTCTCATTTTCGCC |
| | | AG |
| SON169 | Cas_R_U | ATGCATCUATCCTTACTCGAGTTAGT |
| | | CACC |
| SON176 | 1236empty_F | AGTCGGUGCTTTTTTTGAAG |
| SON177 | 1236empty_R | ACCGACUACTAGTCTTTTCTCTATCA |
| | | CTG |
| SON172 | lib_F | CTCCCTATCAGTGATAGAGAAAAGAC |
| | | T |
| SON173 | lib_R | CGGGCCCAAGCTTCAAAAAAAGCACC |
| | | GACTCGGTGCCACTTTTTCAAGTTGA |
| | | TAACGGAC |
| SON178 | 6lib_F | agtcggtgctttttttgaag |
| SON179 | 6lib_R | ACTAGTCTTTTCTCTATCACTGATAG |
| | | GGAG |
| SON203 | t1_F | ACTTCAATTAACACCAGCGGGTTTTA |
| | | GAGCTAGAAATAGCAAGTTAAAATAA |
| | | GGC |
| SON204 | t1_R | CCGCTGGTGTTAATTGAAGTACTAGT |
| | | CTTTTCTCTATCACTGATAGGGA |
| SON205 | t2_F | AGACGCGTTAGTGTCTTATCGTTTTA |
| | | GAGCTAGAAATAGCAAGTTAAAATAA |
| | | GGC |
| SON206 | t2_R | GATAAGACACTAACGCGTCTACTAGT |
| | | CTTTTCTCTATCACTGATAGGGA |
| SON207 | t3_F | AGCTCTTCCTCAATGTTGACGTTTTA |
| | | GAGCTAGAAATAGCAAGTTAAAATAA |
| | | GGC |
| SON208 | t3_R | GTCAACATTGAGGAAGAGCTACTAGT |
| | | CTTTTCTCTATCACTGATAGGGA |
| SON209 | t4_F | ATTACCTTTTGTGAAGGCAGGTTTTA |
| | | GAGCTAGAAATAGCAAGTTAAAATAA |
| | | GGC |
| SON210 | t4_R | CTGCCTTCACAAAAGGTAATACTAGT |
| | | CTTTTCTCTATCACTGATAGGGA |
| SON211 | t5_F | CGTCAGGGTGACTTTCTTGCGTTTTA |
| | | GAGCTAGAAATAGCAAGTTAAAATAA |
| | | GGC |
| SON212 | t5_R | GCAAGAAAGTCACCCTGACGACTAGT |
| | | CTTTTCTCTATCACTGATAGGGA |
| SON213 | t6_F | CGTCGCGGTTCAGACATGAAGTTTTA |
| | | GAGCTAGAAATAGCAAGTTAAAATAA |
| | | GGC |
| SON214 | t6_R | TTCATGTCTGAACCGCGACGACTAGT |
| | | CTTTTCTCTATCACTGATAGGGA |
| SON215 | t7_F | GAGAAGCAGATGACTTCCGGGTTTTA |
| | | GAGCTAGAAATAGCAAGTTAAAATAA |
| | | GGC |
| SON216 | t7_R | CCGGAAGTCATCTGCTTCTCACTAGT |
| | | CTTTTCTCTATCACTGATAGGGA |
| SON217 | t9_F | GTTGAATGTCCAGAACGGGTGTTTTA |
| | | GAGCTAGAAATAGCAAGTTAAAATAA |
| | | GGC |
| SON218 | t9_R | ACCCGTTCTGGACATTCAACACTAGT |
| | | CTTTTCTCTATCACTGATAGGGA |
| SON219 | t10_F | TAAACTGTGGCGGATAGGATGTTTTA |
| | | GAGCTAGAAATAGCAAGTTAAAATAA |
| | | GGC |
| SON220 | t10_R | ATCCTATCCGCCACAGTTTAACTAGT |
| | | CTTTTCTCTATCACTGATAGGGA |
| SON221 | t11_F | TACTAAGACTACCAGGGCGGGTTTTA |
| | | GAGCTAGAAATAGCAAGTTAAAATAA |
| | | GGC |
| SON222 | t11_R | CCGCCCTGGTAGTCTTAGTAACTAGT |
| | | CTTTTCTCTATCACTGATAGGGA |
| SON223 | t13_F | AATCCTGCGCCTGACAGGCCGTTTTA |
| | | GAGCTAGAAATAGCAAGTTAAAATAA |
| | | GGC |
| SON224 | t13_R | GGCCTGTCAGGCGCAGGATTACTAGT |
| | | CTTTTCTCTATCACTGATAGGGA |
| SON225 | t14_F | ATAGGTAAATTTCTGGGTCCGTTTTA |
| | | GAGCTAGAAATAGCAAGTTAAAATAA |
| | | GGC |
| SON226 | t14_R | GGACCCAGAAATTTACCTATACTAGT |
| | | CTTTTCTCTATCACTGATAGGGA |
| SON227 | t15_F | CGGCATATACATTTGGGTCCGTTTTA |
| | | GAGCTAGAAATAGCAAGTTAAAATAA |
| | | GGC |
| SON228 | t15_R | GGACCCAAATGTATATGCCGACTAGT |
| | | CTTTTCTCTATCACTGATAGGGA |
| SON229 | t16_F | CTTCGGTTATTGCCGGGTCCGTTTTA |
| | | GAGCTAGAAATAGCAAGTTAAAATAA |
| | | GGC |
| SON230 | t16_R | GGACCCGGCAATAACCGAAGACTAGT |
| | | CTTTTCTCTATCACTGATAGGGA |
| SON231 | t17_F | TGTTTAACAAATGGGGGCACGTTTTA |
| | | GAGCTAGAAATAGCAAGTTAAAATAA |
| | | GGC |
| SON232 | t17_R | GTGCCCCCATTTGTTAAACAACTAGT |
| | | CTTTTCTCTATCACTGATAGGGA |
| SON233 | NGS_Primer_F | TCGTCGGCAGCGTCAGATGTGTATAA |
| | | GAGACAGCACTCCCTATCAGTGATAG |
| | | AGAAAAG |
| SON234 | NGS_Primer_R | GTCTCGTGGGCTCGGAGATGTGTATA |
| | | AGAGACAGATTCAGATCCTCTTCTGA |
| | | GATGAG |
| SON63 | D_pyrF_F | acctgtttcgcgccacttcc |
| SON64 | D_pyrF_R | GTAGACCAGACGGCTGTTGG |

### Example 5—Inhibition of Growth can Significantly Increase Production of Heterologous Proteins in B. subtilis

Background

In order to demonstrate that the method of decoupling growth from production also works in other organisms, an experiment was carried out in Bacillus subtilis, a strain commonly used for production of heterologous proteins. In this experiment, it is demonstrated that inhibition of pyrimidine biosynthesis can result in significantly increased production of the heterologous protein, GFP. In the particular experiments, CRISPRi was used to repress the expression of pyrH.

Cloning

In this experiment, a strain, B. subtilis 168 thrC::pDG1731-PS1-sfGFP, was engineered to constitutively express a heterologous protein, GFP, from the genome (Table 8). This strain was designed to be the control in the experiment. Another strain, B. subtilis 168 lacA::pJMP1 amyE::pJMP222 thrC::pDG1731-PS1-sfGFP (Growth switch), additionally carries a xylose inducible gene encoding pdCas9, and a constitutively expressed sgRNA targeting pyrH. Induction of this strain with xylose will result in the inhibition of transcription of pyrH. The different strains were generated as described in detail below.

In order to express GFP heterologous in B. subtilis, a copy of sfGFP was cloned into the integration vector pDG1731 as described below. The plasmid pDG1731 (Table 9) was amplified using the primers pDG1731_VR and pDG1731_PS1_VF, and pS003 was amplified using the primers sfGFP_UF and sfGFP_UR (Table 10), both with Phusion U polymerase (New England Biolabs, United States) following the manufacturer's instructions. The sizes of the products were confirmed by gel electrophoresis, and the fragments were purified using a NucleoSpin PCR clean-up gel extraction kit (Macherey-Nagel, Germany) following the “PCR cleanup” protocol supplied with the kit. The pDG1731 backbone was treated with FastDigest DpnI (Thermo Fisher Scientific, United States) following the manufacturer's instructions, followed by a second purification. The backbone and sfGFP insert were assembled by mixing the fragments in a 1:3 ratio (backbone:insert). 2 μL HF buffer and 1 μL USER enzyme (New England Biolabs, United States) was added to the mixture, and MilliQ water was added to a total volume of 12 μL. The reactions were incubated at 37° C. for 25 minutes, 18° C. for 25 minutes and 10° C. for 10 minutes. 8 μL MilliQ water was added to the reactions, and 5 μL was used to transform chemically competent TOP10 cells. The cells were prepared as described in Winstel et al. (2016), and the transformation was performed as described in Froger & Hall (2007). The cells were plated on LB plates supplemented with 100 μgmL⁻¹ ampicillin, and incubated overnight at 37° C. The resulting colonies were screened for the presence of the desired constructs by suspending individual colonies in 20 μL MilliQ water, and using this as template in PCRs with the primers thrC_seqF and sfGFP_seqR and OneTaq polymerase (New England Biolabs, United States) following the manufacturer's instructions. The sizes of the products were checked by gel electrophoresis, and confirmed colonies were inoculated in LB media supplemented with 100 μgmL⁻¹ ampicillin and incubated overnight at 37° C. with 250 RPM shaking. The plasmids were purified from the cultures using a NucleoSpin Plasmid EasyPure kit (Macherey-Nagel, Germany), following the manufacturer's instructions. The constructs were confirmed by Sanger sequencing with the primers sfGFP_seqF and sfGFP_seqR, using a Mix2Seq kit (Eurofins Genomics, Luxembourg) following the manufacturer's instructions. The resulting construct was named “pDG1731-PS1-sfGFP” (Table 9).

The three integrative plasmids, pJMP1, pJMP222, and pDG1731-PS1-sfGFP (Table 9) were transformed into B. subtilis 168 by natural competence as described in Vojcic et al. (2012), although without adding histidine to the SM1 and SM2 medium. The transformations were plated on LB plates supplemented with 10 μg/mL erythromycin, 10 μg/mL chloramphenicol, and 100 μg/mL spectinomycin, respectively. The resulting strain, B. subtilis 168 thrC::pDG1731-PS1-sfGFP, constitutively expresses GFP from the genome and was used as a control in the experiments. The other strain, B. subtilis 168 lacA::pJMP1 amyE::pJMP222 thrC::pDG1731-PS1-sfGFP (Growth switch), additionally carries a xylose inducible gene encoding pdCas9, and a constitutively expressed sgRNA targeting pyrH. Induction of this strain with xylose results in the inhibition of the transcription of pyrH.

TABLE 8
Strains

| Name | Genotype |
|---|---|
| E. coli TOP10 | F- mcrA Δ(mrr-hsdRMS- mcrBC) |
| | φ80lacZΔM15ΔlacX74 nupG recA1 |
| | araD139 Δ(ara-leu)7697 galE15 galK16 rpsL(StrR) |
| | endA1 λ- |
| B. subtilis 168 | trpC2 |
| E. coli TOP10 pDG1731-PS1- | TOP10; pDG1731-PS1-sfGFP |
| sfGFP | |
| B. subtilis 168 thrC::pDG1731- | 168; thrC::pDG1731-PS1-sfGFP |
| PS1-sfGFP | |
| B. subtilis 168 lacA::pJMP1 | 168; lacA::pJMP1 amyE::pJMP222 thrC::pDG1731-PS1- |
| amyE::pJMP222 thrC::pDG1731- | sfGFP |
| PS1-sfGFP | |

TABLE 9
Plasmids

| | | | Reference/ |
|---|---|---|---|
| Name | Features | Selection marker | source |
| pJMP1 | pAX01 derived construct, harbouring | AmpR/EryR | Peters et al. |
| | dCas9 from pdCas9-bacteria (Addgene | | (2016)/BGSC |
| | #44249) under control of the xylose | | |
| | inducible Pxyl promoter | | |
| pJMP222 | pDG1662 derived amyE integration | AmpR/CamR/SpcR | Peters et al. |
| | construct, harbouring the pyrH sgRNA for | | (2016)/BGSC |
| | B. subtilis (AATACGATACGTTTGTATTT) | | |
| | under control of the constitutive Pveg | | |
| | promoter. | | |
| pDG1731 | thrC integration plasmid | AmpR/EryR/SpcR | Peters et al. |
| | | | (2016)/BGSC |
| pS003 | Plasmid containing the sfGFP sequence | KanR | This study |
| pDG1731- | pDG1731 derived construct, harbouring | AmpR/EryR/SpcR | This study |
| PS1-sfGFP | sfGFP under control of the synthetic | | |
| | constitutive PS1 promoter from Guiziou et | | |
| | al. (2016). | | |

TABLE 10
Primers

| Name | Sequence (5′ to 3′) |
|---|---|
| pDG1731_VF | AGCTGAAAUAGCTGCGCTTTTTTGTGTCA |
| | TAACTAATAACGTAACGTGACTGGC |
| pDG1731_PS1_VR | AATCTTTTCUCCCTGATAATTTAACACAC |
| | TTTCAAAAGAGTGTCAACGTGTATTGACG |
| | CAGTCGAACGAAAATCGCCATTCGC |
| sfGFP_UF | AAACATGAGTAAAGGCGAAGAGCTG |
| sfGFP_UR | ATTTCAGCUGCGCTTTTTTTATTTGTACA |
| | GTTCATCCATACCATGCG |
| thrC_seqF | GTGTAGAAGGGAACGGTTGG |
| sfGFP_seqR | TTGTATTCCAGCTTATGGCCC |
| sfGFP_seqF | CGTGCGGAAGTGAAATTTGAAGG |

Physiological Characterization

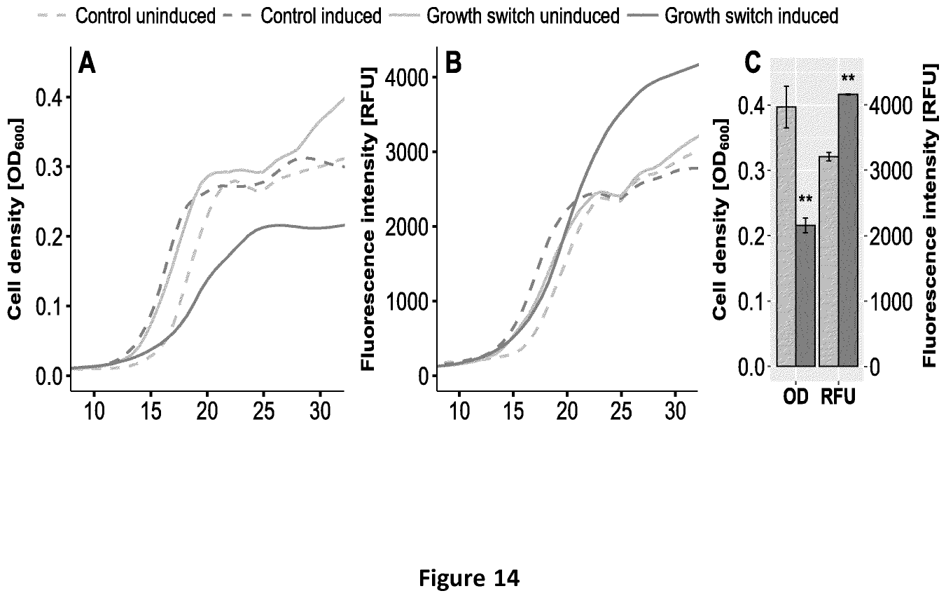

The fluorescence intensities of B. subtilis 168 thrC::pDG1731-PS1-sfGFP (Control) and B. subtilis 168 lacA::pJMP1 amyE::pJMP222 thrC::pDG1731-PS1-sfGFP (Growth switch), was measured during growth in minimal media either under induced (1% xylose) or uninduced conditions. This was performed by inoculating the strains in M9 minimal medium supplemented with 0.2% yeast extract and appropriate antibiotics, and letting them grow overnight. Cultures were diluted to an optical density (OD₆₀₀) of 0.01 in M9 minimal medium supplemented with appropriate antibiotics, inducer, 50 μg/mL L-tryptophan, and 50 μg/mL L-threonine. Cultures were dispensed in a Greiner CELLSTAR 96 flat bottom well plates in volumes of 200 μL per well. The plates were placed in a Synergy HM1 absorbance and fluorescence reader (BioTek Instruments, United States). Every 6 minutes the absorbance of the cultures were measured at 600 nm, and the fluorescence was measured using an excitation wavelength of 480 nm, an emission wavelength of 528 nm, and a gain value of 70. Between measurements the plates were shaken, and the temperature was kept at 37° C. The results from this experiment are shown in FIG. 14. The induced strain carrying the growth switch exhibited a 45.7±3.9% reduction in biomass accumulation compared to the uninduced culture. The absolute fluorescence of the induced growth decoupling sample was similar to that of both the control samples and the uninduced growth decoupling sample until approximately 20 hours, despite the cell density was significantly lower. After this time point the total fluorescence of the WT samples and the uninduced growth decoupling sample leveled off at a fluorescence intensity of around 3200 RFU, while the induced sample increases the entire time series to around 4200 RFU after 32 hours. This amounted to a 30±4.5% increase in fluorescence, which was shown to be statistically significant with a 1% significance level.

Conclusion

These results show that a CRISPRi system, can be used to reduce the biomass accumulation and increase the production of a heterologous protein of interest in B. subtilis.

### Example 6—Inhibition of Genes Involved in Nucleotide Biosynthesis Decreases Growth and Increases Production of Heterologous Proteins

Background

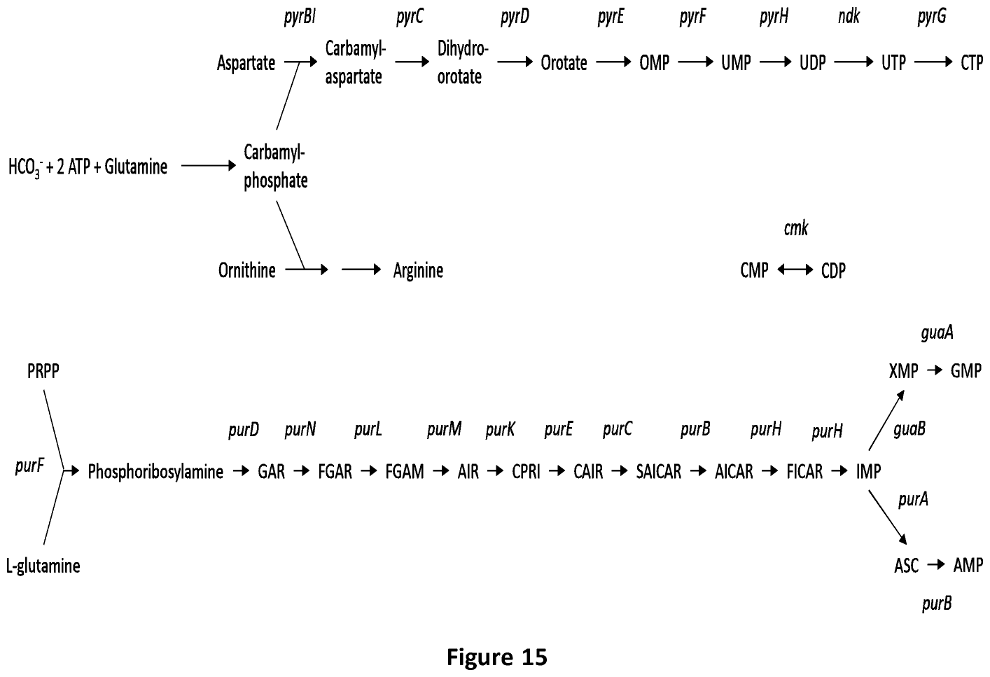

In order to further investigate the genes involved in nucleotide biosynthesis (FIG. 15) as potential targets for decoupling growth from production of biochemicals and recombinant proteins, individual guide RNA's were designed and cloned to enable CRISPRi based inhibition of the expression of the genes in these pathways. To investigate the effect on production of heterologous protein, a strain expressing GFP was used as a base strain. A range of related daughter strains expressing the selected gRNA's along with dCas9 were analyzed as described below.

Materials and Methods

Strains, Medium and Plasmids

Escherichia coli strains and plasmids used in this study are listed in Tables S4 and S5. Primer sequences are listed in Table S6. E. coli Sii17 was used as the parental strain for the characterization of growth and fluorescence. Different growth switches as well as a negative control system were transformed into Sii17 together with pdCas9 in order to create test strains JL86-105, JL114, 115 and JL122. Carbenicillin and chloramphenicol were used to select for maintenance of plasmids in concentrations of 100 μg/mL and 25 μg/mL, respectively.

The growth switches as well as the control switch consist of a pdCas9-bacteria plasmid (Addgene; Plasmid #44249) and one derivative of the pSLQ1236 plasmid (obtained as a gift from Professor Stanley Qi, Stanford University) (Larson et al., 2013). Derivatives of pSLQ1236 were obtained by modifying the original plasmid to target different locations as described in example 2. The targeting sequences are listed in Table 11. The complete sequences of the selected sgRNAs are listed in Table 12.

TABLE 11
Target sequence of sgRNAs for selected genes.

| Target | |
|---|---|
| gene | Sequence |
| purA | TTTACCTTCGTCACCCCATT |
| purB | TCCATCGACAGGGGAAACGG |
| purC | CGGGTTTTCCGTGCTGTATA |
| purD | CGGCGACTGGGCCGCTTTCC |
| purE | GACACGCGCCGGATTATTGC |
| purF | TCATAAATCGACTGGTTAAC |
| purH | AAACACTGAGCAGAGCGCGG |
| purK | GGCCTAACTGCCCGTTACCG |
| purL | ATTCGGAATGCCGACAGTGC |
| purM | GGCATCTTTGTAGCTAAGAG |
| purN | CTGTAAATTACTTCCGTTGC |
| guaA | GAGAACCGAAGTCCAGAATG |
| guaB | CGGTAGAGTGAGCAGGAACG |
| pyrB | ATGATATGTTTCTGATATAG |
| pyrD | AAAAGGGCTTTACGAACGAA |
| pyrE | TAAGCGCAAATTCAATAAAC |
| pyrF | AGGAGAATTCGTAACAGCGC |
| pyrG | GGCAATGCCTTTACCCAGAG |
| pyrH | TTTATAGACGGGTTTTGCAT |
| cmk | GGCCATCAATGGTAATAACC |
| ndk | ACGTTTTTTGCTACCGCGTT |

TABLE 12
Complete sequence of selected sgRNAs.

| Target | |
|---|---|
| gene | Sequence |
| purA | TTTACCTTCGTCACCCCATTGTTTTAGAGCTAGAAATAGC |
| | AAGTTAAAATAAGGCTAGTCCGTTATCAACTTGAAAAAGT |
| | GGCACCGAGTCGGTGCTTTTTT |
| purB | TCCATCGACAGGGGAAACGGGTTTTAGAGCTAGAAATAGC |
| | AAGTTAAAATAAGGCTAGTCCGTTATCAACTTGAAAAAGT |
| | GGCACCGAGTCGGTGCTTTTTT |
| purC | CGGGTTTTCCGTGCTGTATAGTTTTAGAGCTAGAAATAGC |
| | AAGTTAAAATAAGGCTAGTCCGTTATCAACTTGAAAAAGT |
| | GGCACCGAGTCGGTGCTTTTTT |
| purD | CGGCGACTGGGCCGCTTTCCGTTTTAGAGCTAGAAATAGC |
| | AAGTTAAAATAAGGCTAGTCCGTTATCAACTTGAAAAAGT |
| | GGCACCGAGTCGGTGCTTTTTT |
| purE | GACACGCGCCGGATTATTGCGTTTTAGAGCTAGAAATAGC |
| | AAGTTAAAATAAGGCTAGTCCGTTATCAACTTGAAAAAGT |
| | GGCACCGAGTCGGTGCTTTTTT |
| purF | TCATAAATCGACTGGTTAACGTTTTAGAGCTAGAAATAGC |
| | AAGTTAAAATAAGGCTAGTCCGTTATCAACTTGAAAAAGT |
| | GGCACCGAGTCGGTGCTTTTTT |
| purH | AAACACTGAGCAGAGCGCGGGTTTTAGAGCTAGAAATAGC |
| | AAGTTAAAATAAGGCTAGTCCGTTATCAACTTGAAAAAGT |
| | GGCACCGAGTCGGTGCTTTTTT |
| purK | GGCCTAACTGCCCGTTACCGGTTTTAGAGCTAGAAATAGC |
| | AAGTTAAAATAAGGCTAGTCCGTTATCAACTTGAAAAAGT |
| | GGCACCGAGTCGGTGCTTTTTT |
| purL | ATTCGGAATGCCGACAGTGCGTTTTAGAGCTAGAAATAGC |
| | AAGTTAAAATAAGGCTAGTCCGTTATCAACTTGAAAAAGT |
| | GGCACCGAGTCGGTGCTTTTTT |
| purM | GGCATCTTTGTAGCTAAGAGGTTTTAGAGCTAGAAATAGC |
| | AAGTTAAAATAAGGCTAGTCCGTTATCAACTTGAAAAAGT |
| | GGCACCGAGTCGGTGCTTTTTT |
| purN | CTGTAAATTACTTCCGTTGCGTTTTAGAGCTAGAAATAGC |
| | AAGTTAAAATAAGGCTAGTCCGTTATCAACTTGAAAAAGT |
| | GGCACCGAGTCGGTGCTTTTTT |
| guaA | GAGAACCGAAGTCCAGAATGGTTTTAGAGCTAGAAATAGC |
| | AAGTTAAAATAAGGCTAGTCCGTTATCAACTTGAAAAAGT |
| | GGCACCGAGTCGGTGCTTTTTT |
| guaB | CGGTAGAGTGAGCAGGAACGGTTTTAGAGCTAGAAATAGC |
| | AAGTTAAAATAAGGCTAGTCCGTTATCAACTTGAAAAAGT |
| | GGCACCGAGTCGGTGCTTTTTT |
| pyrB | ATGATATGTTTCTGATATAGGTTTTAGAGCTAGAAATAGC |
| | AAGTTAAAATAAGGCTAGTCCGTTATCAACTTGAAAAAGT |
| | GGCACCGAGTCGGTGCTTTTTT |
| pyrD | AAAAGGGCTTTACGAACGAAGTTTTAGAGCTAGAAATAGC |
| | AAGTTAAAATAAGGCTAGTCCGTTATCAACTTGAAAAAGT |
| | GGCACCGAGTCGGTGCTTTTTT |
| pyrE | TAAGCGCAAATTCAATAAACGTTTTAGAGCTAGAAATAGC |
| | AAGTTAAAATAAGGCTAGTCCGTTATCAACTTGAAAAAGT |
| | GGCACCGAGTCGGTGCTTTTTT |
| pyrF | AGGAGAATTCGTAACAGCGCGTTTTAGAGCTAGAAATAGC |
| | AAGTTAAAATAAGGCTAGTCCGTTATCAACTTGAAAAAGT |
| | GGCACCGAGTCGGTGCTTTTTT |
| pyrG | GGCAATGCCTTTACCCAGAGGTTTTAGAGCTAGAAATAGC |
| | AAGTTAAAATAAGGCTAGTCCGTTATCAACTTGAAAAAGT |
| | GGCACCGAGTCGGTGCTTTTTT |
| pyrH | TTTATAGACGGGTTTTGCATGTTTTAGAGCTAGAAATAGC |
| | AAGTTAAAATAAGGCTAGTCCGTTATCAACTTGAAAAAGT |
| | GGCACCGAGTCGGTGCTTTTTT |
| cmk | GGCCATCAATGGTAATAACCGTTTTAGAGCTAGAAATAGC |
| | AAGTTAAAATAAGGCTAGTCCGTTATCAACTTGAAAAAGT |
| | GGCACCGAGTCGGTGCTTTTTT |
| ndk | ACGTTTTTTGCTACCGCGTTGTTTTAGAGCTAGAAATAGC |
| | AAGTTAAAATAAGGCTAGTCCGTTATCAACTTGAAAAAGT |
| | GGCACCGAGTCGGTGCTTTTTT |

Characterization of Growth Inhibition and GFP Production

Biological triplicates of each strain were grown overnight as pre-cultures at 37° C., 250 rpm in M9 media with 0.5% (w/v) glucose and 0.02% (w/v) yeast extract (M9G0.5YE). The cultures were diluted 100 fold into 800 μL M9 media with 0.5% (w/v) glucose (M9G0.5) in 96-deep well plates and incubated at 37° C., 300 rpm. For each strain, six cultures were prepared, of which three were induced with 200 ng/μL aTc (anhydrotetracycline) one hour after inoculation. After 12 hours of growth, samples were diluted ten fold and fluorescence and OD was measured using a Synergy Mx plate reader (BioTek, USA). The GFP fluorescence was measured using an excitation at 485 nm and emission at 528 nm with a gain set to 100. The OD was measured at 630 nm. Samples were analyzed by flow cytometry using a Fortessa instrument (Becton Dickinson, San Jose, USA). Forward-scatter and side-scatter were detected as small- and large-angle scatters of the 488 nm laser, respectively. GFP fluorescence was detected with a 488 nm long-pass and a 530/30 nm band-pass filter set. For each sample, 100,000 events were counted.

Results and Conclusion

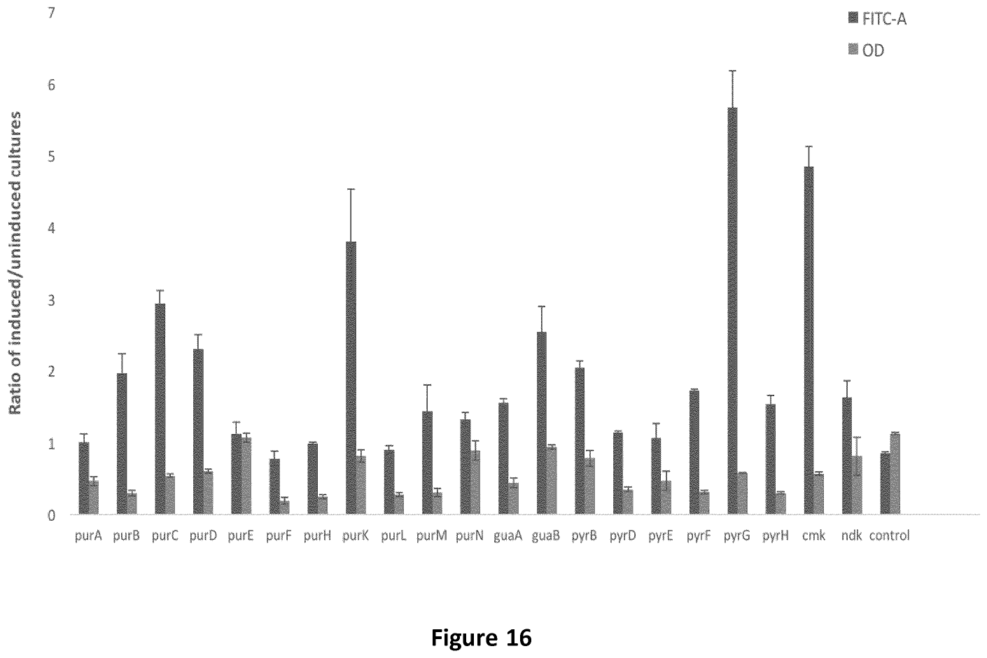

In FIG. 16, it can be seen that many of the genes involved in nucleotide biosynthesis were found to effectively inhibit growth and at the same time improve specific GFP production (production per cell). In particular, cmk and all pyr genes involved in CTP production were all found to increase specific GFP production.

TABLE S4
Strains used for the experiments.

| No. | Strains | Description | Source |
|---|---|---|---|
| JL86 | E. coli Sii17[Tcrispri-purA] | Sii17 with pdCas9-bacteria and | This work |
| | | pSLQ1236-purA | |
| JL87 | E. coli Sii17 [Tcrispri-purB] | Sii17 with pdCas9-bacteria and | This work |
| | | pSLQ1236-purB | |
| JL88 | E. coli Sii17 [Tcrispri-purC] | Sii17 with pdCas9-bacteria and | This work |
| | | pSLQ1236-purC | |
| JL89 | E. coli Sii17 [Tcrispri-purD] | Sii17 with pdCas9-bacteria and | This work |
| | | pSLQ1236-purD | |
| JL90 | E. coli Sii17 [Tcrispri-purE] | Sii17 with pdCas9-bacteria and | This work |
| | | pSLQ1236-purE | |
| JL91 | E. coli Sii17 [Tcrispri-purF] | Sii17 with pdCas9-bacteria and | This work |
| | | pSLQ1236-purF | |
| JL92 | E. coli Sii17 [Tcrispri-purH] | Sii17 with pdCas9-bacteria and | This work |
| | | pSLQ1236-purH | |
| JL93 | E. coli Sii17 [Tcrispri-purK] | Sii17 with pdCas9-bacteria and | This work |
| | | pSLQ1236-purC | |
| JL94 | E. coli Sii17 [Tcrispri-purL] | Sii17 with pdCas9-bacteria and | This work |
| | | pSLQ1236-purL | |
| JL95 | E. coli Sii17 [Tcrispri-purM] | Sii17with pdCas9-bacteria and | This work |
| | | pSLQ1236-purM | |
| JL96 | E. coli Sii17 [Tcrispri-purN] | Sii17 with pdCas9-bacteria and | This work |
| | | pSLQ1236-purN | |
| JL97 | E. coli Sii17 [Tcrispri-guaA] | Sii17 with pdCas9-bacteria and | This work |
| | | pSLQ1236-guaA | |
| JL98 | E. coli Sii17 [Tcrispri-guaB] | Sii17 with pdCas9-bacteria and | This work |
| | | pSLQ1236-guaB | |
| JL99 | E. coli Sii17 [Tcrispri-pyrB] | Sii17 with pdCas9-bacteria and | This work |
| | | pSLQ1236-pyrB | |
| JL101 | E. coli Sii17 [Tcrispri-pyrD] | Sii17 with pdCas9-bacteria and | This work |
| | | pSLQ1236-pyrD | |
| JL102 | E. coli Sii17 [Tcrispri-pyrE] | Sii17 with pdCas9-bacteria and | This work |
| | | pSLQ1236-pyrE | |
| JL103 | E. coli Sii17 [Tcrispri-pyrF] | Sii17 with pdCas9-bacteria and | This work |
| | | pSLQ1236-pyrF | |
| JL104 | E. coli Sii17[Tcrispri-pyrG] | Sii17 with pdCas9-bacteria and | This work |
| | | pSLQ1236-pyrG | |
| JL105 | E. coli Sii17 [Tcrispri-pyrH] | Sii17 with pdCas9-bacteria and | This work |
| | | pSLQ1236-pyrH | |
| JL114 | E. coli Sii17 [Tcrispri-cmk] | Sii17 with pdCas9-bacteria and | This work |
| | | pSLQ1236-cmk | |
| JL115 | E. coli Sii17 [Tcrispri-ndk] | Sii17 with pdCas9-bacteria and | This work |
| | | pSLQ1236-ndk | |
| JL122 | E. coli Sii17 [Tcrispri-nc] | Sii17 with pdCas9-bacteria and | This work |
| | | pSLQ1236-nc | |

TABLE S5
Plasmids used for the experiments.

| No. | Plasmids | Description | Reference/source | Antibiotics |
|---|---|---|---|---|
| pJL45 | pSLQ1236-purA | pSLQ1236 with sgRNA | This work | AmpR |
| | | targeting purA | | |
| pJL46 | pSLQ1236-purB | pSLQ1236 with sgRNA | This work | AmpR |
| | | targeting purB | | |
| pJL51 | pSLQ1236-purC | pSLQ1236 with sgRNA | This work | AmpR |
| | | targeting purC | | |
| pJL52 | pSLQ1236-purD | o pSLQ1236 with sgRNA | This work | AmpR |
| | | targeting purD | | |
| pJL53 | pSLQ1236-purE | pSLQ1236 with sgRNA | This work | AmpR |
| | | targeting purE | | |
| pJL54 | pSLQ1236-purF | pSLQ1236 with sgRNA | This work | AmpR |
| | | targeting purF | | |
| pJL55 | pSLQ1236-purH | pSLQ1236 with sgRNA | This work | AmpR |
| | | targeting purH | | |
| pJL56 | pSLQ1236-purK | pSLQ1236 with sgRNA | This work | AmpR |
| | | targeting purK | | |
| pJL57 | pSLQ1236-purL | pSLQ1236 with sgRNA | This work | AmpR |
| | | targeting purL | | |
| pJL58 | pSLQ1236-purM | pSLQ1236 with sgRNA | This work | AmpR |
| | | targeting purM | | |
| pJL59 | pSLQ1236-purN | pSLQ1236 with sgRNA | This work | AmpR |
| | | targeting purN | | |
| pJL60 | pSLQ1236-guaA | pSLQ1236 with sgRNA | This work | AmpR |
| | | targeting guaA | | |
| pJL61 | pSLQ1236-guaB | pSLQ1236 with sgRNA | This work | AmpR |
| | | targeting guaB | | |
| pJL62 | pSLQ1236-pyrB | pSLQ1236 with sgRNA | This work | KanR |
| | | targeting pyrB | | |
| pJL64 | pSLQ1236-pyrD | pSLQ1236 with sgRNA | This work | AmpR |
| | | targeting pyrD | | |
| pJL65 | pSLQ1236-pyrE | pSLQ1236 with sgRNA | This work | AmpR |
| | | targeting pyrE | | |
| pJL66 | pSLQ1236-pyrF | pSLQ1236 with sgRNA | This work | AmpR |
| | | targeting pyrF | | |
| pJL67 | pSLQ1236-pyrG | pSLQ1236 with sgRNA | This work | AmpR |
| | | targeting pyrB | | |
| pJL68 | pSLQ1236-pyrH | pSLQ1236 with sgRNA | This work | AmpR |
| | | targeting pyrH | | |
| pJL69 | pSLQ1236-cmk | pSLQ1236 with sgRNA | This work | AmpR |
| | | targeting cmk | | |
| pJL70 | pSLQ1236-ndk | pSLQ1236 with sgRNA | This work | AmpR |
| | | targeting ndk | | |

TABLE S6
Primer sequences used in the experiments.

| No. | Primers | Sequence(5′-3′) |
|---|---|---|
| jl37 | FP purA sgRNA | TTTACCTTCGTCACCCCATTGTTTTAGA |
| | | GCTAGAAATAGCAAGTTAAAATAAGGC |
| jl38 | RP purA sgRNA | AATGGGGTGACGAAGGTAAAACTAGTCT |
| | | TTTCTCTATCACTGATAGGGA |
| jl39 | FP purB sgRNA | TCCATCGACAGGGGAAACGGGTTTTAGA |
| | | GCTAGAAATAGCAAGTTAAAATAAGGC |
| jl40 | RP purB sgRNA | CCGTTTCCCCTGTCGATGGAACTAGTCT |
| | | TTTCTCTATCACTGATAGGGA |
| jl41 | FP purC sgRNA | CGGGTTTTCCGTGCTGTATAGTTTTAGA |
| | | GCTAGAAATAGCAAGTTAAAATAAGGC |
| jl42 | RP purC sgRNA | TATACAGCACGGAAAACCCGACTAGTCT |
| | | TTTCTCTATCACTGATAGGGA |
| jl43 | FP purD sgRNA | CGGCGACTGGGCCGCTTTCCGTTTTAGA |
| | | GCTAGAAATAGCAAGTTAAAATAAGGC |
| jl44 | RP purD sgRNA | GGAAAGCGGCCCAGTCGCCGACTAGTCT |
| | | TTTCTCTATCACTGATAGGGA |
| jl45 | FP purE sgRNA | GACACGCGCCGGATTATTGCGTTTTAGA |
| | | GCTAGAAATAGCAAGTTAAAATAAGGC |
| jl46 | RP purE sgRNA | GCAATAATCCGGCGCGTGTCACTAGTCT |
| | | TTTCTCTATCACTGATAGGGA |
| jl47 | FP purF sgRNA | TCATAAATCGACTGGTTAACGTTTTAGA |
| | | GCTAGAAATAGCAAGTTAAAATAAGGC |
| jl48 | RP purF sgRNA | GTTAACCAGTCGATTTATGAACTAGTCT |
| | | TTTCTCTATCACTGATAGGGA |
| jl49 | FP purH sgRNA | AAACACTGAGCAGAGCGCGGGTTTTAGA |
| | | GCTAGAAATAGCAAGTTAAAATAAGGC |
| jl50 | RP purH sgRNA | CCGCGCTCTGCTCAGTGTTTACTAGTCT |
| | | TTTCTCTATCACTGATAGGGA |
| jl51 | FP purK sgRNA | GGCCTAACTGCCCGTTACCGGTTTTAGA |
| | | GCTAGAAATAGCAAGTTAAAATAAGGC |
| jl52 | RP purK sgRNA | CGGTAACGGGCAGTTAGGCCACTAGTCT |
| | | TTTCTCTATCACTGATAGGGA |
| jl53 | FP purL sgRNA | ATTCGGAATGCCGACAGTGCGTTTTAGA |
| | | GCTAGAAATAGCAAGTTAAAATAAGGC |
| jl54 | RP purL sgRNA | GCACTGTCGGCATTCCGAATACTAGTCT |
| | | TTTCTCTATCACTGATAGGGA |
| jl55 | FP purM sgRNA | GGCATCTTTGTAGCTAAGAGGTTTTAGA |
| | | GCTAGAAATAGCAAGTTAAAATAAGGC |
| jl56 | RP purM sgRNA | CTCTTAGCTACAAAGATGCCACTAGTCT |
| | | TTTCTCTATCACTGATAGGGA |
| jl57 | FP purN sgRNA | CTGTAAATTACTTCCGTTGCGTTTTAGA |
| | | GCTAGAAATAGCAAGTTAAAATAAGGC |
| jl58 | RP purN sgRNA | GCAACGGAAGTAATTTACAGACTAGTCT |
| | | TTTCTCTATCACTGATAGGGA |
| jl59 | FP guaA sgRNA | GAGAACCGAAGTCCAGAATGGTTTTAGA |
| | | GCTAGAAATAGCAAGTTAAAATAAGGC |
| jl60 | RP guaA sgRNA | CATTCTGGACTTCGGTTCTCACTAGTCT |
| | | TTTCTCTATCACTGATAGGGA |
| jl61 | FP guaB sgRNA | CGGTAGAGTGAGCAGGAACGGTTTTAGA |
| | | GCTAGAAATAGCAAGTTAAAATAAGGC |
| jl62 | RP guaB sgRNA | CGTTCCTGCTCACTCTACCGACTAGTCT |
| | | TTTCTCTATCACTGATAGGGA |
| jl63 | FP pyrB sgRNA | ATGATATGTTTCTGATATAGGTTTTAGA |
| | | GCTAGAAATAGCAAGTTAAAATAAGGC |
| jl64 | RP pyrB sgRNA | CTATATCAGAAACATATCATACTAGTCT |
| | | TTTCTCTATCACTGATAGGGA |
| jl67 | FP pyrD sgRNA | AAAAGGGCTTTACGAACGAAGTTTTAGA |
| | | GCTAGAAATAGCAAGTTAAAATAAGGC |
| jl68 | RP pyrD sgRNA | TTCGTTCGTAAAGCCCTTTTACTAGTCT |
| | | TTTCTCTATCACTGATAGGGA |
| jl69 | FP pyrE sgRNA | TAAGCGCAAATTCAATAAACGTTTTAGA |
| | | GCTAGAAATAGCAAGTTAAAATAAGGC |
| jl70 | RP pyrE sgRNA | GTTTATTGAATTTGCGCTTAACTAGTCT |
| | | TTTCTCTATCACTGATAGGGA |
| jl71 | FP pyrF sgRNA | AGGAGAATTCGTAACAGCGCGTTTTAGA |
| | | GCTAGAAATAGCAAGTTAAAATAAGGC |
| jl72 | RP pyrF sgRNA | GCGCTGTTACGAATTCTCCTACTAGTCT |
| | | TTTCTCTATCACTGATAGGGA |
| jl73 | FP pyrG sgRNA | GGCAATGCCTTTACCCAGAGGTTTTAGA |
| | | GCTAGAAATAGCAAGTTAAAATAAGGC |
| jl74 | RP pyrG sgRNA | CTCTGGGTAAAGGCATTGCCACTAGTCT |
| | | TTTCTCTATCACTGATAGGGA |
| jl75 | FP pyrH sgRNA | TTTATAGACGGGTTTTGCATGTTTTAGA |
| | | GCTAGAAATAGCAAGTTAAAATAAGGC |
| jl76 | RP pyrH sgRNA | ATGCAAAACCCGTCTATAAAACTAGTCT |
| | | TTTCTCTATCACTGATAGGGA |
| jl77 | FP cmk sgRNA | GGCCATCAATGGTAATAACCGTTTTAGA |
| | | GCTAGAAATAGCAAGTTAAAATAAGGC |
| jl78 | RP cmk sgRNA | GGTTATTACCATTGATGGCCACTAGTCT |
| | | TTTCTCTATCACTGATAGGGA |
| jl79 | FP ndk sgRNA | ACGTTTTTTGCTACCGCGTTGTTTTAGA |
| | | GCTAGAAATAGCAAGTTAAAATAAGGC |
| jl80 | RP ndk sgRNA | AACGCGGTAGCAAAAAACGTACTAGTCT |
| | | TTTCTCTATCACTGATAGGGA |

### Example 7—Growth Switch Enhanced Serine Production

This experiment was carried out to demonstrate that decoupling of growth from production can be used to increase the production titer and yield of an amino acid such as L-serine. In this example, an L-serine tolerant E. coli strain, ALE-5(DE3) (Mundhada et. al. 2017), was used as described in detail below. dCas9 was initially integrated into the genome of the strain. A gRNA cassette was subsequently cloned into a plasmid that also contains two pathway genes required for serine production, while a third pathway gene was encoded on a separate plasmid. These plasmids were then transformed into the above dCas9 integrated strain. The production of L-serine production was investigated, and it was shown that inhibition of the expression of different targets resulted in growth inhibition as well as significantly improved L-serine production.

Materials and Methods

Construction of ALE-5 (DE3) O::tet-dcas9 Strain

The inducible dCas9 expression cassette described earlier was introduced into the genome of ALE-5 (DE3) to create strain E. coli ALE-5 (DE3) O::tet-dcas9. The expression cassette of dCas9 was amplified and cloned into pOSIP (StPierre et al., 2013) by USER cloning and was integrated into the phage 186 attachment site (the primary O site) in the genome. The kanamycin marker was subsequently looped out using pE-FLP according to the published protocol (St-Pierre et al., 2013).

Construction of pCDF-Duet1-serAmut-serC gRNA Plasmid

The plasmid pCDF-Duet1-serAmut-serC (Mundhada et. al. 2016) was amplified using pCDF_gRNA_UF and UR (Table S9). The 100 μl PCR mixture contained 250 nM each of forward pCDFgRNA_UF and UR primer reverse primer, 250 μM of dNTP, 2 U of Phusion polymerase, 1×HF buffer, 25 ng of plasmid pCDF-Duet1-serAmut-serC. The PCR protocol: An initial denaturation step at 98° C. for 40, followed by 20 cycles of denaturation at 98° C. for 10 seconds, annealing at 60° C. for 30 seconds, extension at 72° C. for 240 seconds the cycle was repeated 20 times.

The gRNA's were amplified from respective plasmids by using the gRNA UF and UR primers (Table S9). The 100 μL of reaction contained 250 nM each of forward gRNA_UF and UR primer reverse primer, 250 μM of dNTP, 2 U of Phusion polymerase, 1×HF buffer, 25 ng of respective plasmid templates. The PCR protocol: An initial denaturation step at 98° C. for 40, followed by 20 cycles of denaturation at 98° C. for 10 seconds, annealing at 60° C. for 30 seconds, extension at 72° C. for 60 seconds the cycle was repeated 20 times.

All the above PCR products were column purified and subjected to DpnI digestion as described in previous examples. USER cloning was carried out by adding 100 ng of pCDF-Duet 1-serAmut-serC PCR template to 100 ng of respective gRNA templates. The reaction mixture also contained 1× T4 DNA ligase buffer and 2 U of USER enzyme. The reaction was carried out at 37° C. for 30 min followed by 25° C. for 20 min and stored at 8° C. 5 μl of each USER product was transformed in 100 μL of chemically competent XL-2 blue cells.

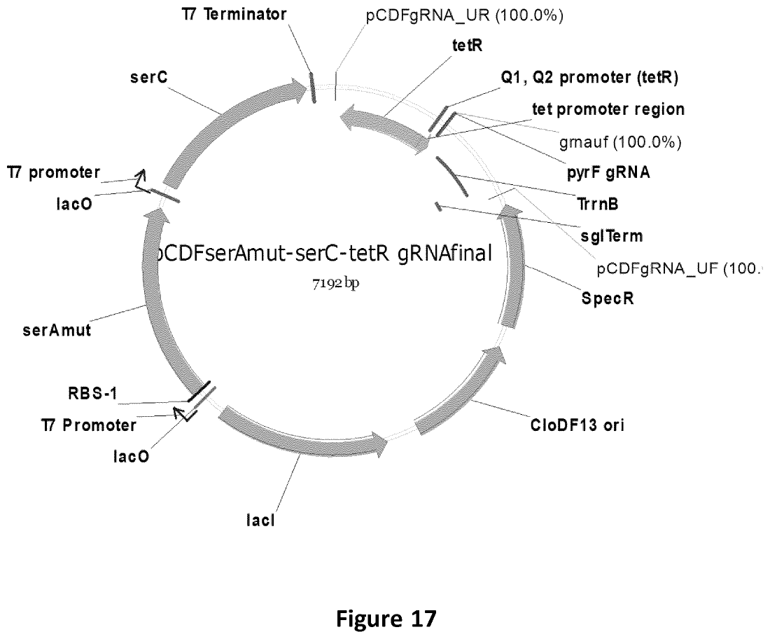

Two plasmids from each construct were isolated and then transformed in ALE-5 (DE3)O::tetR-dCas9 along with pACYC-serB plasmid to finally make strains 537, 538, 539 and 540 (Table S7). The representative plasmid map is shown in FIG. 17.

Evaluation of Growth and Serine Production

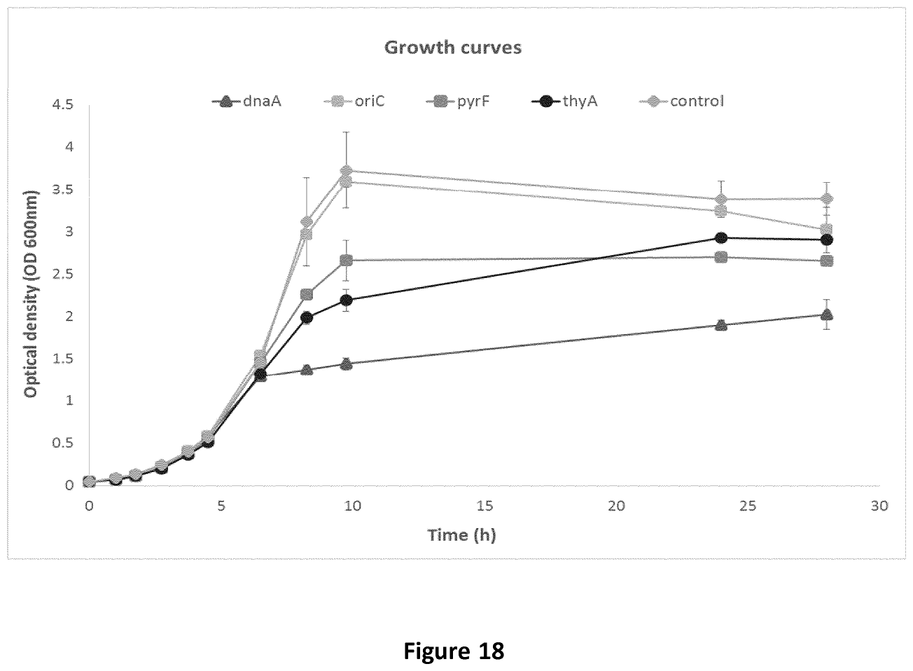

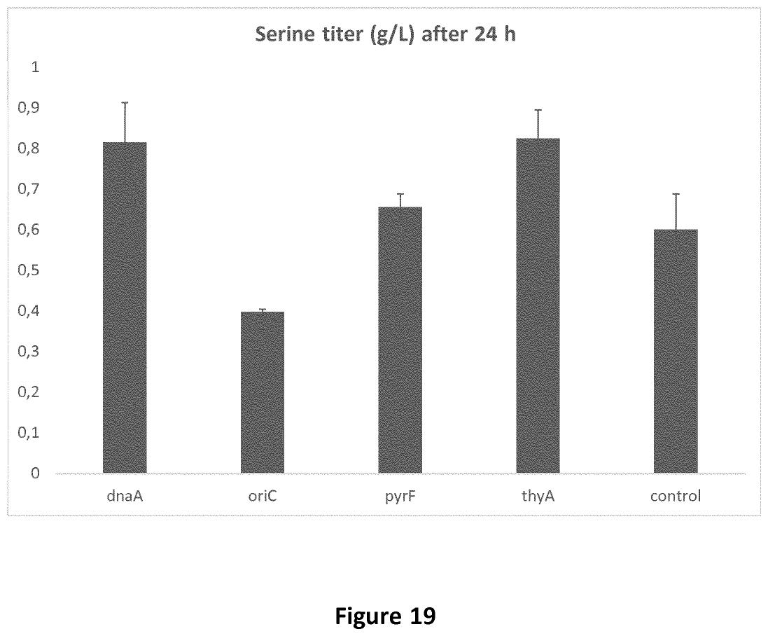

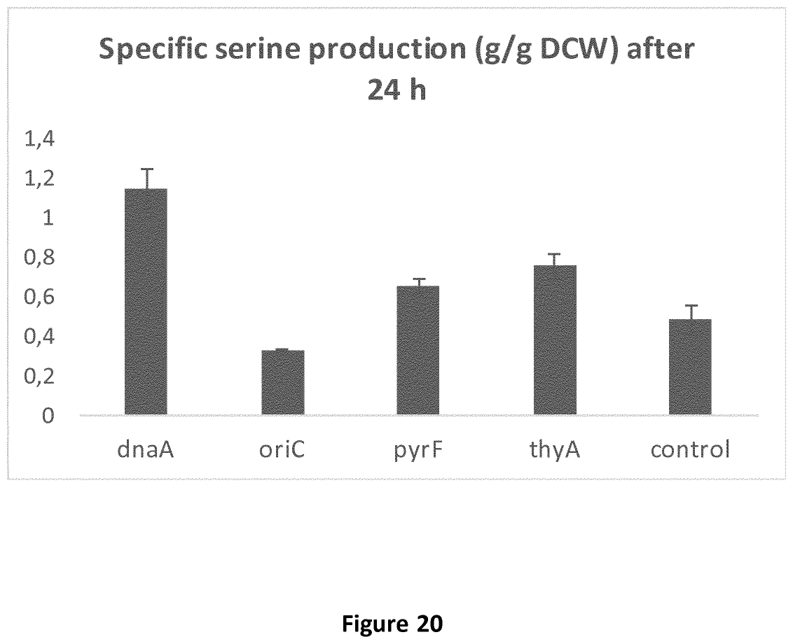

As a control, a L-serine producing strain (ALE-5 (DE3) transformed with pCDF-Duet1-serAmut-serC and pACYC-serB) not carrying dCas9 or a gRNA was used and tested together with the strains expressing gRNA's inhibiting the expression of the selected genomic targets. Biological duplicates were grown overnight in 3 mL 2×YT medium containing 16 g/L bacto-tryptone, 10 g/L yeast extract, 5 g/L NaCL, 2 g/L glucose and appropriate antibiotics. The overnight cultures were inoculated to an optical density (OD) of 0.05 in 500 mL shake flasks with 50 mL M9 minimal medium containing 4 g/L glucose, 2.0 mM glycine 0.1 mM CaCl₂), 2.0 mM MgSO4, 1× trace element solution, lx M9 salts and appropriate antibiotics. The 1× trace element stock and the 1×M9 salts were prepared as previously described (Mundhada et al., 2016). The growth switch, consisting of dCas9 and sgRNA, was induced 1.5 hours after inoculation by addition of 200 ng/mL anhydrotetracycline (aTc). Cell growth was continuously monitored by OD measurements at 600 nm. The serine pathway was induced at OD600 nm=0.6 by addition of 80 μM IPTG. The cell dry weight (cdw) was calculated from the OD using a conversion factor of 0.374, previously determined by Mundhada et al., 2016. Samples for serine production were taken continuously after serine pathway induction. Briefly, 200 uL sample was filtered, diluted to appropriate concentration and analyzed by LC-MS as previously described (Mundhada et al., 2016). Growth curves for the different strains can be seen in FIG. 18. Serine titer after 24 h of growth is shown in FIG. 19. The specific serine production (g serine/g cdw) is shown in FIG. 20.

### RESULTS AND CONCLUSIONS

In conclusion, it can be seen that different targets for inhibition of growth, including pyrF, thyA and dnaA, resulted in increased production titer and specific production of the amino acid, L-serine. Since all glucose was consumed in the experiment, this also translates into an increased production yield.

TABLE S7
Strains used for the experiments

| No. | Strains | Description | Source |
|---|---|---|---|
| ALE-5 (DE3) | MG1655 (DE3) ΔsdaA ΔsdaB | Serine tolerant MG1655 strain | Mundhada |
| | ΔtdcG ΔglyA additional | where in serine degradation is | et al., 2017 |
| | serine tolerant mutations | attenuated | |
| | and duplications | | |
| | ALE-5 (DE3)O::tetR-dCas9 | The above strains with dcas9 | This work |
| | | under atC promoter is genome | |
| | | integrated | |
| 537 | ALE-5 (DE3)O::tetR-dCas9 | Above strain with plasmid | This work |
| | pCDF-Duet1-serAmut-serC | enconding gene for serine | |
| | gRNA-dnaA pACYC-serB | biosynthesis along with gRNA | |
| | | of dnaA | |
| 538 | ALE-5 (DE3)O::tetR-dCas9 | Above strain with plasmid | This work |
| | pCDF-Duet1-serAmut-serC | enconding gene for serine | |
| | gRNA-oriC pACYC-serB | biosynthesis along with gRNA | |
| | | of oriC | |
| 539 | ALE-5 (DE3)O::tetR-dCas9 | Above strain with plasmid | This work |
| | pCDF-Duet1-serAmut-serC | enconding gene for serine | |
| | gRNA-pyrF pACYC-serB | biosynthesis along with gRNA | |
| | | of pyrF | |
| 540 | ALE-5 (DE3)O::tetR-dCas9 | Above strain with plasmid | This work |
| | pCDF-Duet1-serAmut-serC | enconding gene for serine | |
| | gRNA-thyA pACYC-serB | biosynthesis along with gRNA | |
| | | of thyA | |

TABLE S8
Plasmids used for the experiments.

| No. | Plasmids | Description | Reference/source | Antibiotics |
|---|---|---|---|---|
| | pCDF-Duet1- | Plasmid containing first | Mundhada et. al. | specR |
| | serAmut-serC | two genes of serine | 2016 | |
| | | production pathway. | | |
| | pACYC-serB | Plasmid containing last | Mundhada et. Al. | chlorR |
| | | gene of serine production | 2016 | |
| | | pathway | | |
| 537 | | pCDF-Duet1-serAmut-serC- | This work | |
| | | gRNA-dnaA | | |
| 538 | | pCDF-Duet1-serAmut-serC- | This work | |
| | | gRNA-oriC | | |
| 539 | | pCDF-Duet1-serAmut-serC- | This work | |
| | | gRNA-pyrF | | |
| 540 | | pCDF-Duet1-serAmut-serC- | This work | |
| | | gRNA-thyA | | |

TABLE S9
Primer sequences used in the experiments.

| Primers | Sequence (5′-3′) |
|---|---|
| grna_UR | ACC GCC TUT GAG TGA GCT GAT ACC |
| pCDF_UF | AAG GCG GUA AAC GAC CGG GTC ATC GT |
| grna_UF | AAC CGT UCA AGA TCT TTA AGA CCC AC |
| pCDF_UR | AAC GGT UCA GGG CAG GGT CGT TAA ATA G |

### LIST OF REFERENCES CITED IN THE DESCRIPTION

Lee, J. W., Na, D., Park, J. M., Lee, J., Choi, S., Lee, S. Y., 2012. Systems metabolic engineering of microorganisms for natural and non-natural chemicals. Nat. Chem. Biol. 8, 536-546.
Luli, G. W., Strohl, W. R., 1990. Comparison of growth, acetate production, and acetate inhibition of Escherichia coli strains in batch and fed-batch fermentations. Appl. Environ. Microbiol. 56, 1004-1011.
Chubukov, V., Sauer, U., 2014. Environmental Dependence of Stationary-Phase Metabolism in Bacillus subtilis and Escherichia coli. Appl. Environ. Microbiol. 80, 2901-2909.
Suzuki, M., Mao, L., Inouye, M., 2007. Single protein production (SPP) system in Escherichia coli. Nat. Protoc. 2, 1802-1810.
Brockman, I. M., Prather, K. L. J., 2015. Dynamic knockdown of E. coli central metabolism for redirecting fluxes of primary metabolites. Metab. Eng. 28, 104-113.
Soma, Y., Tsuruno, K., Wada, M., Yokota, A., Hanai, T., 2014. Metabolic flux redirection from a central metabolic pathway toward a synthetic pathway using a metabolic toggle switch. Metab Eng. 23, 174-184.
Haseloff and Gerlach (1988). “Simple RNA enzymes with new and highly specific endoribonuclease activities.” Nature 334: 585-591.
Carroll, D. (2012). A CRISPR approach to gene targeting. Molecular Therapy: the Journal of the American Society of Gene Therapy, 20(9), 1658-1660.
Deltcheva, E., Chylinski, K., Sharma, C. M., Gonzales, K., Chao, Y., Pirzada, Z. A., et al. (2011). CRISPR RNA maturation by trans-encoded small RNA and host factor RNase III. Nature, 471(7340), 602-607.
Datsenko K A, Wanner B L (2000): One-step inactivation of chromosomal genes in Escherichia coli K-12 using PCR products. Proc Natl Acad Sci USA, 97, 6640-6645.
Aghdama E. M., Hejazia M. S., Barzegard A. (2016). Riboswitches: From living biosensors to novel targets of antibiotics. Gene, in press, available online 16 Jul. 2016.
DiCarlo, J. E., Norville, J. E., Mali, P., Rios, X., Aach, J., & Church, G. M. (2013). Genome engineering in Saccharomyces cerevisiae using CRISPR-Cas systems. Nucleic Acids Research, 41(7), 4336-4343.
Jinek, M., Chylinski, K., Fonfara, I., Hauer, M., Doudna, J. A., & Charpentier, E. (2012). A programmable dual-RNA-guided DNA endonuclease in adaptive bacterial immunity. Science (New York, N.Y.), 337(6096), 816-821.
Mali, P., Yang, L., Esvelt, K. M., Aach, J., Guell, M., DiCarlo, J. E., et al. (2013). RNA-guided human genome engineering via Cas9. Science (New York, N.Y.), 339(6121), 823-826.
Qi, L. S., Larson, M. H., Gilbert, L. A., Doudna, J. A., Weissman, J. S., Arkin, A. P., & Lim, W. A. (2013). Repurposing CRISPR as an RNA-guided platform for sequence-specific control of gene expression. Cell, 152(5), 1173-1183.
Baba, T., Ara, T., Hasegawa, M., Takai, Y., Okumura, Y., Baba, M., Datsenko, K. A., Tomita, M., Wanner, B. L., Mori, H., 2006. Construction of Escherichia coli K-12 in-frame, single-gene knockout mutants: the Keio collection. Mol. Syst. Biol. 2, 2006.0008.
Beck, Z. Q., Miller, M. C., Peres, C. M., PRIMAK, Y. A., Pucci, J. P., Wells, D. H., 2012. Production of mevalonate, isoprene, and isoprenoids using genes encoding polypeptides having thiolase, hmg-coa synthase and hmg-coa reductase enzymatic activities.
Bonde, M. T., Pedersen, M., Klausen, M. S., Jensen, S J., Wulff, T., Harrison, S., Nielsen, A. T., Herrgård, M. J., Sommer, M. O. A., 2016. Predictable tuning of protein expression in bacteria. Nat. Methods. 13, 233-236.
Jendresen, C. B., Stahlhut, S. G., Li, M., Gaspar, P., Siedler, S., Forster, J., Maury, J., Borodina, I., Nielsen, A. T., 2015. Novel highly active and specific tyrosine ammonia-lyases from diverse origins enable enhanced production of aromatic compounds in bacteria and yeast. Appl. Environ. Microbiol. 81; 13: 4458-4476.
Juminaga, D., Baidoo, E. E. K., Redding-Johanson, A. M., Batth, T. S., Burd, H., Mukhopadhyay, A., Petzold, C. J., Keasling, J. D., 2012. Modular Engineering of I-Tyrosine Production in Escherichia coli. Appl. Environ. Microbiol. 78, 89-98.
Larson, M. H., Gilbert, L. A., Wang, X., Lim, W. A., Weissman, J. S., Qi, L. S., 2013. CRISPR interference (CRISPRi) for sequence-specific control of gene expression. Nat. Protoc. 8, 2180-2196.
Martin, V. J. J., Pitera, D. J., Withers, S. T., Newman, J. D., Keasling, J. D., 2003. Engineering a mevalonate pathway in Escherichia coli for production of terpenoids. Nat. Biotechnol. 21, 796-802.
Mundhada, H., Schneider, K., Christensen, H. B., Nielsen, A. T., 2015. Engineering of high yield production of L-serine in Escherichia coli. Biotechnol. Bioeng.
Sambrook, J., Russell, D. W., 2001. Molecular Cloning: A Laboratory Manual, Molecular Cloning: A Laboratory Manual. Cold Spring Harbor Laboratory Press.
St-Pierre, F., Cui, L., Priest, D. G., Endy, D., Dodd, I. B., Shearwin, K. E., 2013. One-Step Cloning and Chromosomal Integration of DNA. ACS Synth. Biol. 2, 537-541.
Sun, J., Nishiyama, T., Shimizu, K., Kadota, K., 2013. TCC: an R package for comparing tag count data with robust normalization strategies. BMC Bioinformatics 14, 219.
von Stockar, U., Liu, J.-S., 1999. Does microbial life always feed on negative entropy? Thermodynamic analysis of microbial growth. Biochim. Biophys. Acta-Bioenerg. 1412, 191-211.
Guiziou, S., Sauveplane, V., Chang, H.-J., Clerté, C., Declerck, N., Jules, M., & Bonnet, J. (2016). A part toolbox to tune genetic expression in Bacillus subtilis. Nucleic Acids Research, 44(10), 7495-7508.
Peters, J. M., Colavin, A., Shi, H., Qi, L. S., Huang, K. C., Gross, C. A., Peters, J. M., Colavin, A., Shi, H., Czarny, T. L., & Lar-son, M. H. (2016). A comprehensive, CRISPR-based functional analysis of essential genes in bacteria. Cell, 165(6), 1493-1506.
Winstel, V., Kuhner, P., Rohde, H., & Peschel, A. (2016). Genetic engineering of untransformable coagulase-negative staphylococcal pathogens. Nat. Protocols, 11(5), 949-959.
Froger, A., & Hall, J. E. (2007). Transformation of plasmid DNA into E. coli using the heat shock method. Journal of Visualized Experiments: JoVE, (6), 253.
Vojcic, L., Despotovic, D., Martinez, R., Maurer, K.-h., & Schwaneberg, U. (2012). An efficient transformation method for Bacillus subtilis DB104. Applied Microbiology and Biotechnology, 94(2), 487-493.
Mundhada H, Seoane J M, Schneider K, Koza A, Christensen H B, Klein T, Phaneuf P V, Herrgard M. Feist A, Nielsen A T. 2017. Increased production of L-serine in Escherichia coli through Adaptive Laboratory Evolution. Metabolic Engineering. 39:141-150.
Mundhada H, Schneider K, Christensen H B, Nielsen A T. 2016. Engineering of high yield production of L-serine in Escherichia coli. Biotechnology and Bioengineering. 113(4):807-816.

1. A method for decoupling cell growth from production of a biochemical compound or recombinant polypeptide in a microorganism having the ability to produce said biochemical compound or recombinant polypeptide, the method comprising inhibiting the expression or activity of at least one enzyme involved in the biosynthesis of at least one type of nucleotide.

2-15. (canceled)

16. A method for the production of a biochemical compound or recombinant polypeptide, the method comprising:
a) growing a microorganism having the ability to produce said biochemical compound or recombinant polypeptide, in a culture medium; and
b) reducing the growth of the microorganism by inhibiting the expression or activity of at least one enzyme involved in the biosynthesis of at least one type of nucleotide in the microorganism.

17. The method according to claim 1, wherein the biochemical compound is L-tyrosine or a derivative thereof.

18. The method according to claim 1, wherein the biochemical compound is mevalonate or a derivative thereof.

19. The method according to claim 1, wherein the method comprises inhibiting the expression or activity of at least one enzyme involved in the biosynthesis of a pyrimidine nucleotide.

20. The method according to claim 1, wherein the method comprises inhibiting the expression or activity of at least one enzyme involved in the biosynthesis of a pyrimidine nucleotide selected from the group consisting of an enzyme having orotidine-5′-phosphate decarboxylase activity, an enzyme having carbamoyl phosphate synthase activity, an enzyme having aspartate carbamoyltransferase activity, an enzyme having dihydroorotase activity, an enzyme having dihydroorotate dehydrogenase activity, an enzyme having orotate phosphoribosyltransferase activity, an enzyme having UMP kinase activity, an enzyme having nucleoside diphosphate kinase activity and an enzyme having CTP synthase activity.

21. The method according to claim 1, wherein the method comprises inhibiting the expression or activity of an enzyme having orotidine-5′-phosphate decarboxylase activity.

22. The method according to claim 1, wherein the method comprises inhibiting the expression or activity of at least one enzyme involved in the biosynthesis of a purine nucleotide.

23. The method according to claim 1, wherein the method comprises inhibiting the expression or activity of at least one enzyme involved in the biosynthesis of a purine nucleotide selected from the group consisting of an enzyme having amidophosphoribosyltransferase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N⁵-carboxyaminoimidazoleribonucleotide synthetase activity, an enzyme having N⁵-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, an enzyme having IMP cyclohydolase activity, an enzyme having adenylosuccinate synthase activity, an enzyme having adenylate kinase activity, an enzyme having ATP synthase activity, an enzyme having IMP dehydrogenase activity, an enzyme having GMP synthase activity, an enzyme having guanylate kinase activity, and an enzyme having nucleoside-diphosphate kinase activity.

24. The method according to claim 1, wherein the expression of the at least one enzyme is inhibited by introducing or expressing in the microorganism an inhibitory nucleic acid molecule that specifically hybridizes under cellular conditions with cellular mRNA or genomic DNA encoding said enzyme.

25. The method according to claim 1, wherein the expression of the at least one enzyme is inhibited by introducing or expressing in the microorganism a catalytically inactive RNA-guided endonuclease and a single guide RNA (sgRNA) specifically hybridizing under cellular conditions with the genomic DNA encoding said enzyme.

26. The method according to claim 1, wherein the at least one enzyme is encoded by a gene, the regulatory sequence of which comprises a repressible promoter.

27. The method according to claim 1, wherein the activity of the at least one enzyme is inhibited by exposing the microorganism to an inhibitor of the enzyme.

28. A genetically modified microorganism, which comprises one or more of the following modifications:
a) an exogenous nucleic acid molecule comprising a nucleotide sequence encoding an inhibitory nucleic acid molecule that specifically hybridizes under cellular conditions with cellular mRNA or genomic DNA encoding an enzyme involved in the biosynthesis of a pyrimidine nucleotide;
b) an exogenous nucleic acid molecule comprising a nucleotide sequence encoding an inhibitory nucleic acid molecule that specifically hybridizes under cellular conditions with cellular mRNA or genomic DNA encoding an enzyme involved in the biosynthesis of a purine nucleotide;
c) an exogenous nucleic acid molecule comprising a nucleotide sequence encoding a catalytically inactive RNA-guided endonuclease and a nucleotide sequence encoding a single guide RNA (sgRNA), which specifically hybridizes under cellular conditions with genomic DNA encoding an enzyme involved in the biosynthesis of a pyrimidine nucleotide; or an exogenous nucleic acid molecule comprising a nucleotide sequence encoding a catalytically inactive RNA-guided endonuclease and an exogenous nucleic acid molecule comprising a nucleotide sequence encoding a single guide RNA (sgRNA), which specifically hybridizes under cellular conditions with genomic DNA encoding an enzyme involved in the biosynthesis of a pyrimidine nucleotide;
d) an exogenous nucleic acid molecule comprising a nucleotide sequence encoding a catalytically inactive RNA-guided endonuclease and a nucleotide sequence encoding a single guide RNA (sgRNA), which specifically hybridizes under cellular conditions with genomic DNA encoding an enzyme involved in the biosynthesis of a purine nucleotide; or an exogenous nucleic acid molecule comprising a nucleotide sequence encoding a catalytically inactive RNA-guided endonuclease and an exogenous nucleic acid molecule comprising a nucleotide sequence encoding a single guide RNA (sgRNA), which specifically hybridizes under cellular conditions with genomic DNA encoding an enzyme involved in the biosynthesis of a purine nucleotide;
e) a gene encoding an enzyme involved in the biosynthesis of a pyrimidine nucleotide, the regulatory sequence of said gene comprises a repressible promoter;
f) a gene encoding an enzyme involved in the biosynthesis of a pyrimidine nucleotide, the regulatory sequence of said gene comprises an operator; wherein the genetically modified microorganism further comprises an exogenous nucleic acid molecule comprising a nucleotide sequence encoding a repressor that is capable of binding to the operator;
g) a gene encoding an enzyme involved in the biosynthesis of a purine nucleotide, the regulatory sequence of said gene comprises a repressible promoter;
h) a gene encoding an enzyme involved in the biosynthesis of a purine nucleotide, the regulatory sequence of said gene comprises an operator; wherein the genetically modified microorganism further comprises an exogenous nucleic acid molecule comprising a nucleotide sequence encoding a repressor that is capable of binding to the operator; and
i) an inactivated gene encoding an enzyme involved in the biosynthesis of a pyrimidine nucleotide;
j) an inactivated gene encoding an enzyme involved in the biosynthesis of a purine nucleotide;
k) a gene encoding an enzyme involved in the biosynthesis of a pyrimidine nucleotide, wherein the gene comprises within the region encoding an UTR, such as a 5′-UTR, a nucleotide sequence encoding a riboswitch; or
l) a gene encoding an enzyme involved in the biosynthesis of a purine nucleotide, wherein the gene comprises within the region encoding an UTR, such as a 5′-UTR, a nucleotide sequence encoding a riboswitch.

29. The genetically modified microorganism according to claim 28, which further comprises a heterologous polypeptide having tyrosine ammonia lyase activity or a heterologous polypeptide having an aryl sulfotransferase activity.
